(19) United States
(12) Patent Application Publication
(10) Pub. No.: US 2026/0199302 A1
(43) Pub. Date: Jul. 16, 2026

(54) HETEROCYCLIC INHIBITORS OF IGF-1R AND USES THEREOF

(71) Applicant: AMGEN INC., THOUSAND OAKS, CA (US)

(72) Inventors: Ashley FENWICK, NAPERVILLE, IL (US); Marta E. WENZLER, CHICAGO, IL (US); Denis BILLEN, BARRINGTON, IL (US); Yeeman K. RAMTOHUL, MONTREAL (CA); William BROWN, MONTREAL (CA); Thomas PINTER, MONTREAL (CA); Thomas JOHNSON, SAINT-LAURENT (CA); Jacob SOLEY, SAINT-LAURENT (CA); Ken Loon CHOO, MONTREAL (CA); Nicholas Willet Mitchell MICHEL, MONTREAL (CA); Naresh VEMULA, VAUDREUIL-DORION (CA); Sacha NGUYEN, MONTREAL (CA); Geoffrey S. SINCLAIR, MONTREAL (CA); Vincent ALBERT, RIGAUD (CA)

(73) Assignee: AMGEN INC., THOUSAND OAKS, CA (US)

(21) Appl. No.: 19/446,419

(22) Filed: Jan. 12, 2026

Related U.S. Application Data
(60) Provisional application No. 63/744,488, filed on Jan. 13, 2025.

(51) Int. Cl. A61K 31/437; A61K 31/4439; A61P 35/00; C07D 405/14; C07D 471/04

(52) U.S. Cl. CPC A61K 31/437; A61K 31/4439; A61P 35/00; C07D 405/14; C07D 471/04

(57) ABSTRACT

Disclosed herein are compounds useful for the inhibition of insulin-like growth factor 1 receptor (IGF-1R). The compounds have a general Formula (I):

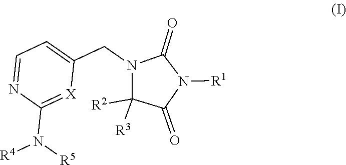

or pharmaceutically acceptable salts thereof, wherein the variables of Formula (I) are as defined herein. Also provided herein are pharmaceutical compositions comprising the compounds, uses of the compounds, and compositions for treatment of, for example, thyroid-associated ophthalmopathy, Graves' opthalmopathy or Graves' orbitopathy. Further, the disclosure provides intermediates useful in the synthesis of compounds of Formula (I).

### CROSS REFERENCE TO RELATED APPLICATIONS

This application claims the benefit of priority to U.S. Provisional Patent Application No. 63/744,488, filed Jan. 13, 2025.

### FIELD

Provided herein are compounds, pharmaceutical compositions comprising such compounds, and methods of using such compounds and compositions in treating insulin-like growth factor 1 receptor (IGF-1R)-mediated disease. Also provided are methods of making such compounds and intermediates thereof.

### BACKGROUND

Thyroid eye disease (TED) is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. Although the molecular mechanisms underlying TED remain unclear, it is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate IGF-1R have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R activity might reduce both TSHR- and IGF-1-dependent signaling and therefore interrupt the pathological activities of autoantibodies acting as agonists on either receptor.

IGF-1R is a widely expressed heterotetrameric protein involved in the regulation of proliferation and metabolic function of many cell types. It is a tyrosine kinase receptor comprising two subunits. IGF-1Rα contains a ligand-binding domain while IGF-1Rβ is involved in signaling and contains tyrosine phosphorylation sites.

Management of hyperthyroidism due to Graves' disease is imperfect because therapies targeting the specific underlying pathogenic autoimmune mechanisms of the disease are lacking. Even more complex is the treatment of moderate-to-severe active TED. Although progress has been made in recent years towards understanding of its pathogenesis, TED remains a therapeutic challenge and dilemma. Intravenous glucocorticoids (ivGCs) and oral glucocorticoids are used to treat patients with moderate-to-severe active TED, but results are seldom satisfactory. Partial responses are frequent and relapses (rebound) after drug withdrawal are not uncommon. Adverse events do occur, and many patients eventually require rehabilitative surgery when their condition has transitioned to inactive TED. Teprotumumab, a fully human IGF-1R inhibitory monoclonal antibody, is the only therapeutic specifically approved for patients with active, moderate-to-severe TED. Thus, there is a need for novel therapeutics, specifically small molecules, that inhibit IGF-1R activity.

### SUMMARY

One aspect of the disclosure provides a compound of Formula (I):

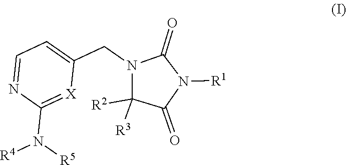

or a pharmaceutically acceptable salt thereof,
wherein R¹ is 6-membered monocyclic aromatic ring or a 6,5-bicyclic ring having 0-2 ring heteroatoms selected from N and O, the 6-membered monocyclic aromatic ring is substituted with R⁶, and the 6-membered monocyclic aromatic ring or the 6,5-bicyclic ring is substituted with 0-3 R⁷; R⁶ is

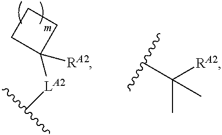

a spiro C₃₋₆cylcoalkyl, a bridged C₅₋₇cylcoalkyl, a C₃₋₆ cycloalkyl, halo, C₁₋₄alkyl, C₁₋₃haloalkyl, C₁₋₃alkoxy, C₁₋₃haloalkoxy, SO₂C₁₋₃alkyl, SO₂C₁₋₃haloalkyl, or Si(C₁₋₃alkyl)₃; L^{A2} is a bond or O; R^{A2} is H, C₁₋₃alkyl, C₁₋₃haloalkyl, C₀₋₃alkylene-CN, or C₁₋₃alkylene-O—C₁₋₃alkyl; m is 0, 1, or 2; each R⁷ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂-C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het; or two geminal R⁷ groups, together with the atom to which they are attached, form a spiro-C₃₋₇cycloalkyl group; Het is a 3-8-membered monocyclic, bicyclic, bridged, or Spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; L³ is C₀₋₃alkylene, O—C₀₋₃alkylene-, —C(O)—, N(R^{N}), or N(R^{N})C(O)C₀₋₃alkylene-; each R^{N} is independently H, C₁₋₃alkyl, C₁₋₃alkylene-CN, or C₁₋₃haloalkyl; each of R² and R³ independently is C₁₋₆ alkyl; R⁴ is H, —C(O)C₁₋₃alkyl, Het¹, C₁₋₃alkylene-O—C₁₋₃alkyl, C(O)C₁₋₃alkylene-N(R^{N})₂; Het¹ is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; R⁵ is H, C₁₋₆alkyl, C₁₋₃ alkylene-CN, C₁₋₃alkylene-O—C₁₋₃alkyl, C₁₋₆alkoxy, C(O)C₀₋₃alkylene-N(R^{N})₂, C₀₋₃alkylene-heterocyclyl having 3-6 total ring atoms and 1-2 heteroatoms selected from N, S, and O, —C(O)—, or —C(OH)═, and the heterocycle or cycloalkyl ring is substituted with 0-3 substituents independently selected from C₁₋₆alkyl, C₁₋₆alkoxy, OC(O)C₁₋₃alkyl, and C₀₋₃ alkylene-CN; X is N or CR⁸; and R⁸ is H, halo, OH, C(O)C(O)OH, or C₁₋₆ alkyl, or when X is CR⁸, R⁵ and R⁸ together with the atoms to which they are attached form a fused 5-membered heterocycle, the fused heterocycle substituted with 0-2 substituents independently selected from OH, oxo, C₁₋₆alkyl, and a 3-8-membered monocyclic heterocycle having 1-2 ring heteroatoms selected from N, O, and S, and is substituted with 0-2 substituents independently selected from halo, OH, oxo, CN, and C₁₋₃alkyl.

Another aspect of the disclosure provides a pharmaceutical composition comprising a compound or salt of Formula (I) and a pharmaceutically acceptable excipient.

Yet another aspect of the disclosure provides a method of treating an insulin-like growth factor-1 receptor-mediated disease in a subject in need of treatment, the method comprising administering to the subject a therapeutically effective amount of the compound or salt of Formula (I) or a pharmaceutical composition comprising the compound or salt of Formula (I).

Still another aspect of the disclosure provides a compound or salt of Formula (I) for use as a medicament. Another aspect of the disclosure provides a compound or salt disclosed herein, or the pharmaceutical composition disclosed herein for use in the treatment of an insulin-like growth factor-1 receptor-mediated disease.

Yet another aspect of the disclosure provides a compound or salt of Formula (I), or the pharmaceutical composition comprising a compound or salt of Formula (I), for the manufacture of a medicament for the treatment of an insulin-like growth factor-1 receptor-mediated disease. Another aspect of the disclosure provides the use of a compound or salt disclosed herein, or the pharmaceutical composition of the disclosure, wherein the insulin-like growth factor-1 receptor-mediated disease is cancer, psoriasis, thyroid eye disease, Graves' disease, acromegaly, gigantism, atherosclerosis, diabetes, neuropathy or osteoporosis, or any combination of the foregoing. In some cases, the disease is thyroid eye disease.

Yet another aspect of the disclosure provides a compound of Formula (A)

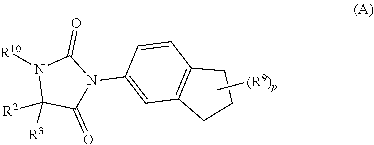

or a salt thereof, wherein p is 0, 1, 2, 3, 4, 5, or 6; each of R² and R³ independently is C₁₋₆ alkyl; and each R⁹ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂-C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het; or two geminal R⁹ groups, together with the atom to which they are attached, form a spiro-C₃₋₇cycloalkyl group; Het is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; L³ is C₀₋₃alkylene, O—C₀₋₃alkylene-, —C(O)—, N(R^{N}), or N(R^{N})C(O)C₀₋₃alkylene-; R¹⁰ is H or C₁₋₃alkylene-R¹¹;
R¹¹ is

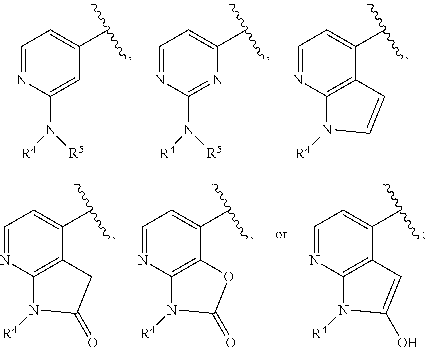

R⁴ is H, —C(O)C₁₋₃alkyl, C₁₋₃alkylene-O—C₁₋₃alkyl, C(O)C₁₋₃alkylene-N(R^{N})₂; R⁵ is H, C₁₋₆ alkyl, C₁₋₃ alkylene-CN, C₁₋₃alkylene-O—C₁₋₃alkyl, C₁₋₆alkoxy, C(O)C₀₋₃alkylene-N(R^{N})₂; and each R^{N} is independently H, C₁₋₃alkyl, C₁₋₃alkylene-CN, or C₁₋₃haloalkyl.

Yet another aspect of the disclosure provides a process for preparing a compound or salt of Formula (I), comprising providing an intermediate of Formula (A) and converting it to a compound of Formula (I).

Further aspects and advantages will be apparent to those of ordinary skill in the art from a review of the following detailed description. The description hereafter includes specific cases, embodiments, and examples with the understanding that the disclosure is illustrative and is not intended to limit the embodiments of the present disclosure to the specific cases, embodiments, and examples described herein.

### DETAILED DESCRIPTION

Disclosed herein are compounds having activity as inhibitors of IGF-1R, pharmaceutical compositions comprising the compounds, and uses and methods of treating disorders, such as cancer, psoriasis, thyroid eye disease, Graves' disease, acromegaly, gigantism, atherosclerosis, diabetes, neuropathy or osteoporosis, with the compounds and pharmaceutical composition described herein.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

The term “alkyl” refers to a saturated straight chain hydrocarbon or saturated branched chain hydrocarbon containing the indicated number of carbon atoms. For example, C₃alkyl means an alkyl group that has 3 carbon atoms (e.g., n-propyl or isopropyl). For example, a C₁₋₆alkyl refers to an alkyl group having 1 to 6 carbon atoms. Where a range is indicated, all members of that range and all subgroups within that range are envisioned. For example, a C₁₋₆alkyl includes alkyl groups having 1, 2, 3, 4, 5, or 6 carbon atoms (or any combination of the foregoing), as well as all subgroups in the indicated range (e.g., 1-2, 1-3, 1-4, 1-5, 1-6, 2-3, 2-4, 2-5, 2-6, 3-4, 3-5, 3-6, 4-5, 4-6, or 5-6 carbon atoms, or any combination of the foregoing ranges)). A “C₁₋₄ alkyl” includes, for example, methyl, ethyl, n-propyl, isopropyl, n-butyl, isobutyl, sec-butyl, or t-butyl. Nonlimiting examples of alkyl groups include methyl, ethyl, n-propyl, isopropyl, n-butyl, sec-butyl, isobutyl, tert-butyl, n-pentyl, and n-hexyl.

The term “alkenyl” refers to a straight or branched chain hydrocarbon containing the indicated number of carbon atoms and having one or more carbon-carbon double bonds. For example, C₃alkenyl means the alkenyl group has 3 carbon atoms (e.g., 1-propenyl or 2-propenyl). For example, a C₂₋₆alkenyl refers to an alkenyl group having 2 to 6 carbon atoms. Where a range is indicated, all members of that range and all subgroups within that range are envisioned. For example, a C₂₋₆alkenyl includes alkenyl groups having 2, 3, 4, 5, or 6 carbon atoms (or any combination of the foregoing), as well as all subgroups in the indicated range (e.g., 2-3, 2-4, 2-5, 2-6, 3-4, 3-5, 3-6, 4-5, 4-6, or 5-6 carbon atoms, or any combination of the foregoing ranges). A C₂₋₄alkenyl includes, for example, ethenyl, 1-propenyl, 2-propenyl, isopropenyl, 1-butenyl, 2-butenyl, or 3-butenyl. Non-limiting examples of alkenyl groups include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

The term “cycloalkyl” refers to a saturated, hydrocarbon monocyclic ring, or a saturated, hydrocarbon polycyclic ring system containing the indicated number of carbon atoms as ring members in the ring or ring system. No ring in a cycloalkyl ring or ring system has a double bond, a heteroatom, or is aromatic. When a cycloalkyl is a ring system, two or more rings may be joined together in a fused-, bridged-, or spiro-connected fashion. For example, C₅cycloalkyl refers to a cycloalkyl group that has 5 carbon atoms in the ring or ring system. Where a range is indicated, all members of that range and all subgroups within that range are envisioned. For example, a C₃₋₇cycloalkyl includes cycloalkyl groups having 3, 4, 5, 6, or 7 carbon atoms in the ring (or any combination of the foregoing), as well as all subgroups in the indicated range (e.g., 3-4, 3-5, 3-6, 3-7, 4-5, 4-6, 4-7, 5-6, 5-7, or 6-7 carbon atom ring members, or any combination of the foregoing ranges). Nonlimiting examples of cycloalkyl groups include cyclopropyl, cyclobutyl, cyclopentyl, cyclohexyl, cycloheptyl, cyclooctyl, adamantyl, norbornyl, decalinyl, and 7,7-dimethylbicyclo[2.2.1]heptanyl.

The term “cycloalkenyl” refers to a monocyclic or polycyclic hydrocarbon ring or ring system containing the indicated number of carbon atoms as ring members and having one or more carbon-carbon double bonds in the ring or ring system. No ring in a cycloalkenyl ring or ring system contains a double bond or is aromatic. When a cycloalkenyl is a ring system, two or more rings may be joined together in a fused-, bridged-, or spiro-connected fashion. For example, C₅cycloalkenyl refers to a cycloalkenyl group that has 5 carbon atoms in the ring or ring system. Where a range is indicated, all members of that range and all subgroups within that range are envisioned. For example, a C₅₋₇cycloalkenyl includes cycloalkenyl groups having 5, 6, or 7 carbon atoms in the ring or ring system (or any combination of the foregoing), as well as all subgroups in the indicated range (e.g., 5-6, 5-7, or 6-7 carbon atom ring members in the ring or ring system, or combinations of the foregoing ranges). Nonlimiting examples of cycloalkenyl groups include cyclopentenyl, cyclohexenyl, cycloheptenyl, cycloctenyl, and bicyclo[2.2.1]hept-2-enyl.

The term “aryl” refers to a monocyclic aromatic, hydrocarbon ring

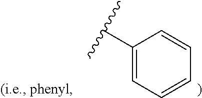 [SMILES: *c1ccccc1]

or a polycyclic (e.g., bicyclic, tricyclic, or tetracyclic) aromatic hydrocarbon ring system containing the indicated number of carbon atoms. For example, C₁₀aryl refers to an aryl group that has 10 carbon atoms in the ring system (e.g., naphthyl). When an aryl group is a polycyclic ring system, each ring in the ring system is aromatic, and no ring in the ring system contains a heteroatom. Where a range is indicated, all members of that range and all subgroups within that range are envisioned. For example, a C₆₋₁₄aryl includes aryl groups having 6-14 (e.g., 6, 10, or 14) carbon atoms in the ring or ring system (or combinations of the foregoing), as well as all subgroups in the indicated range (e.g., 6-10 or 10-14 carbon atom ring members in the ring or ring system, or combinations of the foregoing). Nonlimiting examples of aryl groups include phenyl, naphthyl, and anthracenyl.

The term “heteroatom,” unless otherwise stated herein, refers to oxygen, sulfur, nitrogen, and phosphorus.

The term “heterocyclyl” or “heterocycle” refers to a ring or ring system containing carbon atoms and one or more heteroatoms (e.g., one or more of N, O, and S) in the ring or ring system, and having the indicated number of total ring atoms (the sum of carbon atoms and heteroatoms in the ring or ring system). “Heterocyclyl” groups include, for example, heterocycloalkyl, heterocycloalkenyl, heteroaryl groups, or other ring systems having at least one heteroatom. Where the heterocyclyl is a ring system (e.g., a bicyclic, a tricyclic, or a tetracyclic system), two or more rings may be joined together in a fused-, bridged-, or spiro-connected fashion. For illustration, a heterocyclyl having 6 total ring atoms and 1, 2, or 3 heteroatoms independently selected from N, O, and S includes, for example, piperidinyl, piperazinyl, tetrahydropyranyl, dioxanyl, tetrahydrothipyranyl, dithianyl, morpholinyl, thiomorpholinyl, pyridinyl (or pyridyl), pyridazinyl, pyrimidinyl, and triazinyl. A heterocyclyl having 5-7 total ring atoms and 1, 2, or 3 heteroatoms independently selected from N, O, and S refers to a ring or ring system having a total number of ring atoms in the indicated range (e.g., 5, 6, or 7 total atoms, or any combination of the foregoing), as well as all subgroups in the indicated range (e.g., 5-6 or 6-7 total ring atoms, or any combination of the foregoing), wherein 1, 2, or 3 of the atoms in the ring are heteroatoms and each heteroatom independently is selected from N, O, and S. Thus, a heterocyclyl having 5-7 total ring atoms and 1-3 heteroatoms independently selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Examples of heterocyclyl groups include but are not limited to azetidinyl, aziridinyl, 1,3-dioxinyl, 1,3-dioxanyl, 1,4-dioxanyl, 1,3-oxathianyl, 1,3-oxathiolanyl, 1,3-dithiolyl, 1,3-dithiolanyl, 1,4-oxathianyl, tetrahydro-1,4-thiazinyl, dioxolanyl, decahydroisoquinolyl, imidazolidinyl, isothiazolidinyl, isoxazolidinyl, maleimidyl, morpholinyl, octahydroindolyl, octahydroisoindolyl, indolinyl, isoindolinyl, 2,3-dihydrobenzofuranyl, oxetanyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, succinimidyl, thiazolidinyl, tetrahydrofuranyl, trithianyl, tetrahydropyranyl, thiomorpholinyl, thiamorpholinyl, 1-oxothiomorpholinyl, 1,1-dioxothiomorpholinyl, and 1,4-dihydroquinolinyl.

The term “heterocycloalkyl” or refers to a saturated, monocyclic ring or saturated, polycyclic ring system comprising carbon atoms and one or more heteroatoms (e.g., one or more of N, O, and S), and having the indicated number of total ring atoms (the sum of carbon atoms and heteroatoms in the ring). When a heterocycloalkyl is a ring system, two or more rings may be joined together in a fused-, bridged-, or spiro-connected fashion. No ring in a heterocycloalkyl ring or ring system contains a double bond or is aromatic. For example, a heterocycloalkyl group having 5 total atoms and 2 heteroatoms independently selected from N, O, and S, refers to a ring having 3 carbon atoms and 2 heteroatoms, wherein each heteroatom of the ring independently is N, O, or S. Where a range is indicated, all members of that range and all subgroups within that range are envisioned. For example, a heterocycloalkyl group having 5-7 total ring atoms and 1-3 heteroatoms independently selected from N, O, and S includes rings having 5, 6, or 7 total atoms, or any combination of the foregoing, as well as all subgroups in the indicated range (e.g., 5-6 or 6-7 total ring atoms, or any combination of the foregoing), wherein 1, 2, or 3 of the atoms in the ring are heteroatoms and each heteroatom independently is selected from N, O, and S. Thus, a heterocycloalkyl having 5-7 total ring atoms and 1-3 heteroatoms independently selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing is independently selected from N, O, and S. Nonlimiting examples of heterocycloalkyl groups include but are not limited to aziridinyl, oxiranyl, thiiranyl, azetidinyl, oxetanyl, thietanyl, pyrrolidinyl, tetrahydrofuranyl, tetrahydrothiophene-yl, pyrazolidinyl, imidazolidinyl, isoxazolidinyl, oxazolidinyl, isothiazolidinyl, thiazolidinyl, oxathiolidinyl, piperidinyl, tetrahydropyranyl, tetrahydrothiopyranyl, piperazinyl, dioxanyl, dithianyl, morpholinyl, thiomorpholinyl, azepanyl, and 1,4-diazepanyl.

The term “heteroaryl” refers to a monocyclic aromatic ring comprising carbon and one or more heteroatoms, and having the indicated number of total ring atoms (the sum of carbon atoms and heteroatoms in the ring), or a polycyclic (e.g., bicyclic, tricyclic, or tetracyclic) aromatic ring system having one or more heteroatoms and the indicated number of total ring atoms (the sum of carbon atoms and heteroatoms in the ring system). When a heteroaryl group is a polycyclic ring system, each ring in the ring system is aromatic. For example, a heteroaryl group having 5 total atoms and 2 heteroatoms independently selected from N, O, and S, refers to an aromatic ring having 3 carbon atoms and 2 heteroatoms, wherein each heteroatom of the ring independently is N, O, or S. Where a range is indicated, all members of that range and all subgroups within that range are envisioned. For example, a heteroaryl having 5-7 total ring atoms and 1-3 heteroatoms independently selected from N, O, and S refers to an aromatic ring having a total number of ring atoms in the indicated range (e.g., 5, 6, or 7 total atoms, or any combination of the foregoing), as well as encompassing all subgroups (e.g., 5-6 or 6-7 total ring atoms, or any combination of the foregoing), wherein 1, 2, or 3 of the atoms in the ring are heteroatoms and each heteroatom is independently selected from N, O, and S. A heteroaryl having 5-7 total ring atoms and 1-3 heteroatoms independently selected from N, O, and S encompasses rings containing, for example, 4 carbon atoms and 1 heteroatom, 3 carbon atoms and 2 heteroatoms, 2 carbon atoms and 3 heteroatoms, 5 carbon atoms and 1 heteroatom, 4 carbon atoms and 2 heteroatoms, 3 carbon atoms and 3 heteroatoms, 6 carbon atoms and 1 heteroatom, 5 carbon atoms and 2 heteroatoms, and 4 carbon atoms and 3 heteroatoms, wherein each heteroatom of the foregoing independently is selected from N, O, and S. Nonlimiting examples of monocyclic heteroaryl groups include: pyrrolyl, furanyl, thiophene-yl (or thienyl), pyrazolyl, imidazolyl, isoxazolyl, oxazolyl, isothiazolyl, thiazolyl, triazolyl, oxadiazolyl, 1,3,4-oxadiazolyl, thiadiazolyl, tetrazolyl, pyridinyl (or pyridyl), pyridazinyl, pyrimidinyl, pyrazinyl, and triazinyl. Nonlimiting examples of bicyclic heteroaryl groups include benzofuranyl, benzothienyl, benzimidazolyl, benzoisoxazolyl, benzoxazolyl, benzothiazolyl, benzothiadiazolyl, benzotriazolyl, furopyridinyl (e.g., furo[2,3-b]pyridinyl), imidazopyridinyl (imidazo[4,5-b]pyridinyl), imidazothiazolyl (e.g., imidazo[4,5-d]thiazolyl), indolizinyl, indolyl, indazolyl, isobenzofuranyl, isoindolyl, isoquinolinyl, naphthyridinyl, oxazolopyridinyl (e.g., oxazolo[5,4-b]pyridinyl), phthalazinyl, pteridinyl, purinyl, pyrrolopyridyl (e.g., pyrrolo[2,3-b]pyridyl), quinolinyl, quinoxalinyl, quinazolinyl, benzoxazolyl, cinnolinyl, isoquinolyl, pyrazolopyridinyl (e.g., pyrazolo[3,4-b]pyridinyl), and thiazolopyrindinyl (e.g., thiazolo[5,4-b]pyridinyl). Nonlimiting examples of tricyclic heteroaryl groups include carbazolyl, 4,5-benzindolyl, dibenzofuranyl, dibenzothiophene-yl, phenazinyl, and acridinyl.

The term “alkylene” refers to a divalent saturated, straight or branched hydrocarbon chain diradical containing the indicated number of carbon atoms. For example, C₃alkylene means the alkylene group has 3 carbon atoms. Where a range is indicated, all members of that range and all subgroups within that range are envisioned. For example, C₁₋₆alkylene means an alkylene group having a 1, 2, 3, 4, 5, or 6 carbon atoms, or any combination of the foregoing), as well as all subgroups in the indicated range (e.g., 1-2, 1-3, 1-4, 1-5, 1-6, 2-3, 2-4, 2-5, 2-6, 3-4, 3-5, 3-6, 4-5, 4-6, and 5-6 carbon atoms, or any combination of the foregoing). When the number of carbon atoms in an alkylene group is indicated as “C₀,” then the alkylene group is not present and the recited substituent is directly attached to the rest of the compound. For example, the term C₀₋₆alkylene-OH indicates that the OH group can be directly attached to the compound or through a C₁₋₆alkylene linker. Examples of alkylene groups include methylene (—CH₂—), ethylene (—CH₂CH₂—), n-propylene (—CH₂CH₂CH₂—), isopropylene (—CH(CH₃)CH₂—), 1-butylene (—CH₂CH₂CH₂CH₂—), 1-methylbutylene (—CH(CH₃)CH₂CH₂—), 2-methylbutylene (—CH₂CH(CH₃)CH₂—), and 3-methylbutylene (—CH₂CH₂CH₂(CH₃)—).

The term “halogen” or “halo” refers to fluoro (F), chloro (Cl), bromo (Br), or iodo (I).

The term “haloalkyl” refers to an alkyl group in which one or more of the hydrogen atoms is replaced by a halogen. The halogen is independently selected at each occurrence. The term includes, for example, monohaloalkyl (e.g., CH₂F, CH(CH₂F)CH₃) dihaloalkyl (e.g., CHF₂, CH(CHF₂)CH₃), trihaloalkyl (e.g., CF₃, CH(CF₃)CH₃), and polyhaloalkyl (e.g., CF(CF₃)CH₃). A haloalkyl group may or may not be perhalogenated (e.g., perfluorinated, such as CF(CF₃)CF₃). For example, the term “C₁₋₄haloalkyl” refers to a C₁₋₄alkyl, wherein one or more hydrogen atoms is substituted with a halogen. For illustration, C₁₋₄haloalkyl includes, for example, CH₂F, CHF₂, CF₃, CHFCl, CH₂CF₃, CFHCF₃, CF₂CF₃, CH(CF₃)₂, CF(CHF₂)₂, CH(CH₂F)(CF₃), CH₂Cl, CHCl₂, CCl₃, CHFCl, CH₂CCl₃, CClHCCl₃, CCl₂CCl₃, CH(CCl₃)₂, CCl(CHCl₂)₂, CH(CH₂C₁)CCl₃, and CH₂CF(CH₃)₂.

The term “haloalkenyl” refers to an alkenyl group in which one or more of the hydrogen atoms is replaced by a halogen. The halogen is independently selected at each occurrence. The term includes, for example, monohaloalkenyl (e.g., CH═CHF) dihaloalkenyl (e.g., CH═CF₂), and trihaloalkenyl (e.g., CF═CF₂).

The term “oxo” refers to a substituent oxygen atom connected to another atom by a double bond (e.g., ═O). For example, an oxo substituent on a cyclopentyl ring can be depicted as:

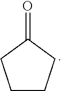

The terms “hydroxy” and “hydroxyl” are interchangeable and refer to a —OH group.

The terms “alkoxy” and “alkoxyl” are interchangeable and refer to an —O-alkyl group, where the alkyl group is as defined elsewhere herein. For example, a C₃alkoxy group means the alkoxy group has 3 carbon atoms (e.g., OCH₂CH₂CH₃). Where a range is indicated, all members of that range and all subgroups within that range are envisioned. For example, a C₁₋₆alkoxy includes alkoxy groups having 2, 3, 4, 5, or 6 carbon atoms, or any combination of the foregoing, as well as all subgroups in the indicated range (e.g., 2-3, 2-4, 2-5, 2-6, 3-4, 3-5, 3-6, 4-5, 4-6, and 5-6 carbon atoms, or any combination of the foregoing). Non-limiting examples of alkoxy groups include methoxy, ethoxy, n-propoxy, 1-methylethyloxy (iso-propoxy), n-butoxy, isobutoxy, sec-butoxy, and tert-butoxy.

The terms “haloalkoxy” and “haloalkoxyl” are interchangeable and refer to an alkoxy group in which one or more of the hydrogen atoms is replaced by a halogen. The halogen is independently selected at each occurrence. The term includes monohaloalkoxy (e.g., OCH₂F, OCH(CH₂F)CH₃) dihaloalkoxy (e.g., OCHF₂, OCH(CHF₂)CH₃), trihaloalkoxy (e.g., OCF₃, OCH(CF₃)CH₃), and polyhaloalkoxy (e.g., OCF(CF₃)CH₃). A haloalkoxy group may or may not be perhalogenated (e.g., perfluorinated, such as OCF(CF₃)CF₃). For example, the term “C₁₋₄haloalkoxy” refers to a C₁₋₄ alkoxy as defined herein, wherein one or more hydrogen atoms is substituted with a halogen. Representative examples of C₁₋₄haloalkoxy include OCH₂F, OCHF₂, OCF₃, OCHFCl, OCH₂CF₃, OCFHCF₃, OCF₂CF₃, OCH(CF₃)₂, OCF(CHF₂)₂, OCH(CH₂F)(CF₃), OCH₂C₁, OCHCl₂, OCF₃, OCHFCl, OCH₂CCl₃, OCClHCCl₃, OCCl₂CCl₃, OCH(CCl₃)₂, OCCl(CHCl₂)₂, OCH(CH₂Cl)CCl₃, and OCH₂CF(CH₃)₂.

The term “geminal” refers to substituents that are attached to the same atom. Geminal R groups on a chain and ring can be depicted as

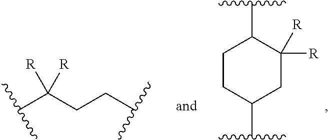

respectively.

As used herein, if any variable occurs more than one time in a chemical formula, its definition on each occurrence is independent of its definition at every other occurrence.

The term “substituted” refers to the replacement of one or more hydrogen radicals in a given structure or functional group with the radical of a specified substituent. A substituted structure or functional group may have a substituent at any substitutable position of the structure or functional group. When more than one position in a given structure can be substituted with more than one substituent, the substituent may be either the same or different at each position.

The term “pharmaceutically acceptable” refers to a species or component that is generally safe, non-toxic, and neither biologically nor otherwise undesirable for use in a subject.

The term “pharmaceutically acceptable salt” refers to a salt of a compound that possesses the desired pharmacological activity of the parent compound and that is not biologically or otherwise undesirable for its end use. Pharmaceutically acceptable salts include, for example, acid addition salts formed with inorganic acids (e.g., hydrochloric acid, hydrobromic acid, sulfuric acid, nitric acid, phosphoric acid) or formed with organic acids (e.g., acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid). Pharmaceutically acceptable salts also include, for example, salts formed when an acidic proton present in the parent compound either is replaced by a metal ion (e.g., an alkali metal ion, an alkaline earth ion, or an aluminum ion) or associates with an organic base (e.g., ethanolamine, diethanolamine, triethanolamine, N-methylglucamine, dicyclohexylamine). Additionally, the salts of the compounds described herein, can exist in either hydrated or anhydrous form or as solvates with other solvent molecules.

The term “pharmaceutically acceptable excipient” refers to a broad range of ingredients that may be combined with a compound, solvate, or salt disclosed herein to prepare a pharmaceutically acceptable composition or formulation. Excipients include, for example, vehicles (e.g., solvents, dispersion media), coatings, isotonic and absorption delaying agents, diluents, colorants, glidants, disintegrants, flavoring agents, coatings, binders, sweeteners, lubricants, sorbents, and preservatives (e.g., antibacterial and antifungal agents).

The term “therapeutically effective amount” as used herein refers to that amount of a compound disclosed herein that elicits a desired biological or medical response in a cell, a tissue, a system, or a subject.

The term “patient” or “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. Human subjects include neonates, infants, juveniles, adults, and geriatric subjects.

The term “disease” or “disorder” as used herein refers to any condition that impairs the normal functioning of the body, such as a functional abnormality or disturbance that impairs normal functioning.

“IGF-1R inhibitor” is used herein to refer to a compound that exhibits an IC₅₀ with respect to IGF-1R activity of no more than about 10 μM and more typically not more than about 5 μM, as measured in the IGF-1R assay described generally herein. “IC₅₀” is that concentration of inhibitor which reduces the activity of an enzyme (e.g., IGF-1R) to half-maximal level. Certain compounds disclosed herein have been discovered to exhibit inhibition against IGF-1R. In certain embodiments, compounds will exhibit an IC₅₀ with respect to IGF-1R of no more than about 2 μM; in yet further embodiments, compounds will exhibit an IC₅₀ with respect to IGF-1R of not more than about 1 μM; in yet further embodiments, compounds will exhibit an IC₅₀ with respect to IGF-1R of not more than about 500 nM, as measured in the IGF-1R assay described herein.

### Compounds of Formula (I)

Provided herein as Embodiment 1 are compounds of Formula (I):

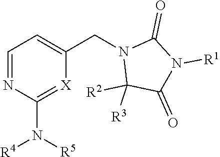

or a pharmaceutically acceptable salt thereof, wherein R¹ is 6-membered monocyclic aromatic ring or a 6,5-bicyclic ring having 0-2 ring heteroatoms selected from N and O, the 6-membered monocyclic aromatic ring is substituted with R⁶, and the 6-membered monocyclic aromatic ring or the 6,5-bicyclic ring is substituted with 0-3 R⁷; R⁶ is

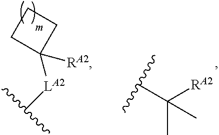

a spiro C₃₋₆cylcoalkyl, a bridged C₅₋₇cylcoalkyl, a C₃₋₆ cycloalkyl, halo, C₁₋₄alkyl, C₁₋₃haloalkyl, C₁₋₃alkoxy, C₁₋₃haloalkoxy, SO₂C₁₋₃alkyl, SO₂C₁₋₃haloalkyl, or Si(C₁₋₃alkyl)₃; L^{A2} is a bond or O; R^{A2} is H, C₁₋₃alkyl, C₁₋₃haloalkyl, C₀₋₃alkylene-CN, or C₁₋₃alkylene-O—C₁₋₃alkyl; m is 0, 1, or 2; each R⁷ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂-C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het; or two geminal R⁷ groups, together with the atom to which they are attached, form a spiro-C₃₋₇cycloalkyl group; Het is a 3-8-membered monocyclic, bicyclic, bridged, or Spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; L³ is C₀₋₃alkylene, O—C₀₋₃alkylene-, —C(O)—, N(R^{N}), or N(R^{N})C(O)C₀₋₃alkylene-; each R^{N} is independently H, C₁₋₃alkyl, C₁₋₃alkylene-CN, or C₁₋₃haloalkyl; each of R² and R³ independently is C₁₋₆ alkyl; R⁴ is H, —C(O)C₁₋₃alkyl, Het¹, C₁₋₃alkylene-O—C₁₋₃alkyl, C(O)C₁₋₃alkylene-N(R^{N})₂; Het¹ is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; R⁵ is H, C₁₋₆alkyl, C₁₋₃ alkylene-CN, C₁₋₃alkylene-O—C₁₋₃alkyl, C₁₋₆alkoxy, C(O)C₀₋₃alkylene-N(R^{N})₂, C₀₋₃alkylene-heterocyclyl having 3-6 total ring atoms and 1-2 heteroatoms selected from N, S, and O, —C(O)—, or —C(OH)═, and the heterocycle or cycloalkyl ring is substituted with 0-3 substituents independently selected from C₁₋₆alkyl, C₁₋₆alkoxy, OC(O)C₁₋₃alkyl, and C₀₋₃ alkylene-CN; X is N or CR⁸; and R⁸ is H, halo, OH, C(O)C(O)OH, or C₁₋₆ alkyl, or when X is CR⁸, R⁵ and R⁸ together with the atoms to which they are attached form a fused 5-membered heterocycle, the fused heterocycle substituted with 0-2 substituents independently selected from OH, oxo, C₁₋₆alkyl, and a 3-8-membered monocyclic heterocycle having 1-2 ring heteroatoms selected from N, O, and S, and is substituted with 0-2 substituents independently selected from halo, OH, oxo, CN, and C₁₋₃alkyl.

In some cases, R¹ is a six-membered monocyclic aromatic ring substituted with R⁶ and one R⁷. In some cases, R⁷ is Br, Cl, or F. In some cases, R⁷ is C₁. In some cases, R⁷ is F. In some cases, R⁷ is C₁₋₄alkyl. In some cases, R⁷ is CH₃, CH₂CH₃, CH₂CH₂CH₃, CH(CH₃)₂, CH₂CH₂CH₂CH₃, CH(CH₃)CH₂CH₃, CH₂CH(CH₃)CH₃, or CH₂CH(CH₃)₂. In some cases, R⁷ is CH₃, CH₂CH₃, CH₂CH₂CH₃, or CH(CH₃)₂.

Provided herein as Embodiment 2 is the compound or salt of Embodiment 1, wherein R¹ is

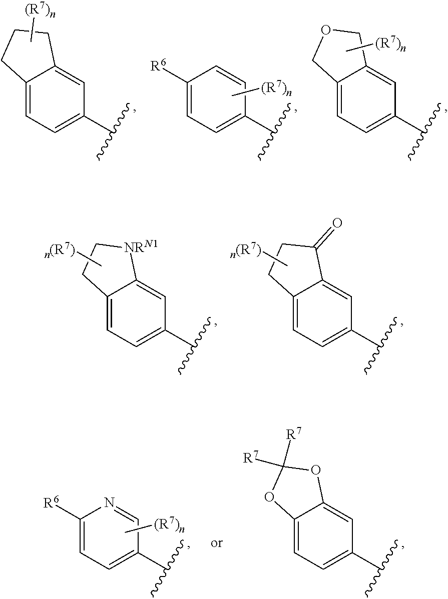

R^{N1} is H, C₁₋₆alkyl or C(O)C₁₋₃alkylene-N(R^{N})₂, each R^{N} is independently H or C₁₋₃alkyl, and n is 0, 1, 2, or 3.

In some cases, each R^{N} is independently CH₃ or CH₂CH₃. In some cases, R¹ is substituted with 1 R⁷ (i.e., n is 1). In some cases, R¹ is substituted with 2 R⁷. In some cases, R⁷ is CH₃ or OH.

Provided herein as Embodiment 3 is the compound or salt of Embodiment 2, wherein n is 0.

Provided herein as Embodiment 4 is the compound or salt of Embodiment 2 wherein n is 1 or 2.

Provided herein as Embodiment 5 is the compound or salt of any one of Embodiments 1 to 4 wherein R⁶ is or

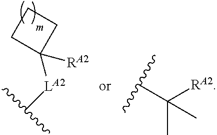

In some cases, m is 0 or 1. In some cases, R⁶ is

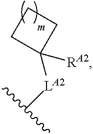

L^{A2} is a bond, m is 0, and R^{A2} is CF₃, CN or CH₃.

Provided herein as Embodiment 6 is the compound or salt of Embodiment 5, wherein R^{A2} is C₁₋₃alkyl, C₁₋₃haloalkyl, C₀₋₃alkylene-CN, or C₁₋₃alkylene-O—C₁₋₃alkyl.

Provided herein as Embodiment 7 is the compound or salt of any one of Embodiments 5 or 6, wherein L^{A2} is a bond. In some cases, R^{A2} is CF₃

Provided herein as Embodiment 8 is the compound or salt of any one of Embodiments 1-7, wherein R⁶ is

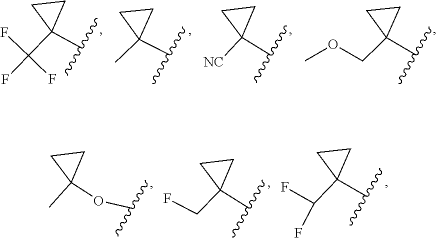

C(CH₃)₂CF₃, C(CH₃)₃, CH(CH₃)₂,

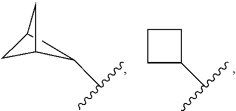

SO₂CF₃, CH(CH₃)(CF₃), C(F)₂CH₃, CH₂CF₃,

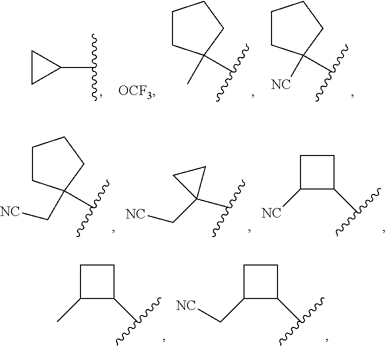

or Si(CH₃)₃.

Provided herein as Embodiment 9 is the compound or salt of any one of Embodiments 1-8, wherein at least one R⁷ is N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, C₁₋₃alkylene-N(R^{N})SO₂C₁₋₃alkyl, O—C₁₋₃alkylene-N(R^{N})SO₂C₁₋₃alkyl, or —C₀₋₃ alkylene-N(R^{N})₂.

Provided herein as Embodiment 10 is the compound or salt of Embodiment 9, wherein an alkyl or alkylene moiety of R⁷ is deuterated. In some cases, R⁷ is CH₂N(CD₃)₂, or CD₂(CD₃)₂.

Provided herein as Embodiment 11 is the compound or salt of any one of Embodiments 1-10, wherein at least one R⁷ is OH, halo, C₁₋₆alkyl, CH₂SO₂NH₂, CH(CH₃)N(CH₃)₂, CH₂NHSO₂CH₃, SO₂CH₃, CH₂SO₂CH₃, OCH₂CH₂N(CH₃)₂, OCH₂CH₂OCH₃, OCH₂C(CH₃)₂OH, CH₂C(O)N(CH₃)₂, CH₂N(CH₃)CH₂CHF₂, CH₂NHCH₂CHF₂, CH₂N(CH₃)₂, CH₂CH₂N(CH₃)₂, CH₂CH₂CH₂N(CH₃)₂, CH₂NH₂, CH₂NHCH₃, CH₂CN, CN, NH₂, CH₂N(CH₃)CH₂CN, N(CH₃)₂, NHC(O)CH(NH₂)CH₂OH, NHC(O)C(CH₃)₂OH, NHC(O)CH₂N(CH₃)₂, NHC(O)CH₃, NHC(O)CH₂OCH₃, NHCH₂CH₂OCH₃, CD₂N(CD₃)₂, CH₂OH, CH(OH)CH₃, CH₂OCH₃, C(O)NHCH₂CH₂OCH₃, C(O)NHCH₂CH₂N(CH₃)₂, OCH₂CH₂NHSO₂CH₃, or two geminal R⁷ groups, together with the atom to which they are attached, form a spiro-C₃₋₇cycloalkyl group.

Provided herein as Embodiment 12 is the compound or salt of any one of Embodiments 1-11, wherein at least one R⁷ is

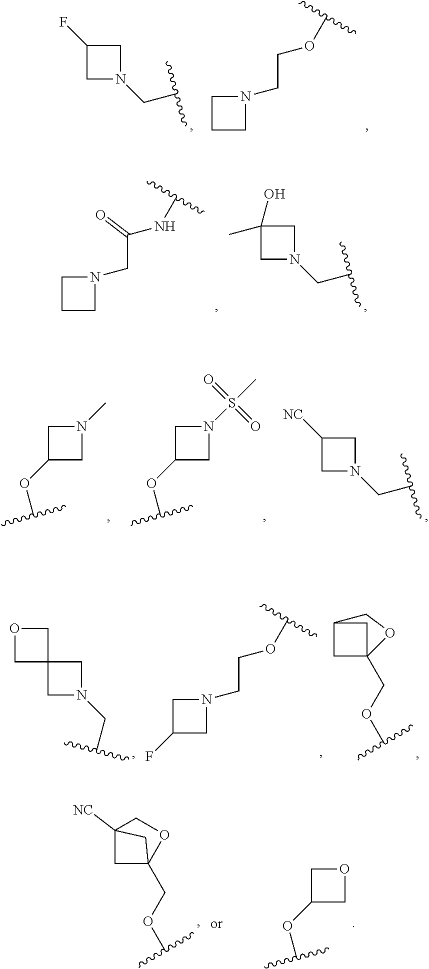

Provided herein as Embodiment 13 is the compound or salt of any one of Embodiments 1-12, wherein at least one R⁷ is

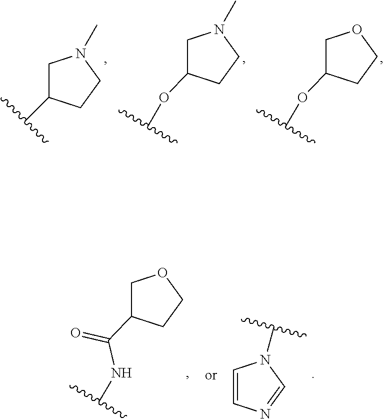

Provided herein as Embodiment 14 is the compound or salt of any one of Embodiments 1-13, wherein at least one R⁷ is

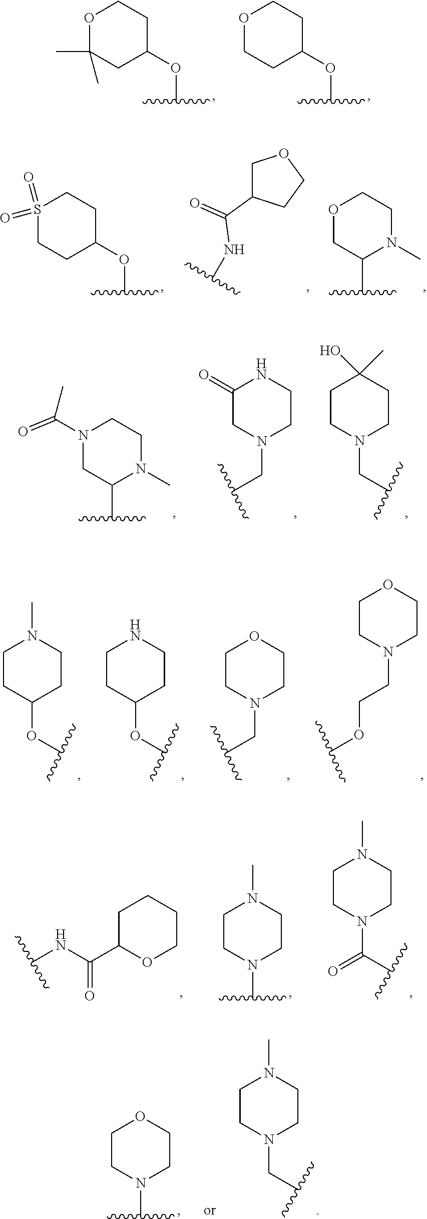

Provided herein as Embodiment 15 is the compound or salt of any one of Embodiments 1-14,

Provided herein as Embodiment 16 is the compound or salt of any one of Embodiments 1-14, wherein R¹ is substituted with 2 R⁷.

Provided herein as Embodiment 17 is the compound or salt of any one of Embodiments 1-16, having a structure of Formula (IA) or Formula (IB):

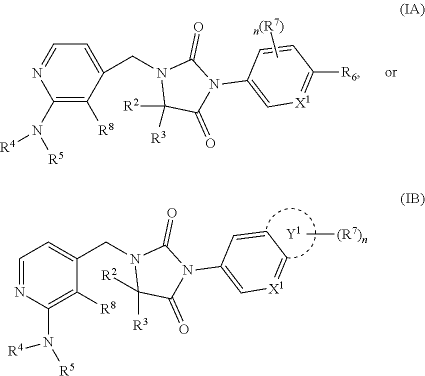

wherein X¹ is CH or N, and Y¹ is a fused 5-membered cycloalkyl, or a fused 5-membered heterocycle having 1-2 ring heteroatoms selected from N, S, and O. In some cases, the compound has a structure of Formula (1A), and X¹ is CH. In some cases, the compound has a structure of Formula (B), and Y¹ is a fused 5-membered heterocycle having one nitrogen atom, one oxygen atom, or two oxygen atoms. In some cases, R is C(CH₃)₃ or CH(CH₃)₂.

Provided herein as Embodiment 18 is the compound or salt of any one of Embodiments 1-16, having a structure of Formula (IC) or Formula (ID):

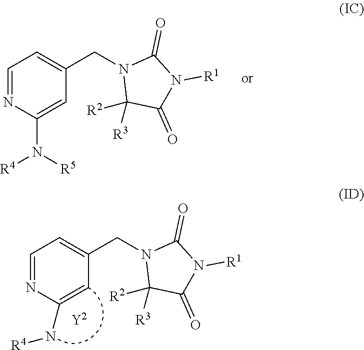

wherein Y² is a fused 5-membered heterocycle, the fused 5-membered heterocycle having 1-2 ring heteroatoms selected from N, O, and S, and substituted with 0-2 substituents independently selected from OH, oxo, C₁₋₆alkyl, or a 3-8-membered monocyclic heterocycle having 1-2 ring heteroatoms selected from N, O, and S, and is substituted with 0-2 substituents independently selected from halo, OH, oxo, CN, and C₁₋₃alkyl. In some cases, Y² has one nitrogen heteroatom. In some cases, Y² is substituted with 1 oxo substituent.

Provided herein as Embodiment 19 is the compound or salt of Embodiment 18, having a structure of Formula (ID-1), (ID-2), (ID-3), or (ID-4):

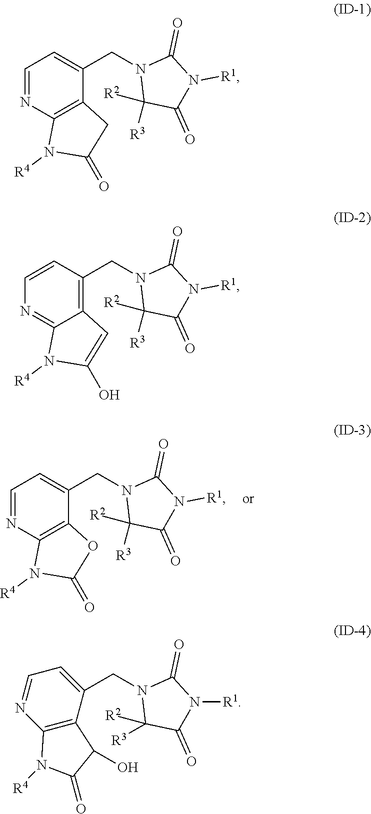

In some cases, the compound has a structure of Formula (1D-1), and R¹ is a 6-membered monocyclic aromatic ring. In some cases, the compound has a structure of Formula (1D-1), and R¹ is a 6,5 bicyclic ring having 0 ring heteroatoms.

Provided herein as Embodiment 20 is the compound or salt of any one of Embodiments 1-19, wherein R⁴ is H.

Provided herein as Embodiment 21 is the compound or salt of any one of Embodiments 1-19, wherein R⁴ is —C(O)C₁₋₃alkyl, C₁₋₃alkylene-O—C₁₋₃alkyl, or C(O)C₁₋₃alkylene-N(R^{N})₂.

Provided herein as Embodiment 22 is the compound or Embodiment 21, wherein R⁴ is C(O)CH(CH₃)₂, C(CH₃)CH₂CN, C(CH₃)CH₂OCH₃, or C(O)NH₂.

Provided herein as Embodiment 23 is the compound or salt of any one of Embodiments 1-19, wherein R⁴ is Het¹.

Provided herein as Embodiment 24 is the compound or salt of Embodiment 23, wherein R⁴ is

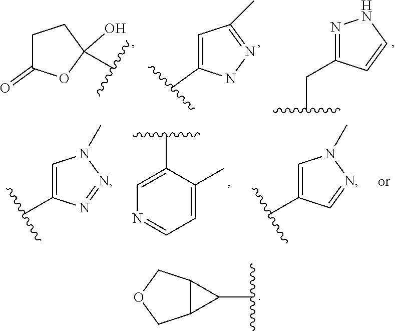

Provided herein as Embodiment 25 is the compound or salt of any one of Embodiments 1-24, wherein each of R² and R³ are methyl.

Provided herein as Embodiment 26 is the compound or salt of Embodiment 1, wherein R¹ comprises phenyl and is substituted with 0 or 1 R¹, R² and R³ are each independently C₁₋₃ alkyl, R⁶ is

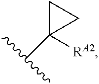

C(CH₃)₃, or CH(CH₃)₂, and R^{A2} is C₁₋₃haloalkyl.

Provided herein as Embodiment 27 is the compound or salt of any one of Embodiments 1-17 and 25-26, wherein R⁴ and R⁵ are each hydrogen.

Provided herein as Embodiment 28 is the compound or salt of Embodiment 1, wherein the compound is a compound listed in Table 1, below.

TABLE 1

| Comp. # | Chemical Structure | Name |
|---|---|---|
| 1 | 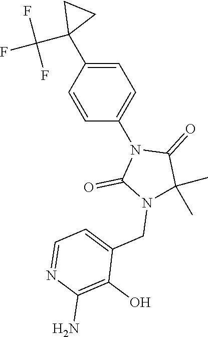 [SMILES: CC1(C)C(=O)N(c2ccc(C3(C(F)(F)F)CC3)cc2)C(=O)N1Cc1ccnc(N)c1O] | 1-[(2-amino-3-hydroxy-4-pyridyl)methyl]-5,5- dimethyl-3-{p-[1- (trifluoromethyl)cyclopropyl]phenyl}-2,4- imidazolidinedione |
| 2 | 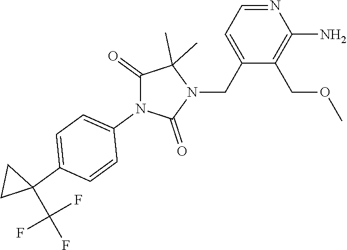 [SMILES: COCc1c(CN2C(=O)N(c3ccc(C4(C(F)(F)F)CC4)cc3)C(=O)C2(C)C)ccnc1N] | 1-{[2-amino-3-(methoxymethyl)-4- pyridyl]methyl}-5,5-dimethyl-3-{p-[1- (trifluoromethyl)cyclopropyl]phenyl}-2,4- imidazolidinedione |
| 3 | 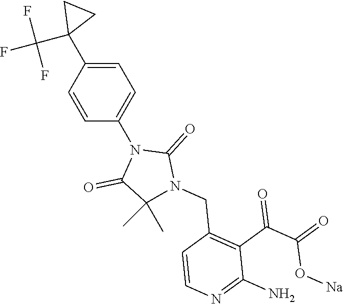 | sodium {2-amino-4-[(5,5-dimethyl-2,4-dioxo-3- {p-[1-(trifluoromethyl)cyclopropyl]phenyl}-1- imidazolidinyl)methyl]-3-pyridyl}oxoacetate |
| 4 | 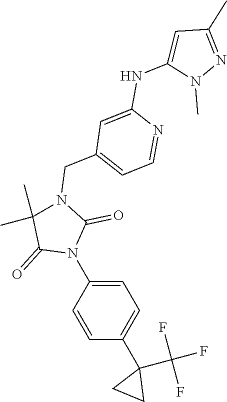 | 5,5-dimethyl-1-{[2-(1-methyl-3-methyl-5- pyrazolylamino)-4-pyridyl]methyl}-3-{p-[1- (trifluoromethyl)cyclopropyl]phenyl}-2,4- imidazolidinedione |
| 5 | 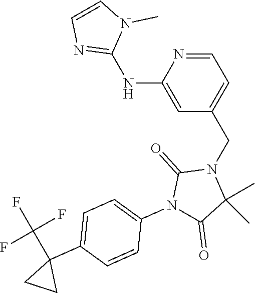 [SMILES: Cn1ccnc1Nc1cc(CN2C(=O)N(c3ccc(C4(C(F)(F)F)CC4)cc3)C(=O)C2(C)C)ccn1] | 5,5-dimethyl-1-{[2-(1-methyl-2- imidazolylamino)-4-pyridyl]methyl}-3-{p-[1- (trifluoromethyl)cyclopropyl]phenyl}-2,4- imidazolidinedione |
| 6 | 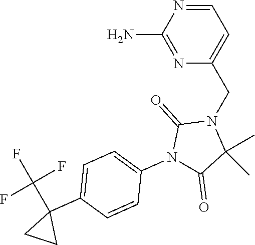 [SMILES: CC1(C)C(=O)N(c2ccc(C3(C(F)(F)F)CC3)cc2)C(=O)N1Cc1ccnc(N)n1] | 1-[(2-amino-4-pyrimidinyl)methyl]-5,5- dimethyl-3-{p-[1- (trifluoromethyl)cyclopropyl]phenyl}-2,4- imidazolidinedione |
| 7 | 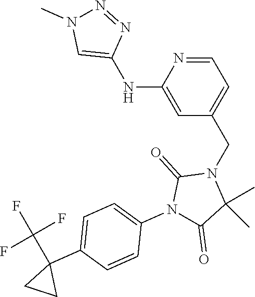 [SMILES: Cn1cc(Nc2cc(CN3C(=O)N(c4ccc(C5(C(F)(F)F)CC5)cc4)C(=O)C3(C)C)ccn2)nn1] | 5,5-dimethyl-1-{[2-(1-methyl-1H-1,2,3-triazol-4- ylamino)-4-pyridyl]methyl}-3-{p-[1- (trifluoromethyl)cyclopropyl]phenyl}-2,4- imidazolidinedione |
| 8 | 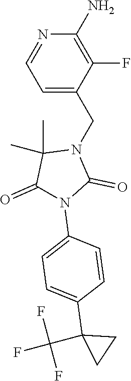 [SMILES: CC1(C)C(=O)N(c2ccc(C3(C(F)(F)F)CC3)cc2)C(=O)N1Cc1ccnc(N)c1F] | 1-[(2-amino-3-fluoro-4-pyridyl)methyl]-5,5- dimethyl-3-{p-[1- (trifluoromethyl)cyclopropyl]phenyl}-2,4- imidazolidinedione |
| 9 | 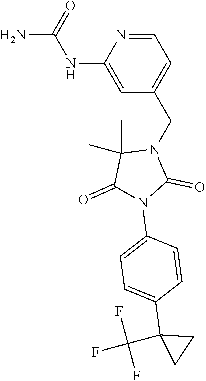 [SMILES: CC1(C)C(=O)N(c2ccc(C3(C(F)(F)F)CC3)cc2)C(=O)N1Cc1ccnc(NC(N)=O)c1] | 5,5-dimethyl-3-{p-[1- (trifluoromethyl)cyclopropyl]phenyl}-1-[(2- ureido-4-pyridyl)methyl]-2,4-imidazolidinedione |
| 10 | 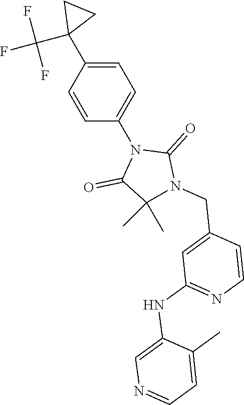 [SMILES: Cc1ccncc1Nc1cc(CN2C(=O)N(c3ccc(C4(C(F)(F)F)CC4)cc3)C(=O)C2(C)C)ccn1] | 5,5-dimethyl-1-{[2-(4-methyl-3-pyridylamino)- 4-pyridyl]methyl}-3-{p-[1- (trifluoromethyl)cyclopropyl]phenyl}-2,4- imidazolidinedione |
| 11 | 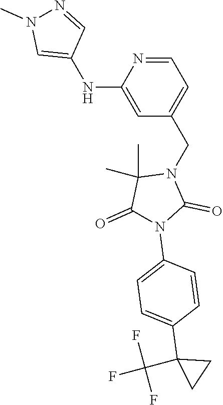 [SMILES: Cn1cc(Nc2cc(CN3C(=O)N(c4ccc(C5(C(F)(F)F)CC5)cc4)C(=O)C3(C)C)ccn2)cn1] | 5,5-dimethyl-1-{[2-(1-methyl-4- pyrazolylamino)-4-pyridyl]methyl}-3-{p-[1- (trifluoromethyl)cyclopropyl]phenyl}-2,4- imidazolidinedione |
| 12 | 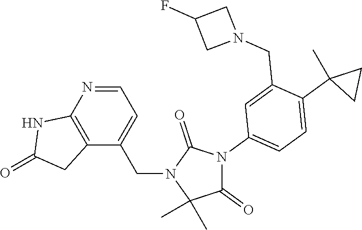 [SMILES: CC1(c2ccc(N3C(=O)N(Cc4ccnc5c4CC(=O)N5)C(C)(C)C3=O)cc2CN2CC(F)C2)CC1] | 3-{3-[(3-fluoro-1-azatidinyl)methyl]-4-(1- methylcyclopropyl)phenyl}-5,5-dimethyl-1-[(2- oxo-1,7-diaza-4-indanyl)methyl]-2,4- imidazolidinedione |
| 13 | 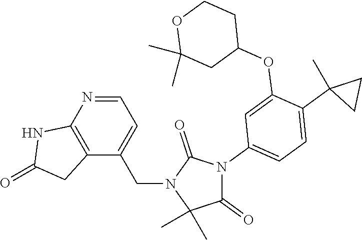 [SMILES: CC1(C)CC(Oc2cc(N3C(=O)N(Cc4ccnc5c4CC(=O)N5)C(C)(C)C3=O)ccc2C2(C)CC2)CCO1] | 3-[3-(2,2-dimethyltetrahydro-2H-pyran-4-yloxy)-4-(1- methylcyclopropyl)phenyl]-5,5-dimethyl-1-[(2-oxo-1,7- diaza-4-indanyl)methyl]-2,4-imidazolidinedione |
| 14 | 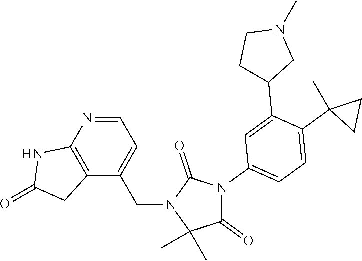 [SMILES: CN1CCC(c2cc(N3C(=O)N(Cc4ccnc5c4CC(=O)N5)C(C)(C)C3=O)ccc2C2(C)CC2)C1] | 5,5-dimethyl-3-[4-(1-methylcyclopropyl)-3-(1- methyl-3-pyrrolidinyl)phenyl]-1-[(2-oxo-1,7- diaza-4-indanyl)methyl]-2,4-imidazolidinedione |
| 15 | 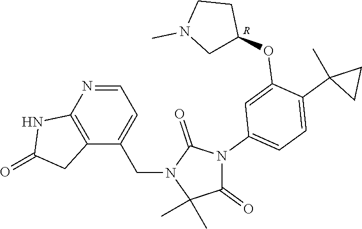 | 3-{3-[(R)-1-methyl-3-pyrrolidinyloxy]-4-(1- methylcyclopropyl)phenyl}-5,5-dimethyl-1-[(2- oxo-1,7-diaza-4-indanyl)methyl]-2,4- imidazolidinedione |
| 16 | 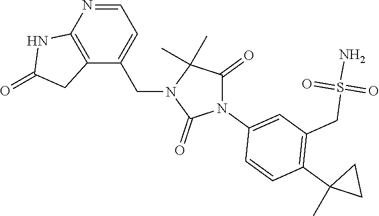 [SMILES: CC1(c2ccc(N3C(=O)N(Cc4ccnc5c4CC(=O)N5)C(C)(C)C3=O)cc2CS(N)(=O)=O)CC1] | (5-{4,4-dimethyl-2,5-dioxo-3-[(2-oxo-1,7-diaza- 4-indanyl)methyl]-1-imidazolidinyl}-2-(1- methylcyclopropyl)phenyl)methanesulfonamide |
| 17 | 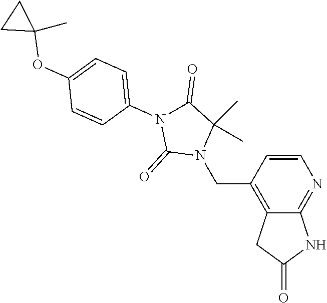 [SMILES: CC1(Oc2ccc(N3C(=O)N(Cc4ccnc5c4CC(=O)N5)C(C)(C)C3=O)cc2)CC1] | 5,5-dimethyl-3-[p-(1- methylcyclopropoxy)phenyl]-1-[(2-oxo-1,7- diaza-4-indanyl)methyl]-2,4-imidazolidinedione |
| 18 | 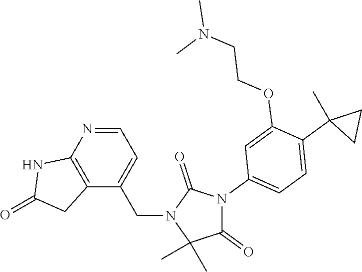 [SMILES: CN(C)CCOc1cc(N2C(=O)N(Cc3ccnc4c3CC(=O)N4)C(C)(C)C2=O)ccc1C1(C)CC1] | 3-{3-[2-(dimethylamino)ethoxy]-4-(1- methylcyclopropyl)phenyl}-5,5-dimethyl-1-[(2- oxo-1,7-diaza-4-indanyl)methyl]-2,4- imidazolidinedione |
| 19 | 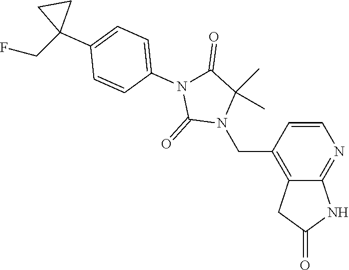 [SMILES: CC1(C)C(=O)N(c2ccc(C3(CF)CC3)cc2)C(=O)N1Cc1ccnc2c1CC(=O)N2] | 3-{p-[1-(fluoromethyl)cyclopropyl]phenyl}-5,5- dimethyl-1-[(2-oxo-1,7-diaza-4-indanyl)methyl]- 2,4-imidazolidinedione |
| 20 | 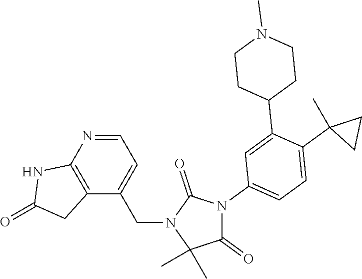 [SMILES: CN1CCC(c2cc(N3C(=O)N(Cc4ccnc5c4CC(=O)N5)C(C)(C)C3=O)ccc2C2(C)CC2)CC1] | 5,5-dimethyl-3-[4-(1-methylcyclopropyl)-3-(1- methyl-4-piperidyl)phenyl]-1-[(2-oxo-1,7-diaza- 4-indanyl)methyl]-2,4-imidazolidinedione |
| 21 | 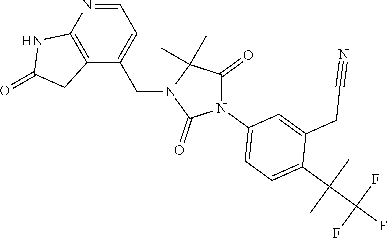 [SMILES: CC1(C)C(=O)N(c2ccc(C(C)(C)C(F)(F)F)c(CC#N)c2)C(=O)N1Cc1ccnc2c1CC(=O)N2] | (5-{4,4-dimethyl-2,5-dioxo-3-[(2-oxo-1,7-diaza- 4-indanyl)methyl]-1-imidazolidinyl}-2-(2,2,2- trifluoro-1,1-dimethylethyl)phenyl)acetonitrile |
| 22 | 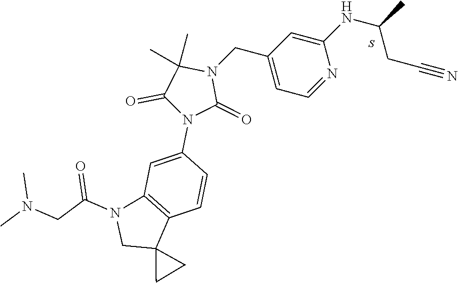 | (S)-3-{4-[(3-{1′-[2- (dimethylamino)acetyl]spiro[cyclopropane-1,3′- indolin]-6′-yl}-5,5-dimethyl-2,4-dioxo-1- imidazolidinyl)methyl]-2- pyridylamino}butyronitrile |
| 23 | 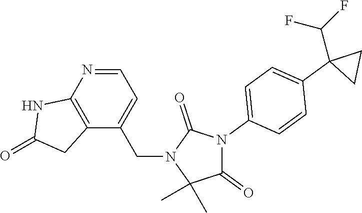 [SMILES: CC1(C)C(=O)N(c2ccc(C3(C(F)F)CC3)cc2)C(=O)N1Cc1ccnc2c1CC(=O)N2] | 3-{p-[1-(difluoromethyl)cyclopropyl]phenyl}- 5,5-dimethyl-1-[(2-oxo-1,7-diaza-4- indanyl)methyl]-2,4-imidazolidinedione |
| 24 | 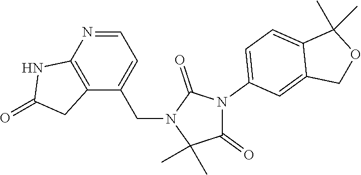 [SMILES: CC1(C)OCc2cc(N3C(=O)N(Cc4ccnc5c4CC(=O)N5)C(C)(C)C3=O)ccc21] | 3-(1,1-dimethyl-1,3-dihydro-5-isobenzofuranyl)- 5,5-dimethyl-1-[(2-oxo-1,7-diaza-4- indanyl)methyl]-2,4-imidazolidinedione |
| 25 | 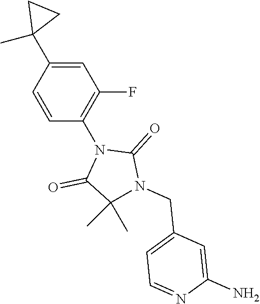 [SMILES: CC1(c2ccc(N3C(=O)N(Cc4ccnc(N)c4)C(C)(C)C3=O)c(F)c2)CC1] | 1-[(2-amino-4-pyridyl)methyl]-3-[2-fluoro-4-(1- methylcyclopropyl)phenyl]-5,5-dimethyl-2,4- imidazolidinedione |
| 26 | 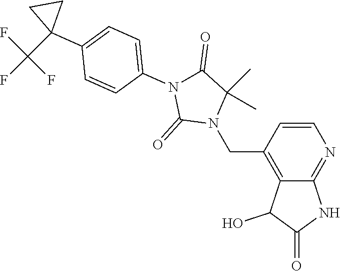 [SMILES: CC1(C)C(=O)N(c2ccc(C3(C(F)(F)F)CC3)cc2)C(=O)N1Cc1ccnc2c1C(O)C(=O)N2] | 1-[(3-hydroxy-2-oxo-1,7-diaza-4- indanyl)methyl]-5,5-dimethyl-3-{p-[1- (trifluoromethyl)cyclopropyl]phenyl}-2,4- imidazolidinedione |
| 27 | 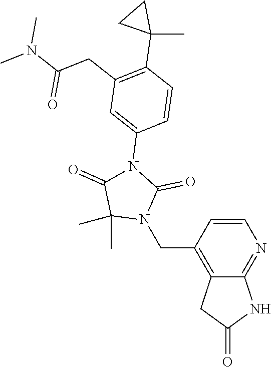 [SMILES: CN(C)C(=O)Cc1cc(N2C(=O)N(Cc3ccnc4c3CC(=O)N4)C(C)(C)C2=O)ccc1C1(C)CC1] | N,N-dimethyl(5-{4,4-dimethyl-2,5-dioxo-3-[(2- oxo-1,7-diaza-4-indanyl)methyl]-1- imidazolidinyl}-2-(1- methylcyclopropyl)phenyl)acetamide |
| 28 | 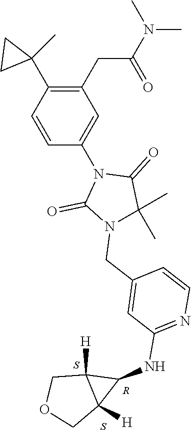 | N,N-dimethyl(5-{3-[(2-{(1R,5S,6r)-3- oxabicyclo[3.1.0]hex-6-ylamino}-4- pyridyl)methyl]-4,4-dimethyl-2,5-dioxo-1- imidazolidinyl}-2-(1- methylcyclopropyl)phenyl)acetamide |
| 29 | 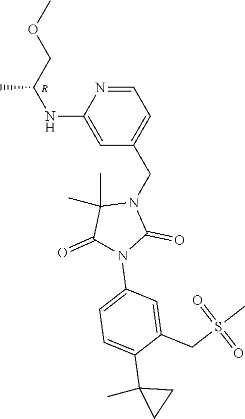 | 1-({2-[(R)-2-methoxy-1-methylethylamino]-4- pyridyl}methyl)-3-[3-(mesylmethyl)-4-(1- methylcyclopropyl)phenyl]-5,5-dimethyl-2,4- imidazolidinedione |
| 30 | 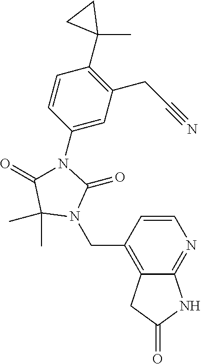 [SMILES: CC1(c2ccc(N3C(=O)N(Cc4ccnc5c4CC(=O)N5)C(C)(C)C3=O)cc2CC#N)CC1] | (5-{4,4-dimethyl-2,5-dioxo-3-[(2-oxo-1,7-diaza- 4-indanyl)methyl]-1-imidazolidinyl}-2-(1- methylcyclopropyl)phenyl)acetonitrile |
| 31 | 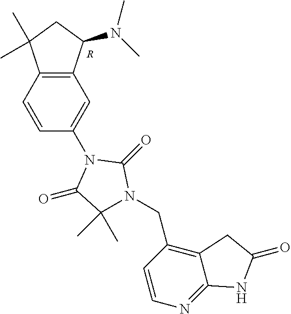 | 3-[(R)-3-(dimethylamino)-1,1-dimethyl-5- indanyl]-5,5-dimethyl-1-[(2-oxo-1,7-diaza-4- indanyl)methyl]-2,4-imidazolidinedione |
| 32 | 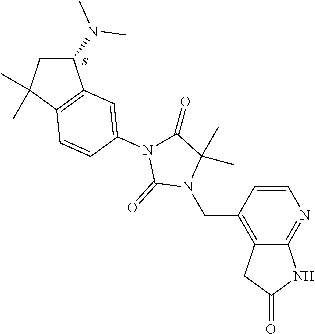 | 3-[(S)-3-(dimethylamino)-1,1-dimethyl-5- indanyl]-5,5-dimethyl-1-[(2-oxo-1,7-diaza-4- indanyl)methyl]-2,4-imidazolidinedione |
| 33 | 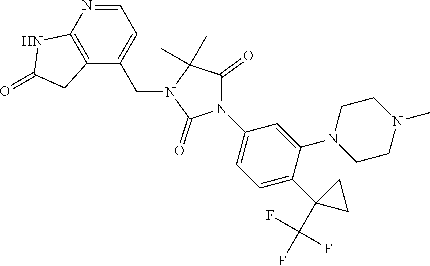 [SMILES: CN1CCN(c2cc(N3C(=O)N(Cc4ccnc5c4CC(=O)N5)C(C)(C)C3=O)ccc2C2(C(F)(F)F)CC2)CC1] | 5,5-dimethyl-3-{3-(4-methyl-1-piperazinyl)-4- [1-(trifluoromethyl)cyclopropyl]phenyl}-1-[(2- oxo-1,7-diaza-4-indanyl)methyl]-2,4- imidazolidinedione |
| 34 | 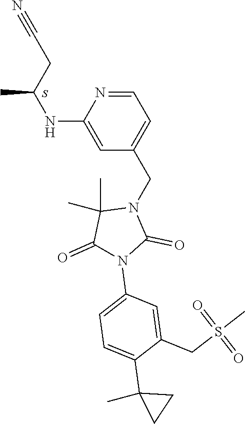 | (S)-3-[4-({3-[3-(mesylmethyl)-4-(1- methylcyclopropyl)phenyl]-5,5-dimethyl-2,4- dioxo-1-imidazolidinyl}methyl)-2- pyridylamino]butyronitrile |
| 35 | 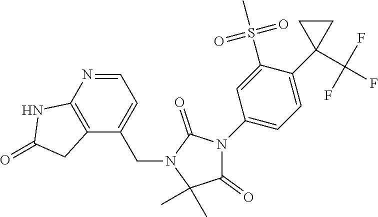 [SMILES: CC1(C)C(=O)N(c2ccc(C3(C(F)(F)F)CC3)c(S(C)(=O)=O)c2)C(=O)N1Cc1ccnc2c1CC(=O)N2] | 3-{3-mesyl-4-[1- (trifluoromethyl)cyclopropyl]phenyl}-5,5- dimethyl-1-[(2-oxo-1,7-diaza-indanyl)methyl]- 2,4-imidazolidinedione |
| 36 | 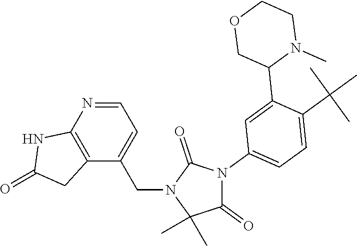 [SMILES: CN1CCOCC1c1cc(N2C(=O)N(Cc3ccnc4c3CC(=O)N4)C(C)(C)C2=O)ccc1C(C)(C)C] | 3-[4-(tert-butyl)-3-(4-methyl-3- morpholinyl)phenyl]-5,5-dimethyl-1-[(2-oxo- 1,7-diaza-4-indanyl)methyl]-2,4- imidazolidinedione |
| 37 | 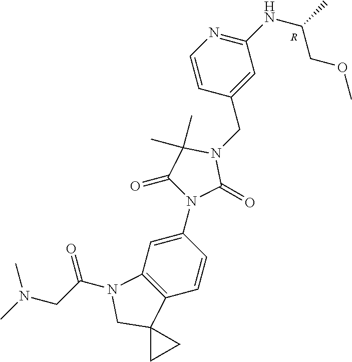 | 1-({2-[(R)-2-methoxy-1-methylethylamino]-4- pyridyl}methyl)-3-{1′-[2- (dimethylamino)acetyl]spiro[cyclopropane-1,3′- indolin]-6′-yl}-5,5-dimethyl-2,4- imidazolidinedione |
| 38 | 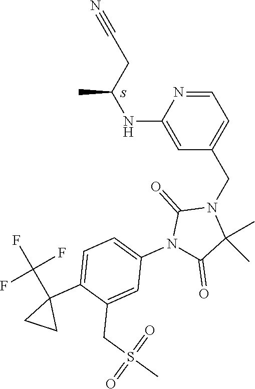 | (S)-3-{4-[(3-{3-(mesylmethyl)-4-[1- (trifluoromethyl)cyclopropyl]phenyl}-5,5- dimethyl-2,4-dioxo-1-imidazolidinyl)methyl]-2- pyridylamino}butyronitrile |
| 39 | 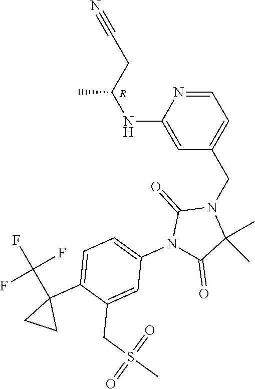 | 1-({2-[(R)-2-methoxy-1-methylethylamino]-4- pyridyl}methyl)-3-{3-(mesylmethyl)-4-[1- (trifluoromethyl)cyclopropyl]phenyl}-5,5- dimethyl-2,4-imidazolidinedione |
| 40 | 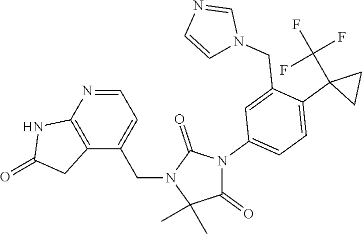 [SMILES: CC1(C)C(=O)N(c2ccc(C3(C(F)(F)F)CC3)c(Cn3ccnc3)c2)C(=O)N1Cc1ccnc2c1CC(=O)N2] | 3-{3-[(1-imidazolyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 41 | 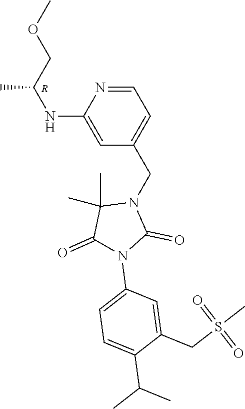 | 1-({2-[(R)-2-methoxy-1-methylethylamino]-4- pyridyl}methyl)-3-[3-(mesylmethyl)-4- cumenyl]-5,5-dimethyl-2,4-imidazolidinedione |
| 42 | 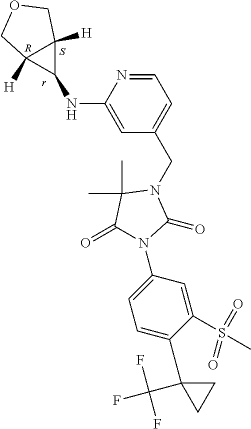 | 1-[(2-{(1R,5S,6r)-3-oxabicyclo[3.1.0]hex-6- ylamino}-4-pyridyl)methyl]-3-{3-mesyl-4-[1- (trifluoromethyl)cyclopropyl]phenyl}-5,5- dimethyl-2,4-imidazolidinedione |
| 43 | 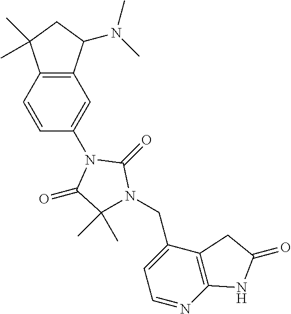 [SMILES: CN(C)C1CC(C)(C)c2ccc(N3C(=O)N(Cc4ccnc5c4CC(=O)N5)C(C)(C)C3=O)cc21] | 3-[3-(dimethylamino)-1,1-dimethyl-5-indanyl]- 5,5-dimethyl-1-[(2-oxo-1,7-diaza-4- indanyl)methyl]-2,4-imidazolidinedione |
| 44 | 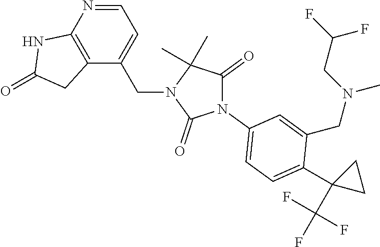 [SMILES: CN(Cc1cc(N2C(=O)N(Cc3ccnc4c3CC(=O)N4)C(C)(C)C2=O)ccc1C1(C(F)(F)F)CC1)CC(F)F] | 3-(3-{[(2,2-difluoroethyl)-N- methylamino]methyl}-4-[1- (trifluoromethyl)cyclopropyl]phenyl)-5,5- dimethyl-1-[(2-oxo-1,7-diaza-4-indanyl)methyl]- 2,4-imidazolidinedione |
| 45 | 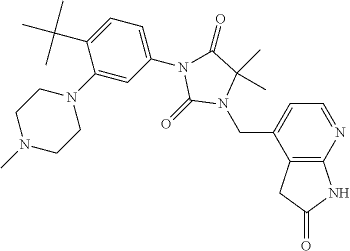 [SMILES: CN1CCN(c2cc(N3C(=O)N(Cc4ccnc5c4CC(=O)N5)C(C)(C)C3=O)ccc2C(C)(C)C)CC1] | 3-[4-(tert-butyl)-3-(4-methyl-1- piperazinyl)phenyl]-5,5-dimethyl-1-[(2-oxo-1,7- diaza-4-indanyl)methyl]-2,4-imidazolidinedione |
| 46 | 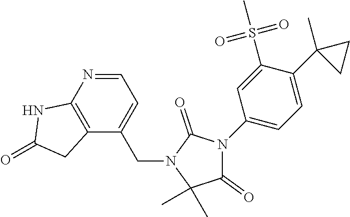 [SMILES: CC1(c2ccc(N3C(=O)N(Cc4ccnc5c4CC(=O)N5)C(C)(C)C3=O)cc2S(C)(=O)=O)CC1] | 3-[3-mesyl-4-(1-methylcyclopropyl)phenyl]-5,5- dimethyl-1-[(2-oxo-1,7-diaza-4-indanyl)methyl]- 2,4-imidazolidinedione |
| 47 | 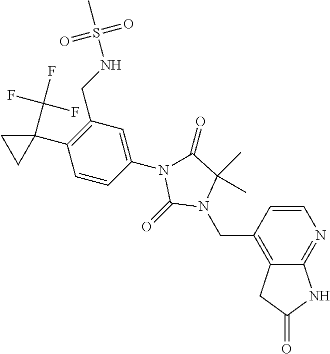 [SMILES: CC1(C)C(=O)N(c2ccc(C3(C(F)(F)F)CC3)c(CNS(C)(=O)=O)c2)C(=O)N1Cc1ccnc2c1CC(=O)N2] | 3-{3-[(mesylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 48 | 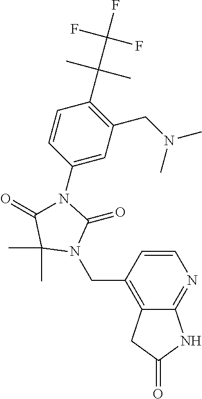 [SMILES: CN(C)Cc1cc(N2C(=O)N(Cc3ccnc4c3CC(=O)N4)C(C)(C)C2=O)ccc1C(C)(C)C(F)(F)F] | 3-{3-[(dimethylamino)methyl]-4-(2,2,2-trifluoro- 1,1-dimethylethyl)phenyl}-5,5-dimethyl-1-[(2- oxo-1,7-diaza-4-indanyl)methyl]-2,4- imidazolidinedione |
| 49 | 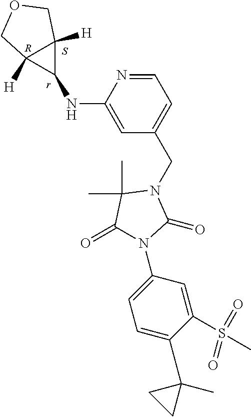 | 1-[(2-{(1R,5S,6r)-3-oxabicyclo[3.1.0]hex-6- ylamino}-4-pyridyl)methyl]-3-[3-mesyl-4-(1- methylcyclopropyl)phenyl]-5,5-dimethyl-2,4- imidazolidinedione |
| 50 | 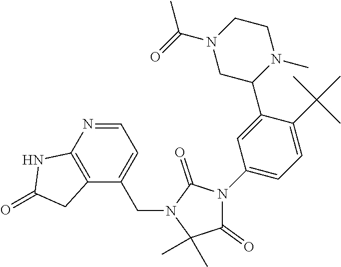 [SMILES: CC(=O)N1CCN(C)C(c2cc(N3C(=O)N(Cc4ccnc5c4CC(=O)N5)C(C)(C)C3=O)ccc2C(C)(C)C)C1] | 3-[3-(4-acetyl-1-methyl-2-piperazinyl)-4-(tert- butyl)phenyl]-5,5-dimethyl-1-[(2-oxo-1,7-diaza- 4-indanyl)methyl]-2,4-imidazolidinedione |
| 51 | 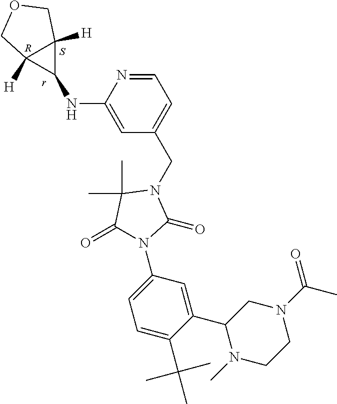 | 1-[(2-{(1R,5S,6r)-3-oxabicyclo[3.1.0]hex-6- ylamino}-4-pyridyl)methyl]-3-[3-(4-acetyl-1- methyl-1-piperazinyl)-4-(tert-butyl)phenyl]-5,5- dimethyl-2,4-imidazolidinedione |
| 52 | 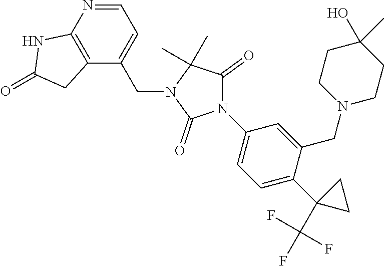 [SMILES: CC1(O)CCN(Cc2cc(N3C(=O)N(Cc4ccnc5c4CC(=O)N5)C(C)(C)C3=O)ccc2C2(C(F)(F)F)CC2)CC1] | 3-{3-[(4-hydroxy-4-methyl-1-piperidyl)methyl]- 4-[1-(trifluoromethyl)cyclopropyl]phenyl}-5,5- dimethyl-1-[(2-oxo-1,7-diaza-4-indanyl)methyl]- 2,4-imidazolidinedione |
| 53 | 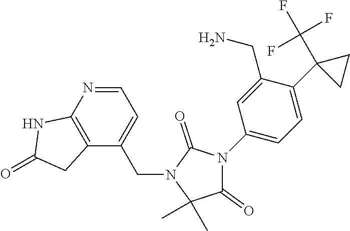 [SMILES: CC1(C)C(=O)N(c2ccc(C3(C(F)(F)F)CC3)c(CN)c2)C(=O)N1Cc1ccnc2c1CC(=O)N2] | 3-{3-(aminomethyl)-4-[1- (trifluoromethyl)cyclopropyl]phenyl}-5,5- dimethyl-1-[(2-oxo-1,7-diaza-4-indanyl)methyl]- 2,4-imidazolidinedione |
| 54 | 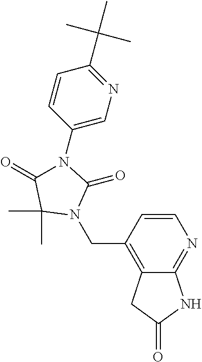 [SMILES: CC(C)(C)c1ccc(N2C(=O)N(Cc3ccnc4c3CC(=O)N4)C(C)(C)C2=O)cn1] | 3-[6-(tert-butyl)-3-pyridyl]-5,5-dimethyl-1-[(2- oxo-1,7-diaza-4-indanyl)methyl]-2,4- imidazolidinedione |
| 55 | 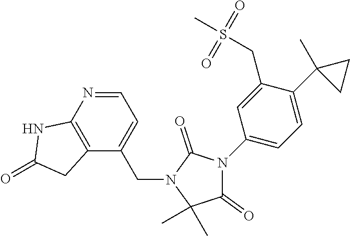 [SMILES: CC1(c2ccc(N3C(=O)N(Cc4ccnc5c4CC(=O)N5)C(C)(C)C3=O)cc2CS(C)(=O)=O)CC1] | 3-[3-(mesylmethyl)-4-(1- methylcyclopropyl)phenyl]-5,5-dimethyl-1-[(2- oxo-1,7-diaza-4-indanyl)methyl]-2,4- imidazolidinedione |
| 56 | 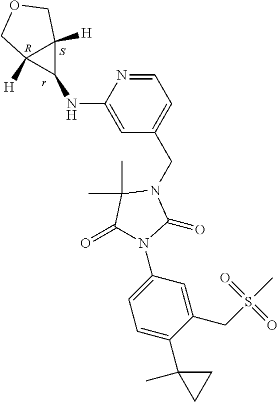 | 1-[(2-{(1R,5S,6r)-3-oxabicyclo[3.1.0]hex-6- ylamino}-4-pyridyl)methyl]-3-[3-(mesylmethyl)- 4-(1-methylcyclopropyl)phenyl]-5,5-dimethyl- 2,4-imidazolidinedione |
| 57 | 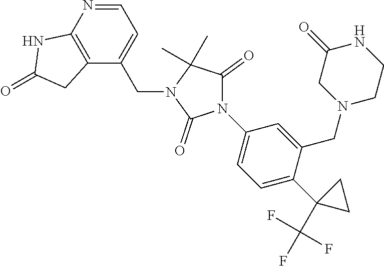 [SMILES: CC1(C)C(=O)N(c2ccc(C3(C(F)(F)F)CC3)c(CN3CCNC(=O)C3)c2)C(=O)N1Cc1ccnc2c1CC(=O)N2] | 5,5-dimethyl-1-[(2-oxo-1,7-diaza-4- indanyl)methyl]-3-{3-[(3-oxo-1- piperazinyl)methyl]-4-[1- (trifluoromethyl)cyclopropyl]phenyl}-2,4- imidazolidinedione |
| 58 | 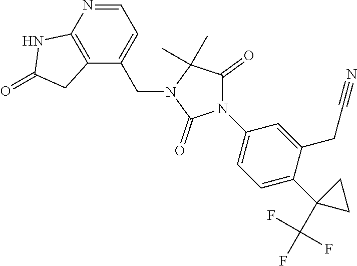 [SMILES: CC1(C)C(=O)N(c2ccc(C3(C(F)(F)F)CC3)c(CC#N)c2)C(=O)N1Cc1ccnc2c1CC(=O)N2] | (5-{4,4-dimethyl-2,5-dioxo-3-[(2-oxo-1,7-diaza- 4-indanyl)methyl]-1-imidazolidinyl}-2-[1- (trifluoromethyl)cyclopropyl]phenyl)acetonitrile |
| 59 | 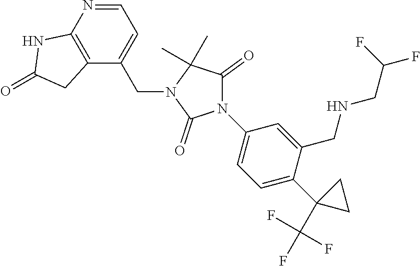 [SMILES: CC1(C)C(=O)N(c2ccc(C3(C(F)(F)F)CC3)c(CNCC(F)F)c2)C(=O)N1Cc1ccnc2c1CC(=O)N2] | 3-{3-[(2,2-difluoroethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 60 | 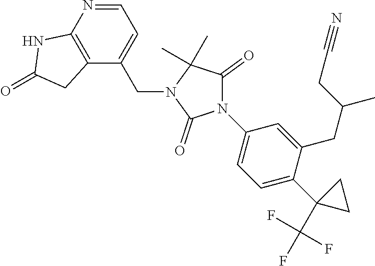 [SMILES: CC(CC#N)Cc1cc(N2C(=O)N(Cc3ccnc4c3CC(=O)N4)C(C)(C)C2=O)ccc1C1(C(F)(F)F)CC1] | {[(5-{4,4-dimethyl-2,5-dioxo-3-[(2-oxo-1,7- diaza-4-indanyl)methyl]-1-imidazolidinyl}-2-[1- (trifluoromethyl)cyclopropyl]phenyl)methyl]-N- methylamino}acetonitrile |
| 61 | 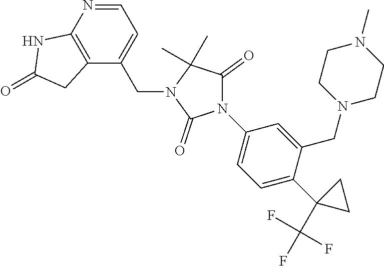 [SMILES: CN1CCN(Cc2cc(N3C(=O)N(Cc4ccnc5c4CC(=O)N5)C(C)(C)C3=O)ccc2C2(C(F)(F)F)CC2)CC1] | 5,5-dimethyl-3-{3-[(4-methyl-1- piperazinyl)methyl]-4-[1- (trifluoromethyl)cyclopropyl]phenyl}-1-[(2-oxo- 1,7-diaza-4-indanyl)methyl]-2,4- imidazolidinedione |
| 62 | 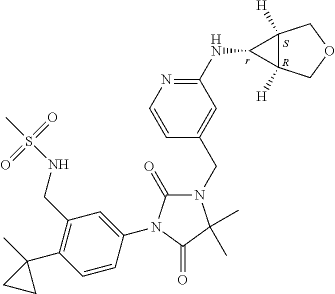 | 1-[(2-{(1R,5S,6r)-3-oxabicyclo[3.1.0]hex-6- ylamino}-4-pyridyl)methyl]-3-{3- [(mesylamino)methyl]-4-(1- methylcyclopropyl)phenyl}-5,5-dimethyl-2,4- imidazolidinedione |
| 63 | 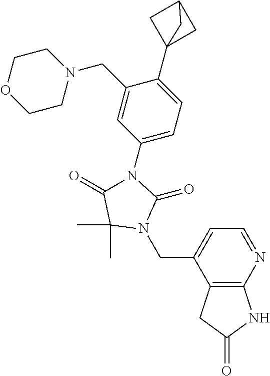 [SMILES: CC1(C)C(=O)N(c2ccc(C34CC(C3)C4)c(CN3CCOCC3)c2)C(=O)N1Cc1ccnc2c1CC(=O)N2] | 3-{4-(bicyclo[1.1.1]pent-1-yl)-3- (morpholinomethyl)phenyl}-5,5-dimethyl-1-[(2- oxo-1,7-diaza-4-indanyl)methyl]-2,4- imidazolidinedione |
| 64 | 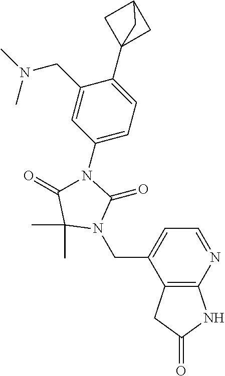 [SMILES: CN(C)Cc1cc(N2C(=O)N(Cc3ccnc4c3CC(=O)N4)C(C)(C)C2=O)ccc1C12CC(C1)C2] | 3-{4-(bicyclo[1.1.1]pent-1-yl)-3- [(dimethylamino)methyl]phenyl}-5,5-dimethyl- 1-[(2-oxo-1,7-diaza-4-indanyl)methyl]-2,4- imidazolidinedione |
| 65 | 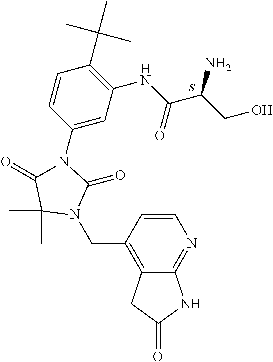 | N-[2-(tert-butyl)-5-{4,4-dimethyl-2,5-dioxo-3- [(2-oxo-1,7-diaza-4-indanyl)methyl]-1- imidazolidinyl}phenyl](S)-2-amino-3- hydroxypropionamide |
| 66 | 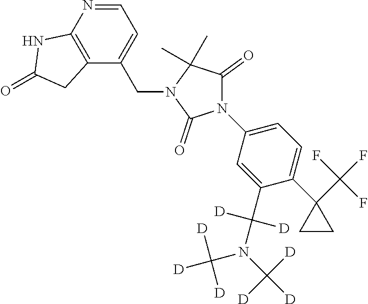 [SMILES: [2H]C([2H])([2H])N(C([2H])([2H])[2H])C([2H])([2H])c1cc(N2C(=O)N(Cc3ccnc4c3CC(=O)N4)C(C)(C)C2=O)ccc1C1(C(F)(F)F)CC1] | 3-(3-{[di(^{²}H₃)methylamino](^{²}H₂)methyl}-4-[1- (trifluoromethyl)cyclopropyl]phenyl)-5,5- dimethyl-1-[(2-oxo-1,7-diaza-4-indanyl)methyl]- 2,4-imidazolidinedione |
| 67 | 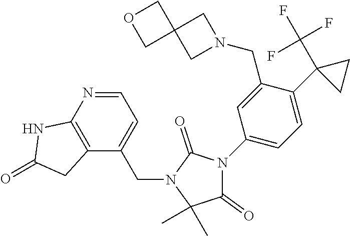 [SMILES: CC1(C)C(=O)N(c2ccc(C3(C(F)(F)F)CC3)c(CN3CC4(COC4)C3)c2)C(=O)N1Cc1ccnc2c1CC(=O)N2] | 5,5-dimethyl-3-(3-{(2-oxa-6-aza-6- spiro[3.3]heptyl)methyl}-4-[1- (trifluoromethyl)cyclopropyl]phenyl)-1-[(2-oxo- 1,7-diaza-4-indanyl)methyl]-2,4- imidazolidinedione |
| 68 | 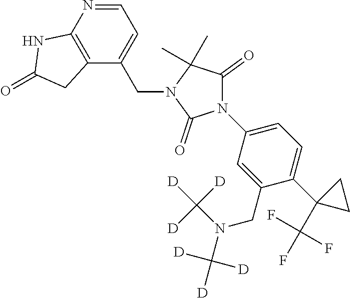 [SMILES: [2H]C([2H])([2H])N(Cc1cc(N2C(=O)N(Cc3ccnc4c3CC(=O)N4)C(C)(C)C2=O)ccc1C1(C(F)(F)F)CC1)C([2H])([2H])[2H]] | 3-(3-{[di(²H₃)methylamino]methyl}-4-[1- (trifluoromethyl)cyclopropyl]phenyl)-5,5- dimethyl-1-[(2-oxo-1,7-diaza-4-indanyl)methyl]- 2,4-imidazolidinedione |
| 69 | 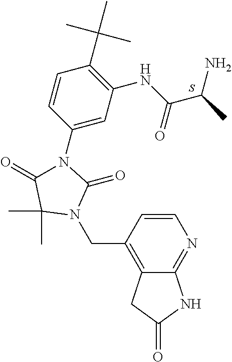 | N-[2-(tert-butyl)-5-{4,4-dimethyl-2,5-dioxo-3- [(2-oxo-1,7-diaza-4-indanyl)methyl]-1- imidazolidinyl}phenyl](S)-2-aminopropionamide |
| 70 | 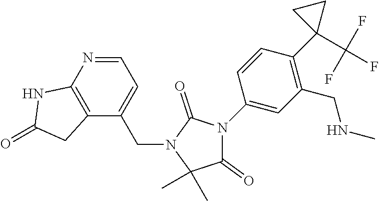 [SMILES: CNCc1cc(N2C(=O)N(Cc3ccnc4c3CC(=O)N4)C(C)(C)C2=O)ccc1C1(C(F)(F)F)CC1] | 5,5-dimethyl-3-{3-[(methylamino)methyl]-4-[1- (trifluoromethyl)cyclopropyl]phenyl}-1-[(2-oxo- 1,7-diaza-4-indanyl)methyl]-2,4- imidazolidinedione |
| 71 | 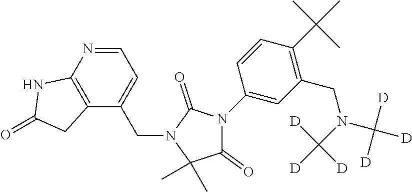 [SMILES: [2H]C([2H])([2H])N(Cc1cc(N2C(=O)N(Cc3ccnc4c3CC(=O)N4)C(C)(C)C2=O)ccc1C(C)(C)C)C([2H])([2H])[2H]] | 3-[4-(tert-butyl)-3- {[di(²H₃)methylamino]methyl}phenyl]-5,5- dimethyl-1-[(2-oxo-1,7-diaza-4-indanyl)methyl]- 2,4-imidazolidinedione |
| 72 | 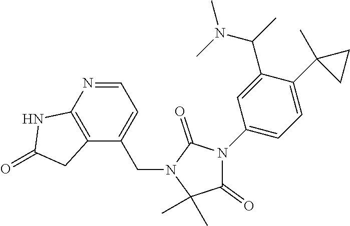 [SMILES: CC(c1cc(N2C(=O)N(Cc3ccnc4c3CC(=O)N4)C(C)(C)C2=O)ccc1C1(C)CC1)N(C)C] | 3-{3-[1-(dimethylamino)ethyl]-4-(1- methylcyclopropyl)phenyl}-5,5-dimethyl-1-[(2- oxo-1,7-diaza-4-indanyl)methyl]-2,4- imidazolidinedione |
| 73 | 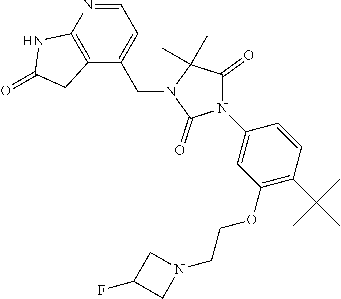 [SMILES: CC(C)(C)c1ccc(N2C(=O)N(Cc3ccnc4c3CC(=O)N4)C(C)(C)C2=O)cc1OCCN1CC(F)C1] | 3-{4-(tert-butyl)-3-[2-(3-fluoro-1- azetidinyl)ethoxy]phenyl}-5,5-dimethyl-1-[(2- oxo-1,7-diaza-4-indanyl)methyl]-2,4- imidazolidinedione |
| 74 | 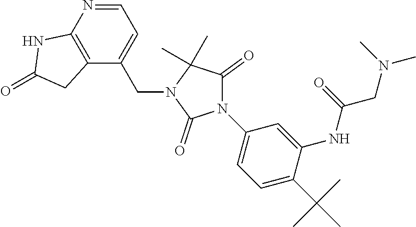 [SMILES: CN(C)CC(=O)Nc1cc(N2C(=O)N(Cc3ccnc4c3CC(=O)N4)C(C)(C)C2=O)ccc1C(C)(C)C] | N-[2-(tert-butyl)-5-{4,4-dimethyl-2,5-dioxo-3- [(2-oxo-1,7-diaza-4-indanyl)methyl]-1- imidazolidinyl}phenyl](dimethylamino)acetamide |
| 75 | 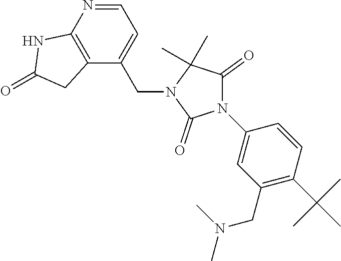 [SMILES: CN(C)Cc1cc(N2C(=O)N(Cc3ccnc4c3CC(=O)N4)C(C)(C)C2=O)ccc1C(C)(C)C] | 3-{4-(tert-butyl)-3- [(dimethylamino)methyl]phenyl}-5,5-dimethyl- 1-[(2-oxo-1,7-diaza-4-indanyl)methyl]-2,4- imidazolidinedione |
| 76 | 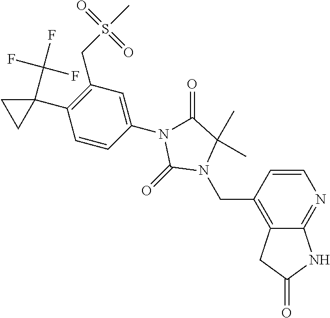 [SMILES: CC1(C)C(=O)N(c2ccc(C3(C(F)(F)F)CC3)c(CS(C)(=O)=O)c2)C(=O)N1Cc1ccnc2c1CC(=O)N2] | 3-{3-(mesylmethyl)-4-[1- (trifluoromethyl)cyclopropyl]phenyl}-5,5- dimethyl-1-[(2-oxo-1,7-diaza-4-indanyl)methyl]- 2,4-imidazolidinedione |
| 77 | 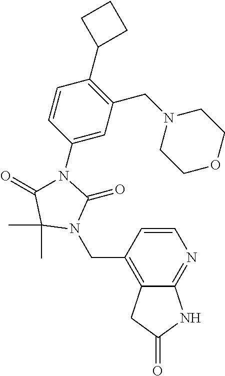 [SMILES: CC1(C)C(=O)N(c2ccc(C3CCC3)c(CN3CCOCC3)c2)C(=O)N1Cc1ccnc2c1CC(=O)N2] | 3-[4-cyclobutyl-3-(morpholinomethyl)phenyl]- 5,5-dimethyl-1-[(2-oxo-1,7-diaza-4- indanyl)methyl]-2,4-imidazolidinedione |
| 78 | 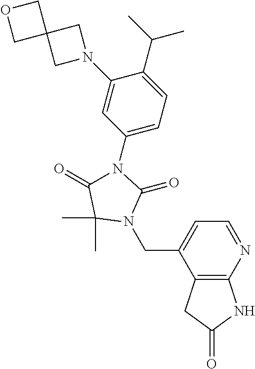 [SMILES: CC(C)c1ccc(N2C(=O)N(Cc3ccnc4c3CC(=O)N4)C(C)(C)C2=O)cc1N1CC2(COC2)C1] | 5,5-dimethyl-3-{3-(2-oxa-6-aza-6- spiro[3.3]heptyl)-4-cumenyl}-1-[(2-oxo-1,7- diaza-4-indanyl)methyl]-2,4-imidazolidinedione |
| 79 | 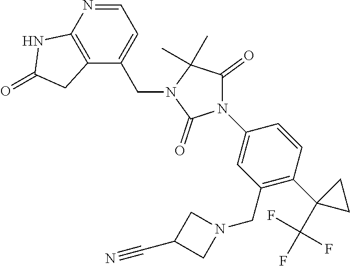 [SMILES: CC1(C)C(=O)N(c2ccc(C3(C(F)(F)F)CC3)c(CN3CC(C#N)C3)c2)C(=O)N1Cc1ccnc2c1CC(=O)N2] | 1-[(5-{4,4-dimethyl-2,5-dioxo-3-[(2-oxo-1,7- diaza-4-indanyl)methyl]-1-imidazolidinyl}-2-[1- (trifluoromethyl)cyclopropyl]phenyl)methyl]-3- azetidinecarbonitrile |
| 80 | 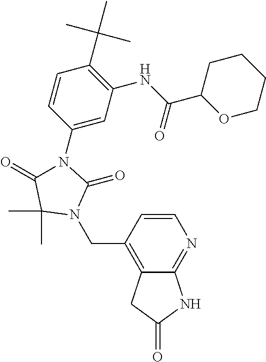 [SMILES: CC(C)(C)c1ccc(N2C(=O)N(Cc3ccnc4c3CC(=O)N4)C(C)(C)C2=O)cc1NC(=O)C1CCCCO1] | N-[2-(tert-butyl)-5-{4,4-dimethyl-2,5-dioxo-3- [(2-oxo-1,7-diaza-4-indanyl)methyl]-1- imidazolidinyl}phenyl]-tetrahydro-2H-pyran-2- carboxamide |
| 81 | 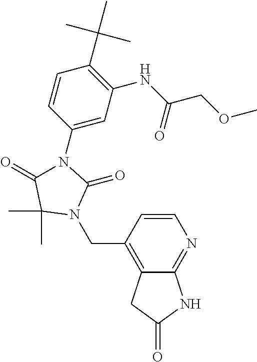 [SMILES: COCC(=O)Nc1cc(N2C(=O)N(Cc3ccnc4c3CC(=O)N4)C(C)(C)C2=O)ccc1C(C)(C)C] | N-[2-(tert-butyl)-5-{4,4-dimethyl-2,5-dioxo-3- [(2-oxo-1,7-diaza-4-indanyl)methyl]-1- imidazolidinyl}phenyl]methoxyacetamide |
| 82 | 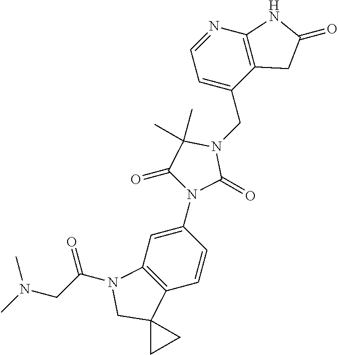 [SMILES: CN(C)CC(=O)N1CC2(CC2)c2ccc(N3C(=O)N(Cc4ccnc5c4CC(=O)N5)C(C)(C)C3=O)cc21] | 3-{1′-[2- (dimethylamino)acetyl]spiro[cyclopropane-1,3′- indolin]-6′-yl}-5,5-dimethyl-1-[(2-oxo-1,7-diaza- 4-indanyl)methyl]-2,4-imidazolidinedione |
| 83 | 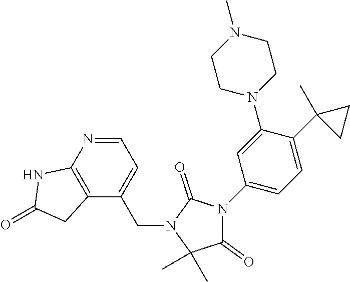 [SMILES: CN1CCN(c2cc(N3C(=O)N(Cc4ccnc5c4CC(=O)N5)C(C)(C)C3=O)ccc2C2(C)CC2)CC1] | 5,5-dimethyl-3-[4-(1-methylcyclopropyl)-3-(4- methyl-1-piperazinyl)phenyl]-1-[(2-oxo-1,7- diaza-4-indanyl)methyl]-2,4-imidazolidinedione |
| 84 | 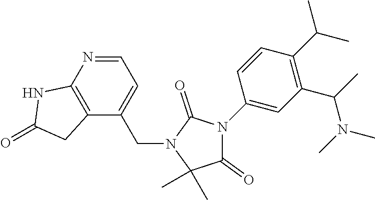 [SMILES: CC(C)c1ccc(N2C(=O)N(Cc3ccnc4c3CC(=O)N4)C(C)(C)C2=O)cc1C(C)N(C)C] | 3-{3-[1-(dimethylamino)ethyl]-4-cumenyl}-5,5- dimethyl-1-[(2-oxo-1,7-diaza-4-indanyl)methyl]- 2,4-imidazolidinedione |
| 85 | 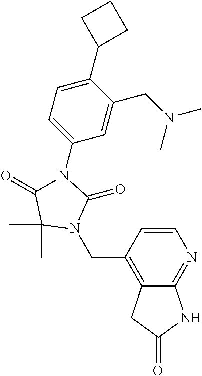 [SMILES: CN(C)Cc1cc(N2C(=O)N(Cc3ccnc4c3CC(=O)N4)C(C)(C)C2=O)ccc1C1CCC1] | 3-{4-cyclobutyl-3- [(dimethylamino)methyl]phenyl}-5,5-dimethyl- 1-[(2-oxo-1,7-diaza-4-indanyl)methyl]-2,4- imidazolidinedione |
| 86 | 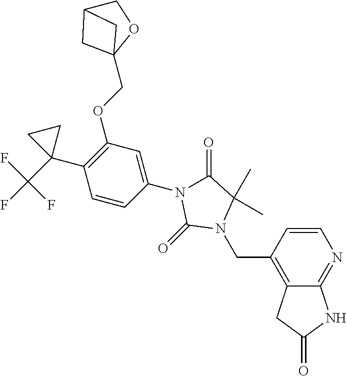 [SMILES: CC1(C)C(=O)N(c2ccc(C3(C(F)(F)F)CC3)c(OCC34CC(CO3)C4)c2)C(=O)N1Cc1ccnc2c1CC(=O)N2] | 5,5-dimethyl-3-(3-{(2-oxabicyclo[2.1.1]hex-1- yl)methoxy}-4-[1- (trifluoromethyl)cyclopropyl]phenyl)-1-[(2-oxo- 1,7-diaza-4-indanyl)methyl]-2,4- imidazolidinedione |
| 87 | 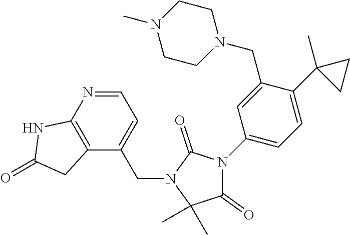 [SMILES: CN1CCN(Cc2cc(N3C(=O)N(Cc4ccnc5c4CC(=O)N5)C(C)(C)C3=O)ccc2C2(C)CC2)CC1] | 5,5-dimethyl-3-{4-(1-methylcyclopropyl)-3-[(4- methyl-1-piperazinyl)methyl]phenyl}-1-[(2-oxo- 1,7-diaza-4-indanyl)methyl]-2,4- imidazolidinedione |
| 88 | 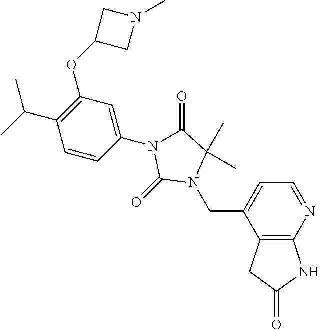 [SMILES: CC(C)c1ccc(N2C(=O)N(Cc3ccnc4c3CC(=O)N4)C(C)(C)C2=O)cc1OC1CN(C)C1] | 5,5-dimethyl-3-[3-(1-methyl-3-azetidinyloxy)-4- cumenyl]-1-[(2-oxo-1,7-diaza-4- indanyl)methyl]-2,4-imidazolidinedione |
| 89 | 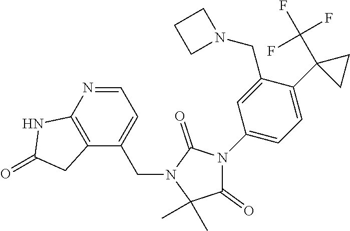 [SMILES: CC1(C)C(=O)N(c2ccc(C3(C(F)(F)F)CC3)c(CN3CCC3)c2)C(=O)N1Cc1ccnc2c1CC(=O)N2] | 3-{3-[(1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 90 | 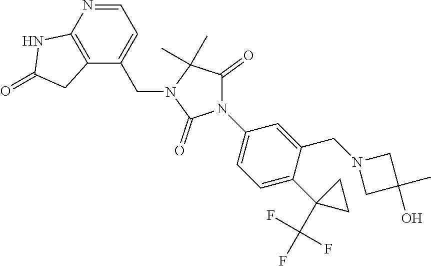 [SMILES: CC1(O)CN(Cc2cc(N3C(=O)N(Cc4ccnc5c4CC(=O)N5)C(C)(C)C3=O)ccc2C2(C(F)(F)F)CC2)C1] | 3-{3-[(3-hydroxy-3-methyl-1-azetidinyl)methyl]- 4-[1-(trifluoromethyl)cyclopropyl]phenyl}-5,5- dimethyl-1-[(2-oxo-1,7-diaza-4-indanyl)methyl]- 2,4-imidazolidinedione |
| 91 | 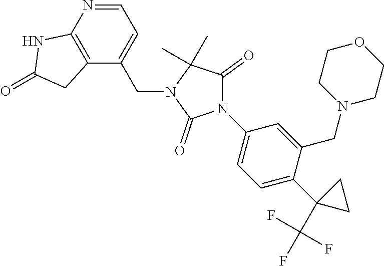 [SMILES: CC1(C)C(=O)N(c2ccc(C3(C(F)(F)F)CC3)c(CN3CCOCC3)c2)C(=O)N1Cc1ccnc2c1CC(=O)N2] | 5,5-dimethyl-3-{3-(morpholinomethyl)-4-[1- (trifluoromethyl)cyclopropyl]phenyl}-1-[(2-oxo- 1,7-diaza-4-indanyl)methyl]-2,4- imidazolidinedione |
| 92 | 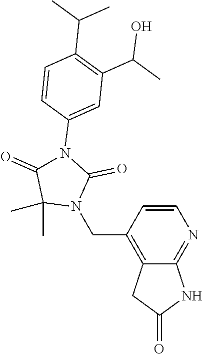 [SMILES: CC(C)c1ccc(N2C(=O)N(Cc3ccnc4c3CC(=O)N4)C(C)(C)C2=O)cc1C(C)O] | 3-[3-(1-hydroxyethyl)-4-cumenyl]-5,5-dimethyl- 1-[(2-oxo-1,7-diaza-4-indanyl)methyl]-2,4- imidazolidinedione |
| 93 | 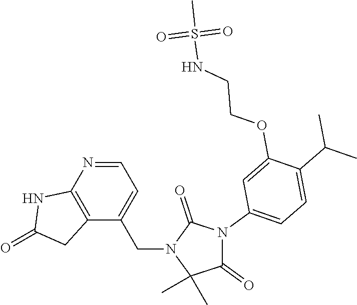 [SMILES: CC(C)c1ccc(N2C(=O)N(Cc3ccnc4c3CC(=O)N4)C(C)(C)C2=O)cc1OCCNS(C)(=O)=O] | 3-{3-[2-(mesylamino)ethoxy]-4-cumenyl}-5,5- dimethyl-1-[(2-oxo-1,7-diaza-4-indanyl)methyl]- 2,4-imidazolidinedione |
| 94 | 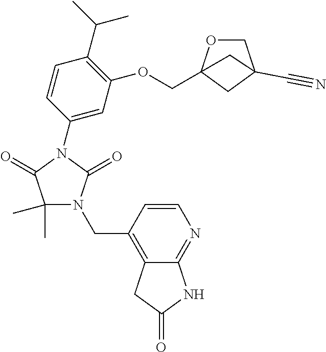 [SMILES: CC(C)c1ccc(N2C(=O)N(Cc3ccnc4c3CC(=O)N4)C(C)(C)C2=O)cc1OCC12CC(C#N)(CO1)C2] | 1-[(5-{4,4-dimethyl-2,5-dioxo-3-[(2-oxo-1,7- diaza-4-indanyl)methyl]-1-imidazolidinyl}-2- cumenyloxy)methyl]-2-oxabicyclo[2.1.1]hexane- 4-carbonitrile |
| 95 | 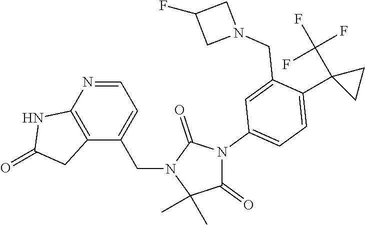 [SMILES: CC1(C)C(=O)N(c2ccc(C3(C(F)(F)F)CC3)c(CN3CC(F)C3)c2)C(=O)N1Cc1ccnc2c1CC(=O)N2] | 3-{3-[(3-fluoro-1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 96 | 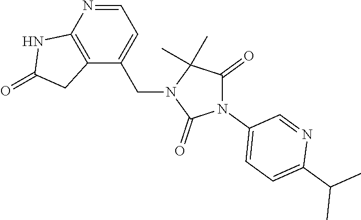 [SMILES: CC(C)c1ccc(N2C(=O)N(Cc3ccnc4c3CC(=O)N4)C(C)(C)C2=O)cn1] | 3-(6-isopropyl-3-pyridyl)-5,5-dimethyl-1-[(2- oxo-1,7-diaza-4-indanyl)methyl]-2,4- imidazolidinedione |
| 97 | 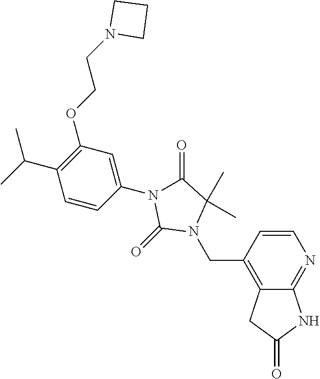 [SMILES: CC(C)c1ccc(N2C(=O)N(Cc3ccnc4c3CC(=O)N4)C(C)(C)C2=O)cc1OCCN1CCC1] | 3-{3-[2-(1-azetidinyl)ethoxy]-4-cumenyl}-5,5- dimethyl-1-[(2-oxo-1,7-diaza-4-indanyl)methyl]- 2,4-imidazolidinedione |
| 98 | 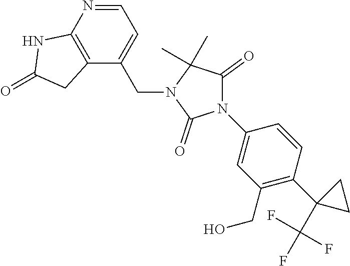 [SMILES: CC1(C)C(=O)N(c2ccc(C3(C(F)(F)F)CC3)c(CO)c2)C(=O)N1Cc1ccnc2c1CC(=O)N2] | 3-{3-(hydroxymethyl)-4-[1- (trifluoromethyl)cyclopropyl]phenyl}-5,5- dimethyl-1-[(2-oxo-1,7-diaza-4-indanyl)methyl]- 2,4-imidazolidinedione |
| 99 | 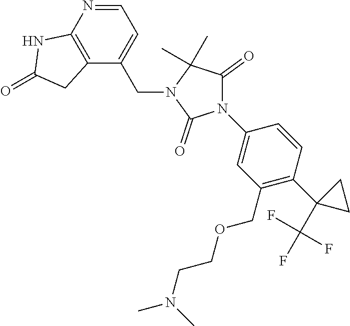 [SMILES: CN(C)CCOCc1cc(N2C(=O)N(Cc3ccnc4c3CC(=O)N4)C(C)(C)C2=O)ccc1C1(C(F)(F)F)CC1] | 3-(3-{[2-(dimethylamino)ethoxy]methyl}-4-[1- (trifluoromethyl)cyclopropyl]phenyl)-5,5- dimethyl-1-[(2-oxo-1,7-diaza-4-indanyl)methyl]- 2,4-imidazolidinedione |
| 100 | 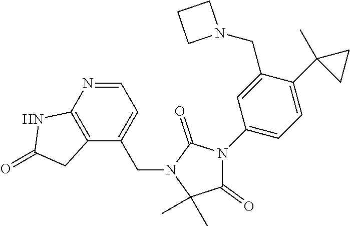 [SMILES: CC1(c2ccc(N3C(=O)N(Cc4ccnc5c4CC(=O)N5)C(C)(C)C3=O)cc2CN2CCC2)CC1] | 3-{3-[(1-azetidinyl)methyl]-4-(1- methylcyclopropyl)phenyl}-5,5-dimethyl-1-[(2- oxo-1,7-diaza-4-indanyl)methyl]-2,4- imidazolidinedione |
| 101 | 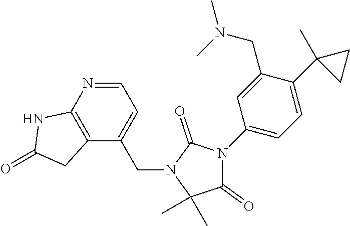 [SMILES: CN(C)Cc1cc(N2C(=O)N(Cc3ccnc4c3CC(=O)N4)C(C)(C)C2=O)ccc1C1(C)CC1] | 3-{3-[(dimethylamino)methyl]-4-(1- methylcyclopropyl)phenyl}-5,5-dimethyl-1-[(2- oxo-1,7-diaza-4-indanyl)methyl]-2,4- imidazolidinedione |
| 102 | 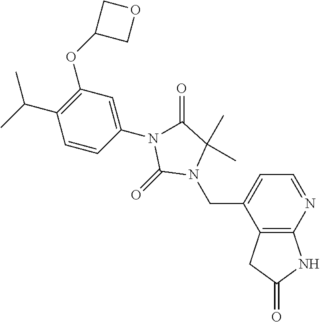 [SMILES: CC(C)c1ccc(N2C(=O)N(Cc3ccnc4c3CC(=O)N4)C(C)(C)C2=O)cc1OC1COC1] | 5,5-dimethyl-3-[3-(3-oxetanyloxy)-4-cumenyl]- 1-[(2-oxo-1,7-diaza-4-indanyl)methyl]-2,4- imidazolidinedione |
| 103 | 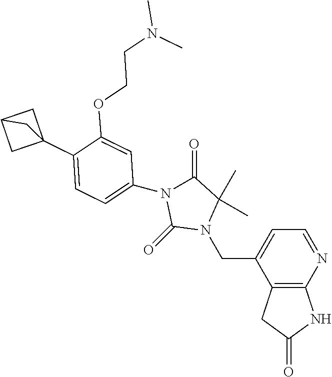 [SMILES: CN(C)CCOc1cc(N2C(=O)N(Cc3ccnc4c3CC(=O)N4)C(C)(C)C2=O)ccc1C12CC(C1)C2] | 3-{4-(bicyclo[1.1.1]pent-1-yl)-3-[2- (dimethylamino)ethoxy]phenyl}-5,5-dimethyl-1- [(2-oxo-1,7-diaza-4-indanyl)methyl]-2,4- imidazolidinedione |
| 104 | 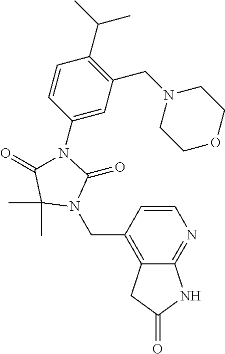 [SMILES: CC(C)c1ccc(N2C(=O)N(Cc3ccnc4c3CC(=O)N4)C(C)(C)C2=O)cc1CN1CCOCC1] | 5,5-dimethyl-3-[3-(morpholinomethyl)-4- cumenyl]-1-[(2-oxo-1,7-diaza-4- indanyl)methyl]-2,4-imidazolidinedione |
| 105 | 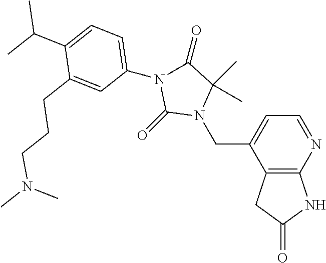 [SMILES: CC(C)c1ccc(N2C(=O)N(Cc3ccnc4c3CC(=O)N4)C(C)(C)C2=O)cc1CCCN(C)C] | 3-{3-[3-(dimethylamino)propyl]-4-cumenyl}- 5,5-dimethyl-1-[(2-oxo-1,7-diaza-4- indanyl)methyl]-2,4-imidazolidinedione |
| 106 | 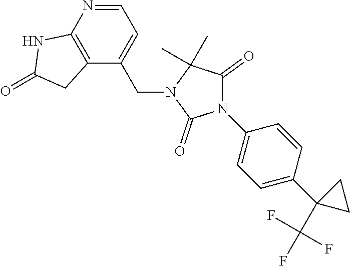 [SMILES: CC1(C)C(=O)N(c2ccc(C3(C(F)(F)F)CC3)cc2)C(=O)N1Cc1ccnc2c1CC(=O)N2] | 5,5-dimethyl-1-[(2-oxo-1-oxa-3,4-diaza-7- indenyl)methyl]-3-{p-[1- (trifluoromethyl)cyclopropyl]phenyl}-2,4- imidazolidinedione |
| 107 | 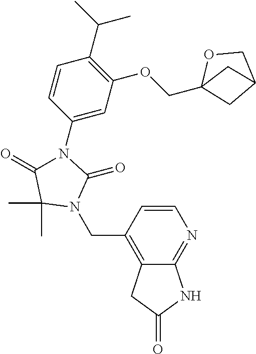 [SMILES: CC(C)c1ccc(N2C(=O)N(Cc3ccnc4c3CC(=O)N4)C(C)(C)C2=O)cc1OCC12CC(CO1)C2] | 5,5-dimethyl-3-(3-{(2-oxabicyclo[2.1.1]hex-1- yl)methoxy}-4-cumenyl)-1-[(2-oxo-1,7-diaza-4- indanyl)methyl]-2,4-imidazolidinedione |
| 108 | 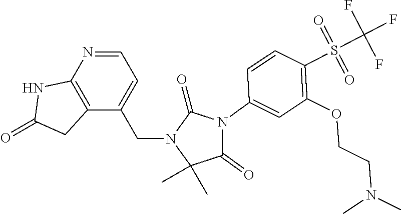 [SMILES: CN(C)CCOc1cc(N2C(=O)N(Cc3ccnc4c3CC(=O)N4)C(C)(C)C2=O)ccc1S(=O)(=O)C(F)(F)F] | 3-{3-[2-(dimethylamino)ethoxy]-4- (trifluoromesyl)phenyl}-5,5-dimethyl-1-[(2-oxo- 1,7-diaza-4-indanyl)methyl]-2,4- imidazolidinedione |
| 109 | 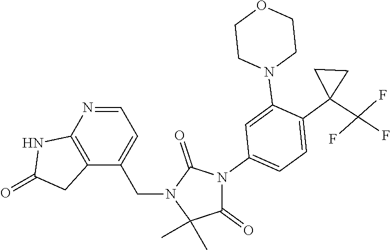 [SMILES: CC1(C)C(=O)N(c2ccc(C3(C(F)(F)F)CC3)c(N3CCOCC3)c2)C(=O)N1Cc1ccnc2c1CC(=O)N2] | 5,5-dimethyl-3-{3-morpholino-4-[1- (trifluoromethyl)cyclopropyl]phenyl}-1-[(2-oxo- 1,7-diaza-4-indanyl)methyl]-2,4- imidazolidinedione |
| 110 | 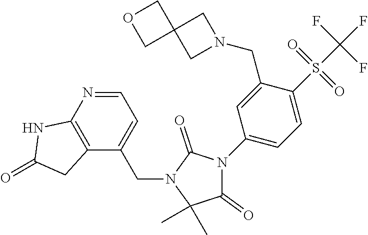 [SMILES: CC1(C)C(=O)N(c2ccc(S(=O)(=O)C(F)(F)F)c(CN3CC4(COC4)C3)c2)C(=O)N1Cc1ccnc2c1CC(=O)N2] | 5,5-dimethyl-3-(3-{(2-oxa-6-aza-6- spiro[3.3]heptyl)methyl}-4- (trifluoromesyl)phenyl)-1-[(2-oxo-1,7-diaza-4- indanyl)methyl]-2,4-imidazolidinedione |
| 111 | 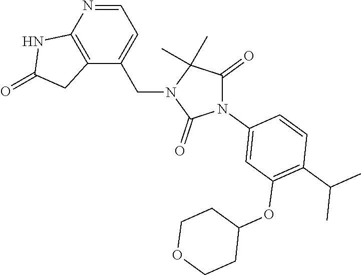 [SMILES: CC(C)c1ccc(N2C(=O)N(Cc3ccnc4c3CC(=O)N4)C(C)(C)C2=O)cc1OC1CCOCC1] | 5,5-dimethyl-1-[(2-oxo-1,7-diaza-4- indanyl)methyl]-3-[3-(tetrahydro-2H-pyran-4- yloxy)-4-cumenyl]-2,4-imidazolidinedione |
| 112 | 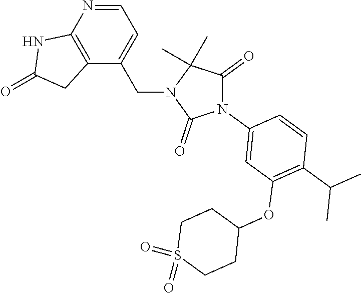 [SMILES: CC(C)c1ccc(N2C(=O)N(Cc3ccnc4c3CC(=O)N4)C(C)(C)C2=O)cc1OC1CCS(=O)(=O)CC1] | 3-[3-(1,1-dioxo-1λ⁶-4-thianyloxy)-4-cumenyl]- 5,5-dimethyl-1-[(2-oxo-1,7-diaza-4- indanyl)methyl]-2,4-imidazolidinedione |
| 113 | 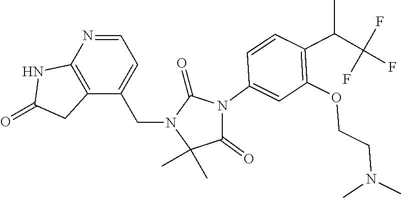 [SMILES: CC(c1ccc(N2C(=O)N(Cc3ccnc4c3CC(=O)N4)C(C)(C)C2=O)cc1OCCN(C)C)C(F)(F)F] | 3-{3-[2-(dimethylamino)ethoxy]-4-(2,2,2- trifluoro-1-methylethyl)phenyl}-5,5-dimethyl-1- [(2-oxo-1,7-diaza-4-indanyl)methyl]-2,4- imidazolidinedione |
| 114 | 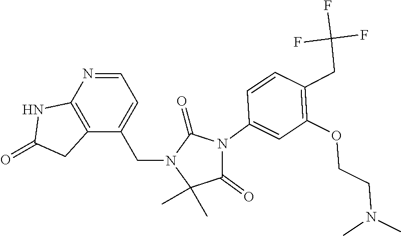 [SMILES: CN(C)CCOc1cc(N2C(=O)N(Cc3ccnc4c3CC(=O)N4)C(C)(C)C2=O)ccc1CC(F)(F)F] | 3-{3-[2-(dimethylamino)ethoxy]-4-(2,2,2- trifluoroethyl)phenyl}-5,5-dimethyl-1-[(2-oxo- 1,7-diaza-4-indanyl)methyl]-2,4- imidazolidinedione |
| 115 | 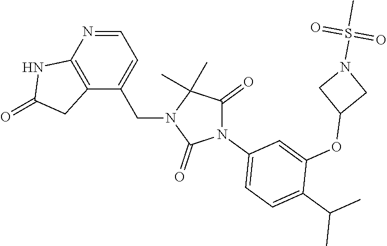 [SMILES: CC(C)c1ccc(N2C(=O)N(Cc3ccnc4c3CC(=O)N4)C(C)(C)C2=O)cc1OC1CN(S(C)(=O)=O)C1] | 3-[3-(1-mesyl-3-azetidinyloxy)-4-cumenyl]-5,5- dimethyl-1-[(2-oxo-1,7-diaza-4-indanyl)methyl]- 2,4-imidazolidinedione |
| 116 | 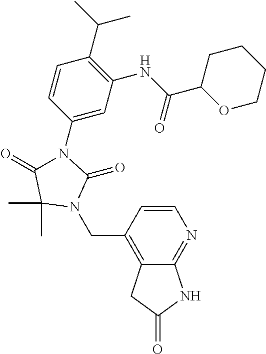 [SMILES: CC(C)c1ccc(N2C(=O)N(Cc3ccnc4c3CC(=O)N4)C(C)(C)C2=O)cc1NC(=O)C1CCCCO1] | N-(5-{4,4-dimethyl-2,5-dioxo-3-[(2-oxo-1,7- diaza-4-indanyl)methyl]-1-imidazolidinyl}-2- cumenyl)-tetrahydro-2H-pyran-2-carboxamide |
| 117 | 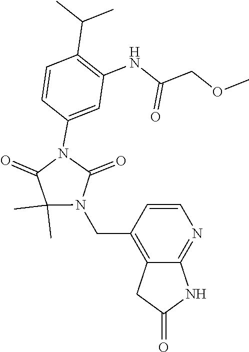 [SMILES: COCC(=O)Nc1cc(N2C(=O)N(Cc3ccnc4c3CC(=O)N4)C(C)(C)C2=O)ccc1C(C)C] | N-(5-{4,4-dimethyl-2,5-dioxo-3-[(2-oxo-1,7- diaza-4-indanyl)methyl]-1-imidazolidinyl}-2- cumenyl)methoxyacetamide |
| 118 | 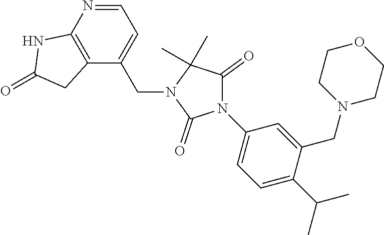 [SMILES: CC(C)c1ccc(N2C(=O)N(Cc3ccnc4c3CC(=O)N4)C(C)(C)C2=O)cc1CN1CCOCC1] | 5,5-dimethyl-3-[3-(2-morpholinoethoxy)-4- cumenyl]-1-[(2-oxo-1,7-diaza-4- indanyl)methyl]-2,4-imidazolidinedione |
| 119 | 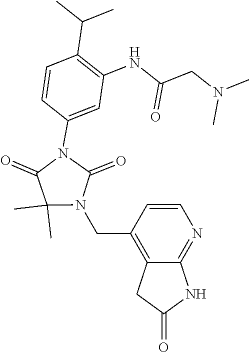 [SMILES: CC(C)c1ccc(N2C(=O)N(Cc3ccnc4c3CC(=O)N4)C(C)(C)C2=O)cc1NC(=O)CN(C)C] | N-(5-{4,4-dimethyl-2,5-dioxo-3-[(2-oxo-1,7- diaza-4-indanyl)methyl]-1-imidazolidinyl}-2- cumenyl)(dimethylamino)acetamide |
| 120 | 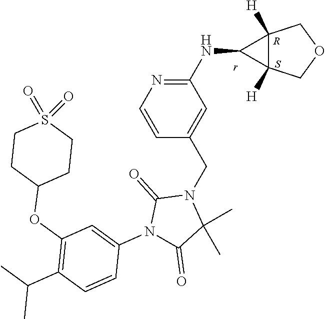 | 1-[(2-{(1R,5S,6r)-3-oxabicyclo[3.1.0]hex-6- ylamino}-4-pyridyl)methyl]-3-[3-(1,1-dioxo-1λ⁶- 4-thianyloxy)-4-cumenyl]-5,5-dimethyl-2,4- imidazolidinedione |
| 121 | 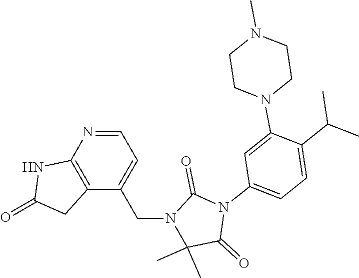 [SMILES: CC(C)c1ccc(N2C(=O)N(Cc3ccnc4c3CC(=O)N4)C(C)(C)C2=O)cc1N1CCN(C)CC1] | 5,5-dimethyl-3-[3-(4-methyl-1-piperazinyl)-4- cumenyl]-1-[(2-oxo-1,7-diaza-4- indanyl)methyl]-2,4-imidazolidinedione |
| 122 | 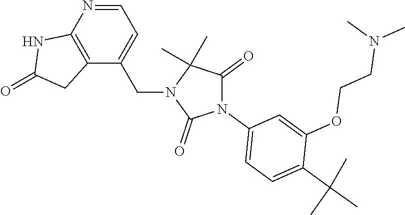 [SMILES: CN(C)CCOc1cc(N2C(=O)N(Cc3ccnc4c3CC(=O)N4)C(C)(C)C2=O)ccc1C(C)(C)C] | 3-{4-(tert-butyl)-3-[2- (dimethylamino)ethoxy]phenyl}-5,5-dimethyl-1- [(2-oxo-1,7-diaza-4-indanyl)methyl]-2,4- imidazolidinedione |
| 123 | 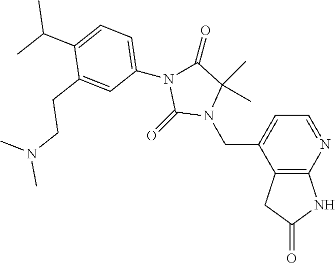 [SMILES: CC(C)c1ccc(N2C(=O)N(Cc3ccnc4c3CC(=O)N4)C(C)(C)C2=O)cc1CCN(C)C] | 3-{3-[2-(dimethylamino)ethyl]-4-cumenyl}-5,5- dimethyl-1-[(2-oxo-1,7-diaza-4-indanyl)methyl]- 2,4-imidazolidinedione |
| 124 | 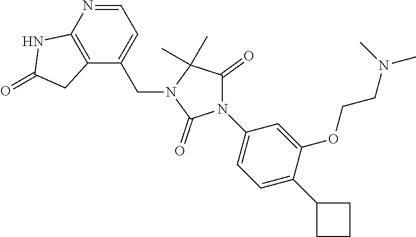 [SMILES: CN(C)CCOc1cc(N2C(=O)N(Cc3ccnc4c3CC(=O)N4)C(C)(C)C2=O)ccc1C1CCC1] | 3-{4-cyclobutyl-3-[2- (dimethylamino)ethoxy]phenyl}-5,5-dimethyl-1- [(2-oxo-1,7-diaza-4-indanyl)methyl]-2,4- imidazolidinedione |
| 125 | 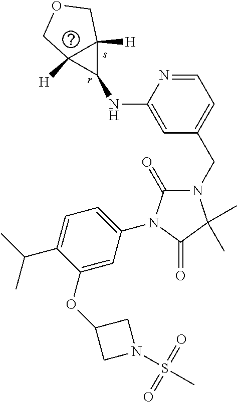 [SMILES: CC(C)c1ccc(N2C(=O)N(Cc3ccnc(N[C@H]4[C@@H]5COC[C@@H]54)c3)C(C)(C)C2=O)cc1OC1CN(S(C)(=O)=O)C1] | 1-[(2-{(1R,5S,6r)-3-oxabicyclo[3.1.0]hex-6- ylamino}-4-pyridyl)methyl]-3-[3-(1-mesyl-3- azetidinyloxy)-4-cumenyl]-5,5-dimethyl-2,4- imidazolidinedione |
| 126 | 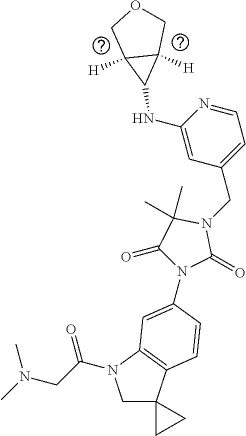 | 1-[(2-{(1R,5S,6r)-3-oxabicyclo[3.1.0]hex-6- ylamino}-4-pyridyl)methyl]-3-{1′-[2- (dimethylamino)acetyl]spiro[cyclopropane-1,3′- indolin]-6′-yl}-5,5-dimethyl-2,4- imidazolidinedione |
| 127 | 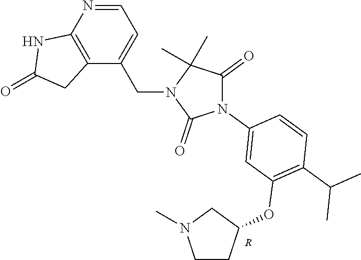 | 3-{3-[(R)-1-methyl-3-pyrrolidinyloxy]-4- cumenyl}-5,5-dimethyl-1-[(2-oxo-1,7-diaza-4- indanyl)methyl]-2,4-imidazolidinedione |
| 128 | 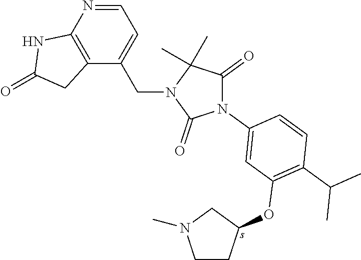 [SMILES: CC(C)c1ccc(N2C(=O)N(Cc3ccnc4c3CC(=O)N4)C(C)(C)C2=O)cc1O[C@H]1CCN(C)C1] | 3-{3-[(S)-1-methyl-3-pyrrolidinyloxy]-4- cumenyl}-5,5-dimethyl-1-[(2-oxo-1,7-diaza-4- indanyl)methyl]-2,4-imidazolidinedione |
| 129 | 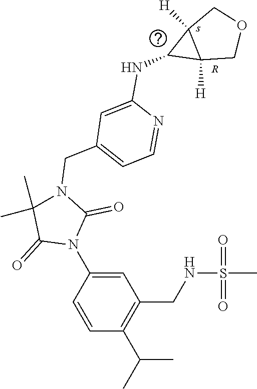 | 1-[(2-{(1R,5S,6r)-3-oxabicyclo[3.1.0]hex-6- ylamino}-4-pyridyl)methyl]-3-{3- [(mesylamino)methyl]-4-cumenyl}-5,5- dimethyl-2,4-imidazolidinedione |
| 130 | 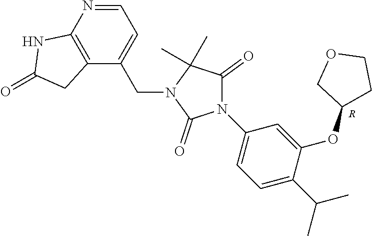 | 3-{3-[(R)-tetrahydro-3-furyloxy]-4-cumenyl}- 5,5-dimethyl-1-[(2-oxo-1,7-diaza-4- indanyl)methyl]-2,4-imidazolidinedione |
| 131 | 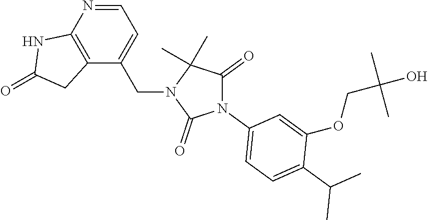 [SMILES: CC(C)c1ccc(N2C(=O)N(Cc3ccnc4c3CC(=O)N4)C(C)(C)C2=O)cc1OCC(C)(C)O] | 3-[3-(2-hydroxy-2-methylpropoxy)-4-cumenyl]- 5,5-dimethyl-1-[(2-oxo-1,7-diaza-4- indanyl)methyl]-2,4-imidazolidinedione |
| 132 | 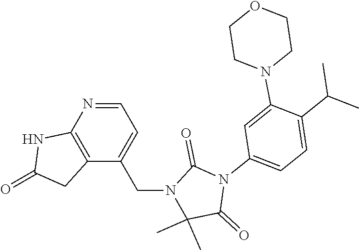 [SMILES: CC(C)c1ccc(N2C(=O)N(Cc3ccnc4c3CC(=O)N4)C(C)(C)C2=O)cc1N1CCOCC1] | 5,5-dimethyl-3-(3-morpholino-4-cumenyl)-1-[(2- oxo-1,7-diaza-4-indanyl)methyl]-2,4- imidazolidinedione |
| 133 | 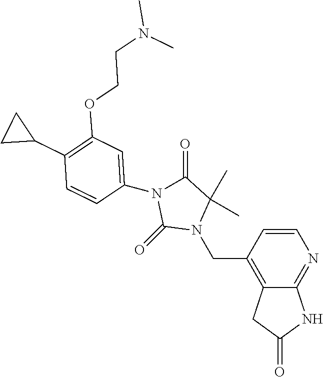 [SMILES: CN(C)CCOc1cc(N2C(=O)N(Cc3ccnc4c3CC(=O)N4)C(C)(C)C2=O)ccc1C1CC1] | 3-{4-cyclopropyl-3-[2- (dimethylamino)ethoxy]phenyl}-5,5-dimethyl-1- [(2-oxo-1,7-diaza-4-indanyl)methyl]-2,4- imidazolidinedione |
| 134 | 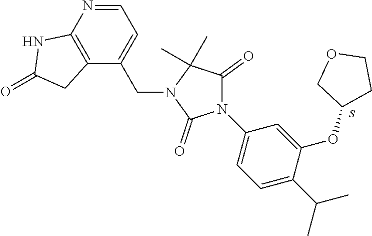 | 3-{3-[(S)-tetrahydro-3-furyloxy]-4-cumenyl}- 5,5-dimethyl-1-[(2-oxo-1,7-diaza-4- indanyl)methyl]-2,4-imidazolidinedione |
| 135 | 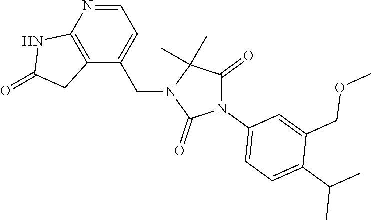 [SMILES: COCc1cc(N2C(=O)N(Cc3ccnc4c3CC(=O)N4)C(C)(C)C2=O)ccc1C(C)C] | 3-[3-(methoxymethyl)-4-cumenyl]-5,5-dimethyl- 1-[(2-oxo-1,7-diaza-4-indanyl)methyl]-2,4- imidazolidinedione |
| 136 | 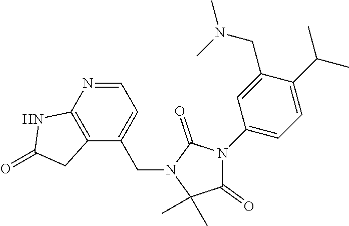 [SMILES: CC(C)c1ccc(N2C(=O)N(Cc3ccnc4c3CC(=O)N4)C(C)(C)C2=O)cc1CN(C)C] | 3-{3-[(dimethylamino)methyl]-4-cumenyl}-5,5- dimethyl-1-[(2-oxo-1,7-diaza-4-indanyl)methyl]- 2,4-imidazolidinedione |
| 137 | 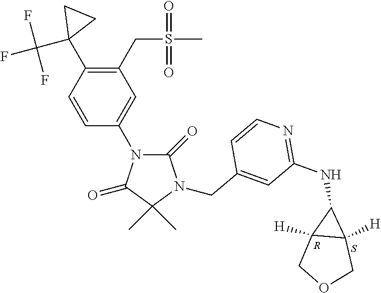 | 1-[(2-{(1R,5S,6r)-3-oxabicyclo[3.1.0]hex-6- ylamino}-4-pyridyl)methyl]-3-{3- (mesylmethyl)-4-[1- (trifluoromethyl)cyclopropyl]phenyl}-5,5- dimethyl-2,4-imidazolidinedione |
| 138 | 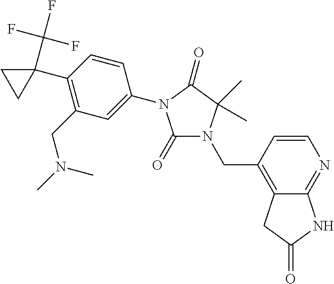 [SMILES: CN(C)Cc1cc(N2C(=O)N(Cc3ccnc4c3CC(=O)N4)C(C)(C)C2=O)ccc1C1(C(F)(F)F)CC1] | 3-{3-[(dimethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione |
| 139 | 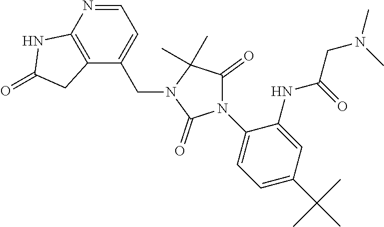 [SMILES: CN(C)CC(=O)Nc1cc(C(C)(C)C)ccc1N1C(=O)N(Cc2ccnc3c2CC(=O)N3)C(C)(C)C1=O] | N-[5-(tert-butyl)-2-{4,4-dimethyl-2,5-dioxo-3- [(2-oxo-1,7-diaza-4-indanyl)methyl]-1- imidazolidinyl}phenyl](dimethylamino)acetamide |
| 140 | 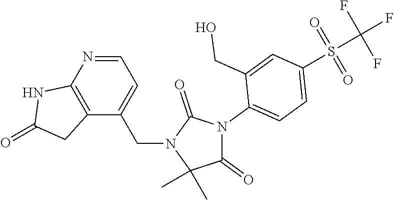 [SMILES: CC1(C)C(=O)N(c2ccc(S(=O)(=O)C(F)(F)F)cc2CO)C(=O)N1Cc1ccnc2c1CC(=O)N2] | 3-[2-(hydroxymethyl)-4-(trifluoromesyl)phenyl]- 5,5-dimethyl-1-[(2-oxo-1,7-diaza-4- indanyl)methyl]-2,4-imidazolidinedione |
| 141 | 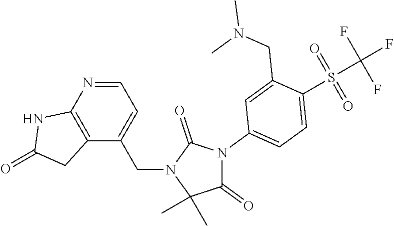 [SMILES: CN(C)Cc1cc(N2C(=O)N(Cc3ccnc4c3CC(=O)N4)C(C)(C)C2=O)ccc1S(=O)(=O)C(F)(F)F] | 3-{3-[(dimethylamino)methyl]-4- (trifluoromesyl)phenyl}-5,5-dimethyl-1-[(2-oxo- 1,7-diaza-4-indanyl)methyl]-2,4- imidazolidinedione |
| 142 | 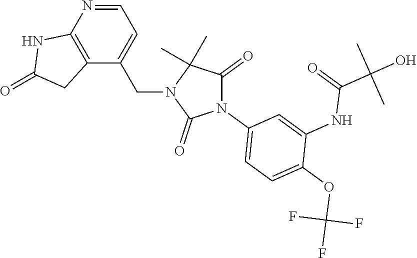 [SMILES: CC(C)(O)C(=O)Nc1cc(N2C(=O)N(Cc3ccnc4c3CC(=O)N4)C(C)(C)C2=O)ccc1OC(F)(F)F] | N-(5-{4,4-dimethyl-2,5-dioxo-3-[(2-oxo-1,7- diaza-4-indanyl)methyl]-1-imidazolidinyl}-2- trifluoromethoxyphenyl)2-hydroxy-2- methylpropionamide |
| 143 | 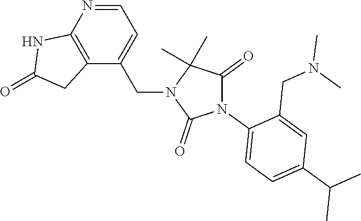 [SMILES: CC(C)c1ccc(N2C(=O)N(Cc3ccnc4c3CC(=O)N4)C(C)(C)C2=O)c(CN(C)C)c1] | 3-{2-[(dimethylamino)methyl]-4-cumenyl}-5,5- dimethyl-1-[(2-oxo-1,7-diaza-4-indanyl)methyl]- 2,4-imidazolidinedione |
| 144 | 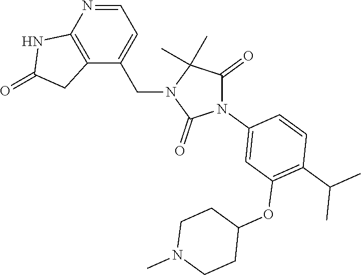 [SMILES: CC(C)c1ccc(N2C(=O)N(Cc3ccnc4c3CC(=O)N4)C(C)(C)C2=O)cc1OC1CCN(C)CC1] | 5,5-dimethyl-3-[3-(1-methyl-4-piperidyloxy)-4- cumenyl]-1-[(2-oxo-1,7-diaza-4- indanyl)methyl]-2,4-imidazolidinedione |
| 145 | 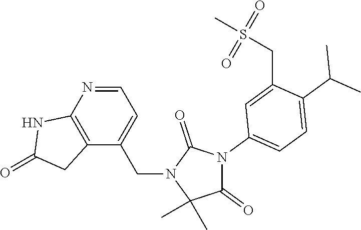 [SMILES: CC(C)c1ccc(N2C(=O)N(Cc3ccnc4c3CC(=O)N4)C(C)(C)C2=O)cc1CS(C)(=O)=O] | 3-[3-(mesylmethyl)-4-cumenyl]-5,5-dimethyl-1- [(2-oxo-1,7-diaza-4-indanyl)methyl]-2,4- imidazolidinedione |
| 146 | 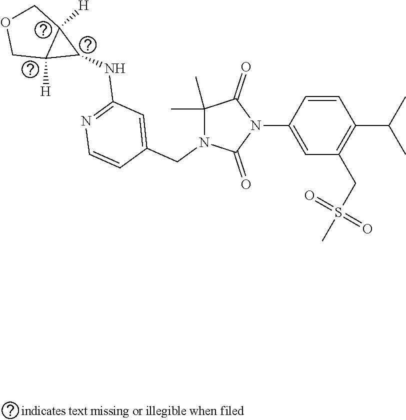 [SMILES: CC(C)c1ccc(N2C(=O)N(Cc3ccnc(N[C@H]4[C@@H]5COC[C@@H]54)c3)C(C)(C)C2=O)cc1CS(C)(=O)=O] | 1-[(2-{(1R,5S,6r)-3-oxabicyclo[3.1.0]hex-6- ylamino}-4-pyridyl)methyl]-3-[3-(mesylmethyl)- 4-cumenyl]-5,5-dimethyl-2,4-imidazolidinedione |
| 147 | 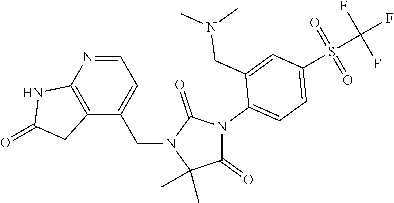 [SMILES: CN(C)Cc1cc(S(=O)(=O)C(F)(F)F)ccc1N1C(=O)N(Cc2ccnc3c2CC(=O)N3)C(C)(C)C1=O] | 3-{2-[(dimethylamino)methyl]-4- (trifluoromesyl)phenyl}-5,5-dimethyl-1-[(2-oxo- 1,7-diaza-4-indanyl)methyl]-2,4-imidazolidinedione |
| 148 | 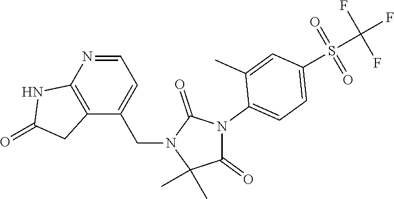 [SMILES: Cc1cc(S(=O)(=O)C(F)(F)F)ccc1N1C(=O)N(Cc2ccnc3c2CC(=O)N3)C(C)(C)C1=O] | 5,5-dimethyl-1-[(2-oxo-1,7-diaza-4- indanyl)methyl]-3-[4-(trifluoromesyl)-2-tolyl]- 2,4-imidazolidinedione |
| 149 | 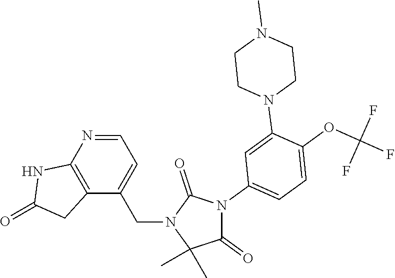 [SMILES: CN1CCN(c2cc(N3C(=O)N(Cc4ccnc5c4CC(=O)N5)C(C)(C)C3=O)ccc2OC(F)(F)F)CC1] | 5,5-dimethyl-3-[3-(4-methyl-1-piperazinyl)-4- trifluoromethoxyphenyl]-1-[(2-oxo-1,7-diaza-4- indanyl)methyl]-2,4-imidazolidinedione |
| 150 | 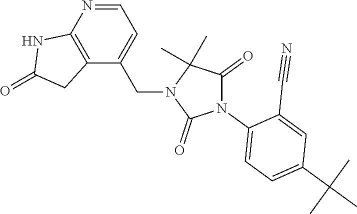 [SMILES: CC(C)(C)c1ccc(N2C(=O)N(Cc3ccnc4c3CC(=O)N4)C(C)(C)C2=O)c(C#N)c1] | 5-(tert-butyl)-2-{4,4-dimethyl-2,5-dioxo-3-[(2- oxo-1,7-diaza-4-indanyl)methyl]-1- imidazolidinyl}benzonitrile |
| 151 | 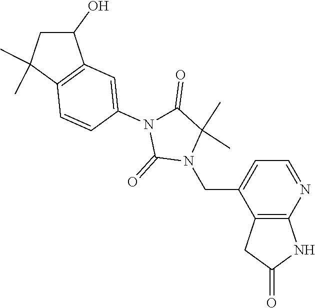 [SMILES: CC1(C)CC(O)c2cc(N3C(=O)N(Cc4ccnc5c4CC(=O)N5)C(C)(C)C3=O)ccc21] | 3-(3-hydroxy-1,1-dimethyl-5-indanyl)-5,5- dimethyl-1-[(2-oxo-1,7-diaza-4-indanyl)methyl]- 2,4-imidazolidinedione |
| 152 | 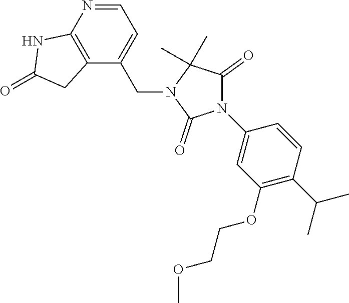 [SMILES: COCCOc1cc(N2C(=O)N(Cc3ccnc4c3CC(=O)N4)C(C)(C)C2=O)ccc1C(C)C] | 3-[3-(2-methoxyethoxy)-4-cumenyl]-5,5- dimethyl-1-[(2-oxo-1,7-diaza-4-indanyl)methyl]- 2,4-imidazolidinedione |
| 153 | 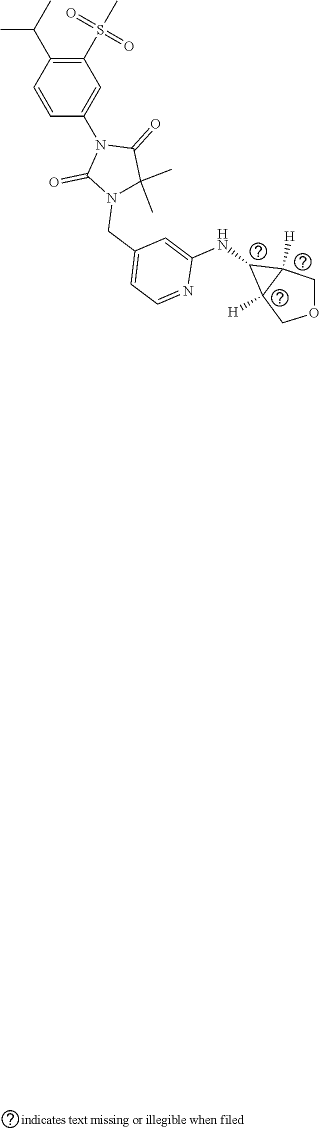 [SMILES: CC(C)c1ccc(N2C(=O)N(Cc3ccnc(N[C@H]4[C@@H]5COC[C@@H]54)c3)C(C)(C)C2=O)cc1S(C)(=O)=O] | 1-[(2-{(1R,5S,6r)-3-oxabicyclo[3.1.0]hex-6- ylamino}-4-pyridyl)methyl]-3-(3-mesyl-4- cumenyl)-5,5-dimethyl-2,4-imidazolidinedione |
| 154 | 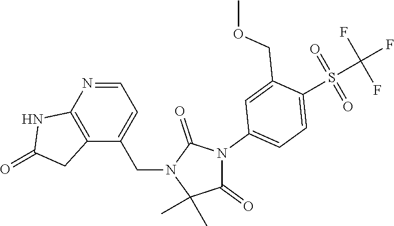 [SMILES: COCc1cc(N2C(=O)N(Cc3ccnc4c3CC(=O)N4)C(C)(C)C2=O)ccc1S(=O)(=O)C(F)(F)F] | 3-[3-(methoxymethyl)-4- (trifluoromesyl)phenyl]-5,5-dimethyl-1-[(2-oxo- 1,7-diaza-4-indanyl)methyl]-2,4- imidazolidinedione |
| 155 | 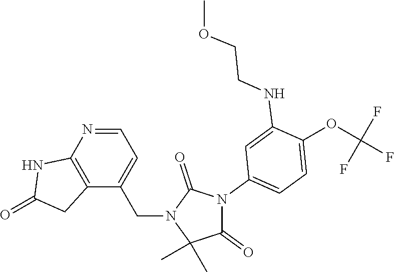 [SMILES: COCCNc1cc(N2C(=O)N(Cc3ccnc4c3CC(=O)N4)C(C)(C)C2=O)ccc1OC(F)(F)F] | 3-[3-(2-methoxyethylamino)-4- trifluoromethoxyphenyl]-5,5-dimethyl-1-[(2-oxo- 1,7-diaza-4-indanyl)methyl]-2,4- imidazolidinedione |
| 156 | 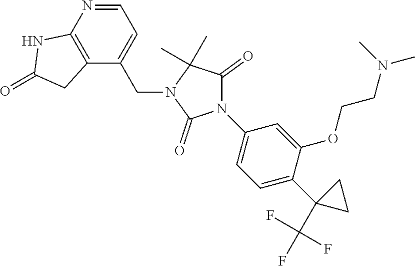 [SMILES: CN(C)CCOc1cc(N2C(=O)N(Cc3ccnc4c3CC(=O)N4)C(C)(C)C2=O)ccc1C1(C(F)(F)F)CC1] | 3-{3-[2-(dimethylamino)ethoxy]-4-[1- (trifluoromethyl)cyclopropyl]phenyl}-5,5- dimethyl-1-[(2-oxo-1,7-diaza-4-indanyl)methyl]- 2,4-imidazolidinedione |
| 157 | 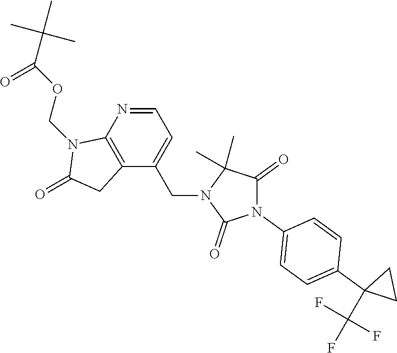 [SMILES: CC(C)(C)C(=O)OCN1C(=O)Cc2c(CN3C(=O)N(c4ccc(C5(C(F)(F)F)CC5)cc4)C(=O)C3(C)C)ccnc21] | {4-[(5,5-dimethyl-2,4-dioxo-3-{p-[1- (trifluoromethyl)cyclopropyl]phenyl}-1- imidazolidinyl)methyl]-2-oxo-1,7-diaza-1- indanyl)methyl] 2,2-dimethylpropionate |
| 158 | 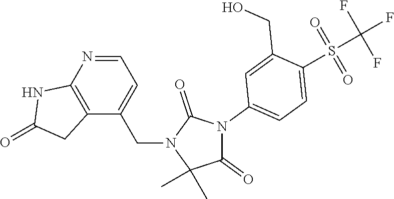 [SMILES: CC1(C)C(=O)N(c2ccc(S(=O)(=O)C(F)(F)F)c(CO)c2)C(=O)N1Cc1ccnc2c1CC(=O)N2] | 3-[3-(hydroxymethyl)-4-(trifluoromesyl)phenyl]- 5,5-dimethyl-1-[(2-oxo-1,7-diaza-4- indanyl)methyl]-2,4-imidazolidinedione |
| 159 | 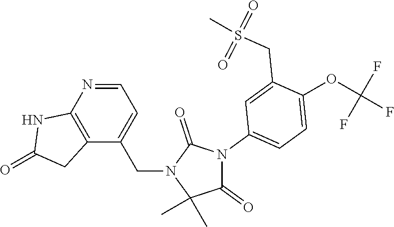 [SMILES: CC1(C)C(=O)N(c2ccc(OC(F)(F)F)c(CS(C)(=O)=O)c2)C(=O)N1Cc1ccnc2c1CC(=O)N2] | 3-[3-(mesylmethyl)-4-trifluoromethoxyphenyl]- 5,5-dimethyl-1-[(2-oxo-1,7-diaza-4- indanyl)methyl]-2,4-imidazolidinedione |
| 160 | 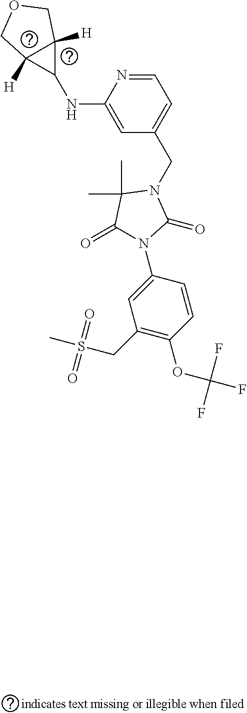 [SMILES: CC1(C)C(=O)N(c2ccc(OC(F)(F)F)c(CS(C)(=O)=O)c2)C(=O)N1Cc1ccnc(NC2[C@H]3COC[C@@H]23)c1] | 1-[(2-{(1R,5S)-3-oxabicyclo[3.1.0]hex-6- ylamino}-4-pyridyl)methyl]-3-[3-(mesylmethyl)- 4-trifluoromethoxyphenyl]-5,5-dimethyl-2,4- imidazolidinedione |
| 161 | 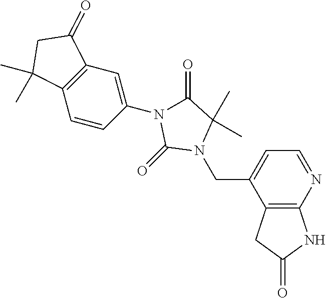 [SMILES: CC1(C)CC(=O)c2cc(N3C(=O)N(Cc4ccnc5c4CC(=O)N5)C(C)(C)C3=O)ccc21] | 3-(1,1-dimethyl-3-oxo-5-indanyl)-5,5-dimethyl- 1-[(2-oxo-1,7-diaza-4-indanyl)methyl]-2,4- imidazolidinedione |
| 162 | 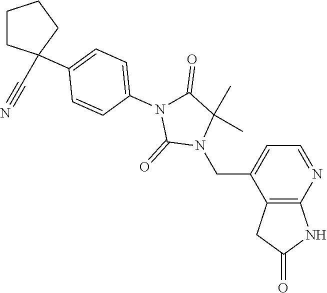 [SMILES: CC1(C)C(=O)N(c2ccc(C3(C#N)CCCC3)cc2)C(=O)N1Cc1ccnc2c1CC(=O)N2] | 1-(p-{4,4-dimethyl-2,5-dioxo-3-[(2-oxo-1,7- diaza-4-indanyl)methyl]-1- imidazolidinyl}phenyl)cyclopentanecarbonitrile |
| 163 | 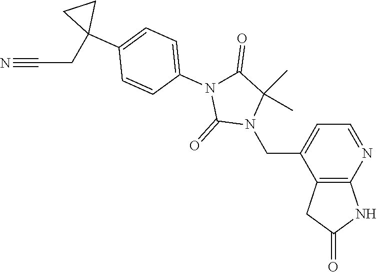 [SMILES: CC1(C)C(=O)N(c2ccc(C3(CC#N)CC3)cc2)C(=O)N1Cc1ccnc2c1CC(=O)N2] | [1-(p-{4,4-dimethyl-2,5-dioxo-3-[(2-oxo-1,7- diaza-4-indanyl)methyl]-1- imidazolidinyl}phenyl)cyclopropyl]acetonitrile |
| 164 | 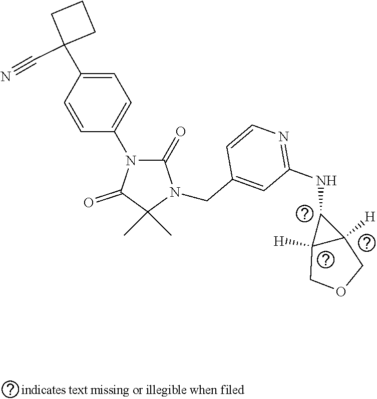 [SMILES: CC1(C)C(=O)N(c2ccc(C3(C#N)CCC3)cc2)C(=O)N1Cc1ccnc(N[C@H]2[C@@H]3COC[C@@H]32)c1] | 1-(p-{3-[(2-{(1R,5S,6r)-3-oxabicyclo[3.1.0]hex- 6-ylamino}-4-pyridyl)methyl]-4,4-dimethyl-2,5- dioxo-1- imidazolidinyl}phenyl)cyclobutanecarbonitrile |
| 165 | 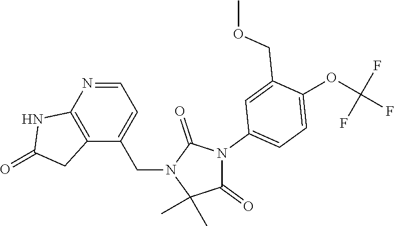 [SMILES: COCc1cc(N2C(=O)N(Cc3ccnc4c3CC(=O)N4)C(C)(C)C2=O)ccc1OC(F)(F)F] | 3-[3-(methoxymethyl)-4- trifluoromethoxyphenyl]-5,5-dimethyl-1-[(2-oxo- 1,7-diaza-4-indanyl)methyl]-2,4- imidazolidinedione |
| 166 | 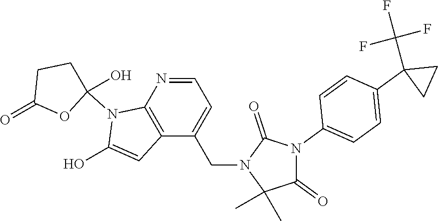 [SMILES: CC1(C)C(=O)N(c2ccc(C3(C(F)(F)F)CC3)cc2)C(=O)N1Cc1ccnc2c1cc(O)n2C1(O)CCC(=O)O1] | 1-{[2-hydroxy-1-(5-hydroxy-2-oxo-4,5-dihydro- 5-furyl)-1H-1,7-diazainden-4-yl]methyl}-5,5- dimethyl-3-{p-[1- (trifluoromethyl)cyclopropyl]phenyl}-2,4- imidazolidinedione |
| 167 | 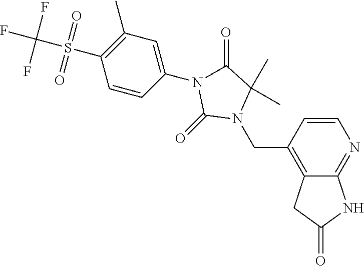 [SMILES: Cc1cc(N2C(=O)N(Cc3ccnc4c3CC(=O)N4)C(C)(C)C2=O)ccc1S(=O)(=O)C(F)(F)F] | 5,5-dimethyl-1-[(2-oxo-1,7-diaza-4- indanyl)methyl]-3-[4-(trifluoromesyl)-3-tolyl]- 2,4-imidazolidinedione |
| 168 | 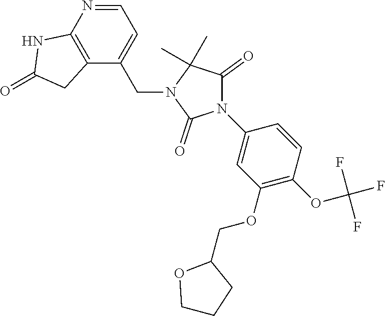 [SMILES: CC1(C)C(=O)N(c2ccc(OC(F)(F)F)c(OCC3CCCO3)c2)C(=O)N1Cc1ccnc2c1CC(=O)N2] | 5,5-dimethyl-1-[(2-oxo-1,7-diaza-4- indanyl)methyl]-3-{3-[(tetrahydro-2- furyl)methoxy]-4-trifluoromethoxyphenyl}-2,4- imidazolidinedione |
| 169 | 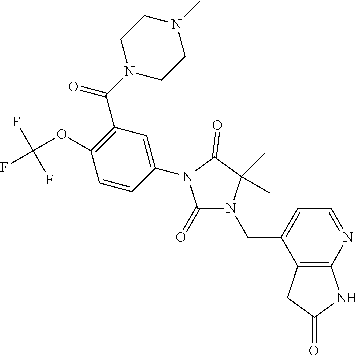 [SMILES: CN1CCN(C(=O)c2cc(N3C(=O)N(Cc4ccnc5c4CC(=O)N5)C(C)(C)C3=O)ccc2OC(F)(F)F)CC1] | 5,5-dimethyl-3-{3-[(4-methyl-1- piperazinyl)carbonyl]-4- trifluoromethoxyphenyl}-1-[(2-oxo-1,7-diaza-4- indanyl)methyl]-2,4-imidazolidinedione |
| 170 | 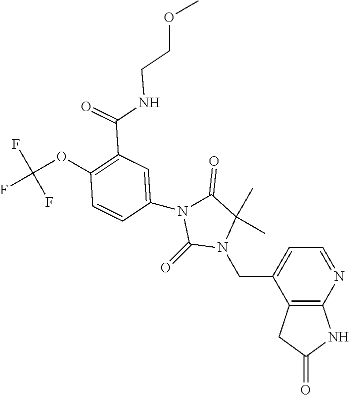 [SMILES: COCCNC(=O)c1cc(N2C(=O)N(Cc3ccnc4c3CC(=O)N4)C(C)(C)C2=O)ccc1OC(F)(F)F] | N-2-methoxyethyl5-{4,4-dimethyl-2,5-dioxo-3- [(2-oxo-1,7-diaza-4-indanyl)methyl]-1- imidazolidinyl}-2-trifluoroanisamide |
| 171 | 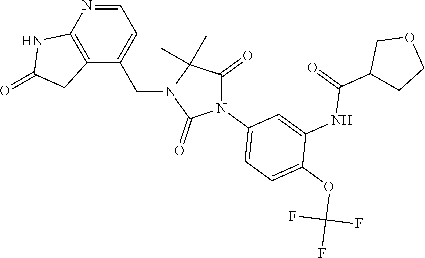 [SMILES: CC1(C)C(=O)N(c2ccc(OC(F)(F)F)c(NC(=O)C3CCOC3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-3-furamide |
| 172 | 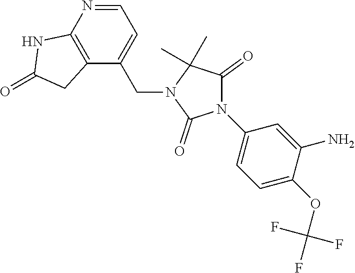 [SMILES: CC1(C)C(=O)N(c2ccc(OC(F)(F)F)c(N)c2)C(=O)N1Cc1ccnc2c1CC(=O)N2] | 3-(3-amino-4-trifluoromethoxyphenyl)-5,5- dimethyl-1-[(2-oxo-1,7-diaza-4-indanyl)methyl]- 2,4-imidazolidinedione |
| 173 | 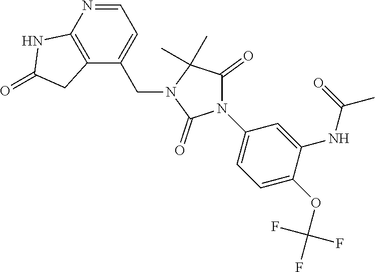 [SMILES: CC(=O)Nc1cc(N2C(=O)N(Cc3ccnc4c3CC(=O)N4)C(C)(C)C2=O)ccc1OC(F)(F)F] | N-(5-{4,4-dimethyl-2,5-dioxo-3-[(2-oxo-1,7- diaza-4-indanyl)methyl]-1-imidazolidinyl}-2- trifluoromethoxyphenyl)acetamide |
| 174 | 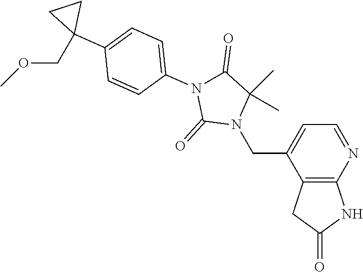 [SMILES: COCC1(c2ccc(N3C(=O)N(Cc4ccnc5c4CC(=O)N5)C(C)(C)C3=O)cc2)CC1] | 3-{p-[1-(methoxymethyl)cyclopropyl]phenyl}- 5,5-dimethyl-1-[(2-oxo-1,7-diaza-4- indanyl)methyl]-2,4-imidazolidinedione |
| 175 | 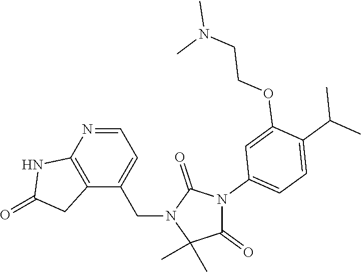 [SMILES: CC(C)c1ccc(N2C(=O)N(Cc3ccnc4c3CC(=O)N4)C(C)(C)C2=O)cc1OCCN(C)C] | 3-{3-[2-(dimethylamino)ethoxy]-4-cumenyl}- 5,5-dimethyl-1-[(2-oxo-1,7-diaza-4- indanyl)methyl]-2,4-imidazolidinedione |
| 176 | 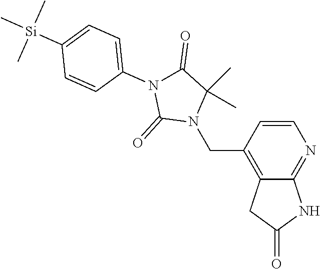 [SMILES: CC1(C)C(=O)N(c2ccc([Si](C)(C)C)cc2)C(=O)N1Cc1ccnc2c1CC(=O)N2] | 5,5-dimethyl-1-[(2-oxo-1,7-diaza-4- indanyl)methyl]-3-[p-(trimethylsilyl)phenyl]-2,4- imidazolidinedione |
| 177 | 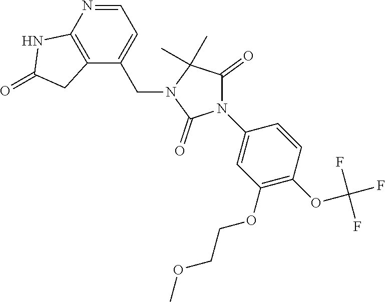 [SMILES: COCCOc1cc(N2C(=O)N(Cc3ccnc4c3CC(=O)N4)C(C)(C)C2=O)ccc1OC(F)(F)F] | 3-[3-(2-methoxyethoxy)-4- trifluoromethoxyphenyl]-5,5-dimethyl-1-[(2-oxo- 1,7-diaza-4-indanyl)methyl]-2,4- imidazolidinedione |
| 178 | 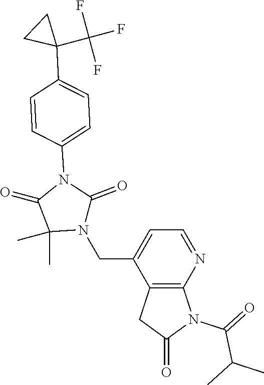 [SMILES: CC(C)C(=O)N1C(=O)Cc2c(CN3C(=O)N(c4ccc(C5(C(F)(F)F)CC5)cc4)C(=O)C3(C)C)ccnc21] | 1-[(1-isobutyryl-2-oxo-1,7-diaza-4- indanyl)methyl]-5,5-dimethyl-3-{p-[1- (trifluoromethyl)cyclopropyl]phenyl}-2,4- imidazolidinedione |
| 179 | 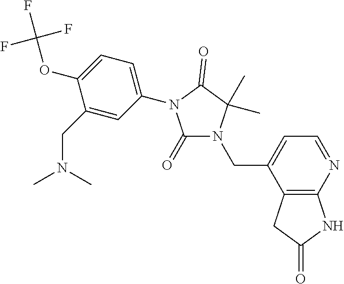 [SMILES: CN(C)Cc1cc(N2C(=O)N(Cc3ccnc4c3CC(=O)N4)C(C)(C)C2=O)ccc1OC(F)(F)F] | 3-{3-[(dimethylamino)methyl]-4- trifluoromethoxyphenyl}-5,5-dimethyl-1-[(2- oxo-1,7-diaza-4-indanyl)methyl]-2,4- imidazolidinedione |
| 180 | 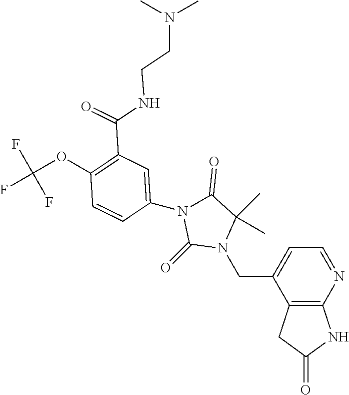 [SMILES: CN(C)CCNC(=O)c1cc(N2C(=O)N(Cc3ccnc4c3CC(=O)N4)C(C)(C)C2=O)ccc1OC(F)(F)F] | N-2-(dimethylamino)ethyl5-{4,4-dimethyl-2,5- dioxo-3-[(2-oxo-1,7-diaza-4-indanyl)methyl]-1- imidazolidinyl}-2-trifluoroanisamide |
| 181 | 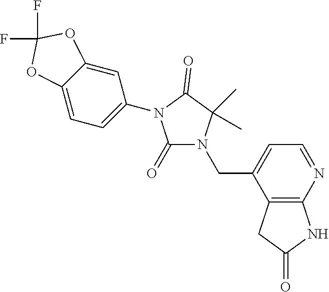 [SMILES: CC1(C)C(=O)N(c2ccc3c(c2)OC(F)(F)O3)C(=O)N1Cc1ccnc2c1CC(=O)N2] | 3-(2,2-difluoro-2H-1,3-benzodioxol-5-yl)-5,5- dimethyl-1-[(2-oxo-1,7-diaza-4-indanyl)methyl]- 2,4-imidazolidinedione |
| 182 | 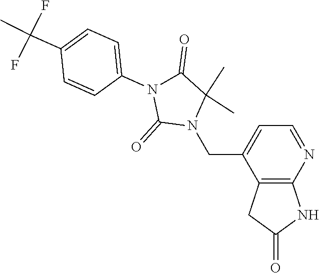 [SMILES: CC(F)(F)c1ccc(N2C(=O)N(Cc3ccnc4c3CC(=O)N4)C(C)(C)C2=O)cc1] | 3-[p-(1,1-difluoroethyl)phenyl]-5,5-dimethyl-1- [(2-oxo-1,7-diaza-4-indanyl)methyl]-2,4- imidazolidinedione |
| 183 | 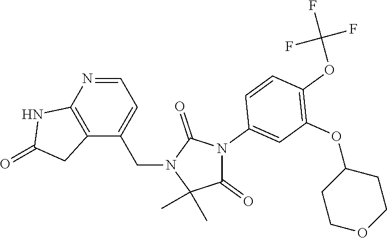 [SMILES: CC1(C)C(=O)N(c2ccc(OC(F)(F)F)c(OC3CCOCC3)c2)C(=O)N1Cc1ccnc2c1CC(=O)N2] | 5,5-dimethyl-1-[(2-oxo-1,7-diaza-4- indanyl)methyl]-3-[3-(tetrahydro-2H-pyran-4- yloxy)-4-trifluoromethoxyphenyl]-2,4- imidazolidinedione |
| 184 | 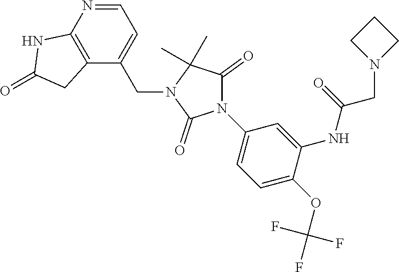 [SMILES: CC1(C)C(=O)N(c2ccc(OC(F)(F)F)c(NC(=O)CN3CCC3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)(1-azetidinyl)acetamide |
| 185 | 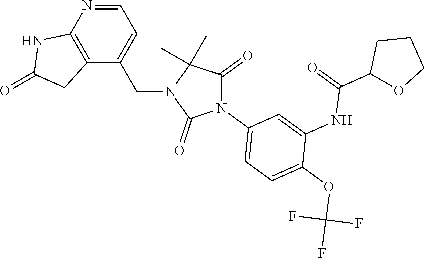 [SMILES: CC1(C)C(=O)N(c2ccc(OC(F)(F)F)c(NC(=O)C3CCCO3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2-furamide |
| 186 | 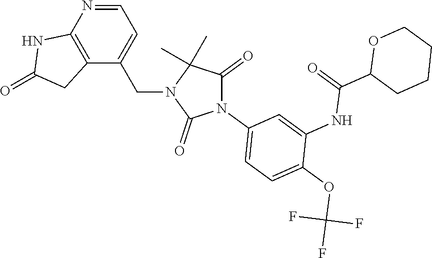 [SMILES: CC1(C)C(=O)N(c2ccc(OC(F)(F)F)c(NC(=O)C3CCCCO3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2H-pyran-2- carboxamide |
| 187 | 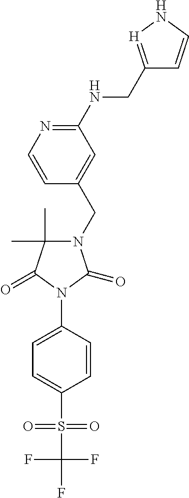 | 5,5-dimethyl-1-[(2-{[(3- pyrazolyl)methyl]amino}-4-pyridyl)methyl]-3- [p-(trifluoromesyl)phenyl]-2,4- imidazolidinedione |
| 188 | 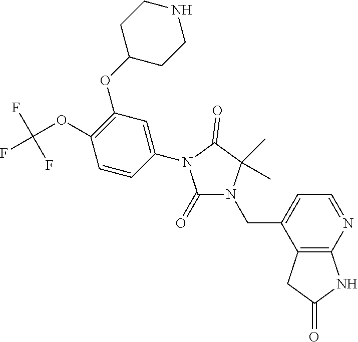 [SMILES: CC1(C)C(=O)N(c2ccc(OC(F)(F)F)c(OC3CCNCC3)c2)C(=O)N1Cc1ccnc2c1CC(=O)N2] | 5,5-dimethyl-1-[(2-oxo-1,7-diaza-4- indanyl)methyl]-3-[3-(4-piperidyloxy)-4- trifluoromethoxyphenyl]-2,4-imidazolidinedione |
| 189 | 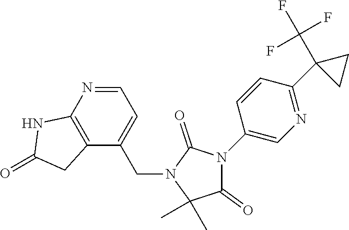 [SMILES: CC1(C)C(=O)N(c2ccc(C3(C(F)(F)F)CC3)nc2)C(=O)N1Cc1ccnc2c1CC(=O)N2] | 5,5-dimethyl-1-[(2-oxo-1,7-diaza-4- indanyl)methyl]-3-{6-[1- (trifluoromethyl)cyclopropyl]-3-pyridyl}-2,4- imidazolidinedione |
| 190 | 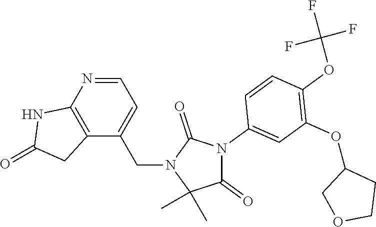 [SMILES: CC1(C)C(=O)N(c2ccc(OC(F)(F)F)c(OC3CCOC3)c2)C(=O)N1Cc1ccnc2c1CC(=O)N2] | 5,5-dimethyl-1-[(2-oxo-1,7-diaza-4- indanyl)methyl]-3-[3-(tetrahydro-3-furyloxy)-4- trifluoromethoxyphenyl]-2,4-imidazolidinedione |
| 191 | 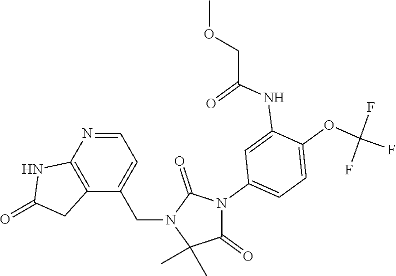 [SMILES: COCC(=O)Nc1cc(N2C(=O)N(Cc3ccnc4c3CC(=O)N4)C(C)(C)C2=O)ccc1OC(F)(F)F] | N-(5-{4,4-dimethyl-2,5-dioxo-3-[(2-oxo-1,7- diaza-4-indanyl)methyl]-1-imidazolidinyl}-2- trifluoromethoxyphenyl)methoxyacetamide |

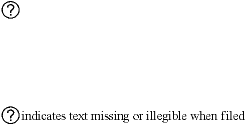

Provided herein as Embodiment 29 is the compound or salt of Embodiment 28, wherein the compound is 1-[(5-{4,4-dimethyl-2,5-dioxo-3-[(2-oxo-1,7-diaza-4-indanyl)methyl]-1-imidazolidinyl}-2-[1-(trifluoromethyl)cyclopropyl]phenyl)methyl]-3-azetidinecarbonitrile, 3-(1,1-dimethyl-1,3-dihydro-5-isobenzofuranyl)-5,5-dimethyl-1-[(2-oxo-1,7-diaza-4-indanyl)methyl]-2,4-imidazolidinedione, 1-[(2-{(1R,5S)-3-oxabicyclo[3.1.0]hex-6-ylamino}-4-pyridyl)methyl]-3-[3-(mesylmethyl)-4-trifluoromethoxyphenyl]-5,5-dimethyl-2,4-imidazolidinedione, 1-[(2-{(1R,5S,6r)-3-oxabicyclo[3.1.0]hex-6-ylamino}-4-pyridyl)methyl]-3-{3-mesyl-4-[1-(trifluoromethyl)cyclopropyl]phenyl}-5,5-dimethyl-2,4-imidazolidinedione, or 3-[(S)-3-(dimethylamino)-1,1-dimethyl-5-indanyl]-5,5-dimethyl-1-[(2-oxo-1,7-diaza-4-indanyl)methyl]-2,4-imidazolidinedione.

It is understood that selections of values of each variable are those that result in the formation of stable or chemically feasible compounds.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the present disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of the structure. If the stereochemistry of a structure or a portion of a structure is indicated with, for example, bold or dashed lines, the structure or portion of the structure is to be interpreted as encompassing only the stereoisomer indicated, unless otherwise noted. For example,

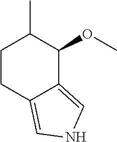

represents

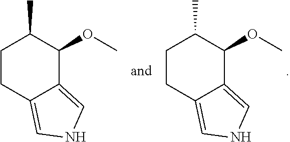

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole. A bond drawn with a wavy line may be used to indicate that both stereoisomers are encompassed. This is not to be confused with a wavy line drawn perpendicular to a bond which indicates the point of attachment of a group to the rest of the molecule.

The term “stereoisomer” or “stereoisomerically pure” compound refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (WileyInterscience, New York, 1981); Wilen et al., Tetrahedron 33:2725; Eliel, Stereochemistry of Carbon Compounds (McGrawHill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example,

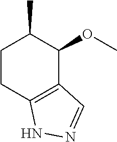

represents

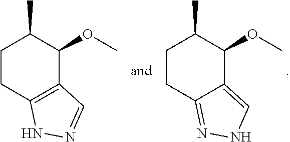

Similarly, for example, the chemical name (4R,5R)-4-methoxy-5-methyl-4,5,6,7-tetrahydro-1H-indazole represents (4R,5R)-4-methoxy-5-methyl-4,5,6,7-tetrahydro-1H-indazole and (4R,5R)-4-methoxy-5-methyl-4,5,6,7-tetrahydro-2H-indazole. Accordingly, the scope of the present disclosure is to be understood to encompass all tautomeric forms of the compounds disclosed herein.

### Isotopically-Labeled Compounds

In some cases, the scope of the present disclosure includes pharmaceutically acceptable isotopically-labelled compounds of the compounds disclosed herein, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of the compounds disclosed herein, such as those incorporating a radioactive isotope, are useful in drug and/or substrate tissue distribution studies. The radioactive isotopes tritium (H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. As such, the term “deuterated” refers to the substitution of one or more hydrogen atoms with one or more deuterium atoms on a particular structure or functional group. Substitution with positron emitting isotopes, such as ¹¹C, ¹⁸F, ¹⁵O and ¹³N, can be useful in Positron Emission Topography (PET) studies, for example, for examining target occupancy. Isotopically labelled compounds of the compounds disclosed herein can generally be prepared by conventional techniques known to those skilled in the art or by processes analogous to those described in the accompanying GENERAL SYNTHETIC PROCEDURES and EXAMPLES sections using an appropriate isotopically-labelled reagent in place of the non-labelled reagent previously employed.

### Biological Activity

In some cases, the compounds or salts disclosed herein (such as compounds of Formulae (I), (IA), (IB), (IC), (ID), (ID-1), (ID-2), (ID-3) and (ID-4) or compounds of Embodiments 1 to 29, or a pharmaceutically acceptable salt of any of the foregoing), have an IC₅₀ value of less than 7 μM, or less than 6 μM, or less than 5 μM, or less than 4 μM, or less than 3 μM, or less than 2 μM, or less than 1 μM, or less than 0.9 μM, or less than 0.7 μM, or less than 0.6 μM, or less than 0.5 μM, or less than 0.4 μM, or less than 0.3 μM, or less than 0.2 μM, or less than 0.1 μM, or less than 0.09 μM, or less than 0.08 μM, or less than 0.07 μM, or less than 0.06 μM, or less than 0.05 μM, or less than 0.04 μM, or less than 0.03 μM, or less than 0.02 μM, or less than 0.01 μM in the assay described in “SECTION 3: Biochemical and Cellular Assays.”

### Formulation and Route of Administration

While it may be possible to administer a compound disclosed herein alone in the uses described, the compound administered normally will be present as an active ingredient in a pharmaceutical composition. Thus, further provided herein is a pharmaceutical composition comprising a compound or salt disclosed herein (such as compounds of Formulae (I), (IA), (IB), (IC), (ID), (ID-1), (ID-2), (ID-3) and (ID-4) or compounds of Table lor a pharmaceutically acceptable salt of any of the foregoing), in combination with one or more pharmaceutically acceptable excipients and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In some cases, the pharmaceutical composition described herein comprises a therapeutically effective amount of a compound disclosed herein, or a pharmaceutically acceptable salt thereof.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, opthalmically, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. In some cases, the pharmaceutical composition is made in the form of a dosage unit containing a particular amount of the active ingredient.

Thus, a further aspect of the disclosure is a pharmaceutical composition comprising one or more of the compounds disclosed herein, or a pharmaceutically acceptable salt thereof, and a pharmaceutically acceptable excipient. Further provided herein is a compound of the disclosure, or a pharmaceutically acceptable salt thereof, or the pharmaceutical composition described herein, for use as a medicament.

Provided herein as Embodiment 30 is pharmaceutical composition comprising the compound or salt of any one of Embodiments 1 to 29, and a pharmaceutically acceptable excipient.

### Methods of Use

The compounds described herein can irreversibly or competitively bind to IGF-1R. Without intending to be bound by any particular theory, the compounds of the disclosure can, in some cases, inhibit the activation of IGF-1R, leading to reduction of inflammation, prevention of muscle and fat tissue remodeling and/or stoppage of tumor cell growth. Besides being useful for human treatment, the compounds provided herein may be useful for veterinary treatment of companion animals, exotic animals, and farm animals, including mammals, rodents, and the like. For example, animals including horses, dogs, and cats may be treated with compounds provided herein.

Provided herein are methods of treating a patient. In several embodiments, the method comprises administering a therapeutic amount of a compound or salt disclosed herein (such as compounds of Formulae (I), (IA), (IB), (IC), (ID), (ID-1), (ID-2), (ID-3) and (ID-4) or compounds of Table 1, or a pharmaceutically acceptable salt of any of the foregoing) to a patient.

Also provided herein are methods for treating IGF-1R-mediated disorders in a mammal in need of such treatment comprising administering to the mammal an amount of a compound disclosed herein effective to halt the further progression of, reduce the severity of, undo the effect of, or prevent the return of the disease or disorder in the subject.

Specific diseases to be treated by the compounds, compositions, and methods disclosed herein include thyroid eye disease (TED), also known as thyroid-associated ophthalmopathy (TAO), or Graves' ophthalmopathy or orbitopathy (GO). In some cases, the compounds, compositions, and methods disclosed herein are used to treat TED.

Accordingly, the present disclosure also relates to a method of inhibiting at least one IGF-1R function comprising the step of contacting IGF-1R with a compound as described herein, or a pharmaceutically acceptable salt thereof. The cell phenotype, cell proliferation, activity of IGF-1R, change in biochemical output produced by active IGF-1R, expression of IGF-1R, or binding of IGF-1R with a natural binding partner may be monitored. Such methods may be modes of treatment of disease, biological assays, cellular assays, biochemical assays, or the like.

Also provided herein is a method of treating an IGF-1R-mediated disease comprising administering a therapeutically effective amount of a compound as disclosed herein, or a pharmaceutically acceptable salt thereof, to a patient in need thereof.

### Thyroid Eye Disease (TED)

As used herein, “Thyroid Eye Disease” (TED), “Thyroid-associated Ophthalmopathy” (TAO), “Thyroid Inflammatory Eye Disease (TIED),” “Graves' Ophthalmopathy” (GO) or “Graves' Orbitopathy” (GO) refer to the same disease or disorder. These terms refer to the inflammatory orbital pathology associated with some autoimmune thyroid disorders, most commonly with “Graves' Disease” (GD), but sometimes with other diseases, e.g., Hashimoto's thyroiditis.

The cause of TED is unknown. TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

The annual incidence rate of TED has been estimated at 16 cases per 100,000 women and 2.9 cases per 100,000 men from a study based in one largely rural Minnesota community. There appears to be a female preponderance in which women are affected 2.5-6 times more frequently than men; however, severe cases occur more often in men than in women. In addition, most patients are aged 30-50 years, with severe cases appearing to be more frequent in those older than 50 years. Although most cases of TED do not result in loss of vision, this condition can cause vision-threatening exposure keratopathy, troublesome diplopia (double vision), and compressive dysthyroid optic neuropathy.

TED may precede, coincide with, or follow the systemic complications of dysthyroidism. The ocular manifestations of TED include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences.

Many of the signs and symptoms of TED, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1β, interferon-γ, platelet-derived growth factor, thyroid stimulating hormone (TSH), and insulin-like growth factor 1 (IGF-1).

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. The molecular mechanisms underlying TED remain unclear. It is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate the insulin-like growth factor 1 receptor (IGF-1R) have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R using an antibody antagonist might reduce both TSHR- and IGF-1-dependent signaling and therefore interrupt the pathological activities of autoantibodies acting as agonists on either receptor.

Immunoglobulins that activate IGF-1R signaling have been detected in patients with GD and TED. Furthermore, IGF-1 synergistically enhances the actions of thyrotropin. IGF-1R, a membrane-spanning tyrosine kinase receptor with roles in development and metabolism, also stimulates immune function and thus might be targeted therapeutically in autoimmune diseases. IGF-1R is overexpressed by orbital fibroblasts and by T cells and B cells in persons with GD and TED. It forms a signaling complex with TSHR through which it is transactivated. In vitro studies of orbital fibroblasts and fibrocytes show that IGF-1R-inhibitory antibodies can attenuate the actions of IGF-1, thyrotropin, thyroid-stimulating immunoglobulins, and immunoglobulins isolated from patients with GD and TED. These observations prompted a trial of teprotumumab, a fully human IGF-1R-inhibitory monoclonal antibody, in patients with active, moderate-to-severe TED.

The terms “proptosis” and “exophthalmos” (also known as exophthalmus, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TED (TAO or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TED (TAO or GO) is currently recognized as the most common cause of proptosis in adults. Exophthalmos can be either bilateral, as is often seen in TED (TAO or GO), or unilateral (as is often seen in an orbital tumor).

Measurement of the degree of exophthalmos can be performed using an exophthalmometer, an instrument used for measuring the degree of forward displacement of the eye. The device allows measurement of the forward distance of the lateral orbital rim to the front of the cornea.

Computed tomography (CT) scanning and Magnetic resonance imaging (MRI) may also be used in evaluating the degree of exophthalmos or proptosis. CT scanning is an excellent imaging modality for the diagnosis of TED (TAO or GO). In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit.

Orbital ultrasonography can also be used for the diagnosis and evaluation of TED (TAO or GO), because it can be performed quickly and with a high degree of confidence. High reflectivity and enlargement of the extraocular muscles are assessed easily, and serial ultrasonographic examinations can also be used to assess progression or stability of the ophthalmopathy.

Based on the technologies currently available, or that will become available in the future, one of skill in the art would be capable of determining the best modality for diagnosing and evaluating the extent of proptosis or exophthalmos.

Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average reading of 13.9 mm versus 18.3 mm for the TED group.

Even within a group of people, there can be variability. Four ethnic groups in Southern Thailand had exophthalmometry measurement averages ranging from 15.4 mm to 16.6 mm. In 2477 Turkish patients, the median measurement was 13 mm, with an upper limit of 17 mm; and in a Dutch study, the upper limit was 20 mm in males and 16 mm in females.

Although the average and upper limits for exophthalmos or proptosis vary widely, it is accepted in the field that a difference greater than 2 mm between the eyes is significant and not normal.

One of skill in the art, for example an ophthalmologist, surgeon or other clinician skilled in the knowledge and treatment of eye disorders would know what a normal value of proptosis is based on the age, gender and race of the subject and have the ability to diagnose or evaluate the presence or absence of proptosis as well as track its progression.

### Activity Measures or Assessments

Several classification systems have been conceived to assess the clinical manifestations of TED (TAO or GO). In 1969, Werner reported the NOSPECS Classification (No physical signs or symptoms, Only signs, Soft tissue involvement, Proptosis, Extraocular muscle signs, Corneal involvement, and Sight loss) (Werner, S. C. American Journal of Ophthalmology, 1969, 68, no. 4, 646-648.)

The modified NOSPECS was also published by Werner in 1977 and has been broadly used since then (Werner, S. C. American Journal of Ophthalmology, 1977, 83, no. 5, 725-727). This classification grades for clinical severity and does not provide a means of distinguishing active TED (inflammatory progressive) from inactive TED (noninflammatory stationary). Therefore, the indication for treatments used to be based exclusively in the severity of symptoms without consideration whether the disease was active or inactive. In 1989, Mourits et al. described the Clinical Activity Score (CAS) (Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mourits et al., Clinical Endocrinology, 1997. 47, no. 1, 9-14). This protocol is further described below.

The Clinical Activity Score (CAS) consists of seven components: 1) spontaneous retrobulbar pain, 2) pain on attempted eye movements (upward, side-to-side, and downward gazes), 3) conjunctival redness, 4) redness of the eyelids, 5) chemosis (conjunctival swelling/edema), 6) swelling of the caruncle/plica, and 7) swelling of the eyelids. One point is given for the presence of each of these parameters (1 if present, 0, if absent). The sum of all points defines clinical activity and provides the CAS. A CAS≥3/7 indicates active GO. A ten-item CAS scale exists as well, but in clinical trials, the 7-item scale is generally used, being more amenable to longitudinal studies involving multiple assessments. A change of >2 points is considered clinically meaningful.

Swelling in TED (TAO or GO) is seen as chemosis (edema of the conjunctiva) and swelling of the caruncule and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncule and/or plica semilunaris.

Other grading systems have also been developed for the assessment of TED (TAO or GO). The VISA Classification (vision, inflammation, strabismus, and appearance) (Dolman, P. J., and Rootman, J., Ophthalmic Plastic and Reconstructive Surgery, 2006, 22, no. 5, 319-324 and Dolman, P. J., Best Practice & Research Clinical Endocrinology & Metabolism, 2012, 26, no. 3, 229-248) and the European Group of Graves' Orbitopathy (EUGOGO) Classification (Bartalena, L., et al., European Journal of Endocrinology, 2008, 158, no. 3, 273-285) are two such examples. Both systems are grounded in the NO SPECS and CAS classifications and use indicators to assess the signs of activity and the degree of severity. More importantly, they allow the clinician to guide the treatment of the patient with GO. VISA is more commonly used in North America and Canada while EUGOGO is in Europe. Since the VISA and EUGOGO protocols are not interchangeable, only one of them should be employed as a reference in a specific patient.

### Graves Ophthalmopathy Quality of Life (GO-QoL)

In addition to proptosis (or exophthalmos) and CAS, quality of life (QoL) can be evaluated with the use of the Graves' ophthalmopathy quality of life (GO-QoL) questionnaire. This questionnaire is designed to determine the improved quality of life after treatment. In some embodiments, the questionnaire may determine the decreased or lack of side effects after being treated with compound disclosed herein, or a pharmaceutically acceptable salt or solvate according to the methods disclosed herein, as compared to treatment with glucocorticoids.

The GO-QoL questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the thereof, according to the methods disclosed herein, as compared to treatment with glucocorticoids.

The GO-QoL questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

### Severity Measures

For lid aperture, the distance between the lid margins is measured (in mm) with the patient looking in the primary position, sitting relaxed, and with distant fixation. For swelling of the eyelids, the measure/evaluation is either “absent/equivocal,” “moderate,” or “severe.” Redness of the eyelids is either absent or present. Redness of the conjunctivae is either absent or present. Conjunctival edema is either absent or present. Inflammation of the caruncle or plica is either absent or present. Exophthalmos was measured in millimeters using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, .i.e., best-corrected visual acuity, color vision, optic disc, relative afferent pupillary defect, the condition is either absent or present. In addition, visual fields are checked if optic nerve compression is suspected.

### Severity Classification

Sight-threatening thyroid eye disease: Patients with dysthyroid optic neuropathy (DON) and/or corneal breakdown. This category warranted immediate intervention.

Moderate-to-severe thyroid eye disease: Patients without sight-threatening disease whose eye disease had sufficient impact on daily life to justify the risks of immunosuppression (if active) or surgical intervention (if inactive). Patients with moderate-to-severe thyroid eye disease usually had any one or more of the following: lid retraction ≥2 mm, moderate or severe soft tissue involvement, exophthalmos ≥3 mm above normal for race and gender, inconstant or constant diplopia.

Mild thyroid eye disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

Additional testing, including clinical trial protocols and criteria and the lead-in study, which can be performed to determine efficacy for the treatment of TED can be found in US20190225696A1.

Further, the IGR-1R inhibitors described herein may be useful for the treatment of TED in subjects who were either proptosis non-responders (<2 mm reduction in proptosis in the study eye) in the lead-in study or were proptosis responders in the lead-in study but meet the criteria for re-treatment due to relapse.

Another aspect of the disclosure provides a compound or salt disclosed herein (such as compounds of Formulae (I), (A), (IA), (IB), (IC), (ID), (ID-1), (ID-2), (ID-3) and (ID-4) or compounds of Table 1, or compounds of Embodiments 1 to 29, 40 or 41, or a pharmaceutically acceptable salt of any of the foregoing), or a pharmaceutical composition disclosed herein, for use in treating thyroid-associated ophthalmopathy, Graves' opthalmopathy or Graves' orbitopathy.

Yet another aspect of the disclosure provides a compound disclosed herein, or a pharmaceutically acceptable salt thereof, or a pharmaceutical composition disclosed herein, in the preparation of a medicament for treating thyroid-associated ophthalmopathy, Graves' opthalmopathy or Graves' orbitopathy.

A further aspect provided by the disclosure is a method of treating thyroid-associated ophthalmopathy, Graves' opthalmopathy or Graves' orbitopathy in a subject in need thereof, the method comprising administering to the subject a therapeutically effective amount of a compound disclosed herein, or a pharmaceutically acceptable salt thereof, or a pharmaceutical composition disclosed herein.

Provided herein as Embodiment 31 is a method of treating an insulin-like growth factor-1 receptor mediated disease in a subject in need of treatment, the method comprising administering to the subject a therapeutically effective amount of the compound or salt of any one of Embodiments 1 to 29, or the composition of Embodiment 30.

Provided herein as Embodiment 32 is the method of Embodiment 31, wherein the disease is cancer, psoriasis, thyroid eye disease, Graves' disease, acromegaly, gigantism, atherosclerosis, diabetes, neuropathy or osteoporosis, or any combination of the foregoing.

Provided herein as Embodiment 33 is the method of Embodiment 31, wherein the disease is thyroid eye disease.

Provided herein as Embodiment 34 is the compound or salt of any one of Embodiments 1 to 29, or the pharmaceutical composition of Embodiment 30 for use as a medicament.

Provided herein as Embodiment 35 is the compound or salt of any one of Embodiments 1 to 29, or the pharmaceutical composition of Embodiment 30 for use in the treatment of an insulin-like growth factor-1 receptor mediated disease.

Provided herein as Embodiment 36 is the use of the compound or salt of any one of Embodiments 1 to 29, or the pharmaceutical composition of Embodiment 30, for the manufacture of a medicament for the treatment of an insulin-like growth factor-1 receptor mediated disease.

Provided herein as Embodiment 37 is the use of the compound or salt of any one of Embodiments 1 to 29, or the pharmaceutical composition of Embodiment 30, wherein the disease is cancer, psoriasis, thyroid eye disease, Graves' disease, acromegaly, gigantism, atherosclerosis, diabetes, neuropathy or osteoporosis, or any combination of the foregoing.

Provided herein as Embodiment 38 is the use of Embodiment 35, wherein the disease is thyroid eye disease.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula (I) can be synthesized according to the following schemes. Variables used in the following schemes are the variables as defined for Formula (I), unless otherwise noted. All starting materials are either commercially available, for example, from Sigma-Aldrich or known in the art and may be synthesized by employing known procedures using ordinary skill. Starting materials may also be synthesized via the procedures disclosed herein. Suitable reaction conditions, such as solvent, reaction temperature, and reagents, for the Schemes discussed in this section, may be found in the examples provided herein.

In general, the compounds of Formula (I) can be synthesized according to Scheme 1 as shown below:

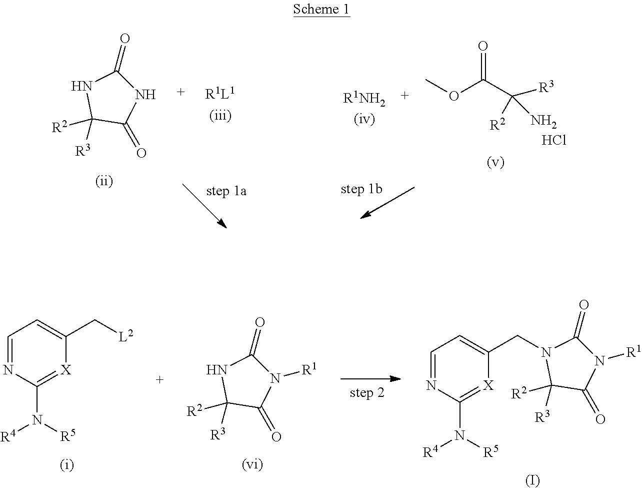

As shown in Scheme 1, hydantoin (vi) may be prepared either by the coupling of hydantoin (ii) to compound (iii) via step 1a; or by cyclizing amino acid derivative (v) and reaction with compound (iv) via step 1b. R¹ is an aryl group which may be a monocyclic or a 6,5-bicyclic group, and which may be substituted. L¹ is a leaving group such as a halogen, R² and R³ are each independently C₁₋₆alkyl. In some cases, step 1a is copper catalyzed arylation reaction. The desired substituents for R¹ may be appended to the aryl group either prior to step 1a or step 1b, or subsequent to step 1a or step 1b.

In step 2, the hydantoin (vi) formed via step 1a or step 1b is coupled with heterocyclic compound (i) in an alkylation reaction to form the compounds of Formula (I). L² is a leaving group such as a halogen, X is N or CR⁸; and R⁸ is H, halo, OH, C(O)C(O)OH, or C₁₋₆ alkyl, or when X is CR⁸, R⁵ and R⁸ together with the atoms to which they are attached form a fused 5-membered heterocycle, the fused heterocycle substituted with 0-2 substituents independently selected from OH, oxo, C₁₋₆alkyl, and a 3-8-membered monocyclic heterocycle having 1-2 ring heteroatoms selected from N, O, and S, and is substituted with 0-2 substituents independently selected from halo, OH, oxo, CN, and C₁₋₃alkyl. R⁴ is H, —C(O)C₁₋₃alkyl, Het¹, C₁₋₃alkylene-O—C₁₋₃alkyl, C(O)C₁₋₃alkylene-N(R^{N})₂; Het¹ is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; and R⁵ is H, C₁₋₆ alkyl, C₁₋₃ alkylene-CN, C₁₋₃alkylene-O—C₁₋₃alkyl, C₁₋₆alkoxy, C(O)C₀₋₃alkylene-N(R^{N})₂, C₀₋₃alkylene-heterocyclyl having 3-6 total ring atoms and 1-2 heteroatoms selected from N, S, and O, —C(O)—, or —C(OH)═, and the heterocycle or cycloalkyl ring is substituted with 0-3 substituents independently selected from C₁₋₆ alkyl, C₁₋₆alkoxy, OC(O)C₁₋₃alkyl, and C₀₋₃ alkylene-CN. The R⁴ and R⁵ nitrogen substituents of heterocyclic compound (I) may be modified (such as via cyclization including R⁵ and R⁸) either prior to, or subsequent to the coupling of (i) and (vi).

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid, gas phase, and supercritical fluid), extraction, distillation, trituration, and reverse phase HPLC.

### Intermediates

The disclosure further encompasses intermediate compounds, including structures produced from the synthetic procedures described, whether isolated or generated in-situ and not isolated, prior to obtaining the finally desired compound. These intermediates are included in the scope of this disclosure.

Provided herein are intermediates of Formula (A):

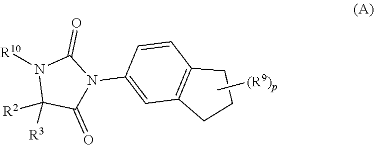

and pharmaceutically acceptable salts of any of the foregoing, wherein p is 0, 1, 2, 3, 4, 5, or 6; each of R² and R³ independently is C₁₋₆ alkyl; and each R⁹ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂-C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het; or two geminal R⁹ groups, together with the atom to which they are attached, form a spiro-C₃-7cycloalkyl group; Het is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; L³ is C₀₋₃alkylene, O—C₀₋₃alkylene-, —C(O)—, N(R^{N}), or N(R^{N})C(O)C₀₋₃alkylene-; R¹⁰ is H or C₁₋₃alkylene-R¹¹; R¹¹ is

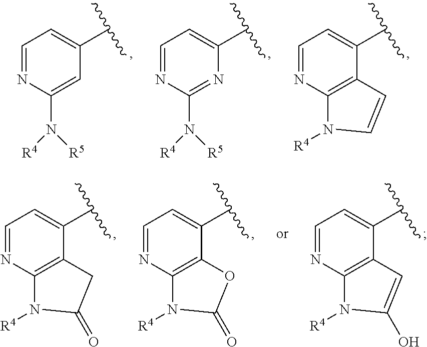

R⁴ is H, —C(O)C₁₋₃alkyl, C₁₋₃alkylene-O—C₁₋₃alkyl, C(O)C₁₋₃alkylene-N(R^{N})₂; R⁵ is H, C₁₋₆ alkyl, C₁₋₃ alkylene-CN, C₁₋₃alkylene-O—C₁₋₃alkyl, C₁₋₆alkoxy, C(O)C₀₋₃alkylene-N(R^{N})₂; and each R^{N} is independently H, C₁₋₃alkyl, C₁₋₃alkylene-CN, or C₁₋₃haloalkyl.

In some cases, p is 0, 1, 2, or 3. In some cases, p is 3. In some cases, R² and R³ are each methyl. In some cases, each R⁹ independently is OH, oxo, C₁₋₆alkyl, C₁₋₆alkoxy, or C₀₋₃ alkylene-N(R^{N})₂. Contemplated examples of intermediates for Formula (A) include the compounds listed in Table INT-A, and pharmaceutically acceptable salts of any of the foregoing.

INT-A

| Intermediate | | |
|---|---|---|
| Ref. | Structure | Name |
| A1 | 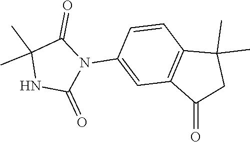 | 3-(1,1-dimethyl-3-oxo-2,3- dihydro-1H-inden-5-yl)- 5,5-dimethylimidazolidine- 2,4-dione |
| A2 | 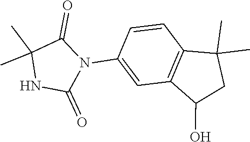 | 3-(3-hydroxy-1,1-dimethyl- 2,3-dihydro-1H-inden-5-yl)- 5,5-dimethylimidazolidine- 2,4-dione |
| A3 | 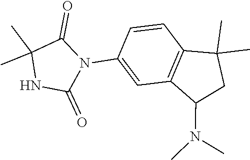 | 3-(3-(dimethylamino)-1,1- dimethyl-2,3-dihydro-1H- inden-5-yl)-5,5-dimethyl- imidazolidine-2,4-dione |

Another aspect of the disclosure is a process for preparing a compound or salt described herein (such as compounds of Formulae (I), (IB), (IC), (ID), (1D-1), (1D-2), (1D-3), or (1D-4), or compounds listed in Table 1, or compounds of Embodiments 1 to 29, or a pharmaceutically acceptable salt of any of the foregoing), comprising converting an intermediate described herein, such as an intermediate of Formula (A), or an intermediate listed in Table INT-A, or a pharmaceutically acceptable salt of any of the foregoing into a compound of the disclosure. In some cases, the intermediate is a compound of Formula (A), or a pharmaceutically acceptable salt of any of the foregoing. In some cases, the intermediate is a compound listed in Table INT-A, or a pharmaceutically acceptable salt of any of the foregoing.

Provided herein as Embodiment 39 is a compound of Formula (A):

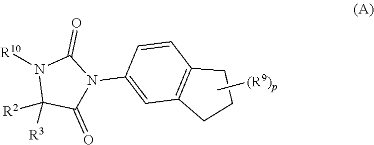

or a salt thereof, wherein p is 0, 1, 2, 3, 4, 5, or 6; each of R² and R³ independently is C₁₋₆ alkyl;
each R⁹ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂-C₁₋₃alkyl, C₀₋₃alkylene-SO₂Cl-3haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het; or two geminal R⁹ groups, together with the atom to which they are attached, form a spiro-C₃₋₇cycloalkyl group; Het is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo; L³ is C₀₋₃alkylene, O—C₀₋₃alkylene-, —C(O)—, N(R^{N}), or N(R^{N})C(O)C₀₋₃alkylene-; R¹⁰ is H or C₁₋₃alkylene-R¹¹;
R¹¹ is

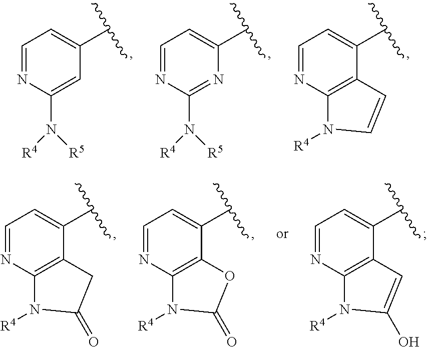

R⁴ is H, —C(O)C₁₋₃alkyl, C₁₋₃alkylene-O—C₁₋₃alkyl, C(O)C₁₋₃alkylene-N(R^{N})₂; R⁵ is H, C₁₋₆ alkyl, C₁₋₃ alkylene-CN, C₁₋₃alkylene-O—C₁₋₃alkyl, C₁₋₆alkoxy, C(O)C₀₋₃alkylene-N(R^{N})₂; and each R^{N} is independently H, C₁₋₃alkyl, C₁₋₃alkylene-CN, or C₁₋₃haloalkyl.

Provided herein as Embodiment 40 is the compound or salt of Embodiment 39, wherein p is 1, 2, or 3; each of R² and R³ are methyl; and at least one R⁹ independently is OH, oxo, C₁₋₆alkyl, C₁₋₆alkoxy, or C₀₋₃ alkylene-N(R^{N})₂.

Provided herein as Embodiment 41 is a process for preparing the compound or salt of any one of Embodiments 1-25 or 28, comprising providing a compound or salt of any one of Embodiments 39-40 and converting it into a compound or salt of any one of Embodiments 1-25 or 28.

The following examples are given for the purpose of illustrating various embodiments of the disclosure and are not meant to limit the present disclosure in any fashion. One skilled in the art will appreciate readily that the present disclosure is well-adapted to carry out the objects and obtain the ends and advantages mentioned, as well as those objects, ends, and advantages inherent herein. Changes therein and other uses which are encompassed within the spirit of the disclosure as defined by the scope of the claims will occur to those skilled in the art.

### EXAMPLES

This section provides specific examples of compounds of Formula (1) and methods of making the same.

### List of Abbreviations

| | |
|---|---|
| Ac | acetyl |
| ACN or MeCN | acetonitrile |
| AcOH | acetic acid |
| AmB | ammonium bicarbonate |
| AmF | ammonium formate |
| aq or aq. | aqueous |
| BOC or Boc | tert-butyloxycarbonyl |
| DCM | dichloromethane |
| DIPEA | N,N-diisopropylethylamine |
| DMA | N,N-dimethylacetamide |
| DMF | N,N-dimethylformamide |
| DMSO | dimethyl sulfoxide |
| DPPF or dppf | bis(diphenylphosphino)ferrocene |
| eq or eq. or equiv. | equivalent |
| ESI or ES | electrospray ionization |
| Et | ethyl |
| EtOAc | ethyl acetate |
| EtOH | ethanol |
| g or gr | gram(s) |
| h | hour(s) |
| HATU | 1-[bis(dimethylamino)methylene]-1H-1,2,3-triazolo[4,5- |
| | b]pyridinium 3-oxide hexafluorophosphate |
| HPLC | high pressure liquid chromatography |
| iPr₂NET or DIPEA | N-ethyl diisopropylamine (Hünig's base) |
| LC MS, LCMS, LC- | liquid chromatography mass spectroscopy |
| MS or LC/MS | |
| m/z | mass divided by charge |
| Me | methyl |
| MeOH | methanol |
| mg | milligrams |
| min | minutes |
| mL | milliliters |
| MS | mass spectra |
| MTBE | methyl tert-butyl ether |
| NiCl₂(bpy) | (2,2′-bipyridine)nickel |
| NMR | nuclear magnetic resonance |
| Ph | phenyl |
| PyBroP | Bromotripyrrolidinophosphonium hexafluorophosphate |
| Rbf or rbf | round-bottomed flask |
| RM | reaction mixture |
| RP-HPLC | reverse phase high pressure liquid chromatography |
| RT or rt or r.t. | room temperature |
| sat. or satd. | saturated |
| tBu | tert-butyl |
| TCFH | N,N,N′,N′-tetramethylchloroformamidium hexafluorophosphate |
| TFA | trifluoroacetic acid |
| THF | tetrahydrofuran |

Provided in this section are descriptions of the general analytical and purification methods used to prepare the specific examples provided herein.

Chromatography: Unless otherwise indicated, product-containing residues were purified by passing the material or concentrate through pre-packed silica cartridge columns and eluting via a flash chromatography system (Biotage or ISCO) with the solvent gradient as indicated.

Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). Typical elution conditions: 45 mL/min with a linear gradient of 1000 (v/v) to 10000 acetonitrile in buffered aqueous solution, either 0.1 v %0 ammonium formate pH: 3.8 (AmF), or 0.100 ammonium bicarbonate pH: 10 (AmB) over 10 min. Conditions can be varied to achieve improved separations.

Proton NMR Spectra: Unless otherwise indicated, all ¹H NMR spectra were collected on a BrukerAvance II Ultrashield spectrometer (500 MHz) or an Agilent spectrometer (400 MHz). All observed protons are reported as parts-per-million (ppm) downfield from tetramethylsilane (TMS) using the internal solvent peak as reference. Some ¹H signals may be missing due to exchange with D from CD₃OD, or due to signal suppression.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

section 1: Synthesis of Intermediates Intermediate A

Provided in this section is the synthesis of various intermediates used to prepare compounds of Formula (I). All starting materials are either commercially available from Fisher Scientific, Combi-Blocks, Strem Chemicals, AK Scientific, Ambeed, Aldrich, Sigma-Aldrich, or similar vendors, unless otherwise noted, or known in the art and may be synthesized by employing known procedures using ordinary skill.

### General Procedure A

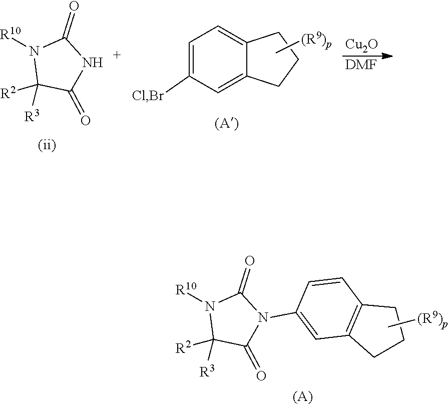

A mixture of hydantoin (ii) (1.0 equiv.), aryl halide A′ (1.0 equiv.) and Cu₂O (1.0 equiv.) in DMF (0.4 M) was heated at 150° C. under N₂ for 16 h. After cooling to rt, DMF was removed. Water (15 vol.) was added and the resulting slurry was stirred at rt for 30 min. Then, NH₄OH (5 vol.) was added and stirring continued for 30 min. The precipitate was collected by filtration, washed with water, and dried. The hydantoin product A was generally >95% pure, or further purified by chromatography (SiO₂, heptanes/EtOAc) as needed.

### Section 2: Synthesis of Example Compounds

### Method 1

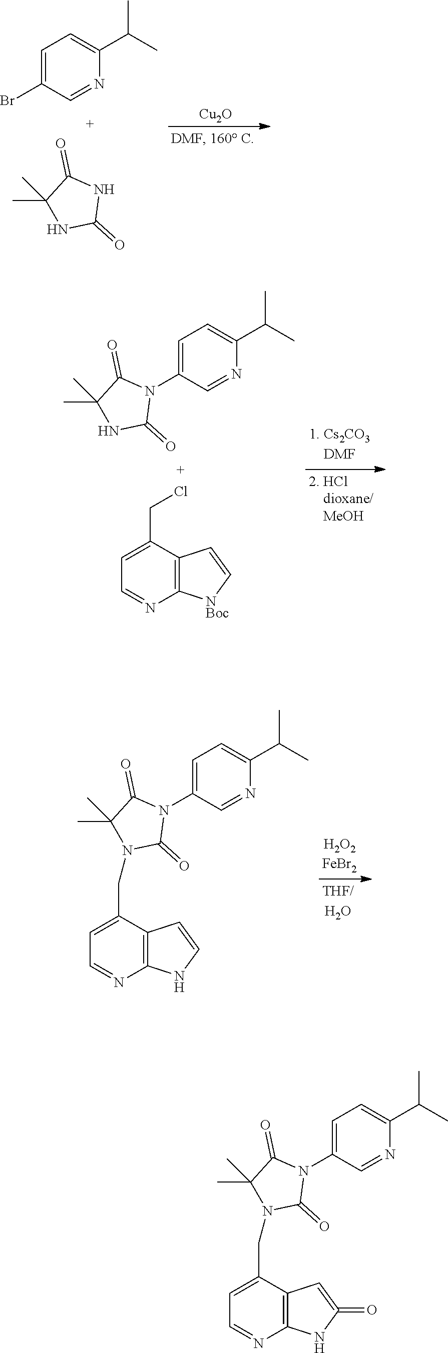

### Compound 96: 3-(6-Isopropyl-3-pyridyl)-5,5-dimethyl-1-[(2-oxo-1,7-diaza-4-indanyl)methyl]-2,4-imidazolidinedione

Step 1: 5-Bromo-2-isopropylpyridine (300 mg, 1.50 mmol), 5,5-dimethyihydantoin (384 mg, 3.00 mmol) and copper(I) oxide (354 mg, 2.40 mmol) were transferred to a tube and purged with Ar. DMF (3.75 mL) was added and then degassed with Ar. The reaction was heated to 150° C. for 16 h. The reaction was filtered, and DMF was removed. The residue was purified by chromatography, eluting with 20% to 70% EtOAc/Hexanes, to give 3-(6-isopropylpyridin-3-yl)-5,5-dimethylimidazolidine-2,4-dione, Intermediate 96.1 (375 mg, 101%). LCMS: [M+H]+=248.2 m/z.

Step 2: Intermediate 96.1 (375 mg, 1.52 mmol), Cs₂CO₃ (749 mg, 2.27 mmol), and tert-butyl 4-(chloromethyl)-1H-pyrrolo[2,3-b]pyridine-1-carboxylate (445 mg, 1.67 mmol) were combined, and DMF (7.58 mL) was added. The reaction was stirred for 2 h. The reaction mixture was filtered and concentrated. The crude mixture was dissolved in MeOH (3.03 mL). HCl 4 M dioxane (5.05 mL, 30.3 mmol) was added. The reaction mixture was stirred for 16 h. The reaction mixture was filtered and concentrated. The reaction mixture was basified with aq NaHCO₃ and extracted with EtOAc (3×). The combined organics were washed with brine, dried with Na₂SO₄, and concentrated. The residue was purified by chromatography, eluting with 0 to 100% EtOAc/Hexanes, to give 1-((1H-pyrrolo[2,3-b]pyridin-4-yl)methyl)-3-(6-isopropylpyridin-3-yl)-5,5-dimethylimidazolidine-2,4-dione Intermediate 96.2 (122 mg, 21%) LCMS: [M+H]+=378.3 m/z.

Step 3: Intermediate 96.2 (112 mg, 0.297 mmol) was dissolved in THF (1.50 mL) and H2O (150 uL). Then, iron(II) bromide (1.41 uL, 0.0297 mmol) was added, followed by hydrogen peroxide (133 uL, 1.31 mmol). The reaction mixture was stirred for 1.5 h. The reaction mixture was quenched with aq NaHCO₃ and extracted with EtOAc (3×). The combined organics were washed with brine, dried with Na₂SO₄, and concentrated. The residue was purified by reverse phase chromatography, eluting with 0 to 100% MeCN/AmF, to give compound 96, 3-(6-isopropyl-3-pyridyl)-5,5-dimethyl-1-[(2-oxo-1,7-diaza-4-indanyl)methyl]-2,4-imidazolidinedione (22.0 mg, 18 S). [M+H]+=394.3 m/z. ¹H NMR (500 MHz, DMSO) δ 8.57 (dd, J=2.5, 0.6 Hz, 1H), 8.03 (d, J=5.5 Hz, 1H), 7.83 (dd, J=8.3, 2.5 Hz, 1H), 7.44 (d, J=8.0 Hz, 1H), 7.00 (d, J=5.5 Hz, 1H), 4.54 (s, 2H), 3.66 (s, 2H), 3.09 (sept, J=6.9 Hz, 1H), 1.41 (s, 6H), 1.27 (d, J=6.9 Hz, 6H).

Compounds in Table 2 were prepared following the procedure described in Method 1, using appropriate starting materials. All starting materials are commercially available or are described in the Intermediates section above.

TABLE 2

| | | LCMS: (ESI + ve ion) |
|---|---|---|
| Ex. # | Chemical Structure & Name | m/z; NMR |
| 54 | 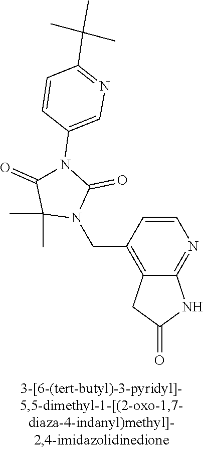 [SMILES: CC(C)(C)c1ccc(N2C(=O)N(Cc3ccnc4c3CC(=O)N4)C(C)(C)C2=O)cn1] | LCMS: 408.3 ¹H NMR (500 MHz, DMSO) δ 11.01 (s, 1H), 8.59 (dd, J = 2.5, 0.7 Hz, 1H), 8.03 (d, J = 5.5 Hz, 1H), 7.84 (dd, J = 8.5, 2.6 Hz, 1H), 7.59 (dd, J = 8.5, 0.7 Hz, 1H), 7.00 (d, J = 5.5 Hz, 1H), 4.55 (s, 2H), 3.66 (s, 2H), 1.41 (s, 6H), 1.34 (d, J = 5.5 Hz, 9H) |

### Method 2

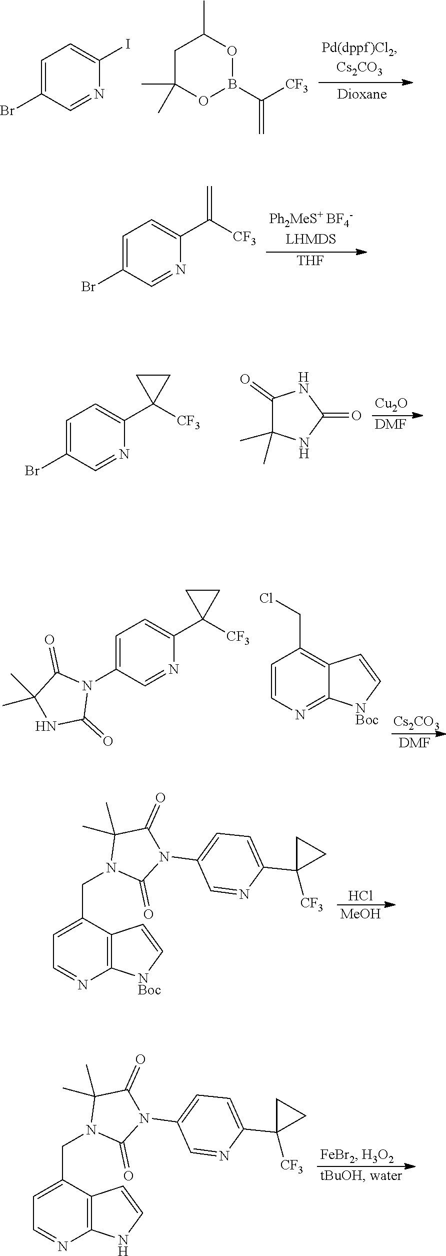

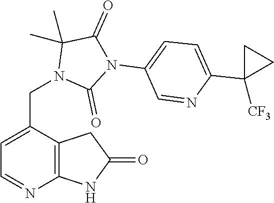 [SMILES: CC1(C)C(=O)N(c2ccc(C3(C(F)(F)F)CC3)nc2)C(=O)N1Cc1ccnc2c1CC(=O)N2]

### Compound 189: 5,5-dimethyl-1-[(2-oxo-1,7-diaza-4-indanyl)methyl]-3-{6-[1-(trifluoromethyl)cyclopropyl]-3-pyridyl}-2,4-imidazolidinedione

Step 1: 5-Bromo-2-iodopyridine (2.51 g, 8.83 mmol), 1-(trifluoromethyl)vinylboronic acid hexylene glycol ester (2.00 g, 8.83 mmol), and cesium carbonate (8.72 g, 26.5 mmol) were dissolved in dioxane (30.0 mL). The resultant mixture was sparged with Ar for 5 min, then treated with 1,1′-bis(diphenylphosphino)ferrocene dichloropalladium (II) (340 mg, 441 umol). The mixture was sparged with Ar for another 5 min, then stirred at 95° C. for 18 hours. The reaction mixture was cooled and filtered. The filtrate was concentrated and purified by chromatography, eluting with hexane/EtOAc=0%-10%, to give 5-bromo-2-(3,3,3-trifluoroprop-1-ene-2-yl)pyridine, Intermediate 189.1 (780 mg, 21%) as a colorless oil.

Step 2: A 1M lithium bis(trimethylsilyl)amide solution (4.64 mL, 4.64 mmol) in THF was added dropwise to a −78° C. solution of Intermediate 189.1 (780 mg, 1.55 mmol) and diphenyl(methyl)sulfonium tetrafluoroborate (704 mg, 2.32 mmol) in THF (30.0 mL). The resultant mixture was stirred at −78° C. for 30 min then quenched with sat aq. NH₄Cl and extracted with EtOAc. The combined organic extracts were washed with brine, dried over Na₂SO₄, filtered, and concentrated. The resulting residue was purified by chromatography, eluting with 0-5% EtOAc in ether, to afford 5-bromo-2-(1-(trifluoromethyl)cyclopropyl)pyridine, Intermediate 189.2 (824 mg, 100%) containing impurities from the previous reaction (assumed as 100% yield). LCMS: [M+H]+Na=288.

Step 3: Intermediate 189.2 (824 mg, 1.55 mmol) was added to a reaction tube. The system was evacuated and backfilled with Ar. DMF (7.00 mL) and 5,5-dimethylhydantoin (794 mg, 6.19 mmol) were added under a counter flow of Ar. Copper(I) oxide (665 mg, 4.65 mmol) was added under a counterflow of Ar. The flask was heated at 155° C. for 18 h. The reaction was cooled to rt and filtered. The crude material was purified by reverse phase chromatography, eluting with 10 mM AmB buffer/MeCN=30%-60%, to give 5,5-dimethyl-3-(6-(1-(trifluoromethyl)cyclopropyl)pyridin-3-yl)imidazolidine-2,4-dione, Intermediate 189.3 (222 mg, 46%) as a yellow solid. LCMS: [M+H]+=314.1, rt=0.95 min

Step 4: Intermediate 189.3 (100 mg, 319 umol), cesium carbonate (212 mg, 638 umol), and tert-butyl 4-(chloromethyl)-1H-pyrrolo[2,3-b]pyridine-1-carboxylate (85.1 mg, 319 umol) were stirred in DMF (1.40 mL) at rt for 3 h. The reaction was poured into ice water and a white solid was precipitated out. The solid was collected by filtration to give tert-butyl 4-((5,5-dimethyl-2,4-dioxo-3-(6-(1-(trifluoromethyl)cyclopropyl)pyridin-3-yl)imidazolidin-1-yl)methyl)-1H-pyrrolo[2,3-b]pyridine-1-carboxylate, Intermediate 189.4. (174 mg, 100%). LCMS: [M+H]+=544.2, rt=1.42 min.

Step 5: Intermediate 189.4 (173 mg, 318 umol) was dissolved in MeOH (1.20 mL), and 4M hydrochloric acid (398 uL, 1.59 mmol) in dioxane was added. The reaction was stirred at 50° C. overnight then concentrated to give 1-((1H-pyrrolo[2,3-b]pyridin-4-yl)methyl)-5,5-dimethyl-3-(6-(1-(trifluoromethyl)cyclopropyl)pyridin-3-yl)imidazolidine-2,4-dione, Intermediate 189.5 as a yellow solid (153 mg, 100%). LCMS: [M+H]+=444.3.

Step 6: To a solution of the third Intermediate 189.5 (153 mg, 345 umol) in tBuOH (1.50 mL) were added water (150 uL) and iron(II) bromide (7.59 mg, 34.5 umol). Hydrogen peroxide (176 uL, 1.73 mmol) was then added. After stirring for 3 h, the reaction was quenched with sat. Na₂S₂O₃ aq. and diluted with EtOAc. The aqueous phase was extracted with EtOAc (3×). The combined organic phase was dried over Na₂SO₄, filtered, and concentrated. The crude was purified by reverse phase chromatography, eluting with 10 mM AmB buffer/MeCN=35%-65%, to give compound 189, 5,5-dimethyl-1-[(2-oxo-1,7-diaza-4-indanyl)methyl]-3-{6-[1-(trifluoromethyl)cyclopropyl]-3-pyridyl}-2,4-imidazolidinedione (17.0 mg, 10%) as a pale blue solid. ¹H NMR (500 MHz, DMSO): δ 11.00 (s, 1H), 8.66 (d, 3=2.5 Hz, 1H), 8.02 (d, 3=5.5 Hz, 1H), 7.97 (dd, 3=8.5, 2.5 Hz, 1H), 7.71 (d, 3=8.4 Hz, 1H), 7.01 (d, 3=5.5 Hz, 1H), 4.54 (s, 2H), 3.65 (s, 2H), 1.47-1.45 (m, 2H), 1.44-1.43 (m, 2H), 1.41 (s, 6H). LCMS: [M+H]+=460.3.

### Method 3

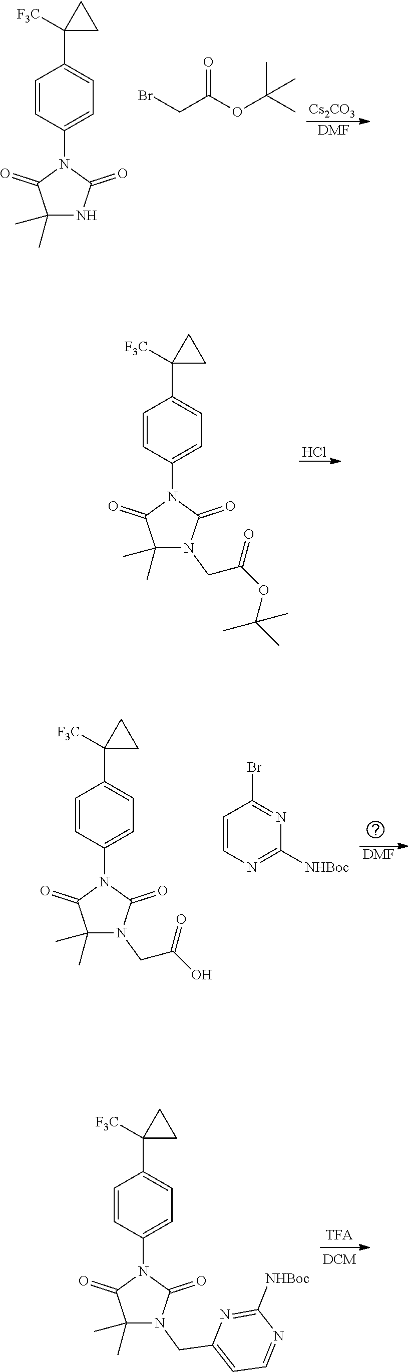

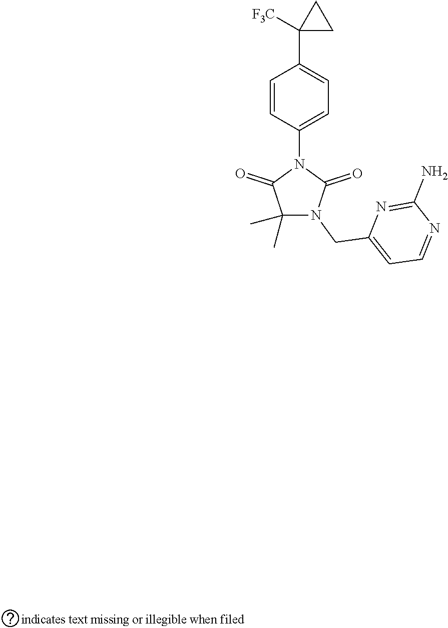 [SMILES: CC1(C)C(=O)N(c2ccc(C3(C(F)(F)F)CC3)cc2)C(=O)N1Cc1ccnc(N)n1]

Step 1: A mixture of 4-(1-methylcyclopropyl)aniline (1 equiv.) and toluene (0.05-0.1 M) was cooled in an ice bath with stirring. The mixture was allowed to warm to rt and then was heated to 70° C. The mixture was then cooled to rt and TEA (3 equiv.) and methyl 2-amino-2-methylpropanoate were added with stirring. The reaction was heated (80-120° C.) with stirring. The reaction was cooled to rt and diluted with EtOAc. The organic layer was washed with sat. aq. NH₄Cl, sat. aq. NaHCO₃, then brine. The organic layer was separated, dried over sodium sulfate, filtered and concentrated to afford 5,5-dimethyl-3-(4-(1-(trifluoromethyl)cyclopropyl)phenyl)imidazolidine-2,4-dione, Intermediate 6.1.

Step 2: Cesium carbonate (428 mg, 1.30 mmol) was added to a solution of Intermediate 6.1 (369 mg, 1.18 mmol) in DMF (3.94 mL). The solution was stirred at rt for 5 min then tert-butyl bromoacetate (196 uL, 1.30 mmol) was added. The reaction mixture was stirred at r.t. for 16 hours. NH₄Cl was added, and the aqueous solution was washed with EtOAc then brine, dried with anhydrous Na₂SO₄ and evaporated. The crude oil was dissolved in DCM, and purified with chromatography, eluting with a gradient of 0-100% EtOAc/hexanes, to afford tert-butyl 2-(5,5-dimethyl-2,4-dioxo-3-(4-(1-(trifluoromethyl)cyclopropyl)phenyl)imidazolidin-1-yl)acetate, Intermediate 6.2 (310 mg, 62%) as a white solid. LCMS: [M+H-tBu]+=371.2.

Step 3: Hydrochloric acid (4.36 mL, 17.4 mmol) (4.0 M in dioxanes) was added to Intermediate 6.2 (310 mg, 727 umol). The reaction mixture was stirred at rt for 3 days. The mixture was evaporated to afford 2-(5,5-dimethyl-2,4-dioxo-3-(4-(1-(trifluoromethyl)cyclopropyl)phenyl)imidazolidin-1-yl)acetic acid, Intermediate 6.3 (297 mg, 110%) as a white solid. LCMS: [M+H]+=371.2. ¹H NMR (400 MHz, DMSO-d6) δ 7.60-7.55 (m, 2H), 7.44-7.39 (m, 2H), 4.12 (s, 2H), 1.41 (s, 6H), 1.38-1.34 (m, 2H), 1.20-1.13 (m, 2H).

Step 4: A vial was charged with Intermediate 6.3 (100 mg, 270 umol), tert-butyl (4-bromopyrimidin-2-yl)carbamate (111 mg, 297 umol), 4,4-di-tert-butyl-2,2-dipyridyl, 98% (7.40 mg, 27.0 umol), nickel(II) chloride ethylene glycol dimethyl ether complex (6.05 mg, 27.0 umol) (Ir[dF(CF₃)ppy]₂(dtbpy))PF₆ (3.03 mg, 2.70 umol), and DMF (2.70 mL) followed by tetramethyl guanidine (103 uL, 810 umol). The vial was purged with nitrogen, sealed, and stirred for 16 h under 450 nm blue LED light irradiation. The mixture was diluted with EtOAc, and NaHCO₃ was added, the aqueous layer was extracted with EtOAc (×3), then the combined organic layers were washed with water (×4) and brine, dried over Na₂SO₄, filtered and evaporated. The crude material was purified by chromatography, eluting with EtOAc in heptanes, 0-100%, to provide tert-butyl (4-((5,5-dimethyl-2,4-dioxo-3-(4-(1-(trifluoromethyl)cyclopropyl)phenyl)imidazolidin-1-yl)methyl)pyrimidin-2-yl)carbamate, Intermediate 6.4 (37.1 mg, 22%) as a clear oil that was used in the next step without any further purification. LCMS:[M+H]+=620.5.

Step 5: To a vial containing Intermediate 6.4 (37.1 mg, 59.9 umol), CH₂Cl₂ (150 uL) was added, and the mixture was cooled to 0° C. then trifluoroacetic acid (46.3 uL, 599 umol) was added, and the mixture was left to stir at r.t. for 1 hour. The mixture was evaporated and the crude material was purified by reverse phase chromatography, eluting with MeCN in water with 10 mM ammonium formate, 10-100%, then the solid was re-purified by reverse phase chromatography, eluting with MeOH in DCM, 0-25%, to provide compound 6,1-[(2-Amino-4-pyrimidinyl)methyl]-5,5-dimethyl-3-{p-[1-(trifluoromethyl)cyclopropyl]phenyl}-2,4-imidazolidinedione (2.70 mg, 11%) as a white solid. LCMS: [M+H]+=420.4. ¹H NMR (400 MHz, MeOD) δ 8.19 (d, J=5.2 Hz, 1H), 7.61 (d, J=8.4 Hz, 2H), 7.50-7.44 (m, 2H), 6.72 (d, J=5.2 Hz, 1H), 4.52 (s, 2H), 1.49 (s, 6H), 1.42-1.36 (m, 2H), 1.16-1.09 (m, 2H). Contains 2.49 wt % heptanes.

### Method 4

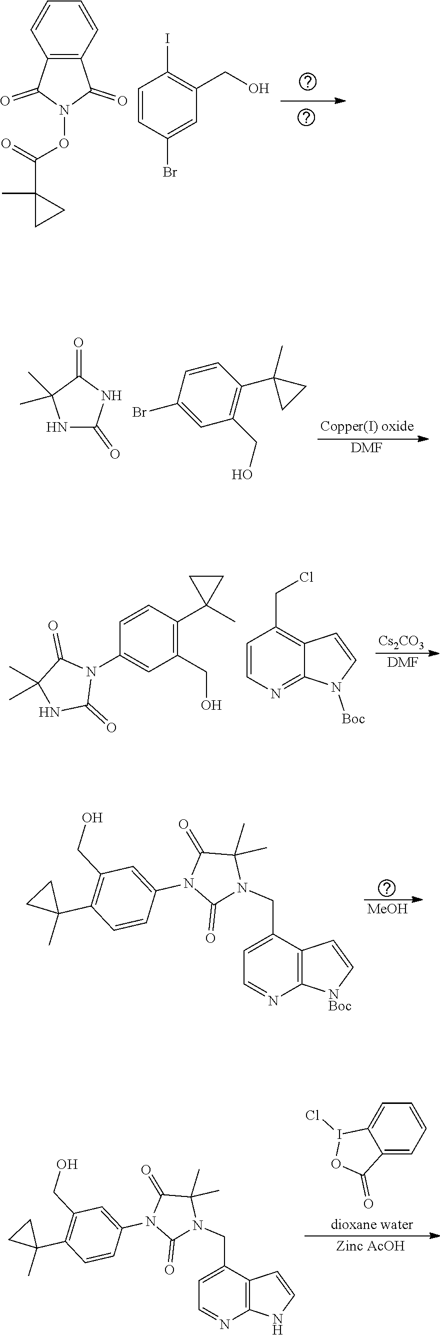

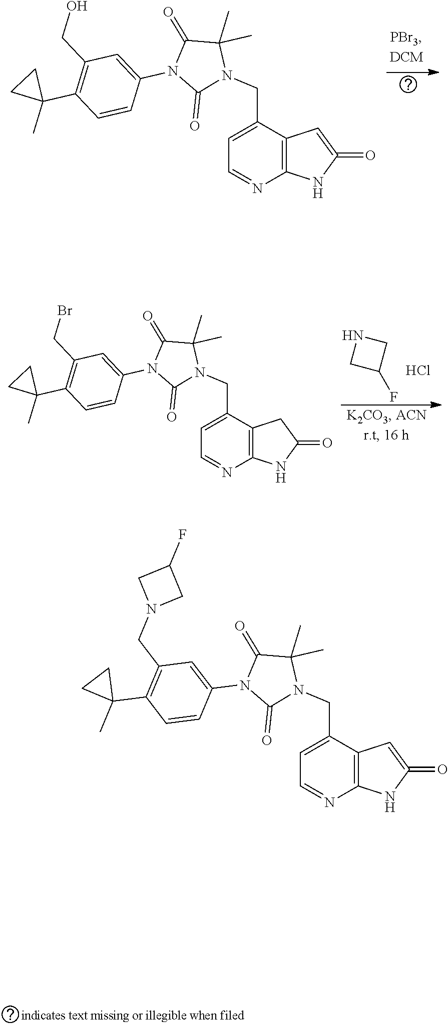

### Compound 12: 3-{3-[(3-fluoro-1-azetidinyl)methyl]-4-(1-methylcyclopropyl)phenyl}-5,5-dimethyl-1-[(2-oxo-1,7-diaza-4-indanyl)methyl]-2,4-imidazolidinedione

Step 1: A flask containing zinc (4.87 g, 74.4 mmol), 1,3-dioxoisoindolin-2-yl 1-methylcyclopropane-1-carboxylate (4.56 g, 18.6 mmol), NiCl₂bpy (399 mg, 1.39 mmol), and (5-bromo-2-iodophenyl)methanol (3.00 g, 9.30 mmol) was sealed with a rubber septum and electrical tape evacuated and backfilled with N₂ (×3). This flask was then submerged in an ice bath and left for 10 minutes in an atmosphere of N₂. A solution of chlorotrimethylsilane (4.22 mL, 32.5 mmol) in DMA (46.5 mL) was cooled in an ice bath for 15 minutes before being added to the chilled reaction vessel in an ice-bath. The reaction was left to stir for 35 min before it was diluted with EtOAc and MeOH, filtered (eluting with EtOAc), and concentrated. 1 M HCl and MeOH were added to the crude material and heated at 40° C. for 5 minutes to ensure full OTMS deprotection, then the volatiles were removed. The crude solution (in DMA) was diluted with EtOAc and washed with brine (×4), dried over MgSO₄, filtered, and concentrated under reduced pressure to a crude yellow solid. This material was purified by chromatography, eluting with 0-30% EtOAc/heptanes, to afford 5-bromo-2-(1-methylcyclopropyl)phenylmethanol, Intermediate 12.1 (2.00 g, 89%) as a yellow oil. ¹H NMR (400 MHz, CDCl₃) δ 7.64 (d, J=1.5 Hz, 1H), 7.33 (dd, J=8.2, 2.0 Hz, 1H), 7.20 (d, J=8.2 Hz, 1H), 4.93 (s, 2H), 1.33-1.24 (m, 4H), 0.79-0.69 (m, 4H). LCMS: no ionization.

Step 2: A flask was charged with 5,5-dimethylhydantoin (1.01 g, 7.68 mmol), and Intermediate 12.1 (926 mg, 3.84 mmol), and copper(I) oxide (850 mg, 5.76 mmol) and evacuated/backfilled with N₂ (×3). DMF (11.0 mL) was then added, and the solvent was sparged with N₂ for 15 minutes before the reaction vessel was heated at 150° C. for 12 h. Then, the reaction mixture was cooled to r.t., diluted with EtOAc, and filtered (eluting with EtOAc). The filtrate was washed with brine (×4), dried over MgSO₄, filtered, and concentrated to afford crude material which was purified by chromatography, eluting with 0-100% EtOAc/heptanes, to afford 3-(3-(hydroxymethyl)-4-(1-methylcyclopropyl)phenyl)-5,5-dimethylimidazolidine-2,4-dione, Intermediate 12.2 (1.17 g, 106%) as a yellow oil. LCMS: 287.2 (M−H).

Step 3: Intermediate 12.2 (500 mg, 1.73 mmol) was dissolved in DMF (8.00 mL). tert-Butyl 4-(chloromethyl)-1H-pyrrolo[2,3-b]pyridine-1-carboxylate (532 mg, 1.99 mmol) and Cs₂CO₃ (1.15 g, 3.47 mmol) were added. The reaction was stirred at 35° C. for 3 hours. The reaction mixture was filtered and concentrated to afford tert-butyl 4-((3-(3-(hydroxymethyl)-4-(1-methylcyclopropyl)phenyl)-5,5-dimethyl-2,4-dioxoimidazolidin-1-yl)methyl)-1H-pyrrolo[2,3-b]pyridine-1-carboxylate, Intermediate 12.3, (764 mg, 85%), which was used as is in the next step.

Step 4: Intermediate 12.3 from step 3 was dissolved in MeOH (8.00 mL), and hydrochloric acid (4.34 mL, 17.3 mmol) was added. The reaction mixture was heated to 75° C. for 1 hour. The reaction mixture was concentrated to afford 1-((1H-pyrrolo[2,3-b]pyridin-4-yl)methyl)-3-(3-(hydroxymethyl)-4-(1-methylcyclopropyl)phenyl)-5,5-dimethylimidazolidine-2,4-dione, Intermediate 12.4 (614 mg, 85%). Yield assumed quantitative. LCMS: (m/z)+=419.2 (M+H).

Step 5: Intermediate 12.4 (726 mg, 1.73 mmol) was dissolved in water (5.00 mL) and dioxane (20.0 mL) before 1-chloro-1λ³-benxo[d][1,2]iodaoxol-3(1H)-one (1.47 g, 5.20 mmol) were added. The reaction was stirred for 2 hours at rt before zinc (1.14 g, 17.3 mmol) and AcOH (12.3 mL) were added. The reduction was stirred for 30 min then the mixture was filtered (washing with MeOH) and concentrated to afford 3-(3-(hydroxymethyl)-4-(1-methylcyclopropyl)phenyl)-5,5-dimethyl-1-((2-oxo-2,3-dihydro-1H-pyrrolo[2,3-b]pyridin-4-yl)methyl)imidazolidine-2,4-dione, Intermediate 12.5. LCMS: 435.2.

Step 6: Intermediate 12.5 (35.0 mg, 80.6 umol) was dissolved in DCM (1.56 mL) and cooled to 0° C. Phosphorus tribromide (7.65 uL, 80.6 umol) was added. The reaction mixture was then stirred at 0° C. for 5 h. The reaction mixture was concentrated under reduced pressure, and then quenched with water and extracted with EtOAc (3×). The combined organics was washed with brine, dried with Na₂SO₄, and concentrated to give 3-(3-(bromomethyl)-4-(1-methylcyclopropyl)phenyl)-5,5-dimethyl-1-((2-oxo-2,3-dihydro-1H-pyrrolo[2,3-b]pyridin-4-yl)methyl)imidazolidine-2,4-dione, Intermediate 12.6, which was used without further purification in the next step.

Step 7: Intermediate 12.6 was dissolved in ACN (1.56 mL). 3-Fluoroazetidine hydrochloride (45.8 mg, 403 umol) and potassium carbonate (66.8 mg, 483 umol) were added. The reaction was stirred for 16 h. The reaction mixture was concentrated then purified by reverse phase chromatography, eluting with 5 to 60% MeCN/AmB, to give compound 12, 3-{3-[(3-fluoro-1-azetidinyl)methyl]-4-(1-methylcyclopropyl)phenyl}-5,5-dimethyl-1-[(2-oxo-1,7-diaza-4-indanyl)methyl]-2,4-imidazolidinedione (15.0 mg, 36%). ¹H NMR (500 MHz, DMSO) δ 10.96 (s, 2H), 8.02 (d, J=5.5 Hz, 1H), 7.42 (d, J=2.1 Hz, 1H), 7.37 (d, J=8.2 Hz, 1H), 7.22 (dd, J=8.1, 2.2 Hz, 1H), 6.96 (d, J=5.5 Hz, 1H), 5.23 (ddt, J=57.9, 10.2, 5.0 Hz, 1H), 4.52 (s, 2H), 3.91 (s, 2H), 3.69-3.61 (m, 4H), 3.21 (ddd, J=24.0, 9.5, 4.5 Hz, 2H), 1.39 (s, 6H), 1.29 (s, 3H), 0.79-0.73 (m, 4H). LCMS: (M+H) 492.2.

### Method 5

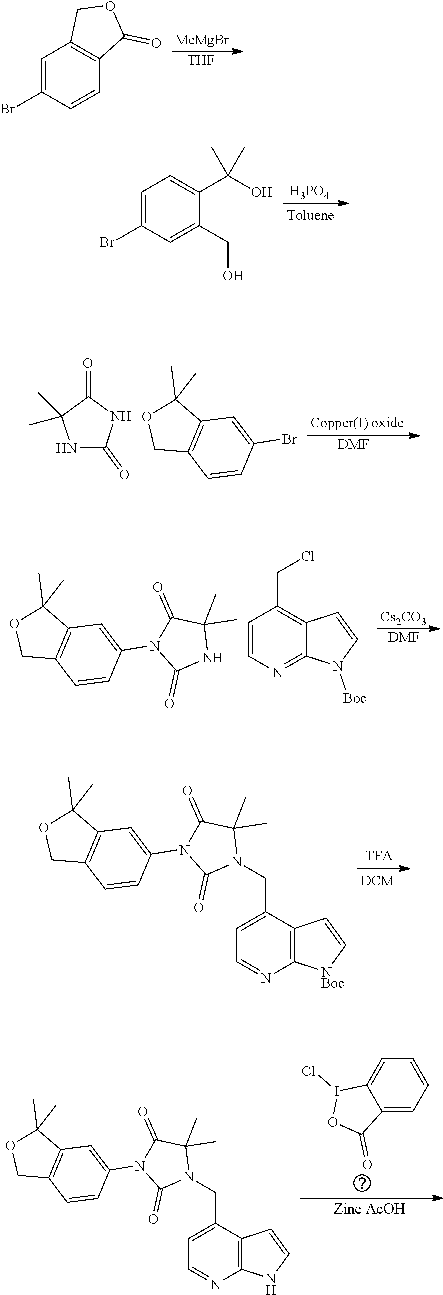

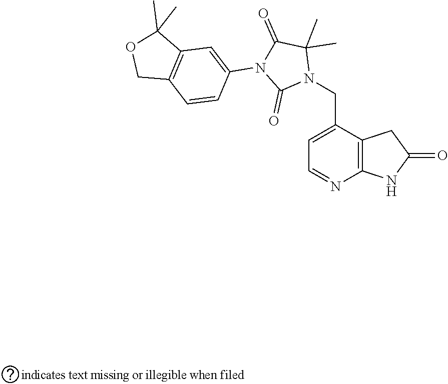 [SMILES: CC1(C)OCc2ccc(N3C(=O)N(Cc4ccnc5c4CC(=O)N5)C(C)(C)C3=O)cc21]

### Compound 24:3-(1,1-Dimethyl-1,3-dihydro-5-isobenzofuranyl)-5,5-dimethyl-1-[(2-oxo-1,7-diaza-4-indanyl)methyl]-2,4-imidazolidinedione

Step 1: To a RBF was added 5-bromophthalide (1.50 g, 6.90 mmol) in THF (60.0 mL). Methylmagnesium bromide (6.90 mL, 20.7 mmol) was added dropwise at 0° C. The resulting solution was stirred at rt overnight. The reaction mixture was cooled to 0° C., and saturated aqueous ammonium chloride was added. The mixture was extracted with EtOAc, and the organics were dried over magnesium sulfate, filtered and concentrated. The product was purified by chromatography (50% EtOAc in n-hexane). The corresponding fractions were combined and dried to afford 2-(4-bromo-2(hydroxymethyl)phenyl) propan-2-ol, Intermediate 24.1 (1.55 g, 92%) as a white solid. 1H NMR (500 MHz, CDCl₃) δ 7.49 (d, J=2.2 Hz, 1H), 7.38 (dd, J=8.5, 2.3 Hz, 1H), 7.16 (d, J=8.5 Hz, 1H), 4.80 (s, 2H), 1.67 (s, 6H).

Step 2: To Intermediate 24.1 (1.00 g, 4.08 mmol) and toluene (13.0 mL) was added phosphoric acid (216 uL, 73.4 mmol), and the resultant solution was stirred at 80° C. for 2 h. The reaction was allowed to cool to rt then cooled to 0° C. The mixture was basified with 2M sodium hydroxide then extracted with EtOAc (×2), dried over magnesium sulfate, filtered and concentrated to afford 5-bromo-1,1-dimethyl-1,3-dihydroisobenzofuran Intermediate 24.2 (904 mg, 98%) as a pale yellow liquid, which was used directly for next step without further purification. ¹H NMR (500 MHz, CDCl₃) δ 7.40-7.37 (m, 1H), 7.33-7.32 (m, 1H), 6.99 (d, J=8.0 Hz, 1H), 5.03 (s, 2H), 1.48 (s, 6H).

Step 3: A sealed vial was charged with Intermediate 24.2 (904 mg, 3.98 mmol), 5,5-dimethylhydantoin (1.05 g, 7.96 mmol), and copper(I) oxide (940 mg, 6.37 mmol). DMF (5.00 mL) was then added, and the reaction mixture was stirred at 150° C. for 17 h under argon atmosphere. The reaction was filtered, and the product was purified by reverse-phase chromatography (0-100% ACN in AmB). The corresponding fractions were combined, concentrated, extracted with EtOAc, and dried to afford 3-(3,3-dimethyl-1,3-dihydroisobenzofuran-5-yl)-5,5-dimethylimidazolidine-2,4-dione, Intermediate 24.3 (913 mg, 84%) as a white solid. ¹H NMR (500 MHz, DMSO) δ 8.52 (s, 1H), 7.34 (d, J=8.7 Hz, 1H), 7.24 (d, J=7.0 Hz, 2H), 4.97 (s, 2H), 1.44 (s, 6H), 1.40 (s, 6H).

Step 4: To a reaction vial containing Intermediate 24.3 (100 mg, 365 umol) in DMF (1.00 mL) was added cesium carbonate (303 mg, 911 umol) and tert-butyl 4-(chloromethyl)-1H-pyrrolo[2,3-b]pyridine-1-carboxylate (102 mg, 383 umol). The reaction mixture was stirred at rt for 2 h. The reaction mixture was quenched with water and extracted with EtOAc (×3). The combined organic layers were collected, then washed with water, brine, dried over MgSO₄, and concentrated to afford 1-((1H-pyrrolo[2,3-b]pyridin-4-yl)methyl)-3-(3,3-dimethyl-1,3-dihydroisobenzofuran-5-yl)-5,5-dimethylimidazolidine-2,4-dione, Intermediate 24.4 (184 mg, 100%), which was used directly for next step, assuming a yield of 100%.

Step 5: To a reaction vial of Intermediate 24.4 (184 mg, 365 umol) in DCM (1.22 mL) was added trifluoroacetic acid (846 uL, 10.9 mmol) dropwise. The reaction mixture was stirred at rt for 3 h. MeOH was added to quench the reaction. The mixture was concentrated to afford 1-((1H-pyrrolo[2,3-b]pyridin-4-yl)methyl)-3-(3,3-dimethyl-1,3-dihydroisobenzofuran-5-yl)-5,5-dimethylimidazolidine-2,4-dione, Intermediate 24.5 (147 mg, 100%) as a yellow solid, which was used directly for the next step, assuming a yield of 100%.

Step 6: To a solution of Intermediate 24.5 (147 mg, 363 umol), dioxane (3.27 mL) and water (363 uL) was added 1-chloro-1λ³-benxo[d][1,2]iodaoxol-3(1H)-one (308 mg, 1.09 mmol). The reaction mixture was stirred at rt for 1 h, and 1-((3,3-dichloro-2-oxo-2,3-dihydro-1H-pyrrolo[2,3-b]pyridin-4-yl)methyl)-3-(3,3-dimethyl-1,3-dihydroisobenzofuran-5-yl)-5,5-dimethylimidazolidine-2,4-dione, Intermediate 24.5 was observed. LCMS: [M−H]−=487.3. AcOH (2.38 mL) was added to the reaction mixture, followed by zinc (238 mg, 3.63 mmol). The reaction was then stirred at rt for 1 h.

The mixture was filtered and concentrated. The product was purified by reverse-phase chromatography. The corresponding fractions were combined and concentrated. The remaining aqueous layer was freeze-dried to afford the compound 24, 3-(1,1-dimethyl-1,3-dihydro-5-isobenzofuranyl)-5,5-dimethyl-1-[(2-oxo-1,7-diaza-4-indanyl)methyl]-2,4-imidazolidinedione (100 mg, 64%) as a white solid. ¹H-NMR (400 MHz, DMSO-d6) δ 11.00 (s, 1H), 8.02 (d, J=5.5 Hz, 1H), 7.39-7.37 (m, 1H), 7.34-7.33 (m, 2H), 6.97 (d, J=5.5 Hz, 1H), 4.99 (s, 2H), 4.53 (s, 2H), 3.64 (s, 2H), 1.45 (s, 6H), 1.39 (s, 6H).

### Method 6

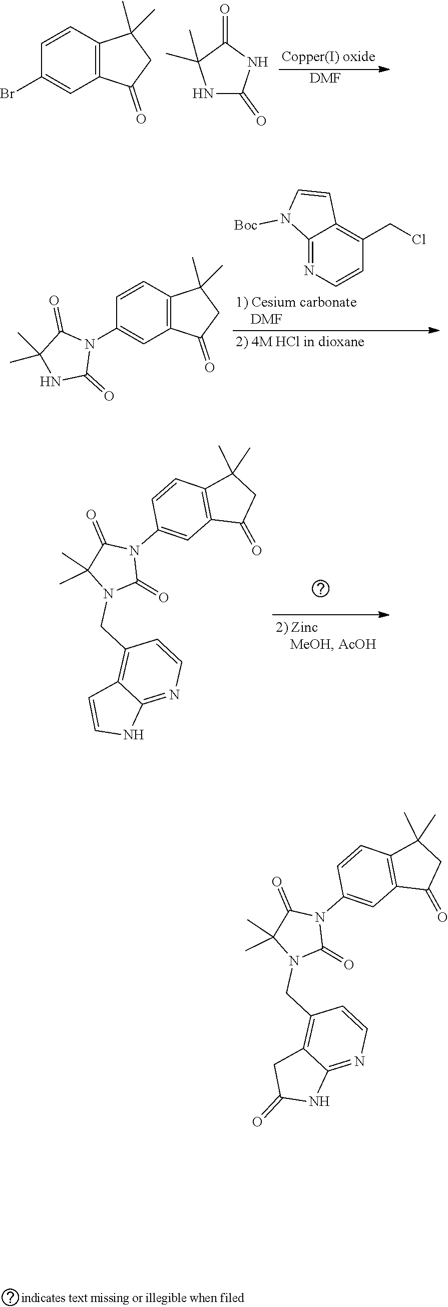

### Compound 161: 3-(1,1-dimethyl-3-oxo-5-indanyl)-5,5-dimethyl-1-[(2-oxo-1,7-diaza-4-indanyl)methyl]-2,4-imidazolidinedione

Step 1: 6-Bromo-3,3-dimethyl-2,3-dihydro-1H-inden-1-one (500 mg, 1.99 mmol), DMF (6.62 mL), and 5,5-dimethylhydantoin (509 mg, 3.97 mmol) were degassed and back filled with N₂. The process was repeated three times, before addition of copper(I) oxide (440 mg, 2.98 mmol). The reaction mixture was then placed in a pre-heated heating block (155° C.), stirring continued for 32 h. The reaction mixture was diluted with MeOH, filtered, washed with MeOH, and concentrated. The resulting residue was purified by reverse phase chromatography (5-100% ACN in 10 mM AmB mobile phase) to afford 3-(1,1-dimethyl-3-oxo-2,3-dihydro-1H-inden-5-yl)-5,5-dimethylimidazolidine-2,4-dione, Intermediate 161.1 (465 mg, 39%) as an off-white powder. LC-MS: 285.2=M−H; 96% purity in 3-min run. ¹H NMR (400 MHz, CDCl₃) δ 7.78 (d, J=8.2 Hz, 1H), 7.63 (d, J=1.6 Hz, 1H), 7.50 (dd, J=8.2, 1.5 Hz, 1H), 5.60 (s, 1H), 2.63 (s, 2H), 1.58 (s, 6H), 1.45 (s, 6H).

Step 2a: Intermediate 161.1 (465 mg, 1.62 mmol), cesium carbonate (1.08 g, 3.25 mmol), tert-butyl 4-(chloromethyl)-1H-pyrrolo[2,3-b]pyridine-1-carboxylate (619 mg, 1.95 mmol) were stirred in DMF (8.12 mL) at 50° C. for 90 min. The RM was cooled down to rt, washed with water, and extracted with CH₂Cl₂ (2×). The combined organics were dried over MgSO₄, and concentrated to provide Intermediate 161.2 which was used without purification.

Step 2b: To Intermediate 161.2 was added 4M HCl in dioxane (4.06 mL, 16.2 mmol), and the mixture stirred for one hour. The RM was concentrated, and the resulting residue was purified via chromatography (0-100%, 2.5% NH₄OH, 25% MeOH, in EtOAc and hexanes mobile phase), to afford 1-((1H-pyrrolo[2,3-b]pyridin-4-yl)methyl)-3-(1,1-dimethyl-3-oxo-2,3-dihydro-1H-inden-5-yl)-5,5-dimethylimidazolidine-2,4-dione, Intermediate 161.3 (400 mg, 45%). LC-MS: 417.3=M+H.

Step 3a: Intermediate 161.3 (400 mg, 960 umol) and t-BuOH (9.60 mL) were heated to 35° C. To this was added pyridinium tribromide (1.37 g, 3.84 mmol) then the resulting mixture was stirred for 30 min. The RM was diluted with EtOAc and washed with half saturated ammonium chloride (2×) and water (25 mL), dried over magnesium sulfate, filtered and concentrated affording an orange solid, which was carried forward as is.

Step 3b: The residue from step 3a was taken up in MeOH (4.80 mL). To this, at rt, was added zinc (641 mg, 9.60 mmol), followed by AcOH (4.80 mL), and the mixture was stirred for 15 min. The RM was diluted in MeOH, filtered, washed with MeOH, and concentrated. The resulting residue was purified over reverse phase chromatography (5-100% ACN in 10 mM AmB mobile phase) to afford compound 161, 3-(1,1-dimethyl-3-oxo-5-indanyl)-5,5-dimethyl-1-[(2-oxo-1,7-diaza-4-indanyl)methyl]-2,4-imidazolidinedione (200 mg, 47%) as an off-white powder. LC-MS: MS (m/z): [M+H]⁺=433.3. NMR (400 MHz, DMSO-D6) 6 11.01 (s, 1H), 8.03 (d, J=5.5 Hz, 1H), 7.78 (d, J=1.2 Hz, 1H), 7.71 (d, J=8.2 Hz, 1H), 7.55 (dd, J=8.2, 1.6 Hz, 1H), 7.01 (d, J=5.5 Hz, 1H), 4.54 (s, 2H), 3.66 (s, 2H), 2.62 (s, 2H), 1.42 (s, 6H), 1.40 (s, 6H).

### Method 7

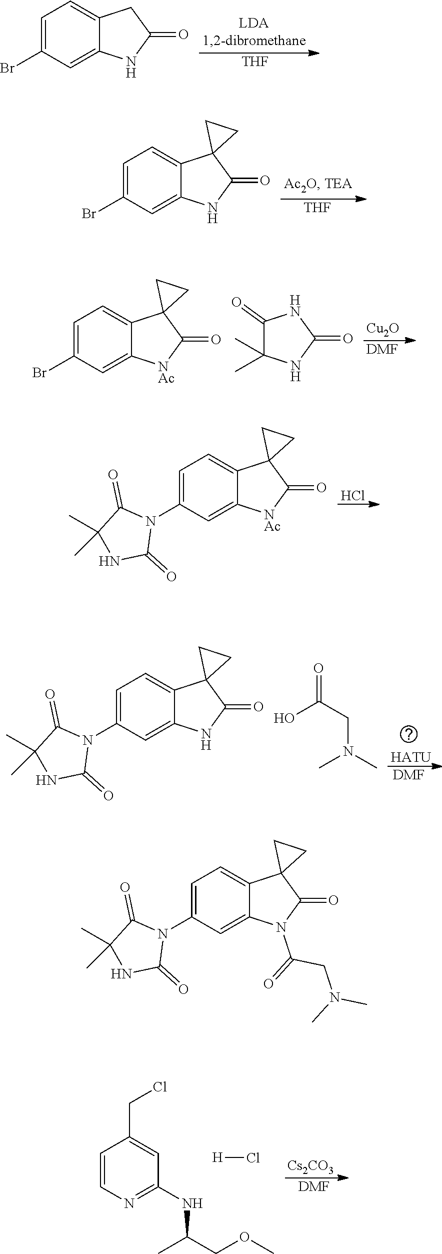

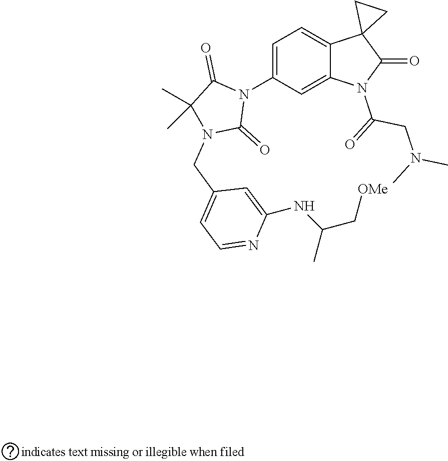

### Compound 37: 1-({2-[(R)-2-methoxy-1-methylethylamino]-4-pyridyl}methyl)-3-{1′-[2-(dimethylamino)acetyl]spiro[cyclopropane-1,3′-indolin]-6′-yl}-5,5-dimethyl-2,4-imidazolidinedione

Step 1: n-Butyllithium (28.7 mL, 71.6 mmol) was added drop wise at −78° C. to a solution of 6-bromoindolin-2-one (5.00 g, 23.1 mmol) and diisopropylamine (10.1 mL, 71.6 mmol) in THF (100 mL). The mixture was stirred for 45 min at that temperature, then 1,2-dibromoethane (7.10 mL, 81.6 mmol) was added dropwise and stirring was continued for 16 h while the temperature was allowed to rise to RT. The reaction mixture was quenched with 4 N HCl until a pH of 7, at which point a solid precipitate had begun to form. The precipitate was collected via filtration to afford 6′-bromospiro[cyclopropane-1,3′-indolin]-2′-one, Intermediate 37.1 (~2 g, 96% pure). LCMS: [M+H]+=238.1

Step 2: To Intermediate 37.1 (1.00 g, 4.46 mmol) dissolved in THF (20.0 mL) was added triethylamine (1.26 mL, 8.92 mmol) and acetic anhydride (473 uL, 4.91 mmol), and then the reaction was stirred at rt for 2 h. The mixture was concentrated, and the residue was redissolved in EtOAc, washed with water and re-extracted with EtOAc. Upon drying with Na₂SO₄, concentration delivered 1′-acetyl-6′-bromospiro[cyclopropane-1,3′-indolin]-2′-one, Intermediate 37.2 without further purification. LCMS: [M+H]+=266.1/268.1.

Step 3: Intermediate 37.2 (1.19 g, 4.47 mmol), copper(I) oxide (1.06 g, 7.15 mmol) and 5,5-dimethylhydantoin (1.18 g, 8.94 mmol) were purged with nitrogen. DMF (29.8 mL) was added, and the mixture was heated to 150° C. for 64 h. The reaction was cooled to rt, filtered and diluted with EtOAc. The organic layer was washed with water (3×) and brine, before drying with Na₂SO₄ and concentrating to deliver 3-(1′-acetyl-2′-oxospiro[cyclopropane-1,3′-indolin]-6′-yl)-5,5-dimethylimidazolidine-2,4-dione, Intermediate 37.3 (1.40 g, used in the next step without further purification). LCMS: [M+H]+=315.1 m/z.

Step 4: Intermediate 37.3 (1.40 g, 4.47 mmol) and HCl (58.3 mL, 233 mmol) (4.0 M in dioxane) were stirred at 70° C. for 40 h. The reaction was then quenched slowly with sat. NaHCO₃ solution to basic pH. The material was extracted with EtOAc (3×) and the organic layers were combined before being dried with anhydrous Na₂SO₄. The crude material was concentrated and purified by chromatography, eluting with a solvent gradient of 0-100% EtOAc/hexanes, to deliver 5,5-dimethyl-3-(2′-oxospiro[cyclopropane-1,3′-indolin]-6′-yl)imidazolidine-2,4-dione, Intermediate 37.4 (654 mg, 54%) LCMS: [M+H]+=272.4.

Step 5: Intermediate 37.4 (150 mg, 553 umol) and HATU (315 mg, 829 umol) dissolved in DMF (2.66 mL) was mixed with N,N-dimethylglycine (62.7 mg, 608 umol) and 4-methylmorpholine (246 uL, 2.21 mmol). The reaction was stirred at rt for 2 h. After 2 h, the reaction was quenched with NH₄Cl and extracted with EtOAc (3×). The combined organic layers were washed with water and brine before drying and concentrating. The crude reaction was purified by reverse phase chromatography, eluting with a solvent gradient of 20-40% MeCN/H₂O (0.1% of 10 mM AmB), to afford 3-(1′-(2-(dimethylamino)acetyl)spiro[cyclopropane-1,3′-indolin]-6′-yl)-5,5-dimethylimidazolidine-2,4-dione, Intermediate 37.5 (266 mg, 135%). LCMS: [M+H]+=356.9.

Step 6: Intermediate 37.5 (50.0 mg, 140 umol), cesium carbonate (93.3 mg, 281 umol), and (R)-4-(chloromethyl)-N-(1-methoxypropan-2-yl)pyridin-2-amine hydrochloride (38.8 mg, 154 umol) were dissolved in DMF (1.50 mL) under an atmosphere of N₂. The reaction was stirred at rt for 18 h. The reaction mixture was quenched with water and extracted with EtOAc (3×). The combined organics were dried with Na₂SO₄, and concentrated. The product was purified by chromatography, eluting with 15-35% MeCN in water (10 mM ammonium bicarbonate), to deliver compound 37, 1-({2-[(R)-2-methoxy-1-methylethylamino]-4-pyridyl}methyl)-3-{1′-[2-(dimethylamino)acetyl]spiro[cyclopropane-1,3′-indolin]-6′-yl}-5,5-dimethyl-2,4-imidazolidinedione (16.0 mg, 21%) as a white solid. LCMS: [M+H]+=535.4. ¹H NMR (500 MHz, DMSO) δ 8.06 (d, J=1.7 Hz, 1H), 7.90 (d, J=5.8 Hz, 1H), 7.00 (dd, J=8.0, 1.9 Hz, 1H), 6.91 (d, J=8.0 Hz, 1H), 6.48-6.43 (m, 2H), 6.32 (d, J=7.8 Hz, 1H), 4.42 (s, 2H), 4.23 (s, 2H), 4.11 (dt, =13.1, 6.5 Hz, 1H), 3.39 (dd, J=9.2, 5.1 Hz, 1H), 3.26 (s, 3H), 3.24-3.20 (m, 3H), 2.28 (s, 6H), 1.41 (d, J=2.6 Hz, 6H), 1.15 (t, J=5.6 Hz, 2H), 1.11 (d, J=6.6 Hz, 3H), 1.11-1.08 (m, 1H).

### Method 8

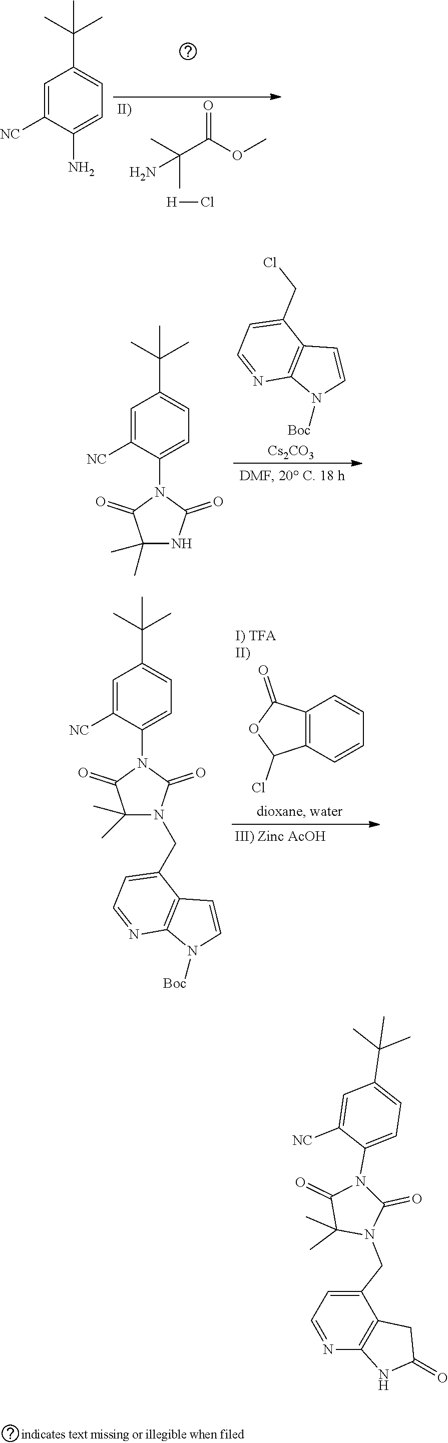

### Compound 150: 5-(tert-butyl)-2-{4,4-dimethyl-2,5-dioxo-3-[(2-oxo-1,7-diaza-4-indanyl)methyl]-1-imidazolidinyl}benzonitrile

Step 1: To a solution of 2-amino-5-tert-butylbenzonitrile (2.00 g, 10.9 mmol) and triethylamine (4.56 mL, 32.7 mmol) in toluene (54.5 mL) at −20° C. was added trichloromethyl chloroformate (1.97 mL, 16.4 mmol) dropwise. The mixture was stirred for 1 hour then heated to 75° C. and stirred an additional 2 hours. The precipitates were filtered off and methyl α-aminoisobutyrate hydrochloride (3.45 g, 21.8 mmol) was added followed by triethylamine (4.56 mL, 32.7 mmol), then the reaction was heated to 100° C. and stirred overnight. The reaction was cooled, diluted with 0.1 M HCl then extracted with EtOAc (3×). The combined organic layers were dried over MgSO₄, filtered and concentrated. The crude material was purified by chromatography, eluting with 0 to 100% EtOAc in heptanes, to provide a residue which was taken up in EtOAc and precipitated with heptanes to give 5-(tert-butyl)-2-(4,4-dimethyl-2,5-dioxoimidazolidin-1-yl)benzonitrile, Intermediate 150.1 (1.95 g, 63%) as a free flowing white solid. ES− observed=284.1 (M−H).

Step 2: To a solution of Intermediate 150.1 (300 mg, 1.05 mmol) in DMF (5.25 mL) was added tert-butyl 4-(chloromethyl)-1H-pyrrolo[2,3-b]pyridine-1-carboxylate (280 mg, 1.05 mmol) and cesium carbonate (1.04 g, 3.15 mmol). The mixture was stirred at rt overnight. The reaction was poured into water, and the precipitate was filtered off The wet solid was dissolved in DCM, dried over MgSO₄, filtered and concentrated to give tert-butyl 4-((3-(4-(tert-butyl)-2-cyanophenyl)-5,5-dimethyl-2,4-dioxoimidazolidin-1-yl)methyl)-1H-pyrrolo[2,3-b]pyridine-1-carboxylate, Intermediate 150.2 (513 mg, 97%) as a white solid that was used without further purification. ES+ observed=516.3

Step 3: TFA (10.0 mL) was added to Intermediate 150.2 (513 mg, 995.0 umol), and the mixture was stirred at rt for 30 minutes then concentrated and reconcentrated from EtOH to afford 2-(3-((1H-pyrrolo[2,3-b]pyridin-4-yl)methyl)-4,4-dimethyl-2,5-dioxoimidazolidin-1-yl)-5-(tert-butyl)benzonitrile, Intermediate 150.3. This was dissolved in dioxane (9.00 mL) and water (1.00 mL) then 1-chloro-1λ³-benxo[d][1,2]iodaoxol-3(1H)-one (843 mg, 2.98 mmol) was added. The mixture was stirred at rt for 30 minutes then AcOH (10.0 mL) and zinc (1.30 g, 19.9 mmol) were added. The mixture was stirred for 30 minutes then was filtered, concentrated, then taken up in DMSO and purified by reverse phase chromatography using a gradient of 5 to 100% CH₃CN in 10 mM AmF, then repurified by chromatography using 0 to 10% MeOH in DCM and finally 30 to 55% CH₃CN in 10 mM AmF. After lyophilization, compound 150, 5-(tert-butyl)-2-{4,4-dimethyl-2,5-dioxo-3-[(2-oxo-1,7-diaza-4-indanyl)methyl]-1-imidazolidinyl}benzonitrile (110 mg, 26%) was obtained as a white solid. ES+ observed=432.5 (M+H)¹H NMR (400 MHz, DMSO) δ 8.28 (s, 1H), 8.06 (d, J=2.0 Hz, 1H), 8.02 (d, J=5.5 Hz, 1H), 7.94 (dd, J=8.5, 2.2 Hz, 1H), 7.67 (d, J=8.5 Hz, 1H), 6.97 (d, J=5.5 Hz, 1H), 4.57 (s, 2H), 3.65 (s, 2H), 1.43 (s, 6H), 1.34 (s, 9H).

### Method 9

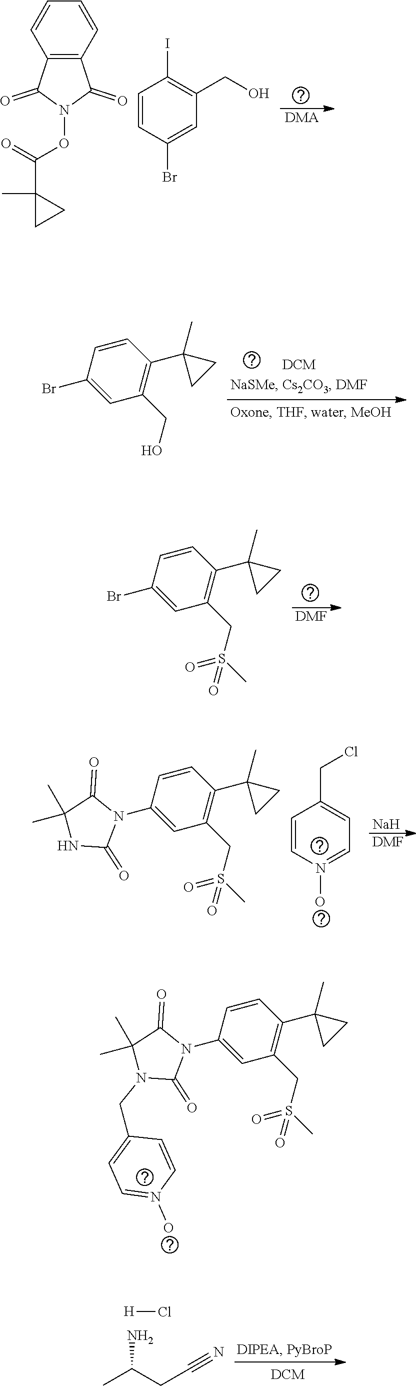

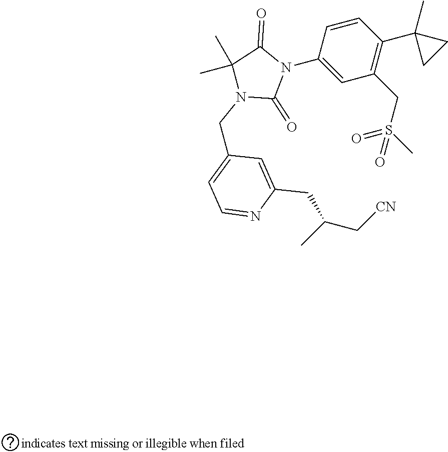 [SMILES: C[C@@H](CC#N)Cc1cc(CN2C(=O)N(c3ccc(C4(C)CC4)c(CS(C)(=O)=O)c3)C(=O)C2(C)C)ccn1]

### Compound 34: (S)-3-[4-({3-[3-(mesylmethyl)-4-(1-methylcyclopropyl)phenyl]-5,5-dimethyl-2,4-dioxo-1-imidazolidinyl}methyl)-2-pyridylamino]butyronitrile

Step 1: To a sealed vial containing NiCl₂(bpy) (177 mg, 620 umol), 1,3-dioxoisoindolin-2-yl 1-methylcyclopropane-1-carboxylate (2.74 g, 11.2 mmol), zinc (3.25 g, 49.6 mmol), (5-bromo-2-iodophenyl)methanol (2.00 g, 6.20 mmol) in DMA (20.3 mL) was added a solution of chlorotrimethylsilane (2.41 mL, 18.6 mmol) at 0° C. The reaction mixture was stirred at 0° C. for 1 h under argon atmosphere. The reaction was diluted with EtOAc and filtered, and the filtrate washed with 1 M HCl to remove the trimethylsilyl group with brine. The organic layer was collected and concentrated. The product was purified by reverse-phase chromatography (5-100% ACN in AmB). The corresponding fractions were combined, concentrated, extracted with EtOAc, and dried to afford (5-bromo-2-(1-methylcyclopropyl)phenyl)methanol, Intermediate 34.1 (1.16 g, 78%) as a colorless liquid, which was used directly for the next step.

Step 2a: To a round-bottom flask was added Intermediate 34.1 (1.16 g, 4.81 mmol) in DCM (5.00 mL). Thionyl chloride (7.09 mL, 96.2 mmol) was added, and the reaction mixture was stirred at rt overnight. The mixture was concentrated, and the residue was re-dissolved in EtOAc and sat. NaHCO₃. The organic layers were obtained and then washed with brine, and dried over MgSO₄, and concentrated to afford 4-bromo-2-(chloromethyl)-1-(1-methylcyclopropyl)benzene, Intermediate 34.2 as a yellow oil.

Step 2b: Intermediate 34.2 was taken up in DMF (9.00 mL). Sodium thiomethoxide (1.01 g, 14.4 mmol) and cesium carbonate (3.20 g, 9.62 mmol) were added. The reaction mixture was stirred at 50° C. for 1 h. The reaction was diluted with EtOAc and washed with water. The organic layers were obtained and then washed with brine, and dried over MgSO₄, and concentrated to afford (5-bromo-2-(1-methylcyclopropyl)benzyl)(methyl)sulfane, Intermediate 34.3.

Step 2c: Intermediate 34.3 was taken up in THF (5.00 mL) and MeOH (5.00 mL). Oxone (8.87 g, 14.4 mmol) and water (5.00 mL) were added. The reaction mixture was stirred at 50° C. overnight. The reaction mixture was concentrated, then diluted with EtOAc and washed with sat. NaHCO₃. The organic layers were washed with brine, dried over MgSO₄, and concentrated to afford 4-bromo-1-(1-methylcyclopropyl)-2-((methylsulfonyl)methyl)benzene, Intermediate 34.4 1.21 g, 83%) as a white solid, which was used for next step without further purification.

Step 3: A sealed vial was charged with Intermediate 34.4 (1.21 g, 3.99 mmol), 5,5-dimethylhydantoin (1.05 g, 7.98 mmol), and copper(I) oxide (942 mg, 6.38 mmol). DMF (5.00 mL) was then added, and the reaction mixture was stirred at 150° C. for 18 h under an argon atmosphere. The reaction mixture was filtered, and the product was purified by reverse-phase chromatography (eluting with 0-100% ACN in AmB). The corresponding fractions were combined, concentrated, extracted with EtOAc, and dried to afford 5,5-dimethyl-3-(4-(1-methylcyclopropyl)-3-((methylsulfonyl)methyl)phenyl)imidazolidine-2,4-dione, Intermediate 34.5 (1.08 g, 77%) as a white solid.

Step 4: To a reaction vial containing Intermediate 34.5 (150 mg, 428 umol) in DMF (0.75 mL) was added sodium hydride 60% in dispersion in mineral oil (20.5 mg, 856 umol). The reaction mixture was stirred at rt for 30 min. 4-(Chloromethyl)pyridine N-oxide (61.5 mg, 428 umol) in DMF (0.75 mL) was added dropwise to the reaction mixture, and the mixture was stirred at rt for 1 h under inert atmosphere. The reaction mixture was quenched with water and extracted with EtOAc (×3). The combined organic layers were collected, then washed with water, brine, dried over MgSO₄, and evaporated to afford 4-((5,5-dimethyl-3-(4-(1-methylcyclopropyl)-3-((methylsulfonyl)methyl)phenyl)-2,4-dioxoimidazolidin-1-yl)methyl)pyridine 1-oxide, Intermediate 34.6 (166 mg, 85%) as a white solid, which was used directly for next step without further purification.

Step 5: To a vial was added Intermediate 34.6 (166 mg, 363 umol), DIPEA (330 uL, 1.89 mmol), (S)-3-aminobutanenitrile hydrochloride (55.0 mg, 443 umol) and DCM (1.29 mL). The solution was treated with PyBroP (224 mg, 472 umol) at 0° C. and stirred at 0° C. for 15 min. The reaction mixture was then stirred at rt under an inert atmosphere. The product was purified by reverse-phase chromatography (eluting with 5-100% ACN in Amb). The corresponding fractions were combined and concentrated then freeze-dried overnight to afford compound 34, (S)-3-[4-({3-[3-(mesylmethyl)-4-(1-methylcyclopropyl)phenyl]-5,5-dimethyl-2,4-dioxo-1-imidazolidinyl}methyl)-2-pyridylamino]butyronitrile (62.0 mg, 32%) as a white solid. ¹H-NMR (500 MHz, DMSO-D6) δ 7.94 (d, J=5.3 Hz, 1H), 7.53 (d, J=2.2 Hz, 1H), 7.52 (d, J=8.3 Hz, 1H), 7.40 (dd, J=8.3, 2.2 Hz, 1H), 6.71 (d, J=7.0 Hz, 1H), 6.54 (dd, J=5.3, 1.4 Hz, 1H), 6.51 (s, 1H), 4.78 (s, 2H), 4.45 (s, 2H), 4.19-4.11 (m, 1H), 3.17 (s, 3H), 2.84 (dd, J=16.7, 5.9 Hz, 1H), 2.72 (dd, J=16.7, 4.9 Hz, 1H), 1.40 (d, J=2.5 Hz, 6H), 1.32 (s, 3H), 1.26 (d, J=6.7 Hz, 3H), 0.88-0.84 (m, 2H), 0.83-0.79 (m, 2H).

### Method 10

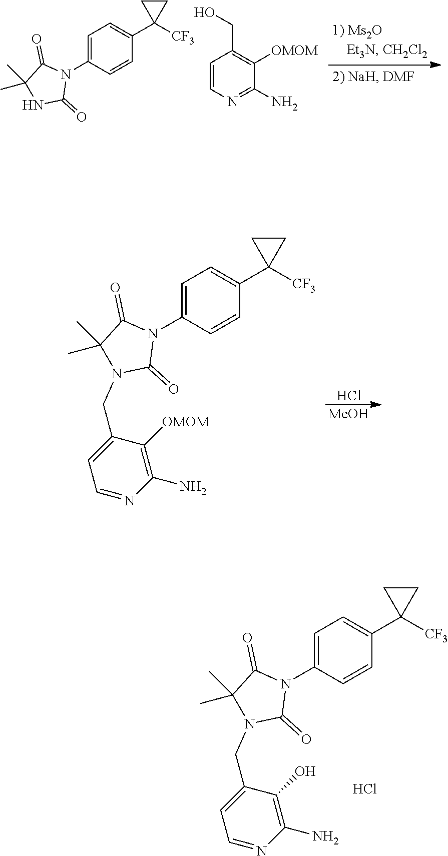

### Compound 1: 1-[(2-amino-3-hydroxy-4-pyridyl)methyl]-5,5-dimethyl-3-{p-[1-(trifluoromethyl)cyclopropyl]phenyl}-2,4-imidazolidinedione

Step 1a: (2-amino-3-((2-methoxyethoxy)methoxy)pyridin-4-yl)methanol (55.0 mg, 299 umol) was dissolved in CH₂Cl₂ (2.50 mL). The flask was cooled in an ice bath, and triethylamine (70.8 uL, 508 umol) was added, followed by a solution of methanesulfonic anhydride (64.3 mg, 358 umol) in CH₂Cl₂ (1.00 mL), dropwise. The reaction was stirred for 1 h. The reaction mixture was diluted with CH₂Cl₂ and added to saturated aqueous NaHCO₃. The layers were separated. The aqueous layer was extracted with CH₂Cl₂. The combined organic layers were dried over Na₂SO₄ and concentrated to give Intermediate 1.1 as a white solid which was used without further purification.

Step 1b: To a solution of 5,5-dimethyl-3-(4-(1-(trifluoromethyl)cyclopropyl)phenyl)imidazolidine-2,4-dione formed as described in step 1 for compound 6 above (83.9 mg, 269 umol) in DMF (2.00 mL) was added sodium hydride (41.8 mg, 1.05 mmol), and the resulting mixture was stirred for 1 h at rt. A solution of Intermediate 1.1 in DMF (1.00 mL) was added dropwise, and the mixture was stirred at rt for 15 min. The reaction was added to ice-cold, stirred NaCl 10% aq. (40 mL). EtOAc (40 mL) was added, and the layers were separated. The aqueous layer was extracted with EtOAc (4×). The combined organic layers were washed with water and brine, dried over Na₂SO₄ and concentrated to an oil, which was purified by chromatography (eluting with EtOAc) to afford 1-((2-amino-3-((2-methoxyethoxy)methoxy)pyridin-4-yl)methyl)-5,5-dimethyl-3-(4-(1-(trifluoromethyl)cyclopropyl)phenyl)imidazolidine-2,4-dione, Intermediate 1.2 (68.0 mg, 48%) as a clear film.

Step 2: Intermediate 1.2 (68.0 mg, 142 umol) was dissolved in MeOH (1.00 mL). HCl 4 M in dioxane (4.00 mL) was added, and the mixture was stirred open to air at rt for 1 h. The mixture was concentrated and lyophilized from MeCN/water to afford a beige solid. The solid was triturated in hexanes, the supernatant was pipetted and the remaining solid was dried. Two additional triturations were performed in Et₂O in the same manner as described above to finally afford compound 1,1-[(2-amino-3-hydroxy-4-pyridyl)methyl]-5,5-dimethyl-3-{p-[1-(trifluoromethyl)cyclopropyl]phenyl}-2,4-imidazolidinedione HCl-salt (30.0 mg, 45%) as a beige solid. ¹H NMR (500 MHz, MeOD) δ 7.61 (d, J=8.4 Hz, 2H), 7.48-7.45 (m, 2H), 7.42 (d, J=6.7 Hz, 1H), 6.93 (d, J=6.7 Hz, 1H), 4.69 (s, 2H), 1.53 (s, 6H), 1.42-1.38 (m, 2H), 1.14-1.10 (m, 2H). LCMS; (M+H) 435.2.

### Method 11

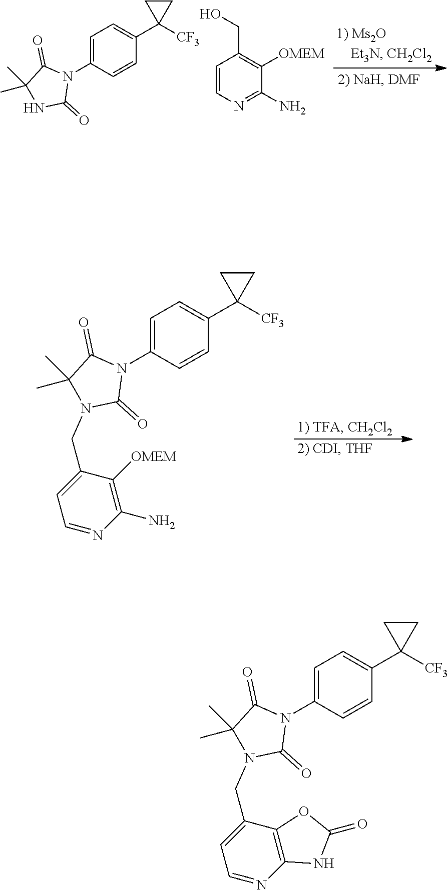

### Compound 106: 5,5-dimethyl-1-[(2-oxo-1-oxa-3,4-diaza-7-indenyl)methyl]-3-{p-[1-(trifluoromethyl)cyclopropyl]phenyl}-2,4-imidazolidinedione

Step 1: To an ice-cold solution of (2-amino-3-((2-methoxyethoxy)methoxy)pyridin-4-yl)methanol (496 mg, 2.17 mmol) in CH₂Cl₂ (10.0 mL) was added triethylamine (454 uL, 3.26 mmol), followed by a solution of methanesulfonic anhydride (449 mg, 2.50 mmol) in CH₂Cl₂ (5.00 mL). The mixture was stirred for 90 min in the ice bath. In a separate flask, a solution of 5,5-dimethyl-3-(4-(1-(trifluoromethyl)cyclopropyl)phenyl)imidazolidine-2,4-dione formed as described in step 1 for compound 6 above (611 mg, 1.96 mmol) in DMF (10.0 mL) was prepared under N₂. Sodium hydride (304 mg, 7.61 mmol) was added in 3 portions. The mixture was stirred for 30 min. Meanwhile, the mesylate solution was added to saturated aqueous NaHCO₃, and extracted with CH₂C₂. The combined organic layers were washed with brine, dried over Na₂SO₄ and concentrated. The residue was diluted with DMF (5.00 mL) and added dropwise to a basic solution, and stirred for 1 h at rt. The reaction was quenched with drops of water and added to water and MTBE. The aqueous layer was extracted with MTBE (2×). The combined organic layers were washed with water and brine, dried and concentrated to afford 1-((2-amino-3-((2-methoxyethoxy)methoxy)pyridin-4-yl)methyl)-5,5-dimethyl-3-(4-(1-(trifluoromethyl)cyclopropyl)phenyl)imidazolidine-2,4-dione, Intermediate 106.1 (580 mg, 51%) as a white foam which was used as is.

Step 2: To a solution of Intermediate 106.1 (312 mg, 597 umol) in CH₂Cl₂ (6.24 mL) was added TFA (6.24 mL). The mixture was stirred open to air for 1 h. The mixture was concentrated. The residue was diluted with THF (6.24 mL). 1,1′-Carbonylimidazole (204 mg, 1.19 mmol) was added. The mixture was heated at 60° C. for 45 min. The reaction was concentrated, and the residue was dissolved in DMSO for purification by reversed phase flash chromatography (30 g C₁₈ column, 30 to 50% MeCN in AmB).

The product was extracted into EtOAc and concentrated to a white solid, which was triturated in Et₂O to afford compound 106, 5,5-dimethyl-1-[(2-oxo-1-oxa-3,4-diaza-7-indenyl)methyl]-3-{p-[1-(trifluoromethyl)cyclopropyl]phenyl}-2,4-imidazolidinedione (50.0 mg, 18%) as a white solid, after drying. ¹H NMR (400 MHz, DMSO) δ 12.47 (brs, 1H), 7.95 (d, J=5.5 Hz, 1H), 7.60 (d, J=8.4 Hz, 2H), 7.48 (d, J=8.5 Hz, 2H), 7.06 (d, J=5.4 Hz, 1H), 4.71 (s, 2H), 1.43 (s, 6H), 1.39-1.35 (m, 2H), 1.22-1.17 (m, 2H). LCMS: (M+H) 461.3.

### Method 12

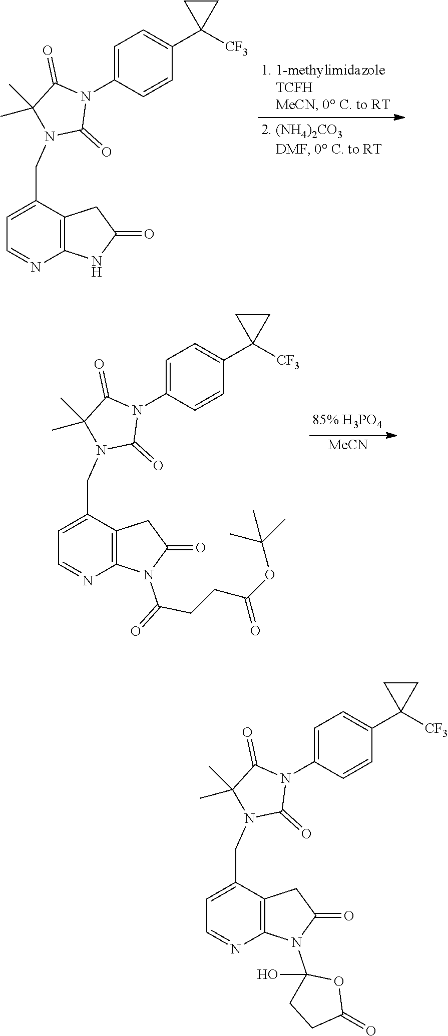

Compound 166: 1-{[2-hydroxy-1-(5-hydroxy-2-oxo-4,5-dihydro-5-furyl)-1H-1,7-diazainden-4-yl]methyl}-5,5-dimethyl-3-{p-[1-(trifluoromethyl)cyclopropyl]phenyl}-2,4-imidazolidinedione

Step 1: 5,5-Dimethyl-3-(4-(1-(trifluoromethyl)cyclopropyl)phenyl)imidazolidine-2,4-dione (formed as described for compound 6 above) (1 equiv.) was dissolved in DMF. Sodium hydride (3.5 equiv.) was added under N₂ and the resulting mixture was stirred at rt for 30 min. Then a solution of 4-(bromomethyl)-1H-pyrrolo[2,3-b]pyridine hydrobromide (1.1 equiv.) in DMF was added dropwise. The resulting mixture was stirred at rt for 30 min, and then the reaction was quenched with water. The reaction was partitioned between EtOAc and water, and the aqueous later was extracted with EtOAc (3×). The combined organic layers were washed with water, brine and dried over Na₂SO₄, then purified by chromatography to afford 1-((1H-pyrrolo[2,3-b]pyridine-4-yl)methyl)-5,5-dimethyl-3-(4-(1-(trifluoromethyl)cyclopropyl)phenylimidazolidine-2,4-dione, Intermediate 166.1.

Step 2: Intermediate 166.1 (110 mg, 69.1 μmol), pyridinium tribromide (353 mg, 994 μmol) and tert-butyl alcohol (5.4 ml) were stirred at 30° C. for 1 h. The reaction was diluted with EtOAc and washed with saturated ammonium chloride and brine, dried over sodium sulfate, filtered and concentrated to afford Intermediate 166.2 which was used without further purification.

Step 3: Intermediate 166.2 was taken up in acetic acid (10 mL), and zinc dust (163 mg, 2.49 mmol) was added. The reaction was stirred at rt for 30 min, then filtered and concentrated. The residue was taken up in EtOAc and added to sat. sodium bicarbonate. The aqueous layer was extracted, dried over sodium sulfate, filtered, concentrated, and then purified by chromatography to afford 5,5-dimethyl-1-((2-oxo-2,3-dihydro-1H-pyrrolo[2,3-b]pyridin-4-yl)methyl)-3-(4-(1-(trifluoromethyl)cyclopropyl)phenyl)imidazolidine-2,4-dione, Intermediate 166.3.

Step 4: TCFH (126 mg, 0.436 mmol) was dissolved in CH₃CN (4.00 mL) and cooled to 0° C. Mono-tert-butyl succinate (76.0 mg, 0.436 mmol) was added, followed by 1-methylimidazole (70.3 uL, 0.873 mmol). The mixture was stirred at 0° C. for 5 min. Intermediate 166.3 (100 mg, 218 umol) was added as a solution in 1 mL of DMF. The reaction mixture was stirred at 0° C. for 1 h, then rt for 16 h. The reaction mixture was concentrated, then quenched with water and extracted with EtOAc (3×). The combined organics were washed with brine, dried with Na₂SO₄, and concentrated. The residue was dissolved in DMF (436 uL) and cooled to 0° C. Ammonium carbonate (1.82 uL, 0.218 mmol) was added and the reaction mixture was stirred at rt for 4 h. The reaction mixture was poured into ice cold water and extracted with EtOAc (3×). The combined organics were washed with brine, dried with Na₂SO₄, and concentrated. Purification of the residue by reverse phase chromatography eluting with 0 to 100% MeCN/AmF gave tert-butyl 4-(4-((5,5-dimethyl-2,4-dioxo-3-(4-(1-(trifluoromethyl)cyclopropyl)phenyl)imidazolidin-1-yl)methyl)-2-oxo-2,3-dihydro-1H-pyrrolo[2,3-b]pyridin-1-yl)-4-oxobutanoate, Intermediate 166.4 (135 mg, 101%). LCMS: [M+H]+=615.2 m/z.

Step 5: Intermediate 166.4 (64.3 mg, 0.105 mmol) was dissolved in MeCN (222 uL) then 85% phosphoric acid aq (301 uL) was added. The reaction mixture was stirred at rt for 2 h. The reaction mixture was quenched with water and extracted with EtOAc (3×). The combined organics were washed with brine, dried with Na₂SO₄, and concentrated. Purification of the residue by reverse phase chromatography eluting with 0 to 100% MeCN/AmF gave compound 166, 1-{[2-hydroxy-1-(5-hydroxy-2-oxo-4,5-dihydro-5-furyl)-1H-1,7-diazainden-4-yl]methyl}-5,5-dimethyl-3-{p-[1-(trifluoromethyl)cyclopropyl]phenyl}-2,4-imidazolidinedione (22.3 mg, 36%). LCMS: [M+H]+=559.4 m/z. 1H NMR (500 MHz, CD₃CN) 6 8.14 (d, J=5.4 Hz, 1H), 7.65 (d, J=8.1 Hz, 2H), 7.50 (d, J=8.0 Hz, 2H), 7.07 (d, J=5.5 Hz, 1H), 4.99 (s, 1H), 4.47 (d, J=17.3 Hz, 1H), 4.33 (d, J=17.2 Hz, 1H), 3.26 (ddd, J=18.9, 6.1 Hz, 1H), 3.07 (ddd, J=18.4, 6.1 Hz, 1H), 2.67-2.57 (m, 2H), 1.45-1.36 (m, 8H), 1.33-1.27 (m, 2H), 1.21-1.13 (m, 2H).

### Method 13

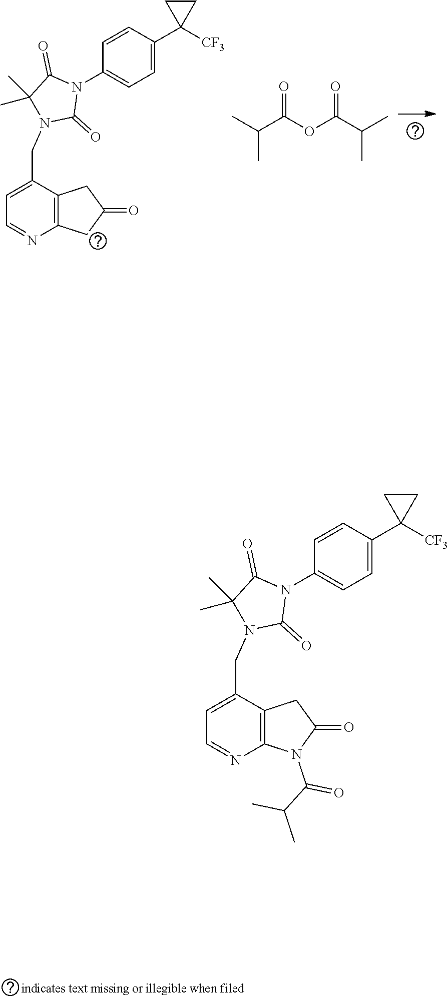 [SMILES: CC1(C)C(=O)N(c2ccc(C3(C(F)(F)F)CC3)cc2)C(=O)N1Cc1ccnc2c1CC(=O)C2 ; CC(C)C(=O)OC(=O)C(C)C ; CC(C)C(=O)N1C(=O)Cc2c(CN3C(=O)N(c4ccc(C5(C(F)(F)F)CC5)cc4)C(=O)C3(C)C)ccnc21]

### Compound 178: 1-[(1-isobutyryl-2-oxo-1,7-diaza-4-indanyl)methyl]-5,5-dimethyl-3-{p-[1-(trifluoromethyl)cyclopropyl]phenyl}-2,4-imidazolidinedione

Step 1: Intermediate 166.3 formed as described in steps 1-3 for compound 166 above (200 mg, 436 umol) was dissolved in toluene (2.40 mL). Isobutyric anhydride (129 uL, 881 umol) was then added to the mixture, which was then stirred at 80° C. for 3 h, then heated at 120° C. for 24 hours, then 130° C. for another 24 hours. After removing solvent, the crude material was purified by reverse phase chromatography (eluting with 10 mM AmF buffer/MeCN) to give pure fractions. MeCN was removed. The aqueous phase was extracted with EtOAc (3×). The combined organic phase was dried over Na₂SO₄, filtered, and concentrated. The obtained solid was lyophilized from MeCN/water to give compound 178, 1-[(1-isobutyryl-2-oxo-1,7-diaza-4-indanyl)methyl]-5,5-dimethyl-3-{p-[1-(trifluoromethyl)cyclopropyl]phenyl}-2,4-imidazolidinedione.

¹H NMR (400 MHz, DMSO): δ 8.21 (d, J=5.4 Hz, 1H), 7.60 (d, J=8.4 Hz, 2H), 7.52-7.46 (m, 2H), 7.24 (d, J=5.4 Hz, 1H), 4.59 (s, 2H), 3.91 (s, 2H), 3.85-3.75 (m, 1H), 1.41 (s, 6H), 1.37 (dd, J=6.7, 5.2 Hz, 2H), 1.19-1.17 (m, 2H), 1.15 (d, J=6.9 Hz, 6H). LCMS: [M+H]+=529.5, rt=3.61 min

Compounds in Table 3 were prepared in a manner similar to the syntheses described for Methods 1-13 above.

TABLE 3

| Compound | | LCMS: (ESI + ve ion) m/z; ¹H |
|---|---|---|
| # | Chemical Structure & Name | NMR |
| 2 | 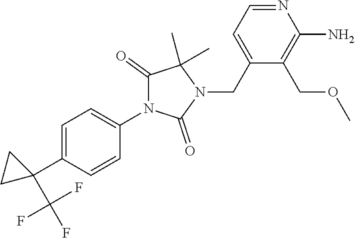 [SMILES: COCc1c(CN2C(=O)N(c3ccc(C4(C(F)(F)F)CC4)cc3)C(=O)C2(C)C)ccnc1N] 1-{[2-amino-3-(methoxymethyl)-4- pyridyl]methyl}-5,5-dimethyl-3-{p-[1- (trifluoromethyl)cyclopropyl]phenyl}-2,4- imidazolidinedione | LCMS: 463.4 (500 MHz, DMSO), 7.87 (d, J = 5.2 Hz, 1H), 7.60 (d, J = 8.5 Hz, 2H), 7.47 (d, J = 8.6 Hz, 2H), 6.6 (d, J = 5.2 Hz, 1H), 5.84 (s, 2H), 4.59 (s, 2H), 4.45 (s, 2H), 3.31 (s, 3H), 1.38 (t, J = 6.0 Hz, 2H), 1.35 (s, 6H), 1.20-1.16 (m, 2H). |
| 3 | 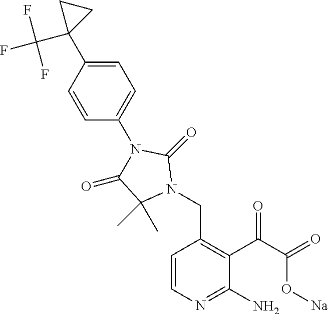 sodium {2-amino-4-[(5,5-dimethyl-2,4-dioxo-3- {p-[1-(trifluoromethyl)cyclopropyl]phenyl}-1- imidazolidinyl)methyl]-3-pyridyl}oxoacetate | LCMS: 489.25 (500 MHz, DMSO) δ 7.93 (d, J = 5.2 Hz, 1H), 7.59 (d, J = 8.4 Hz, 2H), 7.52-7.44 (m, 2H), 6.94 (s, 2H), 6.60 (d, J = 5.2 Hz, 1H), 4.58 (s, 2H), 1.37 (dd, J = 6.7, 5.1 Hz, 2H), 1.34 (s, 6H), 1.20-1.16 (m, 2H). |
| 4 | 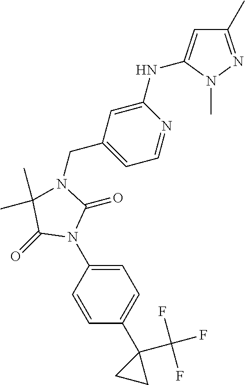 5,5-dimethyl-1-{[2-(1-methyl-3-methyl-5- pyrazolylamino)-4-pyridyl]methyl}-3-{p-[1- (trifluoromethyl)cyclopropyl]phenyl}-2,4- imidazolidinedione | LCMS: 513.3 (500 MHz, DMSO) δ 8.67 (bs, 1H), 8.05 (dd, J1 = 5.3 Hz, J2 = 0.5 Hz, 1H), 7.60 (ddd, J1 = 8.4 Hz, J2 = 3.5 Hz, J3 = 2.2 Hz, 2H), 7.46 (ddd, J1 = 8.6 Hz, J2 = 3.0 Hz, J3 = 1.5 Hz, 2H), 6.78 (dd, J = 5.2, 1.5 Hz, 1H), 6.73 (m, 1H), 5.99 (s, 1H), 4.53 (s, 2H), 3.56 (s, 3H), 2.08 (s, 3H), 1.40 (s, 6H), 1.37 (dd, J = 6.7, 5.1 Hz, 2H), 1.21-1.15 (m, 2H). |
| 5 | 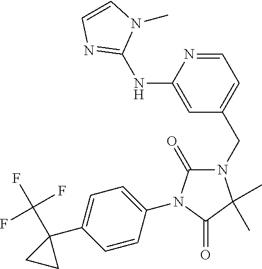 [SMILES: Cn1ccnc1Nc1cc(CN2C(=O)N(c3ccc(C4(C(F)(F)F)CC4)cc3)C(=O)C2(C)C)ccn1] 5,5-dimethyl-1-{[2-(1-methyl-2- imidazolylamino)-4-pyridyl]methyl}-3-{p-[1- (trifluoromethyl)cyclopropyl]phenyl}-2,4- imidazolidinedione | LCMS: 499.7 (500 MHz, CD3CN) δ 8.42- 8.35 (m, 1H), 8.20 (s, 1H), 7.64 (d,J = 8.3 Hz, 2H), 7.50 (d, J = 8.5 Hz, 2H), 7.24 (d,J = 5.0 Hz, 1H), 7.19 (d,J = 2.4 Hz, 1H), 6.55 (d,J = 2.1 Hz, 1H), 4.66 (s, 2H), 3.28 (s, 3H), 1.45 (s, 6H), 1.44-1.40 (m,J = 5.8 Hz, 2H), 1.21-1.15 (m, 2H). |
| 7 | 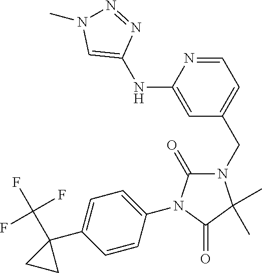 [SMILES: Cn1cc(Nc2cc(CN3C(=O)N(c4ccc(C5(C(F)(F)F)CC5)cc4)C(=O)C3(C)C)ccn2)nn1] 5,5-dimethyl-1-{[2-(1-methyl-1H-1,2,3-triazol-4- ylamino)-4-pyridyl]methyl}-3-{p-[1- (trifluoromethyl)cyclopropyl]phenyl}-2,4- imidazolidinedione | LCMS: 500.5 (500 MHz, DMSO) δ 9.65 (s, 1H), 8.13 (s, 1H), 8.10 (d, J = 5.4 Hz, 1H), 7.63-7.57 (m, 2H), 7.51-7.45 (m, 2H), 6.91 (s, 1H), 6.74 (dd, J = 5.3, 1.5 Hz, 1H), 4.54 (s, 2H), 4.01 (s, 3H), 1.42 (s, 6H), 1.40-1.35 (m, 2H), 1.21-1.16 (m, 2H). |
| 8 | 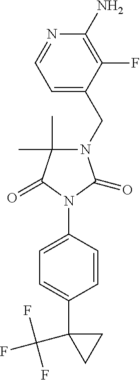 [SMILES: CC1(C)C(=O)N(c2ccc(C3(C(F)(F)F)CC3)cc2)C(=O)N1Cc1ccnc(N)c1F] 1-[(2-amino-3-fluoro-4-pyridyl)methyl]-5,5- dimethyl-3-{p-[1- (trifluoromethyl)cyclopropyl]phenyl}-2,4- imidazolidinedione | LCMS: 437.2 (400 MHz, DMSO) δ 7.69 (d, J = 5.1 Hz, 1H), 7.58 (d, J = 8.4 Hz, 2H), 7.53-7.42 (m, 2H), 6.59 (t, J = 5.0 Hz, 1H), 6.18 (s, 2H), 4.57 (s, 2H), 1.41 (s, 6H), 1.39-1.34 (m, 2H), 1.21-1.14 (m, 2H). |
| 9 | 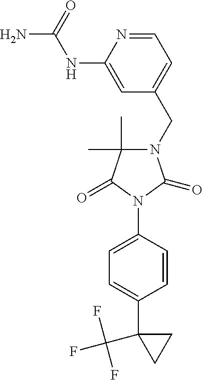 [SMILES: CC1(C)C(=O)N(c2ccc(C3(C(F)(F)F)CC3)cc2)C(=O)N1Cc1ccnc(NC(N)=O)c1] 5,5-dimethyl-3-{p-[1- (trifluoromethyl)cyclopropyl]phenyl}-1-[(2- ureido-4-pyridyl)methyl]-2,4-imidazolidinedione | LCMS: 462.3 (400 MHz, DMSO) δ 9.09 (s, 1H), 8.12 (d, J = 5.3 Hz, 1H), 7.60 (d, J = 8.4 Hz, 2H), 7.51- 7.43 (m, 2H), 7.37 (s, 1H), 6.93 (d, J = 5.3 Hz, 1H), 4.57 (s, 2H), 1.44-1.34 (m, 8H), 1.21-1.14 (m, 2H). |
| 10 | 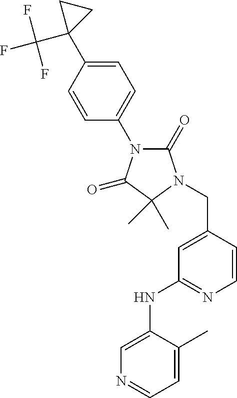 [SMILES: Cc1ccncc1Nc1cc(CN2C(=O)N(c3ccc(C4(C(F)(F)F)CC4)cc3)C(=O)C2(C)C)ccn1] 5,5-dimethyl-1-{[2-(4-methyl-3-pyridylamino)-4- pyridyl]methyl}-3-{p-[1- (trifluoromethyl)cyclopropyl]phenyl}-2,4- imidazolidinedione | LCMS: 510.3 (500 MHz, CD₂Cl₂) δ 8.79 (s, 1H), 8.26 (d, J = 4.8 Hz, 1H), 8.12 (d, J = 5.2 Hz, 1H), 7.59 (d, J = 8.4 Hz, 2H), 7.45-7.39 (m, 2H), 7.22 (d, J = 4.9 Hz, 1H), 6.80 (s, 2H), 6.64 (s, 1H), 4.48 (s, 2H), 2.30 (s, 3H), 1.41 (s, 6H), 1.40-1.38 (m, 2H), 1.11- 1.07 (m, 2H). |
| 11 | 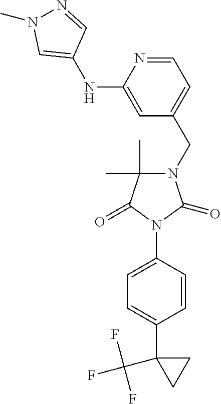 [SMILES: Cn1cc(Nc2cc(CN3C(=O)N(c4ccc(C5(C(F)(F)F)CC5)cc4)C(=O)C3(C)C)ccn2)cn1] 5,5-dimethyl-1-{[2-(1-methyl-4-pyrazolylamino)- 4-pyridyl]methyl}-3-{p-[1- (trifluoromethyl)cyclopropyl]phenyl}-2,4- imidazolidinedione | LCMS: 499.25 (500 MHz, CD₂Cl₂) δ 8.04 (d, J = 5.3 Hz, 1H), 7.63-7.59 (m, 1H), 7.57 (d, J = 8.4 Hz, 2H), 7.44-7.40 (m, 2H), 7.38 (d, J = 0.5 Hz, 1H), 6.65 (dd, J = 5.4, 1.4 Hz, 1H), 6.55 (s, 1H), 6.51 (s, 1H), 4.44 (s, 2H), 3.83 (s, 3H), 1.41-1.35 (m, 8H), 1.10- 1.05 (m, 2H). |
| 13 | 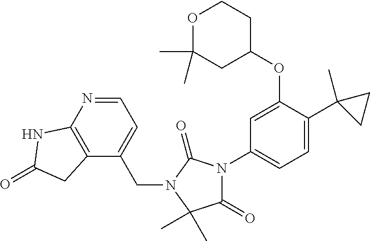 [SMILES: CC1(C)CC(Oc2cc(N3C(=O)N(Cc4ccnc5c4CC(=O)N5)C(C)(C)C3=O)ccc2C2(C)CC2)CCO1] 3-[3-(2,2-dimethyltetrahydro-2H-pyran-4-yloxy)- 4-(1-methylcyclopropyl)phenyl]-5,5-dimethyl-1- [(2-oxo-1,7-diaza-4-indanyl)methyl]-2,4- imidazolidinedione | LCMS: 531.5 (M − H) (500 MHz, DMSO) δ 10.99 (br s, 1H), 8.02 (d, J = 5.5 Hz, 1H), 7.28 (d, J = 8.1 Hz, 1H), 7.05 (d, J = 1.9 Hz, 1H), 6.94 (d, J = 5.5 Hz, 1H), 6.89 (dd, J = 8.0, 1.9 Hz, 1H), 4.73-4.65 (m, 1H), 4.52 (s, 2H), 3.81-3.75 (m, 1H), 3.69-3.58 (m, 3H), 2.05- 1.96 (m, 2H), 1.54-1.45 (m, 2H), 1.43-1.34 (m, 6H), 1.29 (s, 3H), 1.25 (s, 3H), 1.22 (s, 3H), 0.71-0.66 (m, 2H), 0.64 (t, J = 4.5 Hz, 2H). |
| 14 | 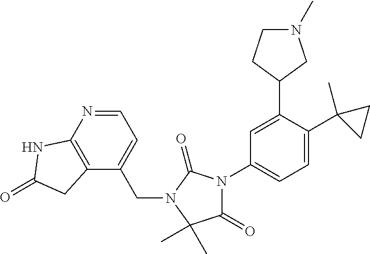 [SMILES: CN1CCC(c2cc(N3C(=O)N(Cc4ccnc5c4CC(=O)N5)C(C)(C)C3=O)ccc2C2(C)CC2)C1] 5,5-dimethyl-3-[4-(1-methylcyclopropyl)-3-(1- methyl-3-pyrrolidinyl)phenyl]-1-[(2-oxo-1,7- diaza-4-indanyl)methyl]-2,4-imidazolidinedione | LCMS: 488.3 (400 MHz, DMSO) δ 10.96 (s, 1H), 7.99 (d, J = 5.5 Hz, 1H), 7.38 (d, J = 2.1 Hz, 1H), 7.29 (d, J = 8.2 Hz, 1H), 7.12 (dd, J = 8.2, 2.1 Hz, 1H), 6.92 (d, J = 5.5 Hz, 1H), 4.48 (s, 2H), 4.10- 3.94 (m, 1H), 3.60 (s, 2H), 2.83 (t, J = 8.6 Hz, 1H), 2.70-2.55 (m, 2H), 2.44-2.38 (m, 1H), 2.28 (s, 4H), 1.74-1.60 (m, 1H), 1.35 (s, 6H), 1.26 (s, 3H), 0.77-0.72 (m, 2H), 0.72-0.64 (m, 2H). |
| 15 | 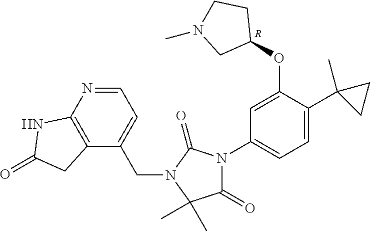 3-{3-[(R)-1-methyl-3-pyrrolidinyloxy]-4-(1- methylcyclopropyl)phenyl}-5,5-dimethyl-1-[(2- oxo-1,7-diaza-4-indanyl)methyl]-2,4- imidazolidinedione | LCMS: 504.7 (500 MHz, DMSO) δ 11.00 (s, 1H), 8.03 (d, J = 5.5 Hz, 1H), 7.27 (d, J = 8.0 Hz, 1H), 6.97- 6.93 (m, 2H), 6.89 (dd, J = 8.0, 1.9 Hz, 1H), 4.91-4.84 (m, 1H), 4.52 (s, 2H), 3.63 (s, 2H), 2.96 (dd, J = 10.3, 6.2 Hz, 1H), 2.62 (dd, J = 15.7, 7.1 Hz, 1H), 2.53 (dd, J = 10.4, 3.1 Hz, 1H), 2.49-2.45 (m, 1H), 2.33-2.24 (m, 4H), 1.87-1.79 (m, 1H), 1.38 (s, 6H), 1.28 (s, 3H), 0.68 (t, J = 4.8 Hz, 2H), 0.63 (t, J = 4.9 Hz, 2H). |
| 16 | 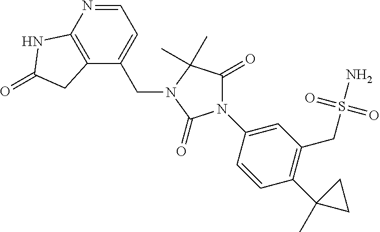 [SMILES: CC1(c2ccc(N3C(=O)N(Cc4ccnc5c4CC(=O)N5)C(C)(C)C3=O)cc2CS(N)(=O)=O)CC1] (5-{4,4-dimethyl-2,5-dioxo-3-[(2-oxo-1,7-diaza- 4-indanyl)methyl]-1-imidazolidinyl}-2-(1- methylcyclopropyl)phenyl)methanesulfonamide | LCMS: 496.4 (M − H) (500 MHz, DMSO) δ 11.00 (s, 1H), 8.32 (s, 1H), 8.02 (d,J = 5.5 Hz, 1H), 7.57 (d,J = 2.2 Hz, 1H), 7.48 (d,J = 8.3 Hz, 1H), 7.36 (dd,J = 8.3, 2.2 Hz, 1H), 7.12 (s, 2H), 6.94 (d,J = 5.5 Hz, 1H), 4.63 (s, 2H), 4.53 (s, 2H), 3.64 (s, 2H), 1.39 (s, 6H), 1.33 (s, 3H), 0.90-0.85 (m, 2H), 0.80- 0.73 (m, 2H) |
| 17 | 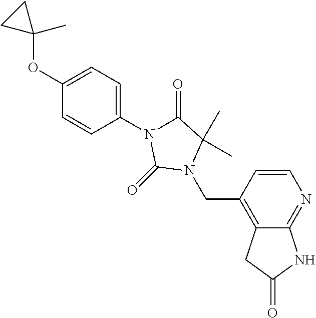 [SMILES: CC1(Oc2ccc(N3C(=O)N(Cc4ccnc5c4CC(=O)N5)C(C)(C)C3=O)cc2)CC1] 5,5-dimethyl-3-[p-(1- methylcyclopropoxy)phenyl]-1-[(2-oxo-1,7- diaza-4-indanyl)methyl]-2,4-imidazolidinedione | LCMS: 421.3 (400 MHz, DMSO-d6) δ 10.99 (s, 1H), 8.02 (d, J = 5.5 Hz, 1H), 7.38-7.29 (m, 2H), 7.12-7.04 (m, 2H), 6.96 (d, J = 5.5 Hz, 1H), 4.51 (s, 2H), 3.63 (s, 2H), 1.51 (s, 3H), 1.38 (s, 6H), 0.93- 0.89 (m, 2H), 0.82-0.74 (m, 2H). |
| 18 | 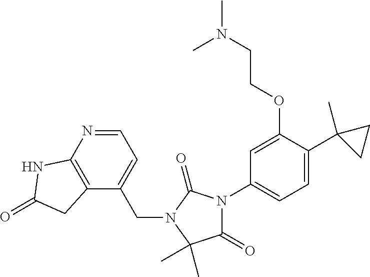 [SMILES: CN(C)CCOc1cc(N2C(=O)N(Cc3ccnc4c3CC(=O)N4)C(C)(C)C2=O)ccc1C1(C)CC1] 3-{3-[2-(dimethylamino)ethoxy]-4-(1- methylcyclopropyl)phenyl}-5,5-dimethyl-1-[(2- oxo-1,7-diaza-4-indanyl)methyl]-2,4- imidazolidinedione | LCMS: 490.5 (M − H) (500 MHz, DMSO) δ 10.99 (br s, 1H), 8.02 (d, J = 5.5 Hz, 1H), 7.28 (d, J = 8.0 Hz, 1H), 7.03 (d, J = 1.9 Hz, 1H), 6.95 (d, J = 5.5 Hz, 1H), 6.89 (dd, J = 8.0, 1.9 Hz, 1H), 4.52 (s, 2H), 4.08 (t, J = 5.7 Hz, 2H), 3.64 (s, 2H), 2.71 (t, J = 5.7 Hz, 2H), 2.27 (s, 6H), 1.38 (s, 6H), 1.29 (s, 3H), 0.72- 0.67 (m, 2H), 0.66-0.60 (m, 2H). |
| 19 | 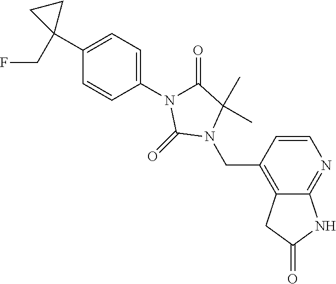 [SMILES: CC1(C)C(=O)N(c2ccc(C3(CF)CC3)cc2)C(=O)N1Cc1ccnc2c1CC(=O)N2] 3-{p-[1-(fluoromethyl)cyclopropyl]phenyl}-5,5- dimethyl-1-[(2-oxo-1,7-diaza-4-indanyl)methyl]- 2,4-imidazolidinedione | LCMS: 423.3 (400 HMz, DMSO-d6): δ 11.00 (s, 1H), 8.02 (d, J = 5.5 Hz, 1H), 7.44 (d, J = 8.4 Hz, 2H), 7.37 (d, J = 8.3 Hz, 2H), 6.97 (d, J = 5.5 Hz, 1H), 4.53 (d, J = 49 Hz, 2H), 4.525 (s, 2H), 3.64 (s, 2H), 1.39 (s, 6H), 1.07-0.94 (m, 4H). |
| 20 | 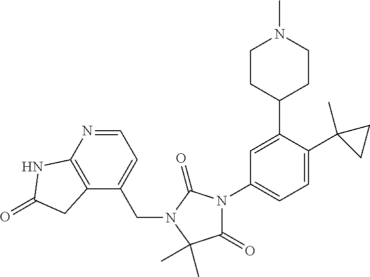 [SMILES: CN1CCC(c2cc(N3C(=O)N(Cc4ccnc5c4CC(=O)N5)C(C)(C)C3=O)ccc2C2(C)CC2)CC1] 5,5-dimethyl-3-[4-(1-methylcyclopropyl)-3-(1- methyl-4-piperidyl)phenyl]-1-[(2-oxo-1,7-diaza- 4-indanyl)methyl]-2,4-imidazolidinedione | LCMS: 502.4 (400 MHz, DMSO) δ 10.96 (s, 1H), 7.99 (d, J = 5.5 Hz, 1H), 7.39-7.27 (m, 2H), 7.11 (dd, J = 8.2, 2.1 Hz, 1H), 6.93 (d, J = 5.5 Hz, 1H), 4.48 (s, 2H), 3.60 (s, 2H), 3.25-3.11 (m, 1H), 2.89 (d, J = 10.5 Hz, 2H), 2.20 (s, 3H), 2.04-1.89 (m, 2H), 1.75-1.56 (m, 4H), 1.35 (s, 6H), 1.25 (s, 3H), 0.73 (s, 2H), 0.71 (s, 2H). |
| 21 | 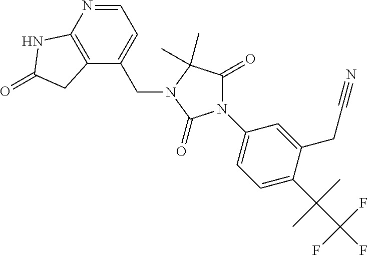 [SMILES: CC1(C)C(=O)N(c2ccc(C(C)(C)C(F)(F)F)c(CC#N)c2)C(=O)N1Cc1ccnc2c1CC(=O)N2] (5-{4,4-dimethyl-2,5-dioxo-3-[(2-oxo-1,7-diaza- 4-indanyl)methyl]-1-imidazolidinyl}-2-(2,2,2- trifluoro-1,1-dimethylethyl)phenyl)acetonitrile | LCMS: 500.5 (400 MHz, DMSO) δ 10.97 (s, 1H), 7.99 (d, J = 5.5 Hz, 1H), 7.73 (d, J = 8.8 Hz, 1H), 7.62 (d, J = 2.3 Hz, 1H), 7.48 (dd, J = 8.7, 2.3 Hz, 1H), 6.96 (d, J = 5.5 Hz, 1H), 4.50 (s, 2H), 4.23 (s, 2H), 3.62 (s, 2H), 1.70 (s, 6H), 1.38 (s, 6H). |
| 22 | 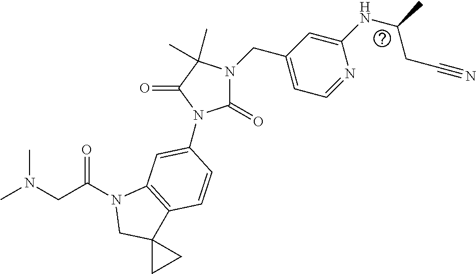 [SMILES: C[C@@H](CC#N)Nc1cc(CN2C(=O)N(c3ccc4c(c3)N(C(=O)CN(C)C)CC43CC3)C(=O)C2(C)C)ccn1] (S)-3-{4-[(3-{1′-[2- (dimethylamino)acetyl]spiro[cyclopropane-1,3′- indolin]-6′-yl}-5,5-dimethyl-2,4-dioxo-1- imidazolidinyl)methyl]-2- pyridylamino}butyronitrile | LCMS: 530.4 (400 MHz, DMSO) δ 8.03 (d,J = 1.8 Hz, 1H), 7.92 (d,J = 5.2 Hz, 1H), 6.97 (dd,J = 8.0, 1.9 Hz, 1H), 6.88 (d,J = 8.0 Hz, 1H), 6.73 (d,J = 6.9 Hz, 1H), 6.54- 6.48 (m, 2H), 4.42 (s, 2H), 4.20 (s, 2H), 4.17-4.09 (m, 1H), 3.19 (s, 2H), 2.83 (dd,J = 16.7, 5.9 Hz, 1H), 2.71 (dd,J = 16.7, 4.8 Hz, 1H), 2.25 (s, 6H), 1.44- 1.33 (m, 6H), 1.24 (d,J = 6.7 Hz, 3H), 1.12 (t,J = 5.6 Hz, 2H), 1.07 (t,J = 5.5 Hz, 2H) |
| 23 | 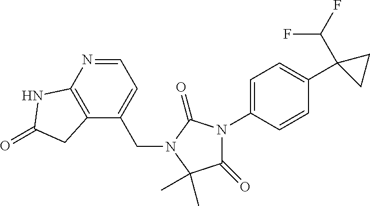 [SMILES: CC1(C)C(=O)N(c2ccc(C3(C(F)F)CC3)cc2)C(=O)N1Cc1ccnc2c1CC(=O)N2] 3-{p-[1-(difluoromethyl)cyclopropyl]phenyl}- 5,5-dimethyl-1-[(2-oxo-1,7-diaza-4- indanyl)methyl]-2,4-imidazolidinedione | 441.3 (400 MHz, DMSO-d6): δ 11.00 (s, 1H), 8.02 (d, J = 5.5 Hz, 1H), 7.52-7.47 (m, 2H), 7.45-7.39 (m, 2H), 6.97 (d, J = 5.5 Hz, 1H), 5.88 (t, J = 56.1 Hz, 1H), 4.53 (s, 2H), 3.64 (s, 2H), 1.39 (s, 6H), 1.15 (dd, J = 6.4 Hz, J = 5.2 Hz, 2H), 1.05-0.98 (m, 2H). |
| 25 | 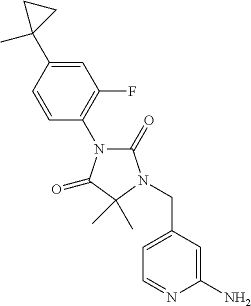 [SMILES: CC1(c2ccc(N3C(=O)N(Cc4ccnc(N)c4)C(C)(C)C3=O)c(F)c2)CC1] 1-[(2-amino-4-pyridyl)methyl]-3-[2-fluoro-4-(1- methylcyclopropyl)phenyl]-5,5-dimethyl-2,4- imidazolidinedione | LCMS: 383.5 (400 MHz, DMSO-d6) δ 7.85 (d, J = 5.2 Hz, 1H), 7.43-7.34 (m, J = 8.1 Hz, 1H), 7.25-7.14 (m, 2H), 6.46 (d, J = 5.3 Hz, 1H), 6.41 (s, 1H), 5.90 (s, 2H), 4.43 (s, 2H), 1.41 (s, 3H), 1.39 (s, 6H), 0.98-0.92 (m, 2H), 0.86- 0.81 (m, 2H). |
| 26 | 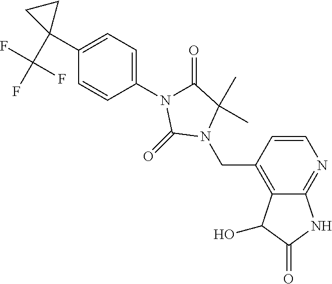 [SMILES: CC1(C)C(=O)N(c2ccc(C3(C(F)(F)F)CC3)cc2)C(=O)N1Cc1ccnc2c1C(O)C(=O)N2] 1-[(3-hydroxy-2-oxo-1,7-diaza-4- indanyl)methyl]-5,5-dimethyl-3-{p-[1- (trifluoromethyl)cyclopropyl]phenyl}-2,4- imidazolidinedione | LCMS: 475.2 (400 MHz, acetone) δ 9.87 (s, 1H), 8.06 (d, J = 5.5 Hz, 1H), 7.61 (d, J = 8.6 Hz, 2H), 7.57- 7.50 (m, 2H), 7.09 (d, J = 5.6 Hz, 1H), 5.70 (s, 1H), 5.33 (s, 1H), 4.85 (d, J = 16.9 Hz, 1H), 4.80 (d, J = 16.9 Hz, 1H), 1.52 (s, 3H), 1.45 (s, 3H), 1.41-1.37 (m, 2H), 1.20-1.14 (m, 2H). |
| 27 | 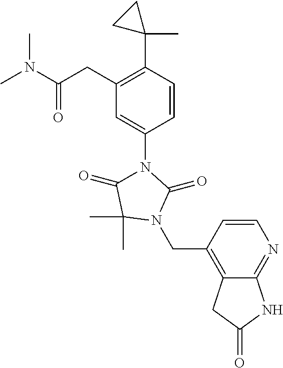 [SMILES: CN(C)C(=O)Cc1cc(N2C(=O)N(Cc3ccnc4c3CC(=O)N4)C(C)(C)C2=O)ccc1C1(C)CC1] N,N-dimethyl(5-{4,4-dimethyl-2,5-dioxo-3-[(2- oxo-1,7-diaza-4-indanyl)methyl]-1- imidazolidinyl}-2-(1- methylcyclopropyl)phenyl)acetamide | LCMS: 490.3 (400 MHz, DMSO) δ 10.99 (s, 1H), 8.02 (d,J = 5.5 Hz, 1H), 7.38 (d, J = 8.2 Hz, 1H), 7.23 (dd,J = 8.2, 2.0 Hz, 1H), 7.10 (d,J = 1.9 Hz, 1H), 6.95 (d,J = 5.5 Hz, 1H), 4.51 (s, 2H), 3.91 (s, 2H), 3.64 (s, 2H), 3.08 (s, 3H), 2.88 (s, 3H), 1.38 (s, 6H), 1.25 (s, 3H), 0.77-0.69 (m, 4H). |
| 28 | 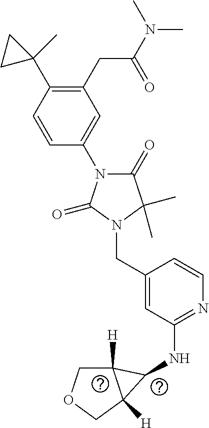 [SMILES: CN(C)C(=O)Cc1cc(N2C(=O)N(Cc3ccnc(N[C@H]4[C@@H]5COC[C@@H]54)c3)C(C)(C)C2=O)ccc1C1(C)CC1] N,N-dimethyl(5-{3-[(2-{(1R,5S,6r)-3- oxabicyclo[3.1.0]hex-6-ylamino}-4- pyridyl)methyl]-4,4-dimethyl-2,5-dioxo-1- imidazolidinyl}-2-(1- methylcyclopropyl)phenyl)acetamide | LCMS: 532.4 (400 MHz, DMSO) δ 7.94 (d,J = 5.3 Hz, 1H), 7.38 (d,J = 8.2 Hz, 1H), 7.19 (dd,J = 8.2, 2.2 Hz, 1H), 7.07 (d,J = 2.2 Hz, 1H), 6.73 (d,J = 2.3 Hz, 1H), 6.57 (dd,J = 5.2, 1.1 Hz, 1H), 6.50 (s, 1H), 4.48 (s, 2H), 3.91 (s, 2H), 3.86 (d,J = 8.4 Hz, 2H), 3.63 (d, J = 8.1 Hz, 2H), 3.08 (s, 3H), 2.87 (s, 3H), 2.26 (s, 1H), 1.75 (s, 2H), 1.38 (s, 6H), 1.25 (s, 3H), 0.79-0.65 (m, 4H) |
| 29 | 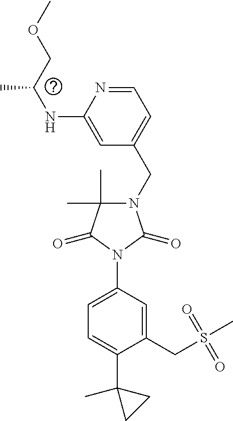 [SMILES: COC[C@@H](C)Nc1cc(CN2C(=O)N(c3ccc(C4(C)CC4)c(CS(C)(=O)=O)c3)C(=O)C2(C)C)ccn1] 1-({2-[(R)-2-methoxy-1-methylethylamino]-4- pyridyl}methyl)-3-[3-(mesylmethyl)-4-(1- methylcyclopropyl)phenyl]-5,5-dimethyl-2,4- imidazolidinedione | LCMS: 527.4 (M − H) (500 MHz, DMSO-d6) δ 7.89 (dd,J = 5.1, 0.7 Hz, 1H), 7.53- 7.51 (m, 2H), 7.39 (dd,J = 8.3, 2.2 Hz, 1H), 6.45-6.44 (m, 2H), 6.31 (d,J = 7.9 Hz, 1H), 4.78 (s, 2H), 4.43 (s, 2H), 4.14- 4.06 (m, 1H), 3.38 (dd,J = 9.2, 5.1 Hz, 1H), 3.25 (s, 3H), 3.21 (dd,J = 9.2, 6.2 Hz, 1H), 3.17 (s, 3H), 1.40 (s, 3H), 1.40 (s, 3H), 1.32 (s, 3H), 1.10 (d,J = 6.6 Hz, 3H), 0.88-0.84 (m, 2H), 0.83- 0.79 (m, 2H). |
| 30 | 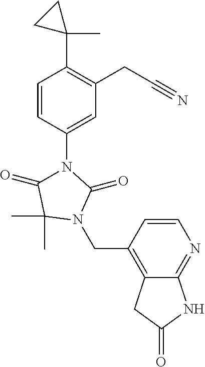 [SMILES: CC1(c2ccc(N3C(=O)N(Cc4ccnc5c4CC(=O)N5)C(C)(C)C3=O)cc2CC#N)CC1] (5-{4,4-dimethyl-2,5-dioxo-3-[(2-oxo-1,7-diaza- 4-indanyl)methyl]-1-imidazolidinyl}-2-(1- methylcyclopropyl)phenyl)acetonitrile | LCMS: 444.2 (400 MHz, DMSO) δ 10.99 (s, 1H), 8.00 (d, J = 5.5 Hz, 1H), 7.49-7.40 (m, 2H), 7.35 (dd, J = 8.2, 1.9 Hz, 1H), 6.96 (d, J = 5.3 Hz, 1H), 4.51 (s, 2H), 4.20 (s, 2H), 3.62 (s, 2H), 1.38 (s, 6H), 1.27 (s, 3H), 0.82-0.73 (m, 4H). |
| 31 | 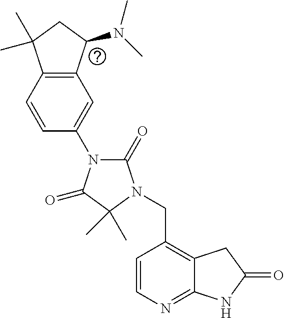 [SMILES: CN(C)[C@@H]1CC(C)(C)c2ccc(N3C(=O)N(Cc4ccnc5c4CC(=O)N5)C(C)(C)C3=O)cc21] 3-[(R)-3-(dimethylamino)-1,1-dimethyl-5- indanyl]-5,5-dimethyl-1-[(2-oxo-1,7-diaza-4- indanyl)methyl]-2,4-imidazolidinedione | LCMS: 462.4 (400 MHz, DMSO) δ 11.01 (s, 1H), 8.03 (d,J = 5.5 Hz, 1H), 7.35-7.21 (m, 3H), 6.98 (d,J = 5.5 Hz, 1H), 4.59-4.45 (m, 2H), 4.42 (t,J = 8.0 Hz, 1H), 3.64 (s, 2H), 2.18 (s, 6H), 1.88 (d,J = 8.2 Hz, 2H), 1.40 (s, 3H), 1.36 (d,J = 6.4 Hz, 6H), 1.17 (s, 3H). |
| 32 | 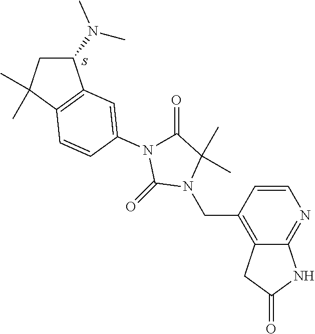 3-[(S)-3-(dimethylamino)-1,1-dimethyl-5- indanyl]-5,5-dimethyl-1-[(2-oxo-1,7-diaza-4- indanyl)methyl]-2,4-imidazolidinedione | LCMS: 462.5 (400 MHz, DMSO) δ 11.01 (s, 1H), 8.03 (d,J = 5.5 Hz, 1H), 7.39-7.18 (m, 3H), 6.98 (d,J = 5.6 Hz, 1H), 4.52 (q,J = 17.0 Hz, 2H), 4.42 (t,J = 8.2 Hz, 1H), 3.64 (s, 2H), 2.18 (s, 6H), 1.88 (d,J = 8.2 Hz, 2H), 1.40 (s, 3H), 1.36 (d,J = 6.3 Hz, 6H), 1.17 (s, 3H). |
| 33 | 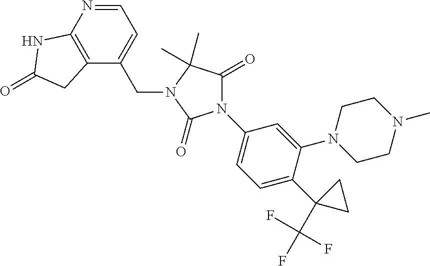 [SMILES: CN1CCN(c2cc(N3C(=O)N(Cc4ccnc5c4CC(=O)N5)C(C)(C)C3=O)ccc2C2(C(F)(F)F)CC2)CC1] 5,5-dimethyl-3-{3-(4-methyl-1-piperazinyl)-4-[1- (trifluoromethyl)cyclopropyl]phenyl}-1-[(2-oxo- 1,7-diaza-4-indanyl)methyl]-2,4- imidazolidinedione | LCMS: 557.3 (400 MHz, DMSO) δ 10.98 (s, 1H), 8.00 (d, J = 5.5 Hz, 1H), 7.53 (d, J = 8.3 Hz, 1H), 7.40 (d, J = 2.1 Hz, 1H), 7.24 (dd, J = 8.3, 2.1 Hz, 1H), 6.95 (d, J = 5.5 Hz, 1H), 4.49 (s, 2H), 3.61 (s, 2H), 2.82 (t, J = 4.6 Hz, 4H), 2.46-2.40 (m, 4H), 2.21 (s, 3H), 1.41-1.32 (m, 8H), 1.20 (s, 2H). |
| 35 | 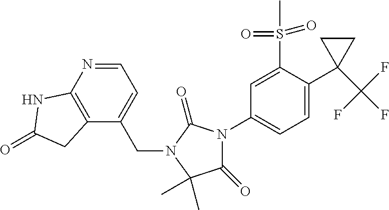 [SMILES: CC1(C)C(=O)N(c2ccc(C3(C(F)(F)F)CC3)c(S(C)(=O)=O)c2)C(=O)N1Cc1ccnc2c1CC(=O)N2] 3-{3-mesyl-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 537.3 (500 MHz, DMSO-d6) δ 10.99 (s, 1H), 8.25 (s, 1H), 8.03 (d,J = 5.5 Hz, 1H), 7.86 (s, 2H), 7.01 (d,J = 5.5 Hz, 1H), 4.55 (s, 2H), 3.66 (s, 2H), 3.27 (s, 3H), 1.68- 1.21 (m, 10H). |
| 36 | 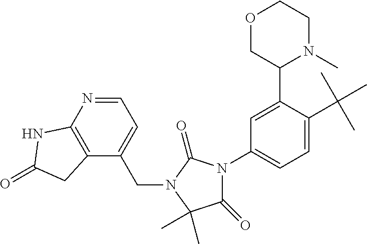 [SMILES: CN1CCOCC1c1cc(N2C(=O)N(Cc3ccnc4c3CC(=O)N4)C(C)(C)C2=O)ccc1C(C)(C)C] 3-[4-(tert-butyl)-3-(4-methyl-3- morpholinyl)phenyl]-5,5-dimethyl-1-[(2-oxo-1,7- diaza-4-indanyl)methyl]-2,4-imidazolidinedione | LCMS: 506.3 (400 MHz, DMSO) δ 10.58 (br s, 1H) 8.00 (d, J = 5.5 Hz, 1H), 7.70 (d, J = 2.5 Hz, 1H), 7.43 (d, J = 8.7 Hz, 1H), 7.24 (dd, J = 8.6, 2.5 Hz, 1H), 6.94 (d, J = 5.5 Hz, 1H), 4.50 (s, 2H), 3.84- 3.78 (m, 2H), 3.69-3.59 (m, 4H), 3.16 (dd, J = 11.3, 9.8 Hz, 1H), 2.84 (d, J = 11.7 Hz, 1H), 2.40 (td, J = 11.8, 3.3 Hz, 1H), 2.00 (s, 3H), 1.41 (s, 9H), 1.37 (s, 6H). |
| 38 | 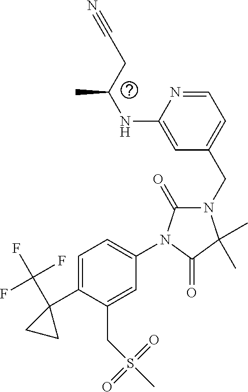 [SMILES: C[C@@H](CC#N)Nc1cc(CN2C(=O)N(c3ccc(C4(C(F)(F)F)CC4)c(CS(C)(=O)=O)c3)C(=O)C2(C)C)ccn1] (S)-3-{4-[(3-{3-(mesylmethyl)-4-[1- (trifluoromethyl)cyclopropyl]phenyl}-5,5- dimethyl-2,4-dioxo-1-imidazolidinyl)methyl]-2- pyridylamino}butyronitrile | LCMS: 578.6 (500 MHz, DMSO) δ 7.95 (d, J = 5.3 Hz, 1H), 7.70-7.64 (m, 2H), 7.56 (dd, J = 8.4, 2.2 Hz, 1H), 6.71 (d, J = 7.0 Hz, 1H), 6.55 (d, J = 5.3 Hz, 1H), 6.53 (s, 1H), 4.81 (s, 2H), 4.47 (s, 2H), 4.20-4.10 (m, 1H), 3.22 (s, 3H), 2.84 (dd, J = 16.7, 5.9 Hz, 1H), 2.73 (dd, J = 16.7, 4.8 Hz, 1H), 1.42 (s, 6H), 1.26 (d, J = 6.7 Hz, 3H). |
| 39 | 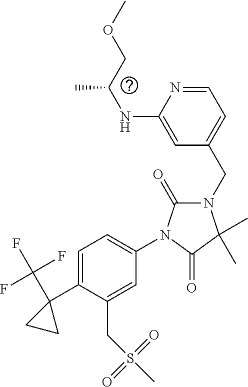 [SMILES: COC[C@@H](C)Nc1cc(CN2C(=O)N(c3ccc(C4(C(F)(F)F)CC4)c(CS(C)(=O)=O)c3)C(=O)C2(C)C)ccn1] 1-({2-[(R)-2-methoxy-1-methylethylamino]-4- pyridyl}methyl)-3-{3-(mesylmethyl)-4-[1- (trifluoromethyl)cyclopropyl]phenyl}-5,5- dimethyl-2,4-imidazolidinedione | LCMS: 583.6 (500 MHz, DMSO) δ 7.92- 7.86 (m, 1H), 7.70-7.65 (m, 2H), 7.55 (dd, J = 8.4, 2.2 Hz, 1H), 6.49-6.43 (m, 2H), 6.30 (d, J = 7.9 Hz, 1H), 4.81 (s, 2H), 4.44 (s, 2H), 4.15-4.06 (m, 1H), 3.38 (dd, J = 9.2, 5.1 Hz, 1H), 3.25 (s, 3H), 3.23-3.19 (m, 4H), 1.56-1.38 (m, 10H), 1.11 (d, J = 6.6 Hz, 3H). |
| 40 | 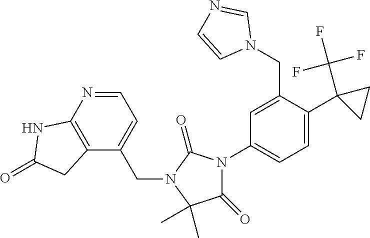 [SMILES: CC1(C)C(=O)N(c2ccc(C3(C(F)(F)F)CC3)c(Cn3ccnc3)c2)C(=O)N1Cc1ccnc2c1CC(=O)N2] 3-{3-[(1-imidazolyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 539.3 (400 MHz, DMSO) δ 10.96 (s, 1H), 7.98 (d, J = 5.3 Hz, 1H), 7.63 (d, J = 8.3 Hz, 1H), 7.47- 7.43 (m, 1H), 7.26-7.03 (m, 2H), 7.01-6.91 (m, 2H), 6.89 (d, J = 5.5 Hz, 1H), 5.44 (s, 2H), 4.46 (s, 2H), 3.58 (s, 2H), 1.55- 1.50 (m, 2H), 1.32 (s, 6H), 1.26- 1.23 (m, 2H). |
| 41 | 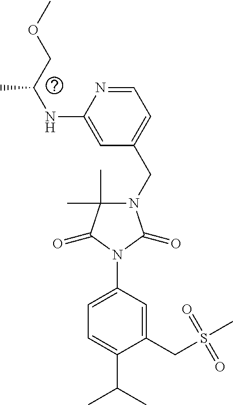 [SMILES: COC[C@@H](C)Nc1cc(CN2C(=O)N(c3ccc(C(C)C)c(CS(C)(=O)=O)c3)C(=O)C2(C)C)ccn1] 1-({2-[(R)-2-methoxy-1-methylethylamino]-4- pyridyl}methyl)-3-[3-(mesylmethyl)-4-cumenyl]- 5,5-dimethyl-2,4-imidazolidinedione | LCMS: 515.5 (M − H) (500 MHz, DMSO-d6) δ 7.89 (d,J = 5.2 Hz, 1H), 7.52 (d,J = 8.4 Hz, 1H), 7.42 (dd,J = 8.4, 2.2 Hz, 1H), 7.39 (d,J = 2.2 Hz, 1H), 6.46-6.44 (m, 2H), 6.30 (d,J = 7.8 Hz, 1H), 4.63 (s, 2H), 4.43 (s, 2H), 4.14-4.06 (m, 1H), 3.37 (ddd,J = 15.6, 9.5, 5.0 Hz, 2H), 3.25 (s, 3H), 3.21 (dd,J = 9.2, 6.2 Hz, 1H), 3.04 (s, 3H), 1.40 (d,J = 2.0 Hz, 6H), 1.20 (d, J = 6.8 Hz, 6H), 1.10 (d,J = 6.6 Hz, 3H). |
| 42 | 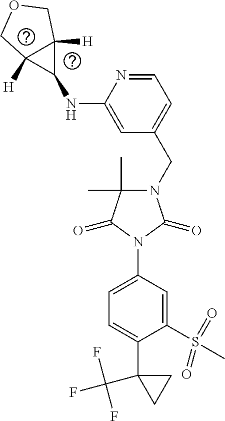 [SMILES: CC1(C)C(=O)N(c2ccc(C3(C(F)(F)F)CC3)c(S(C)(=O)=O)c2)C(=O)N1Cc1ccnc(N[C@H]2[C@@H]3COC[C@@H]32)c1] 1-[(2-{(1R,5S,6r)-3-oxabicyclo[3.1.0]hex-6- ylamino}-4-pyridyl)methyl]-3-{3-mesyl-4-[1- (trifluoromethyl)cyclopropyl]phenyl}-5,5- dimethyl-2,4-imidazolidinedione | LCMS: 577.3 (M − H) (500 MHz, DMSO-d6) δ 8.23 (dd,J = 1.9, 0.5 Hz, 1H), 7.95 (d,J = 5.2 Hz, 1H), 7.86- 7.82 (m, 2H), 6.71 (d,J = 2.3 Hz, 1H), 6.61 (dd,J = 5.2, 1.4 Hz, 1H), 6.53 (s, 1H), 4.51 (s, 2H), 3.86 (d,J = 8.4 Hz, 2H), 3.63 (d, J = 8.2 Hz, 2H), 3.26 (s, 3H), 2.26 (dd,J = 4.6, 2.4 Hz, 1H), 1.75 (t, J = 2.5 Hz, 2H), 1.55-1.43 (m, 10H). |
| 43 | 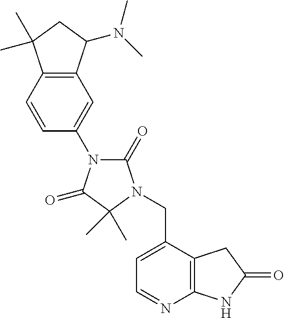 [SMILES: CN(C)C1CC(C)(C)c2ccc(N3C(=O)N(Cc4ccnc5c4CC(=O)N5)C(C)(C)C3=O)cc21] 3-[3-(dimethylamino)-1,1-dimethyl-5-indanyl]- 5,5-dimethyl-1-[(2-oxo-1,7-diaza-4- indanyl)methyl]-2,4-imidazolidinedione | LCMS: 462.4 (400 MHz, DMSO) δ 11.00 (s, 1H), 8.03 (d, J = 5.5 Hz, 1H), 7.34-7.22 (m, 3H), 6.97 (d, J = 5.5 Hz, 1H), 4.58-4.46 (m, 2H), 4.42 (t, J = 8.0 Hz, 1H), 3.64 (s, 2H), 2.18 (s, 6H), 1.88 (d, J = 8.0 Hz, 2H), 1.40 (s, J = 9.5 Hz, 3H), 1.37 (d, J = 6.7 Hz, 6H), 1.18 (s, 3H). |
| 44 | 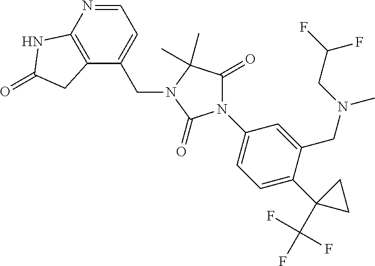 [SMILES: CN(Cc1cc(N2C(=O)N(Cc3ccnc4c3CC(=O)N4)C(C)(C)C2=O)ccc1C1(C(F)(F)F)CC1)CC(F)F] 3-(3-{[(2,2-difluoroethyl)-N- methylamino]methyl}-4-[1- (trifluoromethyl)cyclopropyl]phenyl)-5,5- dimethyl-1-[(2-oxo-1,7-diaza-4-indanyl)methyl]- 2,4-imidazolidinedione | LCMS: 566.5 (500 MHz, DMSO) δ 10.99 (s, 1H), 8.03 (d, J = 5.5 Hz, 1H), 7.71 (d, J = 2.2 Hz, 1H), 7.56 (d, J = 8.3 Hz, 1H), 7.39 (dd, J = 8.3, 2.3 Hz, 1H), 6.97 (d, J = 5.5 Hz, 1H), 6.20 (tt, J = 55.8, 4.3 Hz, 1H), 4.54 (s, 2H), 3.86 (s, 2H), 3.65 (s, 2H), 2.82 (td, J = 15.3, 4.3 Hz, 2H), 2.33 (s, 3H), 1.52-1.44 (m, 2H), 1.40 (s, 6H), 1.26-1.17 (m, 2H). |
| 45 | 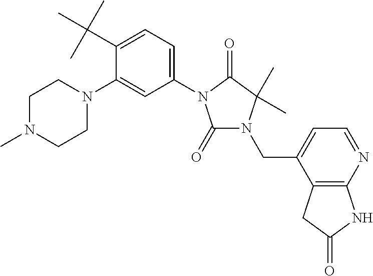 [SMILES: CN1CCN(c2cc(N3C(=O)N(Cc4ccnc5c4CC(=O)N5)C(C)(C)C3=O)ccc2C(C)(C)C)CC1] 3-[4-(tert-butyl)-3-(4-methyl-1- piperazinyl)phenyl]-5,5-dimethyl-1-[(2-oxo-1,7- diaza-4-indanyl)methyl]-2,4-imidazolidinedione | LCMS: 505.5 (500 MHz, DMSO) δ 11.00 (s, 1H), 8.04 (d, J = 5.5 Hz, 1H), 7.52 (d, J = 2.2 Hz, 1H), 7.41 (d, J = 8.6 Hz, 1H), 7.22 (dd, J = 8.5, 2.2 Hz, 1H), 6.98 (d, J = 5.5 Hz, 1H), 4.53 (s, 2H), 3.65 (s, 2H), 2.90 (t, J = 10.5 Hz, 2H), 2.78 (d, J = 11.0 Hz, 2H), 2.70 (d, J = 10.9 Hz, 2H), 2.25 (s, 3H), 2.16 (t, J = 10.1 Hz, 2H), 1.43 (s, 9H), 1.39 (s, 6H). |
| 46 | 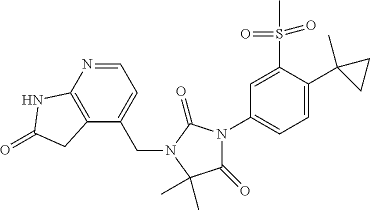 [SMILES: CC1(c2ccc(N3C(=O)N(Cc4ccnc5c4CC(=O)N5)C(C)(C)C3=O)cc2S(C)(=O)=O)CC1] 3-[3-mesyl-4-(1-methylcyclopropyl)phenyl]-5,5- dimethyl-1-[(2-oxo-1,7-diaza-4-indanyl)methyl]- 2,4-imidazolidinedione | LCMS: 483.4 (500 MHz, DMSO-d6) δ 10.99 (br, 1H), 8.10 (d, J = 1.6 Hz, 1H), 8.02 (d, J = 5.5 Hz, 1H), 7.77-7.73 (m, 2H), 7.01 (d, J = 5.5 Hz, 1H), 4.53 (s, 2H), 3.65 (s, 2H), 3.33 (s, 3H), 1.43 (s, 3H), 1.41 (s, 6H), 1.05-1.03 (m, 2H), 0.86-0.84 (m, 2H). |
| 47 | 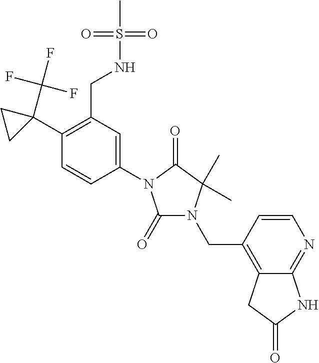 [SMILES: CC1(C)C(=O)N(c2ccc(C3(C(F)(F)F)CC3)c(CNS(C)(=O)=O)c2)C(=O)N1Cc1ccnc2c1CC(=O)N2] 3-{3-[(mesylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 566.1 (400 MHz, DMSO) δ 11.00 (s, 1H), 8.03 (d, J = 5.5 Hz, 1H), 7.65-7.60 (m, 2H), 7.58 (d, J = 8.3 Hz, 1H), 7.42 (dd, J = 8.3, 2.2 Hz, 1H), 6.97 (d, J = 5.5 Hz, 1H), 4.54 (s, 2H), 4.39 (br. s, 2H), 3.64 (s, 2H), 2.95 (s, 3H), 1.55-1.46 (m, 2H), 1.41 (s, 6H), 1.30-1.25 (m, 2H). |
| 48 | 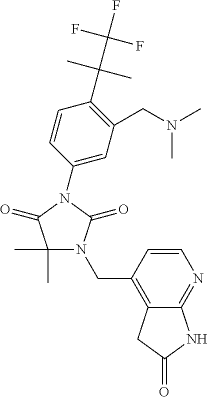 [SMILES: CN(C)Cc1cc(N2C(=O)N(Cc3ccnc4c3CC(=O)N4)C(C)(C)C2=O)ccc1C(C)(C)C(F)(F)F] 3-{3-[(dimethylamino)methyl]-4-(2,2,2-trifluoro- 1,1-dimethylethyl)phenyl}-5,5-dimethyl-1-[(2- oxo-1,7-diaza-4-indanyl)methyl]-2,4- imidazolidinedione | LCMS: 518.3 (400 MHz, DMSO) δ 10.99 (s, 1H), 8.02 (d,J = 5.5 Hz, 1H), 7.76 (d,J = 2.4 Hz, 1H), 7.65 (d, J = 8.8 Hz, 1H), 7.36 (dd,J = 8.7, 2.5 Hz, 1H), 6.97 (d,J = 5.5 Hz, 1H), 4.53 (s, 2H), 3.64 (s, 2H), 3.62 (s, 2H), 2.19 (s, 6H), 1.72 (s, 6H), 1.40 (s, 6H). |
| 49 | 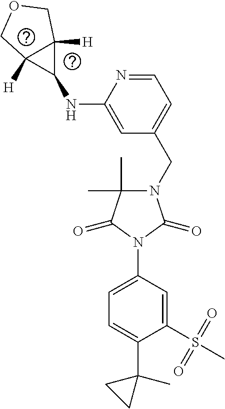 [SMILES: CC1(c2ccc(N3C(=O)N(Cc4ccnc(N[C@H]5[C@@H]6COC[C@@H]65)c4)C(C)(C)C3=O)cc2S(C)(=O)=O)CC1] 1-[(2-{(1R,5S,6r)-3-oxabicyclo[3.1.0]hex-6- ylamino}-4-pyridyl)methyl]-3-[3-mesyl-4-(1- methylcyclopropyl)phenyl]-5,5-dimethyl-2,4- imidazolidinedione | LCMS: 523.4 (M − H) (500 MHz, DMSO-d6) δ 8.07 (d,J = 2.2 Hz, 1H), 7.94 (d,J = 5.2 Hz, 1H), 7.76 (d,J = 8.3 Hz, 1H), 7.71 (dd,J = 8.3, 2.2 Hz, 1H), 6.71 (d,J = 2.3 Hz, 1H), 6.60 (dd,J = 5.2, 1.4 Hz, 1H), 6.52 (s, 1H), 4.50 (s, 2H), 3.86 (d,J = 8.4 Hz, 2H), 3.63 (d,J = 8.3 Hz, 2H), 3.32 (s, 3H), 2.26 (dd,J =4.8, 2.5 Hz, 1H), 1.75 (t, J= 2.8 Hz, 2H), 1.43 (s, 3H), 1.41 (s, 6H), 1.06-1.01 (m, 2H), 0.87-0.82 (m, 2H). |
| 50 | 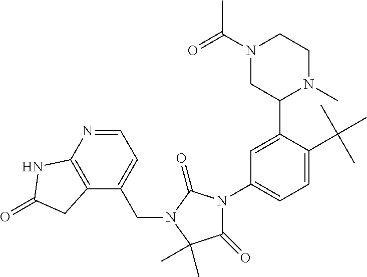 [SMILES: CC(=O)N1CCN(C)C(c2cc(N3C(=O)N(Cc4ccnc5c4CC(=O)N5)C(C)(C)C3=O)ccc2C(C)(C)C)C1] 3-[3-(4-acetyl-1-methyl-2-piperazinyl)-4-(tert- butyl)phenyl]-5,5-dimethyl-1-[(2-oxo-1,7-diaza- 4-indanyl)methyl]-2,4-imidazolidinedione | LCMS: 547.4 (400 MHz, DMSO) δ 11.00 (s, 1H), 8.00 (d, J = 5.5 Hz, 1H), 7.71 (dd, J = 4.5, 2.5 Hz, 1H), 7.45 (t, J = 9.0 Hz, 1H), 7.30- 7.22 (m, 1H), 6.94 (d, J = 5.5 Hz, 1H), 4.50 (s, 2H), 4.43- 4.32 (m, 1H), 3.86-3.56 (m, 4H), 3.31-3.21 (m, 1H), 2.95 (d, J = 11.6 Hz, 1H), 2.80-2.69 and 2.43-2.14 (m, 2H), 2.05- 1.94 (m, 5H), 1.92 (s, 1H), 1.48- 1.31 (m, 15H) |
| 51 | 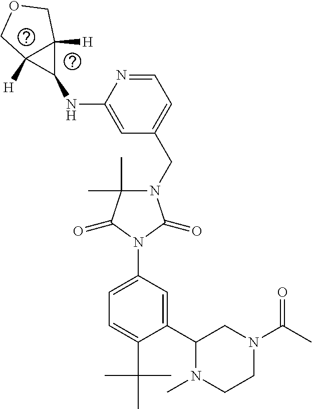 [SMILES: CC(=O)N1CCN(C)C(c2cc(N3C(=O)N(Cc4ccnc(N[C@H]5[C@@H]6COC[C@@H]65)c4)C(C)(C)C3=O)ccc2C(C)(C)C)C1] 1-[(2-{(1R,5S,6r)-3-oxabicyclo[3.1.0]hex-6- ylamino}-4-pyridyl)methyl]-3-[3-(4-acetyl-1- methyl-2-piperazinyl)-4-(tert-butyl)phenyl]-5,5- dimethyl-2,4-imidazolidinedione | LCMS: 589.5 (400 MHz, DMSO) δ 7.92 (d, J = 5.1 Hz, 1H), 7.70 (t, J = 2.6 Hz, 1H), 7.51-7.39 (m, 1H), 7.27-7.18 (m, 1H), 6.74 (s, 1H), 6.56 (d, J = 5.1 Hz, 1H), 6.48 (s, 1H), 4.46 (s, 2H), 4.42- 4.30 (m, 1H), 3.83 (d, J = 7.6 Hz, 2H), 3.81-3.67 (m, 1H), 3.60 (d, J = 8.3 Hz, 2H), 3.30- 3.21 (m, 2H), 2.94 (d, J = 11.7 Hz, 1H), 2.42-2.25 and 2.80- 2.66 (m, 2H, rotamers), 2.21 (s, 1H), 2.05-1.87 (m, 5H), 1.91 (s, 1H), 1.72 (s, 2H) 1.47-1.25 (m, 15H). |
| 52 | 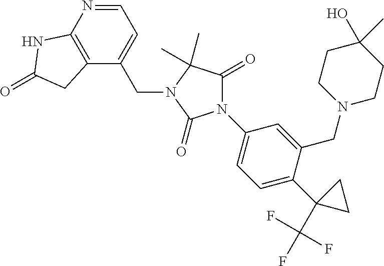 [SMILES: CC1(O)CCN(Cc2cc(N3C(=O)N(Cc4ccnc5c4CC(=O)N5)C(C)(C)C3=O)ccc2C2(C(F)(F)F)CC2)CC1] 3-{3-[(4-hydroxy-4-methyl-1-piperidyl)methyl]- 4-[1-(trifluoromethyl)cyclopropyl]phenyl}-5,5- dimethyl-1-[(2-oxo-1,7-diaza-4-indanyl)methyl]- 2,4-imidazolidinedione | LCMS: 586.7 (500 MHz, DMSO) δ 8.24 (s, 1H), 8.03 (d, J = 5.5 Hz, 1H), 7.69 (d, J = 2.3 Hz, 1H), 7.54 (d, J = 8.3 Hz, 1H), 7.36 (dd, J = 8.3, 2.3 Hz, 1H), 6.97 (d, J = 5.5 Hz, 1H), 4.54 (s, 2H), 3.70- 3.60 (m, J = 6.8 Hz, 4H), 2.46- 2.37 (m, 4H), 1.53-1.44 (m, 6H), 1.40 (s, 6H), 1.27-1.19 (m, 2H), 1.12 (s, 3H). |
| 53 | 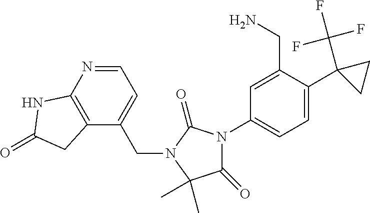 [SMILES: CC1(C)C(=O)N(c2ccc(C3(C(F)(F)F)CC3)c(CN)c2)C(=O)N1Cc1ccnc2c1CC(=O)N2] 3-{3-(aminomethyl)-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 488.5 (500 MHz, DMSO) δ 8.35 (br, 1H), 8.04 (d, J = 5.5 Hz, 1H), 7.70 (s, 1H), 7.52 (d, J = 8.2 Hz, 1H), 7.33 (dd, J = 8.3, 2.1 Hz, 1H), 6.96 (d, J = 5.5 Hz, 1H), 4.54 (s, 2H), 3.97 (br, 1H), 3.65 (s, 2H), 1.46-1.44 (m, 2H), 1.41 (s, 6H), 1.27-1.21 (m, 2H). |
| 55 | 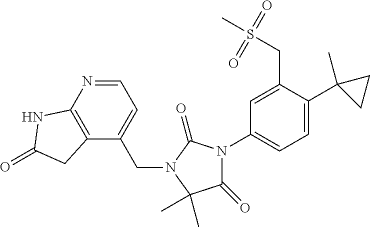 [SMILES: CC1(c2ccc(N3C(=O)N(Cc4ccnc5c4CC(=O)N5)C(C)(C)C3=O)cc2CS(C)(=O)=O)CC1] 3-[3-(mesylmethyl)-4-(1- methylcyclopropyl)phenyl]-5,5-dimethyl-1-[(2- oxo-1,7-diaza-4-indanyl)methyl]-2,4- imidazolidinedione | LCMS: 497.5 (400 MHz, DMSO) δ 10.97 (s, 1H), 8.00 (d, J = 5.4 Hz, 1H), 7.58-7.43 (m, 2H), 7.39 (dd, J = 8.2, 1.6 Hz, 1H), 6.92 (d, J = 5.5 Hz, 1H), 4.75 (s, 2H), 4.50 (s, 2H), 3.61 (s, 2H), 3.14 (s, 3H), 1.37 (s, 6H), 1.30 (s, 3H), 0.89-0.81 (m, 2H), 0.81-0.74 (m, 2H). |
| 56 | 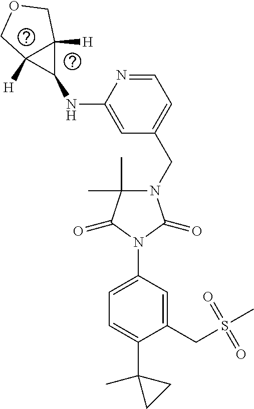 [SMILES: CC1(c2ccc(N3C(=O)N(Cc4ccnc(N[C@H]5[C@@H]6COC[C@@H]65)c4)C(C)(C)C3=O)cc2CS(C)(=O)=O)CC1] 1-[(2-{(1R,5S,6r)-3-oxabicyclo[3.1.0]hex-6- ylamino}-4-pyridyl)methyl]-3-[3-(mesylmethyl)- 4-(1-methylcyclopropyl)phenyl]-5,5-dimethyl- 2,4-imidazolidinedione | LCMS: 539.3 (400 MHz, DMSO) δ 7.91 (d, J = 5.2 Hz, 1H), 7.57-7.46 (m, 2H), 7.35 (dd, J = 8.3, 2.2 Hz, 1H), 6.70 (d, J = 2.2 Hz, 1H), 6.55 (dd, J = 5.2, 1.2 Hz, 1H), 6.48 (s, 1H), 4.75 (s, 2H), 4.47 (s, 2H), 3.83 (d, J = 8.4 Hz, 2H), 3.61 (d, J = 8.2 Hz, 2H), 3.13 (s, 3H), 2.23 (d, J = 2.1 Hz, 1H), 1.72 (s, 2H), 1.38 (d, J = 8.2 Hz, 6H), 1.29 (s, 3H), 0.88-0.81 (m, 2H), 0.81-0.73 (m, 2H). |
| 57 | 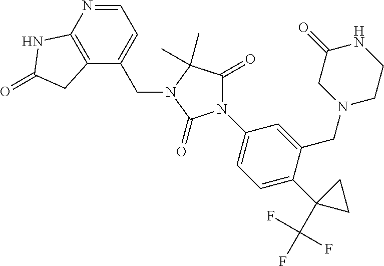 [SMILES: CC1(C)C(=O)N(c2ccc(C3(C(F)(F)F)CC3)c(CN3CCNC(=O)C3)c2)C(=O)N1Cc1ccnc2c1CC(=O)N2] 5,5-dimethyl-1-[(2-oxo-1,7-diaza-4- indanyl)methyl]-3-{3-[(3-oxo-1- piperazinyl)methyl]-4-[1- (trifluoromethyl)cyclopropyl]phenyl}-2,4- imidazolidinedione | LCMS: 571.6 (500 MHz, DMSO) δ 10.99 (br, 1H), 8.02 (d, J = 5.5 Hz, 1H), 7.77 (s, 1H), 7.73 (d, J = 2.2 Hz, 1H), 7.57 (d, J = 8.3 Hz, 1H), 7.40 (dd, J = 8.3, 2.3 Hz, 1H), 6.98 (d, J = 5.5 Hz, 1H), 4.54 (s, 2H), 3.77 (s, 2H), 3.64 (s, 2H), 3.21-3.14 (m, 2H), 3.02 (s, 2H), 2.58 (t, J = 5.2 Hz, 2H), 1.52-1.46 (m, 2H), 1.40 (s, 6H), 1.24-1.18 (m, 2H). |
| 58 | 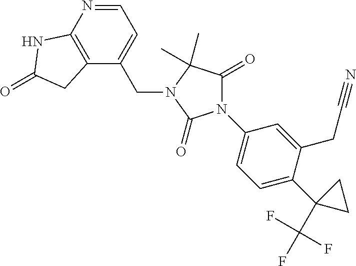 [SMILES: CC1(C)C(=O)N(c2ccc(C3(C(F)(F)F)CC3)c(CC#N)c2)C(=O)N1Cc1ccnc2c1CC(=O)N2] (5-{4,4-dimethyl-2,5-dioxo-3-[(2-oxo-1,7-diaza- 4-indanyl)methyl]-1-imidazolidinyl}-2-[1- (trifluoromethyl)cyclopropyl]phenyl)acetonitrile | LCMS: 498.4 (500 MHz, DMSO) δ 11.01 (br, 1H), 8.02 (d, J = 5.5 Hz, 1H), 7.66-7.61 (m, 2H), 7.51 (dd, J = 8.3, 2.1 Hz, 1H), 7.00 (d, J = 5.5 Hz, 1H), 4.53 (s, 2H), 4.21 (s, 2H), 3.65 (s, 2H), 1.56-1.46 (m, 2H), 1.41 (s, 6H), 1.26- 1.18 (m, 2H). |
| 59 | 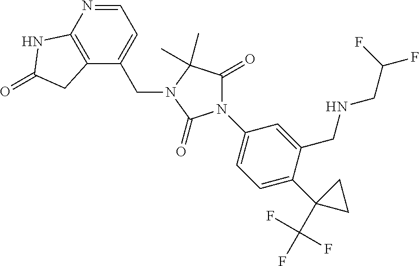 [SMILES: CC1(C)C(=O)N(c2ccc(C3(C(F)(F)F)CC3)c(CNCC(F)F)c2)C(=O)N1Cc1ccnc2c1CC(=O)N2] 3-{3-[(2,2-difluoroethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 552.5 (500 MHz, DMSO) δ 11.00 (s, 1H), 8.03 (d, J = 5.5 Hz, 1H), 7.66 (d, J = 2.2 Hz, 1H), 7.55 (d, J = 8.3 Hz, 1H), 7.37 (dd, J = 8.3, 2.3 Hz, 1H), 6.97 (d, J = 5.5 Hz, 1H), 6.06 (tt, J = 56.3, 4.3 Hz, 1H), 4.54 (s, 2H), 3.97 (s, 2H), 3.65 (s, 2H), 2.94 (td, J = 16.0, 4.2 Hz, 2H), 1.47-1.43 (m, 2H), 1.41 (s, 6H), 1.27- 1.22 (m, 2H). |
| 60 | 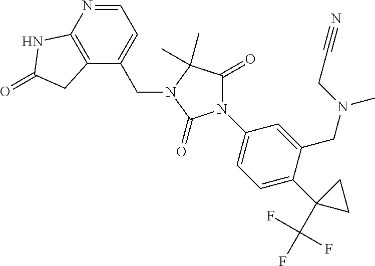 [SMILES: CN(CC#N)Cc1cc(N2C(=O)N(Cc3ccnc4c3CC(=O)N4)C(C)(C)C2=O)ccc1C1(C(F)(F)F)CC1] {[(5-{4,4-dimethyl-2,5-dioxo-3-[(2-oxo-1,7- diaza-4-indanyl)methyl]-1-imidazolidinyl}-2-[1- (trifluoromethyl)cyclopropyl]phenyl)methyl]-N- methylamino}acetonitrile | LCMS: 541.6 (500 MHz, DMSO) δ 11.00 (s, 1H), 8.03 (d, J = 5.5 Hz, 1H), 7.65 (d, J = 2.3 Hz, 1H), 7.59 (d, J = 8.3 Hz, 1H), 7.42 (dd, J = 8.3, 2.3 Hz, 1H), 6.98 (d, J = 5.5 Hz, 1H), 4.53 (s, 2H), 3.86- 3.78 (m, 4H), 3.64 (s, 2H), 2.29 (s, 3H), 1.52-1.46 (m, 2H), 1.40 (s, 6H), 1.26-1.19 (m, 2H) |
| 61 | 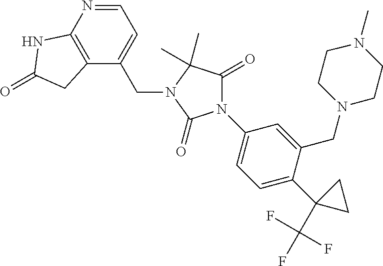 [SMILES: CN1CCN(Cc2cc(N3C(=O)N(Cc4ccnc5c4CC(=O)N5)C(C)(C)C3=O)ccc2C2(C(F)(F)F)CC2)CC1] 5,5-dimethyl-3-{3-[(4-methyl-1- piperazinyl)methyl]-4-[1- (trifluoromethyl)cyclopropyl]phenyl}-1-[(2-oxo- 1,7-diaza-4-indanyl)methyl]-2,4- imidazolidinedione | LCMS: 571.6 (500 MHz, DMSO) δ 11.00 (s, 1H), 8.03 (d, J = 5.5 Hz, 1H), 7.67 (d, J = 2.3 Hz, 1H), 7.55 (d, J = 8.3 Hz, 1H), 7.38 (dd, J = 8.3, 2.3 Hz, 1H), 6.97 (d, J = 5.5 Hz, 1H), 4.54 (s, 2H), 3.71- 3.61 (m, 4H), 2.53-2.52 (m, 2H), 2.49-2.26 (m, 6H), 2.16 (s, 3H), 1.49-1.44 (m, 2H), 1.40 (s, 6H), 1.26-1.20 (m, 2H). |
| 62 | 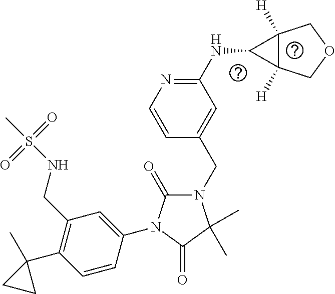 [SMILES: CC1(c2ccc(N3C(=O)N(Cc4ccnc(N[C@H]5[C@@H]6COC[C@@H]65)c4)C(C)(C)C3=O)cc2CNS(C)(=O)=O)CC1] 1-[(2-{(1R,5S,6r)-3-oxabicyclo[3.1.0]hex-6- ylamino}-4-pyridyl)methyl]-3-{3- [(mesylamino)methyl]-4-(1- methylcyclopropyl)phenyl}-5,5-dimethyl-2,4- imidazolidinedione | LCMS: 554.4 (400 MHz, DMSO) δ 7.93 (d, J = 5.2 Hz, 1H), 7.56 (t, J = 6.4 Hz, 1H), 7.44 (d, J = 2.1 Hz, 1H), 7.39 (d, J = 8.2 Hz, 1H), 7.21 (dd, J = 8.2, 2.2 Hz, 1H), 6.72 (d, J = 2.3 Hz, 1H), 6.56 (dd, J = 5.2, 1.3 Hz, 1H), 6.49 (s, 1H), 4.47 (s, 2H), 4.42 (d, J = 6.4 Hz, 2H), 3.84 (d, J = 8.4 Hz, 2H), 3.62 (d, J = 8.2 Hz, 2H), 2.90 (s, 3H), 2.27-2.20 (m, 1H), 1.73 (s, 2H), 1.38 (s, 6H), 1.28 (s, 3H), 0.82-0.71 (m, 4H). |
| 63 | 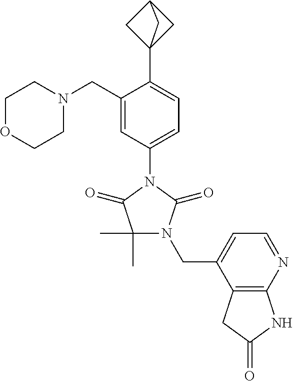 [SMILES: CC1(C)C(=O)N(c2ccc(C34CC(C3)C4)c(CN3CCOCC3)c2)C(=O)N1Cc1ccnc2c1CC(=O)N2] 3-{4-(bicyclo[1.1.1]pent-1-yl)-3- (morpholinomethyl)phenyl}-5,5-dimethyl-1-[(2- oxo-1,7-diaza-4-indanyl)methyl]-2,4- imidazolidinedione | LCMS: 516.4 (400 MHz, DMSO) δ 10.98 (s, 1H), 8.01 (d, J = 5.5 Hz, 1H), 7.35 (d, J = 2.0 Hz, 1H), 7.29- 7.19 (m, 2H), 6.94 (d, J = 5.5 Hz, 1H), 4.50 (s, 2H), 3.62 (s, 2H), 3.59-3.51 (m, 6H), 2.55 (s, 1H), 2.39-2.34 (m, 4H), 2.19 (s, 6H), 1.37 (s, 6H). |
| 64 | 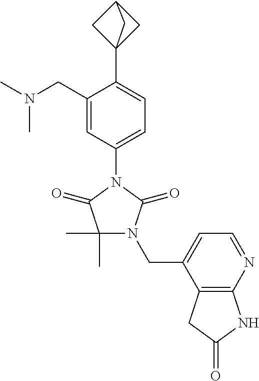 [SMILES: CN(C)Cc1cc(N2C(=O)N(Cc3ccnc4c3CC(=O)N4)C(C)(C)C2=O)ccc1C12CC(C1)C2] 3-{4-(bicyclo[1.1.1]pent-1-yl)-3- [(dimethylamino)methyl]phenyl}-5,5-dimethyl-1- [(2-oxo-1,7-diaza-4-indanyl)methyl]-2,4- imidazolidinedione | LCMS: 474.4 (400 MHz, DMSO) δ 10.98 (s, 1H), 8.01 (d, J = 5.3 Hz, 1H), 7.38 (d, J = 1.9 Hz, 1H), 7.27- 7.13 (m, 2H), 6.95 (d, J = 5.5 Hz, 1H), 4.50 (s, 2H), 3.62 (s, 2H), 3.48 (s, 2H), 2.55 (s, 1H), 2.17 (s, 6H), 2.17 (s, 6H), 1.37 (s, 6H). |
| 65 | 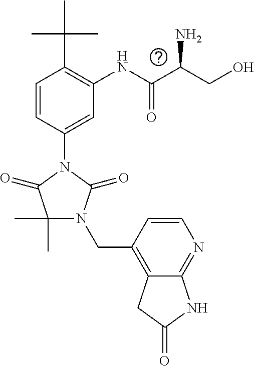 [SMILES: CC(C)(C)c1ccc(N2C(=O)N(Cc3ccnc4c3CC(=O)N4)C(C)(C)C2=O)cc1NC(=O)[C@@H](N)CO] N-[2-(tert-butyl)-5-{4,4-dimethyl-2,5-dioxo-3- [(2-oxo-1,7-diaza-4-indanyl)methyl]-1- imidazolidinyl}phenyl](S)-2-amino-3- hydroxypropionamide | LCMS: 509.3 (500 MHz, DMSO) δ 11.00 (s, 1H), 8.03 (d, J = 5.5 Hz, 1H), 7.96 (d, J = 2.2 Hz, 1H), 7.47 (d, J = 8.6 Hz, 1H), 7.16 (dd, J = 8.5, 2.2 Hz, 1H), 6.94 (d, J = 5.5 Hz, 1H), 4.94 (t, J = 5.3 Hz, 1H), 4.52 (s, 2H), 3.70-3.60 (m, 4H), 3.42 (t, J = 4.8 Hz, 1H), 1.42 (s, 10H), 1.39 (s, 6H). |
| 66 | 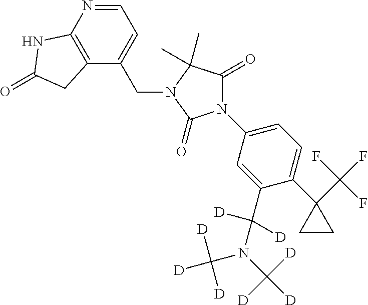 [SMILES: [2H]C([2H])([2H])N(C([2H])([2H])[2H])C([2H])([2H])c1cc(N2C(=O)N(Cc3ccnc4c3CC(=O)N4)C(C)(C)C2=O)ccc1C1(C(F)(F)F)CC1] 3-(3-{[di(²H₃)methylamino](²H₂)methyl}-4-[1- (trifluoromethyl)cyclopropyl]phenyl)-5,5- dimethyl-1-[(2-oxo-1,7-diaza-4-indanyl)methyl]- 2,4-imidazolidinedione | LCMS: 524.6 (500 MHz, DMSO) δ 8.24 (br, 1H), 8.03 (d, J = 5.4 Hz, 1H), 7.68 (d, J = 2.3 Hz, 1H), 7.54 (d, J = 8.3 Hz, 1H), 7.36 (dd, J = 8.3, 2.3 Hz, 1H), 6.98 (d, J = 5.5 Hz, 1H), 4.53 (s, 2H), 3.64 (s, 2H), 1.50-1.45 (m, 2H), 1.40 (s, 6H), 1.23-1.16 (m, 2H). |
| 67 | 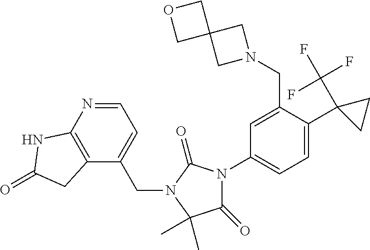 [SMILES: CC1(C)C(=O)N(c2ccc(C3(C(F)(F)F)CC3)c(CN3CC4(COC4)C3)c2)C(=O)N1Cc1ccnc2c1CC(=O)N2] 5,5-dimethyl-3-(3-{(2-oxa-6-aza-6- spiro[3.3]heptyl)methyl}-4-[1- (trifluoromethyl)cyclopropyl]phenyl)-1-[(2-oxo- 1,7-diaza-4-indanyl)methyl]-2,4- imidazolidinedione | LCMS: 570.5 (500 MHz, DMSO) δ 8.28 (br, 1H), 8.02 (d, J = 5.5 Hz, 1H), 7.55-7.50 (m, 2H), 7.35 (dd, J = 8.2, 2.1 Hz, 1H), 6.98 (d, J = 5.5 Hz, 1H), 4.62 (s, 4H), 4.54 (s, 2H), 3.72 (s, 2H), 3.64 (s, 2H), 3.37 (s, 4H), 1.45-1.41 (m, 2H), 1.40 (s, 6H), 1.26- 1.15 (m, 2H). |
| 68 | 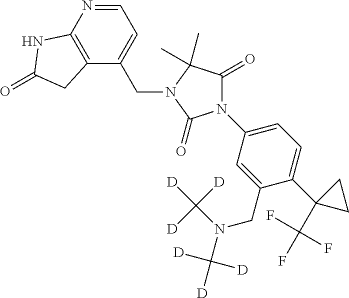 [SMILES: [2H]C([2H])([2H])N(Cc1cc(N2C(=O)N(Cc3ccnc4c3CC(=O)N4)C(C)(C)C2=O)ccc1C1(C(F)(F)F)CC1)C([2H])([2H])[2H]] 3-(3-{[di(²H₃)methylamino]methyl}-4-[1- (trifluoromethyl)cyclopropyl]phenyl)-5,5- dimethyl-1-[(2-oxo-1,7-diaza-4-indanyl)methyl]- 2,4-imidazolidinedione | LCMS: 522.6 (500 MHz, DMSO) δ 8.32 (br, 1H), 8.03 (d, J = 5.5 Hz, 1H), 7.68 (d, J = 2.0 Hz, 1H), 7.54 (d, J = 8.2 Hz, 1H), 7.36 (dd, J = 8.3, 2.2 Hz, 1H), 6.98 (d, J = 5.5 Hz, 1H), 4.53 (s, 2H), 3.64 (s, 2H), 3.61 (s, 2H), 1.50-1.45 (m, 2H), 1.40 (s, 6H), 1.23- 1.18 (m, 2H). |
| 69 | 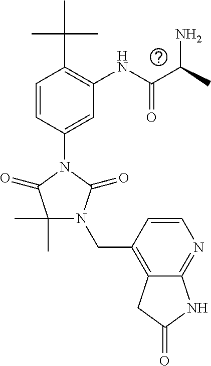 [SMILES: C[C@H](N)C(=O)Nc1cc(N2C(=O)N(Cc3ccnc4c3CC(=O)N4)C(C)(C)C2=O)ccc1C(C)(C)C] N-[2-(tert-butyl)-5-{4,4-dimethyl-2,5-dioxo-3- [(2-oxo-1,7-diaza-4-indanyl)methyl]-1- imidazolidinyl}phenyl](S)-2-aminopropionamide | LCMS: 493.3 (400 MHz, DMSO) δ 10.99 (s, 1H), 8.02 (d, J = 5.5 Hz, 1H), 7.90 (d, J = 2.3 Hz, 1H), 7.47 (d, J = 8.6 Hz, 1H), 7.17 (dd, J = 8.6, 2.3 Hz, 1H), 6.95 (d, J = 5.5 Hz, 1H), 4.52 (s, 2H), 3.63 (s, 2H), 3.46 (q, J = 6.9 Hz, 1H), 1.42 (s, 9H), 1.39 (s, 6H), 1.27 (d, J = 7.0 Hz, 3H). |
| 70 | 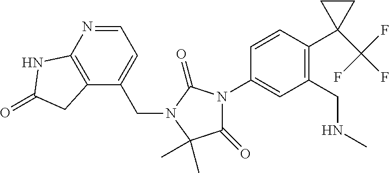 [SMILES: CNCc1cc(N2C(=O)N(Cc3ccnc4c3CC(=O)N4)C(C)(C)C2=O)ccc1C1(C(F)(F)F)CC1] 5,5-dimethyl-3-{3-[(methylamino)methyl]-4-[1- (trifluoromethyl)cyclopropyl]phenyl}-1-[(2-oxo- 1,7-diaza-4-indanyl)methyl]-2,4- imidazolidinedione | LCMS: 502.3 (500 MHz, DMSO) δ 10.93 (s, 1H), 8.14 (s, 1H), 7.96 (d,J = 5.5 Hz, 1H), 7.59 (d,J = 2.1 Hz, 1H), 7.47 (d,J = 8.2 Hz, 1H), 7.28 (dd,J = 8.2, 2.2 Hz, 1H), 6.89 (d,J = 5.5 Hz, 1H), 4.46 (s, 2H), 3.80 (s, 2H), 3.57 (s, 2H), 2.28 (s, 3H), 1.39 (s, 2H), 1.33 (s, 6H), 1.17 (s, 2H) |
| 71 | 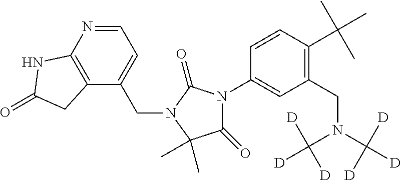 [SMILES: [2H]C([2H])([2H])N(Cc1cc(N2C(=O)N(Cc3ccnc4c3CC(=O)N4)C(C)(C)C2=O)ccc1C(C)(C)C)C([2H])([2H])[2H]] 3-[4-(tert-butyl)-3- {[di(²H₃)methylamino]methyl}phenyl]-5,5- dimethyl-1-[(2-oxo-1,7-diaza-4-indanyl)methyl]- 2,4-imidazolidinedione | LCMS: 470.4 (500 MHz, DMSO) δ 10.99 (s, 1H), 8.02 (d,J = 5.5 Hz, 1H), 7.67 (d,J = 2.5 Hz, 1H), 7.45 (d, J = 8.6 Hz, 1H), 7.22 (dd,J = 8.5, 2.5 Hz, 1H), 6.96 (d,J = 5.5 Hz, 1H), 4.52 (s, 2H), 3.64 (s, 2H), 3.63 (s, 2H), 1.40 (s, 9H), 1.39 (s, 6H). |
| 72 | 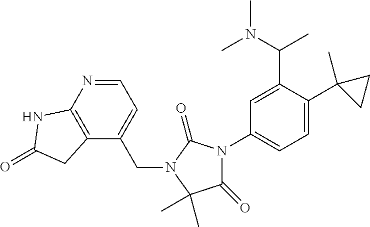 [SMILES: CC(c1cc(N2C(=O)N(Cc3ccnc4c3CC(=O)N4)C(C)(C)C2=O)ccc1C1(C)CC1)N(C)C] 3-{3-[1-(dimethylamino)ethyl]-4-(1- methylcyclopropyl)phenyl}-5,5-dimethyl-1-[(2- oxo-1,7-diaza-4-indanyl)methyl]-2,4- imidazolidinedione | LCMS: 476.4 (400 MHz, DMSO) δ 10.97 (s, 1H), 8.00 (d, J = 5.5 Hz, 1H), 7.50 (d, J = 1.8 Hz, 1H), 7.35 (d, J = 8.2 Hz, 1H), 7.18 (dd, J = 8.2, 1.6 Hz, 1H), 6.94 (d, J = 5.5 Hz, 1H), 4.49 (s, 2H), 3.61 (s, 2H), 2.17 (s, 6H), 1.36 (s, 6H), 1.30 (s, 3H), 1.27-1.17 (m, 4H), 0.85-0.76 (m, 2H), 0.75- 0.67 (m, 2H). |
| 73 | 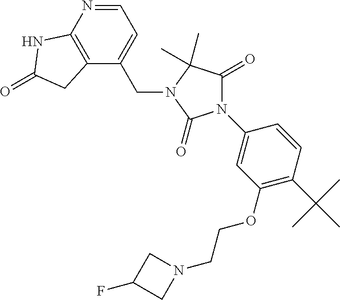 [SMILES: CC(C)(C)c1ccc(N2C(=O)N(Cc3ccnc4c3CC(=O)N4)C(C)(C)C2=O)cc1OCCN1CC(F)C1] 3-{4-(tert-butyl)-3-[2-(3-fluoro-1- azetidinyl)ethoxy]phenyl}-5,5-dimethyl-1-[(2- oxo-1,7-diaza-4-indanyl)methyl]-2,4- imidazolidinedione | LCMS: 524.4 (400 MHz, DMSO) δ 11.00 (s, 1H), 8.00 (d, J = 5.6 Hz, 1H), 7.28 (d, J = 8.3 Hz, 1H), 7.04 (s, 1H), 6.94 (d, J = 5.6 Hz, 1H), 6.91 (dd, J = 8.1, 1.8 Hz, 1H), 5.25-5.02 (m, 1H), 4.50 (s, 2H), 3.92 (t, J = 5.6 Hz, 2H), 3.69-3.54 (m, 4H), 3.25-3.19 (m, 1H), 3.18-3.12 (m, 1H), 2.88 (t, J = 5.8 Hz, 2H), 1.36 (s, 6H), 1.34 (s, 9H). |
| 74 | 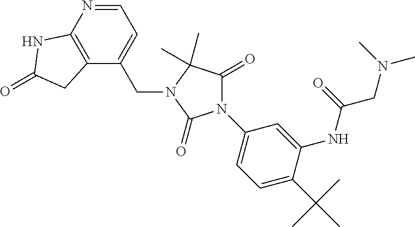 [SMILES: CN(C)CC(=O)Nc1cc(N2C(=O)N(Cc3ccnc4c3CC(=O)N4)C(C)(C)C2=O)ccc1C(C)(C)C] N-[2-(tert-butyl)-5-{4,4-dimethyl-2,5-dioxo-3- [(2-oxo-1,7-diaza-4-indanyl)methyl]-1- imidazolidinyl}phenyl](dimethylamino)acetamide | LCMS: 507.4 (400 MHz, DMSO) δ 10.99 (s, 1H), 9.59 (s, 1H), 8.02 (d, J = 5.5 Hz, 1H), 7.87 (d, J = 2.3 Hz, 1H), 7.49 (d, J = 8.6 Hz, 1H), 7.20 (dd, J = 8.6, 2.3 Hz, 1H), 6.95 (d, J = 5.5 Hz, 1H), 4.52 (s, 2H), 3.63 (s, 2H), 3.11 (s, 2H), 2.36 (s, 6H), 1.40 (s, 9H), 1.39 (s, 6H). |
| 75 | 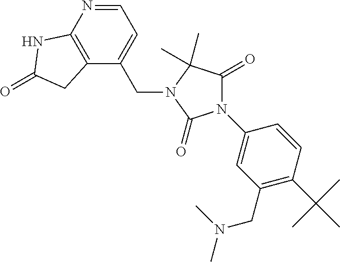 [SMILES: CN(C)Cc1cc(N2C(=O)N(Cc3ccnc4c3CC(=O)N4)C(C)(C)C2=O)ccc1C(C)(C)C] 3-{4-(tert-butyl)-3- [(dimethylamino)methyl]phenyl}-5,5-dimethyl-1- [(2-oxo-1,7-diaza-4-indanyl)methyl]-2,4- imidazolidinedione | LCMS: 464.4 (500 MHz, DMSO) δ 10.99 (s, 1H), 8.02 (d,J = 5.5 Hz, 1H), 7.66 (d,J = 2.4 Hz, 1H), 7.45 (d, J = 8.6 Hz, 1H), 7.22 (dd,J = 8.5, 2.4 Hz, 1H), 6.96 (d,J = 5.5 Hz, 1H), 4.52 (s, 2H), 3.64 (s, 2H), 3.63 (s, 2H), 2.21 (s, 6H), 1.40 (s, 9H), 1.39 (s, 6H). |
| 76 | 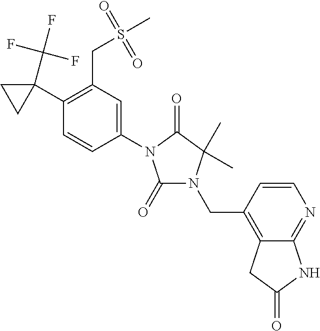 [SMILES: CC1(C)C(=O)N(c2ccc(C3(C(F)(F)F)CC3)c(CS(C)(=O)=O)c2)C(=O)N1Cc1ccnc2c1CC(=O)N2] 3-{3-(mesylmethyl)-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 549.3 (M − H) (500 MHz, DMSO) δ 11.00 (s, 1H), 8.02 (d,J = 5.5 Hz, 1H), 7.68 (s, 1H), 7.67 (d,J = 5.9 Hz, 1H), 7.56 (dd,J = 8.4, 2.1 Hz, 1H), 6.97 (d,J = 5.5 Hz, 1H), 4.80 (s, 2H), 4.54 (s, 2H), 3.65 (s, 2H), 3.21 (s, 3H), 1.59-1.44 (m, 4H), 1.41 (s, 6H). |
| 77 | 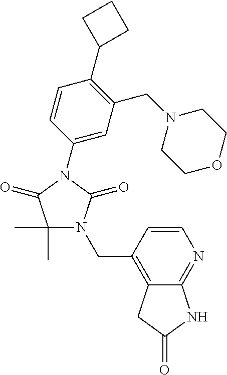 [SMILES: CC1(C)C(=O)N(c2ccc(C3CCC3)c(CN3CCOCC3)c2)C(=O)N1Cc1ccnc2c1CC(=O)N2] 3-[4-cyclobutyl-3-(morpholinomethyl)phenyl]- 5,5-dimethyl-1-[(2-oxo-1,7-diaza-4- indanyl)methyl]-2,4-imidazolidinedione | LCMS: 504.4 (400 MHz, DMSO) δ 10.98 (s, 1H), 8.01 (d, J = 5.5 Hz, 1H), 7.43 (d, J = 8.9 Hz, 1H), 7.35- 7.28 (m, 2H), 6.95 (d, J = 5.5 Hz, 1H), 4.50 (s, 2H), 3.90- 3.77 (m, 1H), 3.62 (s, 2H), 3.57- 3.50 (m, 4H), 3.41 (s, 2H), 2.37-2.24 (m, 7H), 2.15-1.90 (m, 2H), 1.87-1.73 (m, 1H), 1.37 (s, 6H). |
| 78 | 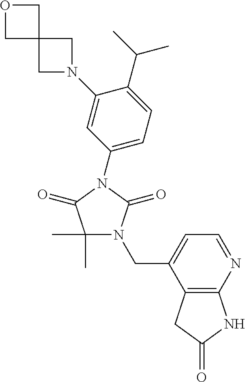 [SMILES: CC(C)c1ccc(N2C(=O)N(Cc3ccnc4c3CC(=O)N4)C(C)(C)C2=O)cc1N1CC2(COC2)C1] 5,5-dimethyl-3-{3-(2-oxa-6-aza-6- spiro[3.3]heptyl)-4-cumenyl}-1-[(2-oxo-1,7- diaza-4-indanyl)methyl]-2,4-imidazolidinedione | LCMS: 490.4 (400 MHz, DMSO) δ = 11.00 (s, 1H), 8.03 (d, J = 5.5, 1H), 7.23 (d, J = 8.2, 1H), 6.94 (d, J = 5.6, 1H), 6.82 (dd, J = 8.2, 2.0, 1H), 6.53 (d, J = 2.0, 1H), 4.72 (s, 4H), 4.51 (s, 2H), 4.04 (s, 4H), 3.63 (s, 2H), 3.10-3.00 (m, 1H), 1.38 (s, 6H), 1.18 (d, J = 6.8, 6H). |
| 79 | 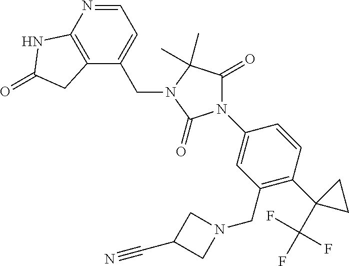 [SMILES: CC1(C)C(=O)N(c2ccc(C3(C(F)(F)F)CC3)c(CN3CC(C#N)C3)c2)C(=O)N1Cc1ccnc2c1CC(=O)N2] 1-[(5-{4,4-dimethyl-2,5-dioxo-3-[(2-oxo-1,7- diaza-4-indanyl)methyl]-1-imidazolidinyl}-2-[1- (trifluoromethyl)cyclopropyl]phenyl)methyl]-3- azetidinecarbonitrile | LCMS: 553.3 (400 MHz, DMSO) δ 11.01 (brs, 1H), 8.02 (d,J = 5.5 Hz, 1H), 7.60-7.52 (m, 2H), 7.38 (dd,J = 8.2, 2.2 Hz, 1H), 6.97 (d,J = 5.5 Hz, 1H), 4.54 (s, 2H), 3.82 (s, 2H), 3.64 (s, 2H), 3.58-3.50 (m,J = 6.7 Hz, 3H), 3.35 (s, 2H), 1.46 (s, 2H), 1.40 (s, 6H), 1.24 (brs, 2H). |
| 80 | 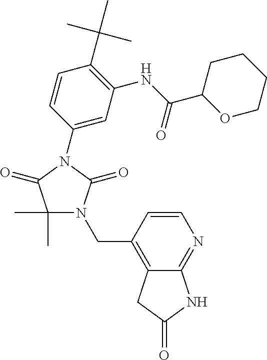 [SMILES: CC(C)(C)c1ccc(N2C(=O)N(Cc3ccnc4c3CC(=O)N4)C(C)(C)C2=O)cc1NC(=O)C1CCCCO1] N-[2-(tert-butyl)-5-{4,4-dimethyl-2,5-dioxo-3- [(2-oxo-1,7-diaza-4-indanyl)methyl]-1- imidazolidinyl}phenyl]-tetrahydro-2H-pyran-2- carboxamide | LCMS: 534.6 (400 MHz, DMSO) δ 10.97 (s, 1H), 8.97 (s, 1H), 8.00 (d, J = 5.5 Hz, 1H), 7.53 (d, J = 2.3 Hz, 1H), 7.47 (d, J = 8.6 Hz, 1H), 7.23 (dd, J = 8.6, 2.3 Hz, 1H), 6.94 (d, J = 5.5 Hz, 1H), 4.49 (s, 2H), 4.07-4.01 (m, 1H), 3.93 (dd, J = 11.3, 2.5 Hz, 1H), 3.61 (s, 2H), 3.58-3.49 (m, 1H), 2.00-1.90 (m, 1H), 1.88-1.80 (m, 1H), 1.62-1.39 (m, 4H), 1.36 (s, 6H), 1.34 (s, 9H). |
| 81 | 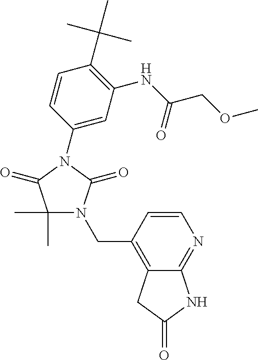 [SMILES: COCC(=O)Nc1cc(N2C(=O)N(Cc3ccnc4c3CC(=O)N4)C(C)(C)C2=O)ccc1C(C)(C)C] N-[2-(tert-butyl)-5-{4,4-dimethyl-2,5-dioxo-3- [(2-oxo-1,7-diaza-4-indanyl)methyl]-1- imidazolidinyl}phenyl]methoxyacetamide | LCMS: 494.6 (400 MHz, DMSO) δ 10.95 (s, 1H), 9.17 (s, 1H), 8.00 (d, J = 5.6 Hz, 1H), 7.49 (d, J = 8.6 Hz, 1H), 7.40 (s, 1H), 7.26 (dd, J = 8.4, 1.9 Hz, 1H), 6.94 (d, J = 5.5 Hz, 1H), 4.49 (s, 2H), 4.00 (s, 2H), 3.61 (s, 2H), 3.41 (s, 3H), 1.37 (s, 6H), 1.34 (s, 9H). |
| 82 | 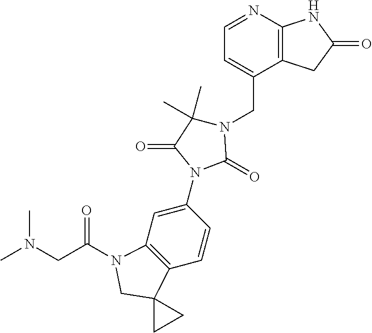 [SMILES: CN(C)CC(=O)N1CC2(CC2)c2ccc(N3C(=O)N(Cc4ccnc5c4CC(=O)N5)C(C)(C)C3=O)cc21] 3-{1′-[2- (dimethylamino)acetyl]spiro[cyclopropane-1,3′- indolin]-6′-yl}-5,5-dimethyl-1-[(2-oxo-1,7-diaza- 4-indanyl)methyl]-2,4-imidazolidinedione | LCMS: 503.4 (500 MHz, DMSO) δ 10.93 (s, 1H), 8.00 (d, J = 1.4 Hz, 1H), 7.96 (d, J = 5.5 Hz, 1H), 6.94 (dd, J = 8.0, 1.7 Hz, 1H), 6.90 (d, J = 5.5 Hz, 1H), 6.83 (d, J = 8.0 Hz, 1H), 4.45 (s, 2H), 4.15 (s, 2H), 3.57 (s, 2H), 3.14 (s, 2H), 2.21 (s, 6H), 1.33 (d, J = 7.0 Hz, 6H), 1.07 (t, J = 5.6 Hz, 2H), 1.02 (t, J = 5.5 Hz, 2H). |
| 83 | 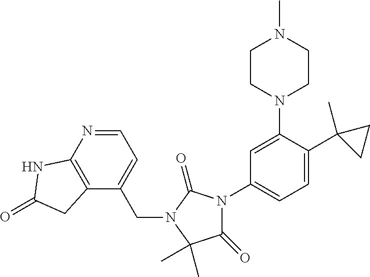 [SMILES: CN1CCN(c2cc(N3C(=O)N(Cc4ccnc5c4CC(=O)N5)C(C)(C)C3=O)ccc2C2(C)CC2)CC1] 5,5-dimethyl-3-[4-(1-methylcyclopropyl)-3-(4- methyl-1-piperazinyl)phenyl]-1-[(2-oxo-1,7- diaza-4-indanyl)methyl]-2,4-imidazolidinedione | LCMS: 503.6 (400 MHz, DMSO) δ 10.97 (s, 1H), 8.00 (d, J = 5.5 Hz, 1H), 7.37 (d, J = 8.2 Hz, 1H), 7.10 (d, J = 2.1 Hz, 1H), 7.03 (dd, J = 8.2, 2.0 Hz, 1H), 6.93 (d, J = 5.5 Hz, 1H), 4.49 (s, 2H), 3.61 (s, 2H), 3.05-2.86 (m, 4H), 2.76- 2.49 (m, 4H), 2.30 (s, 3H), 1.40 (s, 3H), 1.35 (s, 6H), 0.93-0.84 (m, 2H), 0.72-0.63 (m, 2H). |
| 84 | 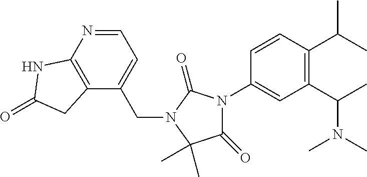 [SMILES: CC(C)c1ccc(N2C(=O)N(Cc3ccnc4c3CC(=O)N4)C(C)(C)C2=O)cc1C(C)N(C)C] 3-{3-[1-(dimethylamino)ethyl]-4-cumenyl}-5,5- dimethyl-1-[(2-oxo-1,7-diaza-4-indanyl)methyl]- 2,4-imidazolidinedione | LCMS: 464.5 (500 MHz, DMSO) δ 11.00 (s, 1H), 8.03 (d,J = 5.5 Hz, 1H), 7.45 (d,J = 2.2 Hz, 1H), 7.38 (d, J = 8.4 Hz, 1H), 7.23 (dd,J = 8.4, 2.3 Hz, 1H), 6.96 (d,J = 5.5 Hz, 1H), 4.51 (s, 2H), 3.64 (s, 2H), 3.54-3.48 (m, 1H), 3.43-3.35 (m, 1H), 2.14 (s, 6H), 1.39 (d,J = 2.0 Hz, 6H), 1.23 (d,J = 6.6 Hz, 3H), 1.21 (d,J = 6.9 Hz, 6H) |
| 85 | 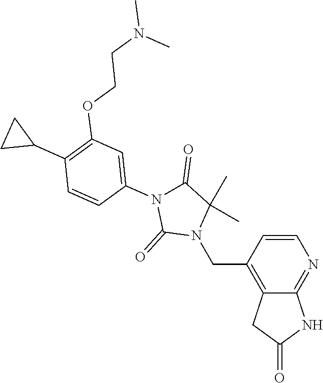 3-{4-cyclobutyl-3- [(dimethylamino)methyl]phenyl}-5,5-dimethyl-1- [(2-oxo-1,7-diaza-4-indanyl)methyl]-2,4- imidazolidinedione | LCMS: 462.4 (400 MHz, DMSO) δ 10.98 (s, 1H), 8.17 (s, 1H), 8.01 (d, J = 5.5 Hz, 1H), 7.40 (d, J = 8.2 Hz, 1H), 7.32 (d, J = 2.1 Hz, 1H), 7.27 (dd, J = 8.2, 2.2 Hz, 1H), 6.95 (d, J = 5.5 Hz, 1H), 4.50 (s, 2H), 3.88-3.76 (m, 1H), 3.62 (s, 2H), 3.33 (s, 2H), 2.33-2.21 (m, 2H), 2.14 (s, 6H), 2.11- 1.72 (m, 4H), 1.37 (s, 6H). |
| 86 | 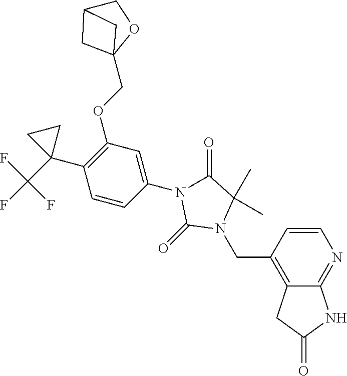 [SMILES: CC1(C)C(=O)N(c2ccc(C3(C(F)(F)F)CC3)c(OCC34CC(CO3)C4)c2)C(=O)N1Cc1ccnc2c1CC(=O)N2] 5,5-dimethyl-3-(3-{(2-oxabicyclo[2.1.1]hex-1- yl)methoxy}-4-[1- (trifluoromethyl)cyclopropyl]phenyl)-1-[(2-oxo- 1,7-diaza-4-indanyl)methyl]-2,4- imidazolidinedione | LCMS: 571.3 (400 MHz, DMSO) δ 11.00 (s, 1H), 8.03 (d, J = 5.5 Hz, 1H), 7.48 (d, J = 8.1 Hz, 1H), 7.20 (d, J = 1.9 Hz, 1H), 7.04 (dd, J = 8.1, 1.9 Hz, 1H), 6.97 (d, J = 5.5 Hz, 1H), 4.53 (s, 2H), 4.21 (s, 2H), 3.73 (s, 2H), 3.64 (s, 2H), 2.96 (t, J = 3.2 Hz, 1H), 1.92 (ddd, J = 4.7, 3.2, 1.8 Hz, 2H), 1.45 (dd, J = 4.4, 1.7 Hz, 2H), 1.42-1.34 (m, 8H), 1.13-1.07 (m, 2H) |
| 87 | 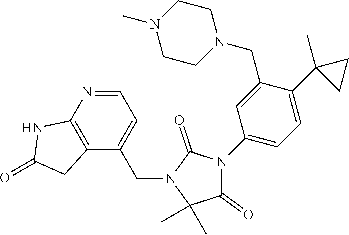 [SMILES: CN1CCN(Cc2cc(N3C(=O)N(Cc4ccnc5c4CC(=O)N5)C(C)(C)C3=O)ccc2C2(C)CC2)CC1] 5,5-dimethyl-3-{4-(1-methylcyclopropyl)-3-[(4- methyl-1-piperazinyl)methyl]phenyl}-1-[(2-oxo- 1,7-diaza-4-indanyl)methyl]-2,4- imidazolidinedione | LCMS: 517.5 (400 MHz, DMSO) δ 10.97 (s, 1H), 8.00 (d, J = 5.5 Hz, 1H), 7.45 (d, J = 2.0 Hz, 1H), 7.36 (d, J = 8.2 Hz, 1H), 7.20 (dd, J = 8.2, 2.1 Hz, 1H), 6.93 (d, J = 5.5 Hz, 1H), 4.50 (s, 2H), 3.67 (s, 2H), 3.61 (s, 2H), 2.47-2.22 (m, 8H), 2.15 (s, 3H), 1.36 (s, 6H), 1.28 (s, 3H), 0.73 (d, J = 4.9 Hz, 4H). |
| 88 | 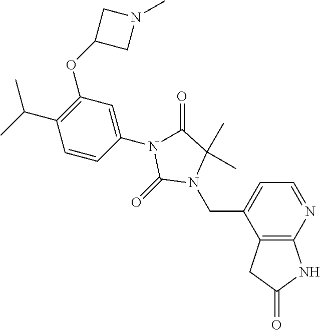 [SMILES: CC(C)c1ccc(N2C(=O)N(Cc3ccnc4c3CC(=O)N4)C(C)(C)C2=O)cc1OC1CN(C)C1] 5,5-dimethyl-3-[3-(1-methyl-3-azetidinyloxy)-4- cumenyl]-1-[(2-oxo-1,7-diaza-4-indanyl)methyl]- 2,4-imidazolidinedione | LCMS: 478.4 (400 MHz, DMSO) δ = 11.00 (s, 1H), 8.03 (d, J = 5.5, 1H), 7.30 (d, J = 8.1, 1H), 6.99 (dd, J = 8.1, 1.9, 1H), 6.96 (d, J = 5.5, 1H), 6.81 (d, J = 1.9, 1H), 4.73 (p, J = 5.6, 1H), 4.52 (s, 2H), 3.76-3.72 (m, 2H), 3.64 (s, 2H), 3.30-3.21 (m, 1H), 2.97-2.90 (m, 2H), 2.28 (s, 3H), 1.38 (s, 6H), 1.20 (d, J = 6.9, 6H). |
| 89 | 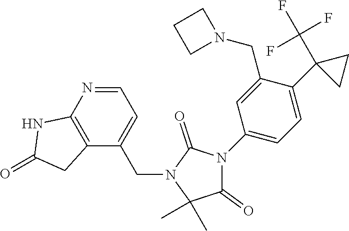 [SMILES: CC1(C)C(=O)N(c2ccc(C3(C(F)(F)F)CC3)c(CN3CCC3)c2)C(=O)N1Cc1ccnc2c1CC(=O)N2] 3-{3-[(1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 528.6 (500 MHz, DMSO) δ 8.26 (s, 1H), 8.03 (d, J = 5.4 Hz, 1H), 7.60 (s, 1H), 7.53 (d, J = 8.3 Hz, 1H), 7.35 (dd, J = 8.1, 1.8 Hz, 1H), 6.98 (d, J = 5.5 Hz, 1H), 4.54 (s, 2H), 3.75 (s, 2H), 3.65 (s, 2H), 3.22-3.16 (m, 4H), 2.04-1.97 (m, 2H), 1.47-1.44 (m, 2H), 1.40 (s, 6H), 1.26- 1.21 (m, 2H). |
| 90 | 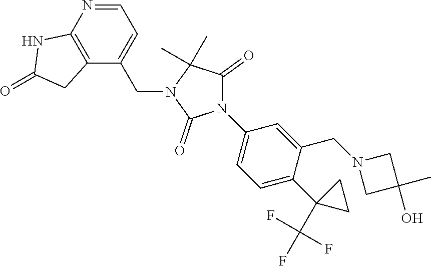 [SMILES: CC1(O)CN(Cc2cc(N3C(=O)N(Cc4ccnc5c4CC(=O)N5)C(C)(C)C3=O)ccc2C2(C(F)(F)F)CC2)C1] 3-{3-[(3-hydroxy-3-methyl-1-azetidinyl)methyl]- 4-[1-(trifluoromethyl)cyclopropyl]phenyl}-5,5- dimethyl-1-[(2-oxo-1,7-diaza-4-indanyl)methyl]- 2,4-imidazolidinedione | LCMS: 558.6 (500 MHz, DMSO) δ 11.01 (s, 1H), 8.34 (s, 1H), 8.02 (d, J = 5.5 Hz, 1H), 7.57 (s, 1H), 7.53 (d, J = 8.2 Hz, 1H), 7.37 (d, J = 8.3 Hz, 1H), 6.97 (d, J = 5.4 Hz, 1H), 4.54 (s, 2H), 3.80 (s, 2H), 3.64 (s, 2H), 3.24 (d, J = 7.1 Hz, 2H), 2.94 (d, J = 6.8 Hz, 2H), 1.47-1.43 (m, 2H), 1.40 (s, 6H), 1.37 (s, 3H), 1.27-1.23 (m, 2H). |
| 91 | 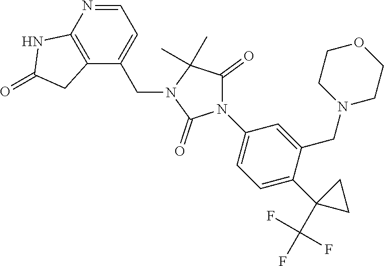 [SMILES: CC1(C)C(=O)N(c2ccc(C3(C(F)(F)F)CC3)c(CN3CCOCC3)c2)C(=O)N1Cc1ccnc2c1CC(=O)N2] 5,5-dimethyl-3-{3-(morpholinomethyl)-4-[1- (trifluoromethyl)cyclopropyl]phenyl}-1-[(2-oxo- 1,7-diaza-4-indanyl)methyl]-2,4- imidazolidinedione | LCMS: 558.6 (500 MHz, DMSO) δ 11.00 (s, 1H), 8.03 (d, J = 5.5 Hz, 1H), 7.70 (d, J = 2.3 Hz, 1H), 7.56 (d, J = 8.3 Hz, 1H), 7.39 (dd, J = 8.3, 2.3 Hz, 1H), 6.98 (d, J = 5.5 Hz, 1H), 4.54 (s, 2H), 3.69 (s, 2H), 3.65 (s, 2H), 3.63-3.58 (m, 4H), 2.46-2.38 (m, 4H), 1.51-1.45 (m, 2H), 1.40 (s, 6H), 1.28-1.21 (m, 2H). |
| 92 | 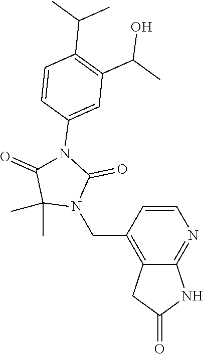 [SMILES: CC(C)c1ccc(N2C(=O)N(Cc3ccnc4c3CC(=O)N4)C(C)(C)C2=O)cc1C(C)O] 3-[3-(1-hydroxyethyl)-4-cumenyl]-5,5-dimethyl- 1-[(2-oxo-1,7-diaza-4-indanyl)methyl]-2,4- imidazolidinedione | LCMS: 435.3 (M − H) (500 MHz, DMSO) δ 10.98 (s, 1H), 8.03 (d,J = 5.5 Hz, 1H), 7.49 (d,J = 2.3 Hz, 1H), 7.37 (d, J = 8.4 Hz, 1H), 7.23 (dd,J = 8.3, 2.3 Hz, 1H), 6.95 (d,J = 5.5 Hz, 1H), 5.21 (s, 1H), 5.06 (q,J = 6.3 Hz, 1H), 4.52 (s, 2H), 3.64 (s, 2H), 3.26-3.17 (m, 1H), 1.39 (d,J = 2.6 Hz, 6H), 1.31 (d,J = 6.4 Hz, 3H), 1.21 (dd,J = 6.8, 2.9 Hz, 6H). |
| 93 | 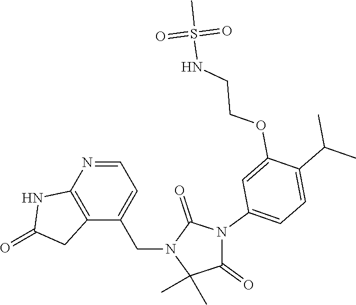 [SMILES: CC(C)c1ccc(N2C(=O)N(Cc3ccnc4c3CC(=O)N4)C(C)(C)C2=O)cc1OCCNS(C)(=O)=O] 3-{3-[2-(mesylamino)ethoxy]-4-cumenyl}-5,5- dimethyl-1-[(2-oxo-1,7-diaza-4-indanyl)methyl]- 2,4-imidazolidinedione | LCMS: 530.3 (400 MHz, DMSO) δ 10.98 (s, 1H), 8.01 (d, J = 5.1 Hz, 1H), 7.33-7.26 (m, 2H), 7.01 (s, 1H), 6.98-6.92 (m, 2H), 4.50 (s, 2H), 4.04-3.92 (m, 2H), 3.62 (s, 2H), 3.39-3.31 (m, 2H), 2.93 (s, 3H), 1.37 (s, 6H), 1.18 (d, J = 6.6 Hz, 6H). |
| 94 | 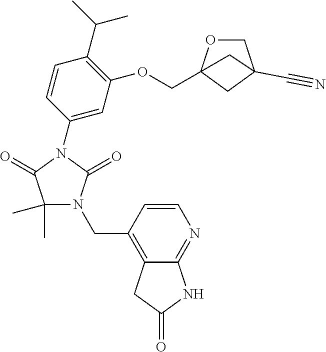 [SMILES: CC(C)c1ccc(N2C(=O)N(Cc3ccnc4c3CC(=O)N4)C(C)(C)C2=O)cc1OCC12CC(C#N)(CO1)C2] 1-[(5-{4,4-dimethyl-2,5-dioxo-3-[(2-oxo-1,7- diaza-4-indanyl)methyl]-1-imidazolidinyl}-2- cumenyloxy)methyl]-2-oxabicyclo[2.1.1]hexane- 4-carbonitrile | LCMS: 530.2 (500 MHz, DMSO) δ 10.96 (s, 1H), 8.03 (d,J = 5.5 Hz, 1H), 7.29 (d,J = 8.1 Hz, 1H), 7.05 (d, J = 1.9 Hz, 1H), 6.98 (dd,J = 8.1, 1.8 Hz, 1H), 6.95 (d,J = 5.5 Hz, 1H), 4.52 (s, 2H), 4.22 (s, 2H), 4.00 (s, 2H), 3.64 (s, 2H), 3.29- 3.20 (m, 1H), 2.45 (dd,J = 4.5, 1.5 Hz, 2H), 1.99 (dd,J = 4.5, 1.6 Hz, 2H), 1.39 (s, 6H), 1.19 (d,J = 6.9 Hz, 6H). |
| 95 | 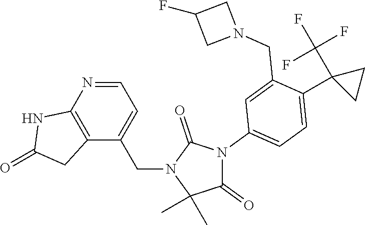 [SMILES: CC1(C)C(=O)N(c2ccc(C3(C(F)(F)F)CC3)c(CN3CC(F)C3)c2)C(=O)N1Cc1ccnc2c1CC(=O)N2] 3-{3-[(3-fluoro-1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 546.2 (500 MHz, DMSO) δ 8.41 (s, 1H), 8.03 (d, J = 5.5 Hz, 1H), 7.59 (d, J = 2.2 Hz, 1H), 7.55 (d, J = 8.2 Hz, 1H), 7.38 (dd, J = 8.3, 2.2 Hz, 1H), 6.98 (d, J = 5.5 Hz, 1H), 5.31-5.14 (m, 1H), 4.54 (s, 2H), 3.87 (s, 2H), 3.68- 3.59 (m, 4H), 3.26-3.15 (m, 2H), 1.49-1.44 (m, 2H), 1.40 (s, 6H), 1.26-1.21 (m, 2H). |
| 97 | 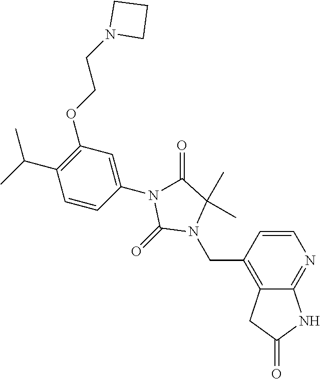 [SMILES: CC(C)c1ccc(N2C(=O)N(Cc3ccnc4c3CC(=O)N4)C(C)(C)C2=O)cc1OCCN1CCC1] 3-{3-[2-(1-azetidinyl)ethoxy]-4-cumenyl}-5,5- dimethyl-1-[(2-oxo-1,7-diaza-4-indanyl)methyl]- 2,4-imidazolidinedione | LCMS: 492.7 (400 MHz, CD₃CN) δ 8.78 (s, 1H), 8.05 (d, J = 5.5 Hz, 1H), 7.34 (d, J = 8.7 Hz, 1H), 6.98 (dd, J = 6.0, 2.2 Hz, 3H), 4.51 (s, 2H), 4.02 (t, J = 5.4 Hz, 2H), 3.56 (s, 2H), 3.48 (t, J = 7.1 Hz, 4H), 3.37 (dt, J = 13.8, 7.0 Hz, 1H), 2.96 (t, J = 5.2 Hz, 2H), 2.10-2.04 (m, 2H), 1.41 (s, 6H), 1.23 (d, J = 6.9 Hz, 6H). |
| 98 | 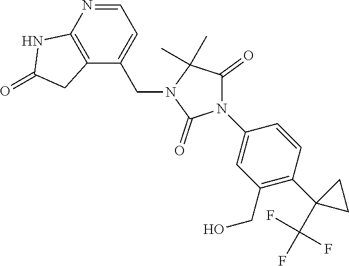 [SMILES: CC1(C)C(=O)N(c2ccc(C3(C(F)(F)F)CC3)c(CO)c2)C(=O)N1Cc1ccnc2c1CC(=O)N2] 3-{3-(hydroxymethyl)-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 489.4 (500 MHz, DMSO) δ 11.00 (s, 1H), 8.03 (d, J = 5.5 Hz, 1H), 7.65 (d, J = 2.2 Hz, 1H), 7.53 (d, J = 8.2 Hz, 1H), 7.36 (dd, J = 8.2, 2.2 Hz, 1H), 6.98 (d, J = 5.5 Hz, 1H), 5.35 (t, J = 5.5 Hz, 1H), 4.73 (d, J = 5.1 Hz, 2H), 4.54 (s, 2H), 3.65 (s, 2H), 1.43 (d, J = 16.9 Hz, 8H), 1.21 (s, 2H). |
| 99 | 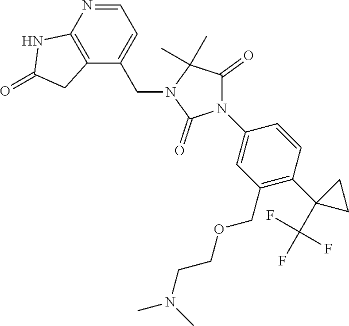 [SMILES: CN(C)CCOCc1cc(N2C(=O)N(Cc3ccnc4c3CC(=O)N4)C(C)(C)C2=O)ccc1C1(C(F)(F)F)CC1] 3-(3-{[2-(dimethylamino)ethoxy]methyl}-4-[1- (trifluoromethyl)cyclopropyl]phenyl)-5,5- dimethyl-1-[(2-oxo-1,7-diaza-4-indanyl)methyl]- 2,4-imidazolidinedione | LCMS: 560.4 (400 MHz, DMSO) δ 10.98 (s, 1H), 8.00 (d, J = 5.5 Hz, 1H), 7.55 (dd, J = 5.2, 2.9 Hz, 2H), 7.40 (dd, J = 8.3, 2.2 Hz, 1H), 6.95 (d, J = 5.5 Hz, 1H), 4.67 (s, 2H), 4.51 (s, 2H), 3.62 (s, 2H), 3.58 (t, J = 5.9 Hz, 2H), 2.44 (t, J = 5.9 Hz, 2H), 2.13 (s, 6H), 1.98 (d, J = 7.5 Hz, 1H), 1.47-1.41 (m, 2H), 1.38 (s, 6H), 0.87- 0.78 (m, 1H). |
| 100 | 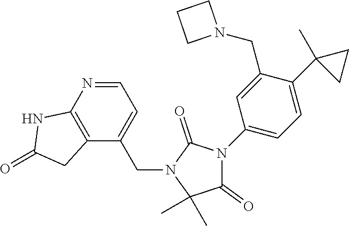 [SMILES: CC1(c2ccc(N3C(=O)N(Cc4ccnc5c4CC(=O)N5)C(C)(C)C3=O)cc2CN2CCC2)CC1] 3-{3-[(1-azetidinyl)methyl]-4-(1- methylcyclopropyl)phenyl}-5,5-dimethyl-1-[(2- oxo-1,7-diaza-4-indanyl)methyl]-2,4- imidazolidinedione | LCMS: 474.3 (400 MHz, DMSO) δ 10.97 (s, 1H), 8.00 (d, J = 5.4 Hz, 1H), 7.41 (d, J = 2.0 Hz, 1H), 7.34 (d, J = 8.2 Hz, 1H), 7.17 (dd, J = 8.1, 2.2 Hz, 1H), 6.93 (d, J = 5.5 Hz, 1H), 4.50 (s, 2H), 3.78 (s, 2H), 3.61 (s, 2H), 3.19 (t, J = 6.8 Hz, 4H), 2.00 (p, J = 6.9 Hz, 2H), 1.36 (s, 6H), 1.27 (s, 3H), 0.73 (s, 4H). |
| 101 | 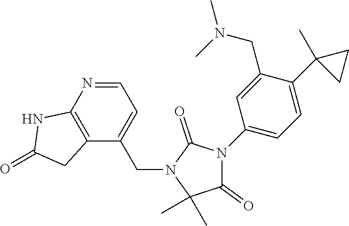 [SMILES: CN(C)Cc1cc(N2C(=O)N(Cc3ccnc4c3CC(=O)N4)C(C)(C)C2=O)ccc1C1(C)CC1] 3-{3-[(dimethylamino)methyl]-4-(1- methylcyclopropyl)phenyl}-5,5-dimethyl-1-[(2- oxo-1,7-diaza-4-indanyl)methyl]-2,4- imidazolidinedione | LCMS: 462.4 (400 MHz, DMSO) δ 10.97 (s, 1H), 8.00 (d, J = 5.5 Hz, 1H), 7.49 (d, J = 2.1 Hz, 1H), 7.36 (d, J = 8.2 Hz, 1H), 7.18 (dd, J = 8.2, 2.2 Hz, 1H), 6.94 (d, J = 5.5 Hz, 1H), 4.49 (s, 2H), 3.63 (s, 2H), 3.61 (s, 2H), 2.20 (s, 6H), 1.36 (s, 6H), 1.27 (s, 3H), 0.73 (s, 4H). |
| 102 | 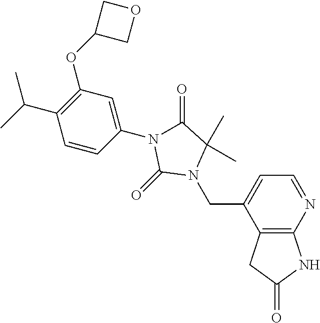 [SMILES: CC(C)c1ccc(N2C(=O)N(Cc3ccnc4c3CC(=O)N4)C(C)(C)C2=O)cc1OC1COC1] 5,5-dimethyl-3-[3-(3-oxetanyloxy)-4-cumenyl]-1- [(2-oxo-1,7-diaza-4-indanyl)methyl]-2,4- imidazolidinedione | LCMS: 465.5 (400 MHz, DMSO) δ = 11.00 (s, 1H), 8.03 (d, J = 5.5, 1H), 7.34 (d, J = 8.1, 1H), 7.02 (dd, J = 8.1, 1.9, 1H), 6.95 (d, J = 5.5, 1H), 6.65 (d, J = 1.9, 1H), 5.28 (p, J = 5.8, 1H), 4.92 (t, J = 6.9, 2H), 4.59-4.54 (m, 2H), 4.52 (s, 2H), 3.63 (s, 2H), 3.41-3.26 (m, 1H), 1.38 (s, 6H), 1.22 (d, J = 6.9, 6H). |
| 103 | 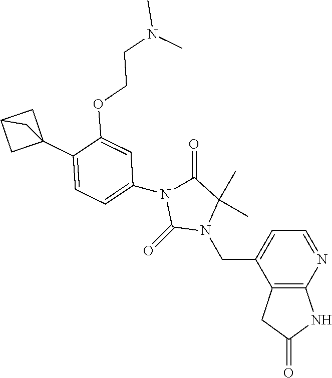 [SMILES: CN(C)CCOc1cc(N2C(=O)N(Cc3ccnc4c3CC(=O)N4)C(C)(C)C2=O)ccc1C12CC(C1)C2] 3-{4-(bicyclo[1.1.1]pent-1-yl)-3-[2- (dimethylamino)ethoxy]phenyl}-5,5-dimethyl-1- [(2-oxo-1, 7-diaza-4-indanyl)methyl]-2,4- imidazolidinedione | LCMS: 504.3 (400 MHz, DMSO) δ 10.98 (s, 1H), 8.01 (d, J = 5.3 Hz, 1H), 7.08 (d, J = 7.9 Hz, 1H), 7.00 (s, 1H), 6.94 (d, J = 5.4 Hz, 1H), 6.90 (d, J = 9.1 Hz, 1H), 4.50 (s, 2H), 3.99 (t, J = 5.7 Hz, 2H), 3.62 (s, 2H), 2.67 (t, J = 5.7 Hz, 2H), 2.54 (s, 1H), 2.22 (s, 6H), 2.10 (s, 6H), 1.37 (s, 6H). |
| 104 | 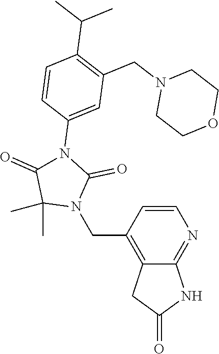 [SMILES: CC(C)c1ccc(N2C(=O)N(Cc3ccnc4c3CC(=O)N4)C(C)(C)C2=O)cc1CN1CCOCC1] 5,5-dimethyl-3-[3-(morpholinomethyl)-4- cumenyl]-1-[(2-oxo-1,7-diaza-4-indanyl)methyl]- 2,4-imidazolidinedione | LCMS: 492.3 (400 MHz, DMSO) δ 8.01 (d, J = 5.5 Hz, 1H), 7.41 (d, J = 8.2 Hz, 1H), 7.33-7.21 (m, 2H), 6.94 (d, J = 5.5 Hz, 1H), 4.50 (s, 2H), 3.62 (s, 2H), 3.53 (s, 4H), 3.48 (s, 2H), 2.35 (s, 4H), 1.37 (s, 6H), 1.20 (d, J = 6.7 Hz, 6H). |
| 105 | 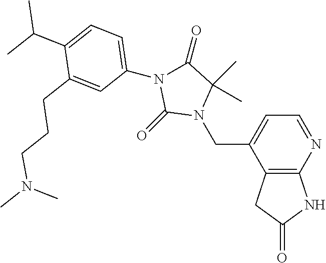 [SMILES: CC(C)c1ccc(N2C(=O)N(Cc3ccnc4c3CC(=O)N4)C(C)(C)C2=O)cc1CCCN(C)C] 3-{3-[3-(dimethylamino)propyl]-4-cumenyl}-5,5- dimethyl-1-[(2-oxo-1,7-diaza-4-indanyl)methyl]- 2,4-imidazolidinedione | LCMS: 477.5 (400 MHz,CD₃CN) δ 8.04 (d,J = 5.5 Hz, 1H), 7.43-7.38 (m, 1H), 7.23-7.17 (m, 2H), 6.96 (d,J = 5.6 Hz, 1H), 4.50 (s, 2H), 3.55 (s, 2H), 3.29-3.22 (m, 1H), 2.74-2.68 (m, 2H), 2.45- 2.37 (m, 2H), 2.24 (s, 6H), 1.78- 1.69 (m, 2H), 1.40 (s, 6H), 1.24 (d,J = 6.8 Hz, 6H). |
| 107 | 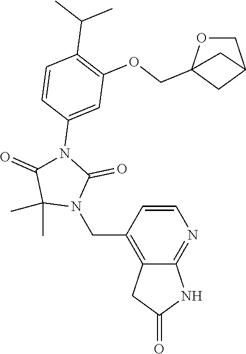 [SMILES: CC(C)c1ccc(N2C(=O)N(Cc3ccnc4c3CC(=O)N4)C(C)(C)C2=O)cc1OCC12CC(CO1)C2] 5,5-dimethyl-3-(3-{(2-oxabicyclo[2.1.1]hex-1- yl)methoxy}-4-cumenyl)-1-[(2-oxo-1,7-diaza-4- indanyl)methyl]-2,4-imidazolidinedione | LCMS: 505.6 (500 MHz, DMSO) δ 11.05 (s, 1H), 8.06 (d, J = 5.5 Hz, 1H), 7.32 (d, J = 8.2 Hz, 1H), 7.10 (d, J = 1.9 Hz, 1H), 7.04-6.93 (m, 2H), 4.55 (s, 2H), 4.22 (s, 2H), 3.76 (s, 2H), 3.67 (s, 2H), 3.33- 3.24 (m, 1H), 2.99 (t, J = 3.1 Hz, 1H), 1.94-1.89 (m, 2H), 1.49 (dd, J = 4.4, 1.7 Hz, 2H), 1.42 (s, 6H), 1.23 (d, J = 6.9 Hz, 6H). |
| 108 | 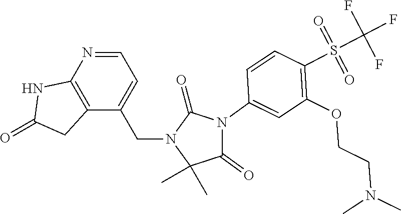 [SMILES: CN(C)CCOc1cc(N2C(=O)N(Cc3ccnc4c3CC(=O)N4)C(C)(C)C2=O)ccc1S(=O)(=O)C(F)(F)F] 3-{3-[2-(dimethylamino)ethoxy]-4- (trifluoromesyl)phenyl}-5,5-dimethyl-1-[(2-oxo- 1,7-diaza-4-indanyl)methyl]-2,4- imidazolidinedione | LCMS: 570.6 (500 MHz, DMSO) δ 11.03 (s, 1H), 8.12 (dd, J = 8.7, 4.6 Hz, 1H), 8.02 (d, J = 5.5 Hz, 1H), 7.68 (dd, J = 10.3, 1.8 Hz, 1H), 7.51 (dd, J = 8.7, 1.7 Hz, 1H), 7.02 (d, J = 5.5 Hz, 1H), 4.56 (s, 2H), 4.29 (t, J = 5.8 Hz, 2H), 3.67 (s, 2H), 2.74 (t, J = 4.6 Hz, 2H), 2.27 (s, 6H), 1.42 (s, 6H) |
| 109 | 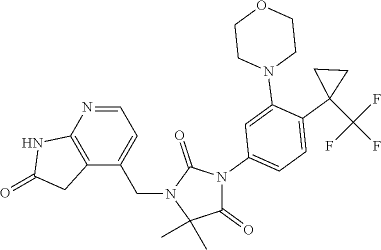 [SMILES: CC1(C)C(=O)N(c2ccc(C3(C(F)(F)F)CC3)c(N3CCOCC3)c2)C(=O)N1Cc1ccnc2c1CC(=O)N2] 5,5-dimethyl-3-{3-morpholino-4-[1- (trifluoromethyl)cyclopropyl]phenyl}-1-[(2-oxo- 1,7-diaza-4-indanyl)methyl]-2,4- imidazolidinedione | LCMS: 544.4 (500 MHz, DMSO) δ 11.03 (s, 1H), 8.03 (d, J = 5.5 Hz, 1H), 7.58 (d, J = 8.3 Hz, 1H), 7.46 (d, J = 2.1 Hz, 1H), 7.30 (dd, J = 8.3, 2.1 Hz, 1H), 6.99 (d, J = 5.5 Hz, 1H), 4.53 (s, 2H), 3.77- 3.71 (m, 4H), 3.65 (s, 2H), 2.86- 2.80 (m, 4H), 1.45-1.42 (m, 2H), 1.40 (s, 6H), 1.27-1.22 (m, 2H). |
| 110 | 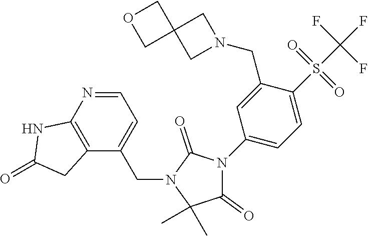 [SMILES: CC1(C)C(=O)N(c2ccc(S(=O)(=O)C(F)(F)F)c(CN3CC4(COC4)C3)c2)C(=O)N1Cc1ccnc2c1CC(=O)N2] 5,5-dimethyl-3-(3-{(2-oxa-6-aza-6- spiro[3.3]heptyl)methyl}-4- (trifluoromesyl)phenyl)-1-[(2-oxo-1,7-diaza-4- indanyl)methyl]-2,4-imidazolidinedione | LCMS: 594.3 (500 MHz, DMSO) δ 11.03 (s, 1H), 8.25 (d, J = 8.7 Hz, 1H), 8.09 (d,J = 2.0 Hz, 1H), 8.02 (d, J = 5.5 Hz, 1H), 7.90 (dd,J = 8.7, 2.1 Hz, 1H), 7.05 (d,J = 5.5 Hz, 1H), 4.61 (s, 4H), 4.56 (s, 2H), 3.96 (s, 2H), 3.67 (s, 2H), 3.43 (s, 4H), 1.42 (s, 6H) |
| 111 | 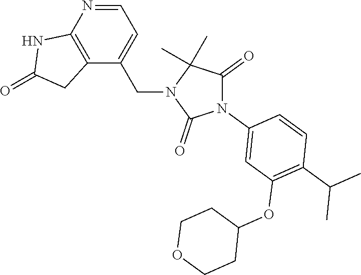 [SMILES: CC(C)c1ccc(N2C(=O)N(Cc3ccnc4c3CC(=O)N4)C(C)(C)C2=O)cc1OC1CCOCC1] 5,5-dimethyl-1-[(2-oxo-1,7-diaza-4- indanyl)methyl]-3-[3-(tetrahydro-2H-pyran-4- yloxy)-4-cumenyl]-2,4-imidazolidinedione | LCMS: 493.5 (400 MHz, DMSO) δ 10.99 (s, 1H), 8.03 (d, J = 5.5 Hz, 1H), 7.30 (d, J = 8.2 Hz, 1H), 7.06 (d, J = 1.8 Hz, 1H), 7.02-6.91 (m, 2H), 4.63-4.55 (m, 1H), 4.52 (s, 2H), 3.89-3.78 (m, 2H), 3.63 (s, 2H), 3.52 (ddd, J = 11.4, 8.3, 3.1 Hz, 2H), 3.30-3.28 (m, 1H), 2.06-1.93 (m, 2H), 1.72- 1.57 (m, 2H), 1.39 (s, 6H), 1.20 (d, J = 6.9 Hz, 6H). |
| 112 | 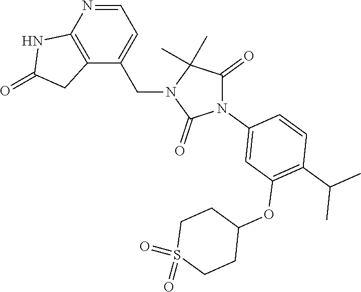 [SMILES: CC(C)c1ccc(N2C(=O)N(Cc3ccnc4c3CC(=O)N4)C(C)(C)C2=O)cc1OC1CCS(=O)(=O)CC1] 3-[3-(1,1-dioxo-126-4-thianyloxy)-4-cumenyl]- 5,5-dimethyl-1-[(2-oxo-1,7-diaza-4- indanyl)methyl]-2,4-imidazolidinedione | LCMS: 541.1 δ 11.00 (s, 1H), 8.03 (d, J = 5.5 Hz, 1H), 7.34 (d, J = 8.2 Hz, 1H), 7.08 (d, J = 1.9 Hz, 1H), 7.00 (dd, J = 8.1, 1.9 Hz, 1H), 6.95 (d, J = 5.5 Hz, 1H), 4.76- 4.66 (m, 1H), 4.52 (s, 2H), 3.64 (s, 2H), 3.27-3.09 (m, 5H), 2.27 (d, J = 5.0 Hz, 4H), 1.39 (s, 6H), 1.21 (d, J = 6.9 Hz, 6H). |
| 113 | 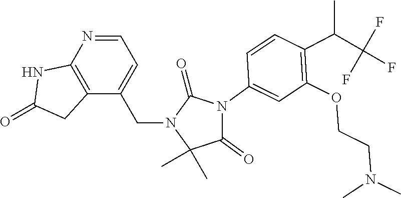 [SMILES: CC(c1ccc(N2C(=O)N(Cc3ccnc4c3CC(=O)N4)C(C)(C)C2=O)cc1OCCN(C)C)C(F)(F)F] 3-{3-[2-(dimethylamino)ethoxy]-4-(2,2,2- trifluoro-1-methylethyl)phenyl}-5,5-dimethyl-1- [(2-oxo-1,7-diaza-4-indanyl)methyl]-2,4- imidazolidinedione | LCMS: 533.6 (500 MHz, DMSO) δ 11.00 (s, 1H), 8.03 (d, J = 5.5 Hz, 1H), 7.48 (d, J = 8.2 Hz, 1H), 7.20 (d, J = 1.9 Hz, 1H), 7.09 (dd, J = 8.2, 1.9 Hz, 1H), 6.97 (d, J = 5.5 Hz, 1H), 4.53 (s, 2H), 4.20- 4.12 (m, 1H), 4.12-4.04 (m, 2H), 3.65 (s, 2H), 2.70 (t, J = 5.3 Hz, 2H), 2.25 (s, 6H), 1.42 (dd, J = 10.3, 4.8 Hz, 3H), 1.40 (s, 6H) |
| 114 | 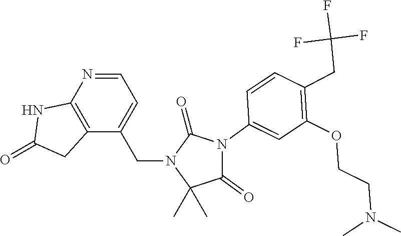 [SMILES: CN(C)CCOc1cc(N2C(=O)N(Cc3ccnc4c3CC(=O)N4)C(C)(C)C2=O)ccc1CC(F)(F)F] 3-{3-[2-(dimethylamino)ethoxy]-4-(2,2,2- trifluoroethyl)phenyl}-5,5-dimethyl-1-[(2-oxo- 1,7-diaza-4-indanyl)methyl]-2,4- imidazolidinedione | LCMS: 519.7 (500 MHz, DMSO) δ 11.00 (s, 1H), 8.03 (d, J = 5.5 Hz, 1H), 7.41 (d, J = 8.1 Hz, 1H), 7.20 (d, J = 1.9 Hz, 1H), 7.06 (dd, J = 8.1, 1.9 Hz, 1H), 6.97 (d, J = 5.5 Hz, 1H), 4.53 (s, 2H), 4.08 (t, J = 5.8 Hz, 2H), 3.68-3.58 (m, 4H), 2.67 (t, J = 5.8 Hz, 2H), 2.23 (s, 6H), 1.40 (s, 6H) |
| 115 | 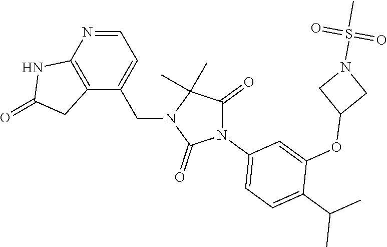 [SMILES: CC(C)c1ccc(N2C(=O)N(Cc3ccnc4c3CC(=O)N4)C(C)(C)C2=O)cc1OC1CN(S(C)(=O)=O)C1] 3-[3-(1-mesyl-3-azetidinyloxy)-4-cumenyl]-5,5- dimethyl-1-[(2-oxo-1,7-diaza-4-indanyl)methyl]- 2,4-imidazolidinedione | LCMS: 542.6 (500 MHz, DMSO) δ 11.00 (s, 1H), 8.04 (d, J = 5.5 Hz, 1H), 7.36 (d, J = 8.2 Hz, 1H), 7.05 (dd, J = 8.1, 1.9 Hz, 1H), 6.96 (d, J = 5.5 Hz, 1H), 6.80 (d, J = 1.9 Hz, 1H), 5.07-4.99 (m, 1H), 4.54 (s, 2H), 4.33 (dd, J = 9.8, 6.4 Hz, 2H), 3.92 (dd, J = 9.7, 4.8 Hz, 2H), 3.65 (s, 2H), 3.08 (s, 3H), 3.00-2.80 (m, 1H, obscured by H2O peak), 1.40 (s, 6H), 1.22 (d, J = 6.9 Hz, 6H). |
| 116 | 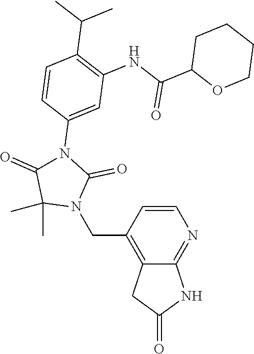 [SMILES: CC(C)c1ccc(N2C(=O)N(Cc3ccnc4c3CC(=O)N4)C(C)(C)C2=O)cc1NC(=O)C1CCCCO1] N-(5-{4,4-dimethyl-2,5-dioxo-3-[(2-oxo-1,7- diaza-4-indanyl)methyl]-1-imidazolidinyl}-2- cumenyl)-tetrahydro-2H-pyran-2-carboxamide | LCMS: 520.7 (400 MHz, DMSO) δ 10.99 (s, 1H), 9.22 (s, 1H), 8.22 (s, 1H), 8.02 (d, J = 5.5 Hz, 1H), 7.44- 7.38 (m, 2H), 7.28 (dd, J = 8.4, 2.1 Hz, 1H), 6.96 (d, J = 5.5 Hz, 1H), 4.52 (s, 2H), 4.08-4.00 (m, 1H), 3.95 (dd, J = 10.9, 2.5 Hz, 1H), 3.64 (s, 2H), 3.57- 3.47 (m, 1H), 3.14-3.03 (m, 1H), 1.99-1.90 (m, 1H), 1.89- 1.80 (m, 1H), 1.60-1.43 (m, 4H), 1.39 (s, 6H), 1.17 (d, J = 6.8 Hz, 6H). |
| 117 | 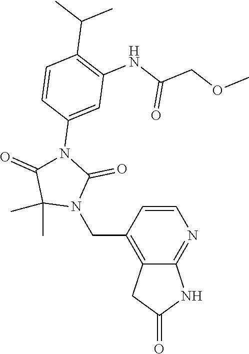 [SMILES: COCC(=O)Nc1cc(N2C(=O)N(Cc3ccnc4c3CC(=O)N4)C(C)(C)C2=O)ccc1C(C)C] N-(5-{4,4-dimethyl-2,5-dioxo-3-[(2-oxo-1,7- diaza-4-indanyl)methyl]-1-imidazolidinyl}-2- cumenyl)methoxyacetamide | LCMS: 480.5 (500 MHz, DMSO) δ 10.99 (s, 1H), 9.42 (s, 1H), 8.02 (d, J = 5.5 Hz, 1H), 7.44 (d, J = 8.4 Hz, 1H), 7.39 (d, J = 2.2 Hz, 1H), 7.30 (dd, J = 8.4, 2.2 Hz, 1H), 6.96 (d, J = 5.5 Hz, 1H), 4.52 (s, 2H), 4.03 (s, 2H), 3.64 (s, 2H), 3.41 (s, 3H), 3.12 (hept, J = 6.7 Hz, 1H), 1.39 (s, 6H), 1.18 (d, J = 6.9 Hz, 6H). |
| 118 | 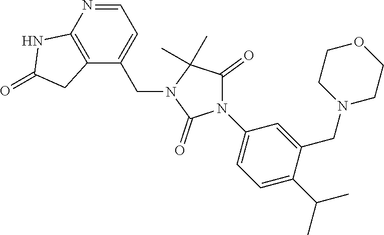 5,5-dimethyl-3-[3-(2-morpholinoethoxy)-4- cumenyl]-1-[(2-oxo-1,7-diaza-4-indanyl)methyl]- 2,4-imidazolidinedione | LCMS: 522.6 (500 MHz, DMSO) δ 8.03 (d, J = 5.5 Hz, 1H), 7.28 (d, J = 8.2 Hz, 1H), 7.05 (d, J = 2.0 Hz, 1H), 6.97-6.93 (m, 2H), 4.52 (s, 2H), 4.08 (t, J = 5.8 Hz, 2H), 3.64 (s, 2H), 3.59-3.54 (m, 4H), 3.29-3.22 (m, 1H), 2.74 (t, J = 5.8 Hz, 2H), 1.39 (s, 6H), 1.19 (d, J = 6.9 Hz, 6H). |
| 119 | 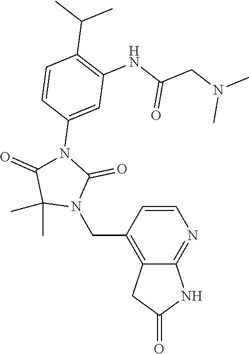 [SMILES: CC(C)c1ccc(N2C(=O)N(Cc3ccnc4c3CC(=O)N4)C(C)(C)C2=O)cc1NC(=O)CN(C)C] N-(5-{4,4-dimethyl-2,5-dioxo-3-[(2-oxo-1,7- diaza-4-indanyl)methyl]-1-imidazolidinyl}-2- cumenyl)(dimethylamino)acetamide | LCMS: 493.2 (400 MHz, DMSO) δ 10.97 (s, 1H), 9.52 (s, 1H), 8.21 (s, 1H), 8.00 (d, J = 5.4 Hz, 1H), 7.60 (d, J = 1.9 Hz, 1H), 7.40 (d, J = 8.4 Hz, 1H), 7.22 (dd, J = 8.3, 2.0 Hz, 1H), 6.94 (d, J = 5.5 Hz, 1H), 4.50 (s, 2H), 3.62 (s, 2H), 3.13-3.01 (m, 3H), 2.31 (s, 6H), 1.37 (s, 6H), 1.19 (d, J = 6.8 Hz, 6H). |
| 120 | 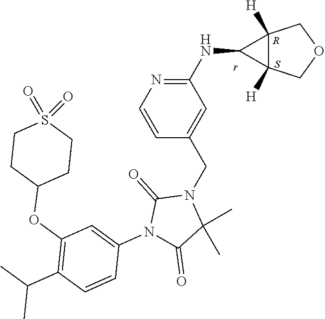 1-[(2-{(1R,5S,6r)-3-oxabicyclo[3.1.0]hex-6- ylamino}-4-pyridyl)methyl]-3-[3-(1,1-dioxo-126- 4-thianyloxy)-4-cumenyl]-5,5-dimethyl-2,4- imidazolidinedione | LCMS: 583.5 (400 MHz, DMSO) δ 7.95 (d, J = 5.2 Hz, 1H), 7.34 (d, J = 8.2 Hz, 1H), 7.05 (d, J = 1.7 Hz, 1H), 6.96 (dd, J = 8.1, 1.7 Hz, 1H), 6.75 (s, 1H), 6.58 (d, J = 5.1 Hz, 1H), 6.52 (s, 1H), 4.72 (d, J = 4.4 Hz, 1H), 4.49 (s, 2H), 3.86 (d, J = 8.3 Hz, 2H), 3.64 (d, J = 8.2 Hz, 2H), 3.30-3.07 (m, 6H), 2.26 (s, J = 5.5 Hz, 1H), 2.26-2.17 (m, 3H), 1.75 (s, 2H), 1.39 (s, 6H), 1.21 (d, J = 6.9 Hz, 6H). |
| 121 | 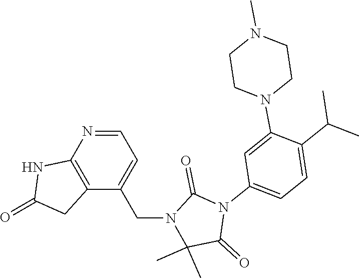 [SMILES: CC(C)c1ccc(N2C(=O)N(Cc3ccnc4c3CC(=O)N4)C(C)(C)C2=O)cc1N1CCN(C)CC1] 5,5-dimethyl-3-[3-(4-methyl-1-piperazinyl)-4- cumenyl]-1-[(2-oxo-1,7-diaza-4-indanyl)methyl]- 2,4-imidazolidinedione | LCMS: 491.3 (400 MHz, acetone) δ 9.85 (s, 1H), 8.02 (d, J = 5.5 Hz, 1H), 7.37 (d, J = 8.3 Hz, 1H), 7.29 (d, J = 2.1 Hz, 1H), 7.21 (dd, J = 8.3, 2.1 Hz, 1H), 7.02 (d, J = 5.5 Hz, 1H), 4.64 (s, 2H), 3.66 (s, 2H), 3.60-3.48 (m, 1H), 3.08- 3.01 (m, 4H), 2.94-2.87 (m, 4H), 2.51 (s, 3H), 1.46 (s, 6H), 1.22 (s, 3H), 1.20 (s, 3H) |
| 122 | 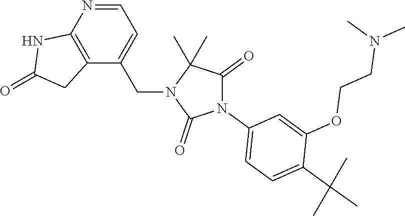 [SMILES: CN(C)CCOc1cc(N2C(=O)N(Cc3ccnc4c3CC(=O)N4)C(C)(C)C2=O)ccc1C(C)(C)C] 3-{4-(tert-butyl)-3-[2- (dimethylamino)ethoxy]phenyl}-5,5-dimethyl-1- [(2-oxo-1,7-diaza-4-indanyl)methyl]-2,4- imidazolidinedione | LCMS: 494.3 (400 MHz, DMSO) δ 11.00 (s, 1H), 8.03 (d, J = 5.5 Hz, 1H), 7.30 (d, J = 8.4 Hz, 1H), 7.08 (d, J = 2.0 Hz, 1H), 6.96 (d, J = 5.5 Hz, 1H), 6.92 (dd, J = 8.3, 2.0 Hz, 1H), 4.52 (s, 2H), 4.05 (t, J = 6.0 Hz, 2H), 3.64 (s, 2H), 2.71 (t, J = 5.9 Hz, 2H), 2.24 (s, 6H), 1.39 (s, 6H), 1.37 (s, 9H). |
| 123 | 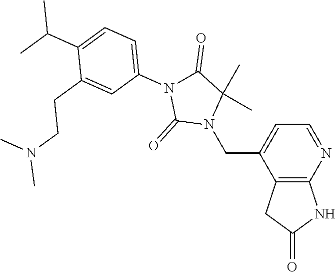 [SMILES: CC(C)c1ccc(N2C(=O)N(Cc3ccnc4c3CC(=O)N4)C(C)(C)C2=O)cc1CCN(C)C] 3-{3-[2-(dimethylamino)ethyl]-4-cumenyl}-5,5- dimethyl-1-[(2-oxo-1,7-diaza-4-indanyl)methyl]- 2,4-imidazolidinedione | LCMS: 464.3 (500 MHz, DMSO) δ 8.29 (s, 1H), 8.03 (d,J = 5.5 Hz, 1H), 7.38 (d,J = 8.3 Hz, 1H), 7.25- 7.18 (m, 2H), 6.95 (d,J = 5.5 Hz, 1H), 4.51 (s, 2H), 3.64 (s, 2H), 3.19-3.11 (m, 2H), 2.83-2.73 (m, 2H), 2.46-2.40 (m, 2H), 2.23 (s, 6H), 1.38 (s, 6H), 1.23 (t,J = 9.4 Hz, 6H). |
| 124 | 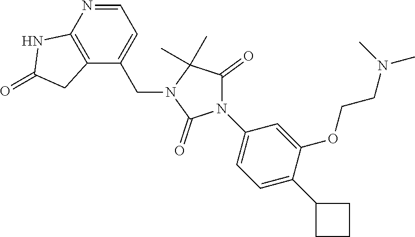 [SMILES: CN(C)CCOc1cc(N2C(=O)N(Cc3ccnc4c3CC(=O)N4)C(C)(C)C2=O)ccc1C1CCC1] 3-{4-cyclobutyl-3-[2- (dimethylamino)ethoxy]phenyl}-5,5-dimethyl-1- [(2-oxo-1,7-diaza-4-indanyl)methyl]-2,4- imidazolidinedione | LCMS: 492.3 (400 MHz, DMSO) δ 10.98 (s, 1H), 8.01 (d, J = 5.4 Hz, 1H), 7.26 (d, J = 7.9 Hz, 1H), 6.99 (d, J = 1.7 Hz, 1H), 6.95 (d, J = 5.8 Hz, 2H), 4.50 (s, 2H), 3.99 (t, J = 5.8 Hz, 2H), 3.71-3.55 (m, 3H), 2.64 (t, J = 5.8 Hz, 2H), 2.31-2.18 (m, 8H), 2.13-1.87 (m, 3H), 1.84-1.68 (m, 1H), 1.37 (s, 6H). |
| 125 | 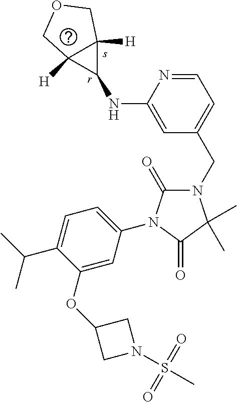 [SMILES: CC(C)c1ccc(N2C(=O)N(Cc3ccnc(N[C@H]4[C@@H]5COC[C@@H]54)c3)C(C)(C)C2=O)cc1OC1CN(S(C)(=O)=O)C1] 1-[(2-{(1R,5S,6r)-3-oxabicyclo[3.1.0]hex-6- ylamino}-4-pyridyl)methyl]-3-[3-(1-mesyl-3- azetidinyloxy)-4-cumenyl]-5,5-dimethyl-2,4- imidazolidinedione | LCMS: 584.7 (500 MHz, DMSO) δ 7.95 (d, J = 5.2 Hz, 1H), 7.35 (d, J = 8.2 Hz, 1H), 7.01 (dd, J = 8.1, 1.9 Hz, 1H), 6.76 (d, J = 1.9 Hz, 1H), 6.73 (d, J = 2.3 Hz, 1H), 6.59 (dd, J = 5.2, 1.4 Hz, 1H), 6.52 (s, 1H), 5.05-4.98 (m, 1H), 4.49 (s, 2H), 4.34-4.28 (m, 2H), 3.94-3.89 (m, 2H), 3.86 (d, J = 8.4 Hz, 2H), 3.66- 3.60 (m, 2H), 3.34-3.26 (m, 1H), 3.06 (s, 3H), 2.27-2.23 (m, 1H), 1.78-1.72 (m, 2H), 1.39 (s, 6H), 1.21 (d, J = 6.9 Hz, 6H). |
| 126 | 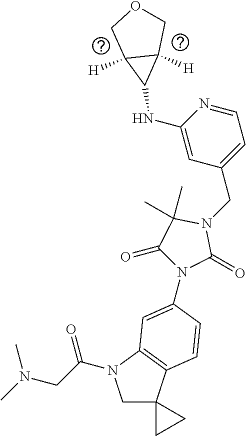 1-[(2-{(1R,5S,6r)-3-oxabicyclo[3.1.0]hex-6- ylamino}-4-pyridyl)methyl]-3-{1′-[2- (dimethylamino)acetyl]spiro[cyclopropane-1,3′- indolin]-6′-yl}-5,5-dimethyl-2,4- imidazolidinedione | LCMS: 545.6 (500 MHz, DMSO) δ 8.05 (s, 1H), 7.95 (d, J = 5.1 Hz, 1H), 6.98 (d, J = 7.8 Hz, 1H), 6.90 (d, J = 8.0 Hz, 1H), 6.74 (s, 1H), 6.59 (d, J = 5.2 Hz, 1H), 6.51 (s, 1H), 4.49 (s, 2H), 4.23 (s, 2H), 3.86 (d, J = 8.4 Hz, 2H), 3.63 (d, J = 8.1 Hz, 2H), 3.21 (s, 2H), 2.28 (s, 6H), 2.25 (s, 1H), 1.76 (s, 2H), 1.40 (s, 6H), 1.15 (d, J = 7.4 Hz, 2H), 1.10 (t, J = 5.3 Hz, 2H). |
| 127 | 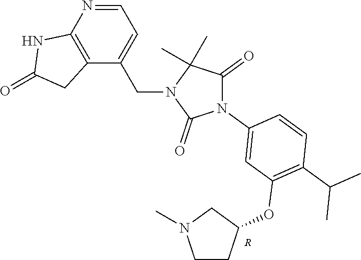 3-{3-[(R)-1-methyl-3-pyrrolidinyloxy]-4- cumenyl}-5,5-dimethyl-1-[(2-oxo-1,7-diaza-4- indanyl)methyl]-2,4-imidazolidinedione | LCMS: 492.2 (500 MHz, DMSO) δ 10.94 (s, 1H), 7.96 (d, J = 5.2 Hz, 1H), 7.21 (d, J = 8.7 Hz, 1H), 6.92- 6.82 (m, 3H), 4.81-4.74 (m, 1H), 4.46 (s, 2H), 3.57 (s, 2H), 3.17 (dt, J = 13.7, 6.8 Hz, 1H), 2.79 (s, 1H), 2.59 (s, 1H), 2.53 (d, J = 9.0 Hz, 1H), 2.36 (d, J = 5.1 Hz, 1H), 2.26-2.17 (m, 4H), 1.73 (ddd, J = 13.4, 9.2, 4.0 Hz, 1H), 1.32 (s, 6H), 1.11 (d, J = 6.9 Hz, 6H). |
| 128 | 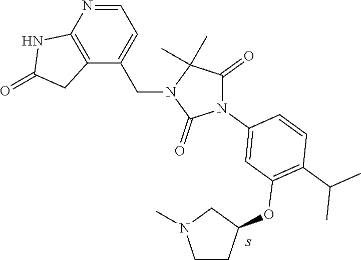 3-{3-[(S)-1-methyl-3-pyrrolidinyloxy]-4- cumenyl}-5,5-dimethyl-1-[(2-oxo-1,7-diaza-4- indanyl)methyl]-2,4-imidazolidinedione | LCMS: 492.2 (500 MHz, DMSO) δ 11.01 (s, 1H), 8.34 (s, 2H), 8.03 (d, J = 5.5 Hz, 1H), 7.28 (d, J = 8.7 Hz, 1H), 6.99-6.93 (m, 3H), 4.88- 4.83 (m, 1H), 4.53 (s, 2H), 3.64 (s, 2H), 3.27-3.21 (m, 1H), 2.85 (dd, J = 10.3, 6.1 Hz, 1H), 2.65 (t, J = 7.0 Hz, 1H), 2.58 (dd, J = 10.4, 2.8 Hz, 1H), 2.41 (dd, J = 14.7, 7.5 Hz, 1H), 2.33- 2.24 (m, 4H), 1.84-1.76 (m, 1H), 1.39 (s, 6H), 1.19 (d, J = 6.9 Hz, 6H). |
| 129 | 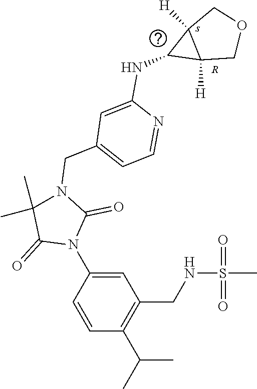 1-[(2-{(1R,5S,6r)-3-oxabicyclo[3.1.0]hex-6- ylamino}-4-pyridyl)methyl]-3-{3- [(mesylamino)methyl]-4-cumenyl}-5,5-dimethyl- 2,4-imidazolidinedione | LCMS: 542.4 (400 MHz, DMSO) δ 7.93 (d, J = 5.4 Hz, 1H), 7.47-7.38 (m, 2H), 7.33 (d, J = 2.2 Hz, 1H), 7.28 (dd, J = 8.3, 2.2 Hz, 1H), 6.72 (d, J = 2.3 Hz, 1H), 6.56 (dd, J = 5.2, 1.4 Hz, 1H), 6.50 (s, 1H), 4.48 (s, 2H), 4.21 (d, J = 6.2 Hz, 2H), 3.85 (d, J = 8.4 Hz, 2H), 3.62 (d, J = 8.2 Hz, 2H), 3.27-3.20 (m, 1H), 2.89 (s, 3H), 2.26-2.21 (m, 1H), 1.73 (s, 2H), 1.38 (s, 6H), 1.20 (d, J = 6.8 Hz, 6H). |
| 130 | 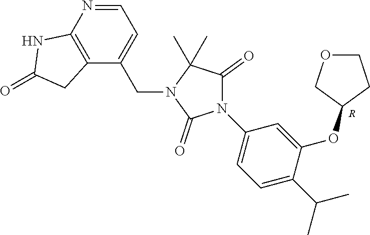 3-{3-[(R)-tetrahydro-3-furyloxy]-4-cumenyl}- 5,5-dimethyl-1-[(2-oxo-1,7-diaza-4- indanyl)methyl]-2,4-imidazolidinedione | LCMS: 479.6 (500 MHz, DMSO) δ 11.01 (s, 1H), 8.04 (d, J = 5.5 Hz, 1H), 7.31 (d, J = 8.2 Hz, 1H), 7.03 (d, J = 1.9 Hz, 1H), 7.01-6.95 (m, 2H), 5.06-5.01 (m, 1H), 4.53 (s, 2H), 3.91 (dd, J = 10.1, 4.4 Hz, 1H), 3.88-3.78 (m, 3H), 3.65 (s, 2H), 3.24 (hept, J = 6.9 Hz, 1H), 2.22 (dddd, J = 14.3, 8.4, 6.0 Hz, 1H), 2.05-1.97 (m, 1H), 1.40 (s, 6H), 1.19 (d, J = 6.9 Hz, 6H). |
| 131 | 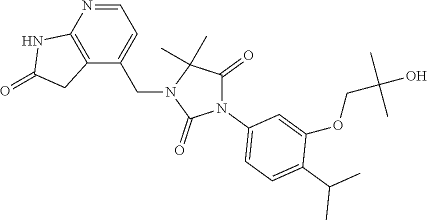 [SMILES: CC(C)c1ccc(N2C(=O)N(Cc3ccnc4c3CC(=O)N4)C(C)(C)C2=O)cc1OCC(C)(C)O] 3-[3-(2-hydroxy-2-methylpropoxy)-4-cumenyl]- 5,5-dimethyl-1-[(2-oxo-1,7-diaza-4- indanyl)methyl]-2,4-imidazolidinedione | LCMS: 481.6 (500 MHz, DMSO) δ 8.35 (s, 1H), 8.04 (d, J = 5.5 Hz, 1H), 7.29 (d, J = 8.1 Hz, 1H), 7.01 (d, J = 1.9 Hz, 1H), 6.99-6.92 (m, 2H), 4.68 (s, 1H), 4.52 (s, 2H), 3.70 (s, 2H), 3.65 (s, 2H), 3.33 (sept, J = 6.7 Hz, 1H), 1.40 (s, 6H), 1.26 (s, 6H), 1.22 (d, J = 6.9 Hz, 6H). |
| 132 | 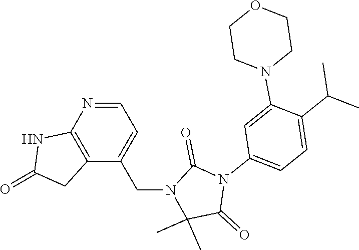 [SMILES: CC(C)c1ccc(N2C(=O)N(Cc3ccnc4c3CC(=O)N4)C(C)(C)C2=O)cc1N1CCOCC1] 5,5-dimethyl-3-(3-morpholino-4-cumenyl)-1-[(2- oxo-1,7-diaza-4-indanyl)methyl]-2,4- imidazolidinedione | LCMS: 478.3 (400 MHz, DMSO) δ 10.99 (br s, 1H), 8.03 (d, J = 5.5 Hz, 1H), 7.39 (d, J = 8.3 Hz, 1H), 7.20 (d, J = 2.0 Hz, 1H), 7.15 (dd, J = 8.3, 2.0 Hz, 1H), 6.97 (d, J = 5.5 Hz, 1H), 4.52 (s, 2H), 3.80- 3.70 (m, 4H), 3.64 (s, 2H), 3.48 (dt, J = 13.8, 6.8 Hz, 1H), 2.87- 2.74 (m, 4H), 1.39 (s, 6H), 1.21 (s, 3H), 1.19 (s, 3H). |
| 133 | 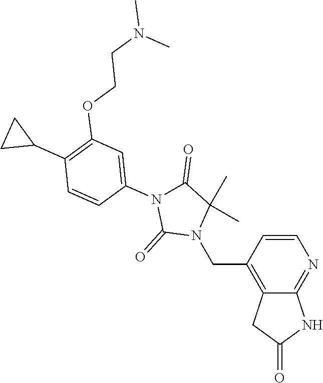 [SMILES: CN(C)CCOc1cc(N2C(=O)N(Cc3ccnc4c3CC(=O)N4)C(C)(C)C2=O)ccc1C1CC1] 3-{4-cyclopropyl-3-[2- (dimethylamino)ethoxy]phenyl}-5,5-dimethyl-1- [(2-oxo-1,7-diaza-4-indanyl)methyl]-2,4- imidazolidinedione | LCMS: 478.4 (400 MHz, DMSO) δ 10.87 (brs, 1H), 8.03 (d, J = 5.5 Hz, 1H), 7.04 (s, 1H), 6.97 (d, J = 5.5 Hz, 1H), 6.91 (s, 2H), 4.52 (s, 2H), 4.07 (t, J = 5.7 Hz, 2H), 3.65 (s, 2H), 2.70 (t, J = 5.7 Hz, 2H), 2.26 (s, 6H), 2.15 (tt, J = 8.5, 5.3 Hz, 1H), 1.39 (s, 6H), 0.96- 0.90 (m, 2H), 0.71-0.65 (m, 2H). |
| 134 | 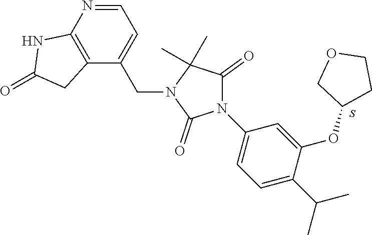 3-{3-[(S)-tetrahydro-3-furyloxy]-4-cumenyl}-5,5- dimethyl-1-[(2-oxo-1,7-diaza-4-indanyl)methyl]- 2,4-imidazolidinedione | LCMS: 479.5 (500 MHz, DMSO) δ 11.00 (s, 1H), 8.03 (d, J = 5.5 Hz, 1H), 7.30 (d, J = 8.2 Hz, 1H), 7.02 (d, J = 1.9 Hz, 1H), 7.00-6.94 (m, 2H), 5.05-4.99 (m, 1H), 4.52 (s, 2H), 3.90 (dd, J = 10.1, 4.4 Hz, 1H), 3.88-3.77 (m, 3H), 3.64 (s, 2H), 3.23 (hept, J = 6.5 Hz, 1H), 2.26-2.17 (m, 1H), 2.04-1.96 (m, 1H), 1.39 (s, 6H), 1.18 (d, J = 6.9 Hz, 6H). |
| 135 | 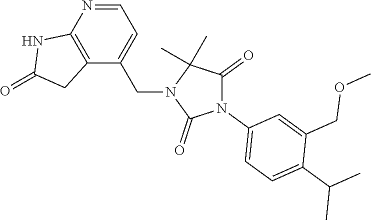 [SMILES: COCc1cc(N2C(=O)N(Cc3ccnc4c3CC(=O)N4)C(C)(C)C2=O)ccc1C(C)C] 3-[3-(methoxymethyl)-4-cumenyl]-5,5-dimethyl- 1-[(2-oxo-1,7-diaza-4-indanyl)methyl]-2,4- imidazolidinedione | LCMS: 437.4 (500 MHz, DMSO) δ 8.45 (s, 1H), 8.03 (d, J = 5.5 Hz, 1H), 7.44 (d, J = 8.3 Hz, 1H), 7.38- 7.30 (m, 2H), 6.97 (d, J = 5.5 Hz, 1H), 4.57-4.45 (m, 4H), 3.64 (s, 2H), 3.35 (s, 3H), 3.17 (sept, J = 6.9 Hz, 1H), 1.40 (s, 6H), 1.22 (d, J = 6.8 Hz, 6H). |
| 136 | 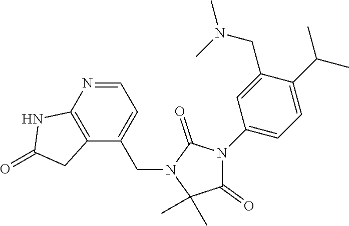 [SMILES: CC(C)c1ccc(N2C(=O)N(Cc3ccnc4c3CC(=O)N4)C(C)(C)C2=O)cc1CN(C)C] 3-{3-[(dimethylamino)methyl]-4-cumenyl}-5,5- dimethyl-1-[(2-oxo-1,7-diaza-4-indanyl)methyl]- 2,4-imidazolidinedione | LCMS: 450.3 (500 MHz, DMSO) δ 11.00 (s, 1H), 8.03 (d,J = 5.5 Hz, 1H), 7.42 (s, 1H), 7.31 (s, 2H), 6.97 (d,J = 5.5 Hz, 1H), 4.53 (s, 2H), 3.65 (s, 2H), 3.38-3.34 (m, 1H), 3.29 (s, 2H), 2.18 (s, 6H), 1.40 (s, 6H), 1.21 (d,J = 6.8 Hz, 6H) |
| 137 | 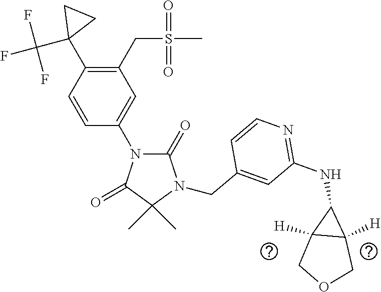 [SMILES: CC1(C)C(=O)N(c2ccc(C3(C(F)(F)F)CC3)c(CS(C)(=O)=O)c2)C(=O)N1Cc1ccnc(N[C@H]2[C@@H]3COC[C@@H]32)c1] 1-[(2-{(1R,5S,6r)-3-oxabicyclo[3.1.0]hex-6- ylamino}-4-pyridyl)methyl]-3-{3-(mesylmethyl)- 4-[1-(trifluoromethyl)cyclopropyl]phenyl}-5,5- dimethyl-2,4-imidazolidinedione | LCMS: 593.4 (400 MHz, DMSO) δ 7.94 (d,J = 5.1 Hz, 1H), 7.72-7.62 (m, 2H), 7.53 (dd,J = 8.3, 2.3 Hz, 1H), 6.73 (s, 1H), 6.59 (d,J = 5.1 Hz, 1H), 6.52 (s, 1H), 4.79 (s, 2H), 4.50 (s, 2H), 3.75 (dd,J = 92.6, 8.2 Hz, 4H), 3.20 (s, 3H), 2.26 (s, 1H), 1.75 (s, 2H), 1.51 (s, 2H), 1.41 (s, 6H), 1.24 (s, 2H). |
| 138 | 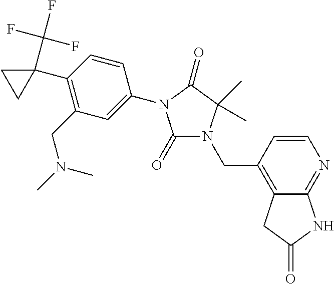 [SMILES: CN(C)Cc1cc(N2C(=O)N(Cc3ccnc4c3CC(=O)N4)C(C)(C)C2=O)ccc1C1(C(F)(F)F)CC1] 3-{3-[(dimethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 516.3 (500 MHz, DMSO) δ 11.00 (s, 1H), 8.03 (d,J = 5.5 Hz, 1H), 7.68 (d,J = 2.0 Hz, 1H), 7.54 (d, J = 8.3 Hz, 1H), 7.36 (dd,J = 8.3, 2.1 Hz, 1H), 6.98 (d,J = 5.5 Hz, 1H), 4.53 (s, 2H), 3.64 (s, 2H), 3.61 (s, 2H), 2.21 (s, 6H), 1.48 (s, 2H), 1.40 (s, 6H), 1.21 (s, 2H). |
| 139 | 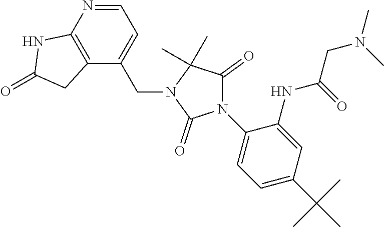 [SMILES: CN(C)CC(=O)Nc1cc(C(C)(C)C)ccc1N1C(=O)N(Cc2ccnc3c2CC(=O)N3)C(C)(C)C1=O] N-[5-(tert-butyl)-2-{4,4-dimethyl-2,5-dioxo-3- [(2-oxo-1,7-diaza-4-indanyl)methyl]-1- imidazolidinyl}phenyl](dimethylamino)acetamide | LCMS: 507.27 (500 MHz, DMSO) δ 11.01 (s, 1H), 9.29 (s, 1H), 8.11 (d, J = 1.9 Hz, 1H), 8.04 (d, J = 5.5 Hz, 1H), 7.34 (d, J = 8.4 Hz, 1H), 7.29 (dd, J = 8.4, 2.1 Hz, 1H), 6.97 (d, J = 5.5 Hz, 1H), 4.56 (d, J = 17.0 Hz, 1H), 4.52 (d, J = 17.0 Hz, 1H), 3.68 (d, J = 22.3 Hz, 1H), 3.62 (d, J = 22.3 Hz, 1H), 3.05 (d, J = 16.0 Hz, 1H), 2.99 (d, J = 16.0 Hz, 1H), 2.21 (s, 6H), 1.44 (s, 3H), 1.40 (s, 3H), 1.31 (s, 9H). |
| 140 | 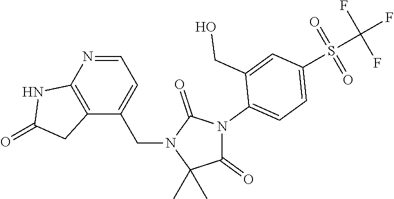 [SMILES: CC1(C)C(=O)N(c2ccc(S(=O)(=O)C(F)(F)F)cc2CO)C(=O)N1Cc1ccnc2c1CC(=O)N2] 3-[2-(hydroxymethyl)-4-(trifluoromesyl)phenyl]- 5,5-dimethyl-1-[(2-oxo-1,7-diaza-4- indanyl)methyl]-2,4-imidazolidinedione | LCMS: 513.1 (400 MHz, DMSO) δ 11.01 (br s, 1H), 8.31-8.20 (m, 2H), 8.03 (d, J = 5.5 Hz, 1H), 7.93 (d, J = 8.4 Hz, 1H), 7.01 (d, J = 5.5 Hz, 1H), 5.72 (s, 1H), 4.68-4.46 (m, 4H), 3.74-3.56 (m, 2H), 1.44 (s, 6H) |
| 141 | 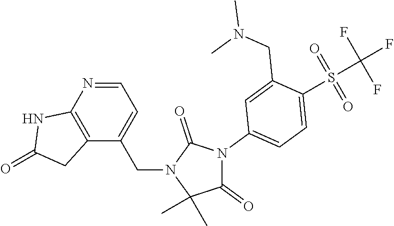 [SMILES: CN(C)Cc1cc(N2C(=O)N(Cc3ccnc4c3CC(=O)N4)C(C)(C)C2=O)ccc1S(=O)(=O)C(F)(F)F] 3-{3-[(dimethylamino)methyl]-4- (trifluoromesyl)phenyl}-5,5-dimethyl-1-[(2-oxo- 1,7-diaza-4-indanyl)methyl]-2,4- imidazolidinedione | LCMS: 540.1 (500 MHz, DMSO) δ 10.99 (s, 1H), 8.27 (d, J = 8.7 Hz, 1H), 8.23 (d, J = 2.2 Hz, 1H), 8.02 (d, J = 5.5 Hz, 1H), 7.91 (d, J = 7.1 Hz, 1H), 7.04 (d, J = 5.5 Hz, 1H), 4.55 (s, 2H), 3.83 (s, 2H), 3.66 (s, 2H), 2.21 (d, J = 20.7 Hz, 6H), 1.42 (s, J = 5.7 Hz, 6H) |
| 142 | 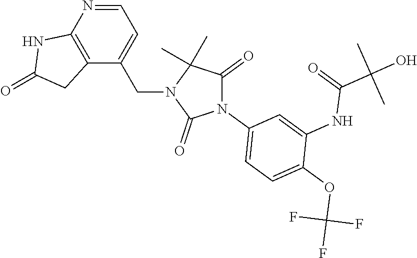 [SMILES: CC(C)(O)C(=O)Nc1cc(N2C(=O)N(Cc3ccnc4c3CC(=O)N4)C(C)(C)C2=O)ccc1OC(F)(F)F] N-(5-{4,4-dimethyl-2,5-dioxo-3-[(2-oxo-1,7- diaza-4-indanyl)methyl]-1-imidazolidinyl}-2- trifluoromethoxyphenyl)2-hydroxy-2- methylpropionamide | LCMS: 536.08 (500 MHz, DMSO) δ 11.00 (s, 1H), 9.52 (s, 1H), 8.32 (d, J = 2.5 Hz, 1H), 8.02 (d, J = 5.5 Hz, 1H), 7.60 (dd, J = 8.8, 1.3 Hz, 1H), 7.36 (dd, J = 8.8, 2.5 Hz, 1H), 7.00 (d, J = 5.5 Hz, 1H), 6.13 (s, 1H), 4.53 (s, 2H), 3.65 (s, 2H), 1.40 (s, 6H), 1.36 (s, 6H). |
| 143 | 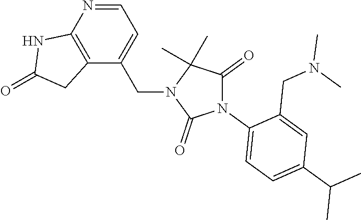 [SMILES: CC(C)c1ccc(N2C(=O)N(Cc3ccnc4c3CC(=O)N4)C(C)(C)C2=O)c(CN(C)C)c1] 3-{2-[(dimethylamino)methyl]-4-cumenyl}-5,5- dimethyl-1-[(2-oxo-1,7-diaza-4-indanyl)methyl]- 2,4-imidazolidinedione | LCMS: 450.3 (500 MHz, DMSO) δ 11.01 (s, 1H), 8.04 (d, J = 5.5 Hz, 1H), 7.30-7.27 (m, 2H), 7.25-7.21 (m, 1H), 6.93 (d, J = 5.5 Hz, 1H), 4.60 (d, J = 17.0 Hz, 1H), 4.46 (d, J = 17.0 Hz, 1H), 3.66 (d, J = 22.4 Hz, 1H), 3.60 (d, J = 22.2 Hz, 1H), 3.35 (d, J = 13.0 Hz, 1H), 3.21 (d, J = 13.0 Hz, 1H), 2.94 (hept, J = 7.1 Hz, 1H), 2.03 (s, 6H), 1.43 (s, 3H), 1.31 (s, 3H), 1.23 (d, J = 6.9 Hz, 6H). |
| 144 | 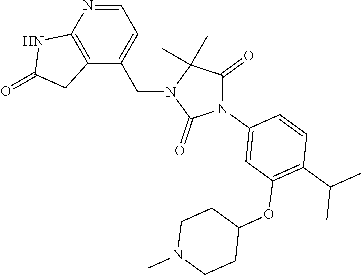 [SMILES: CC(C)c1ccc(N2C(=O)N(Cc3ccnc4c3CC(=O)N4)C(C)(C)C2=O)cc1OC1CCN(C)CC1] 5,5-dimethyl-3-[3-(1-methyl-4-piperidyloxy)-4- cumenyl]-1-[(2-oxo-1,7-diaza-4-indanyl)methyl]- 2,4-imidazolidinedione | LCMS: 506.4 (400 MHz, DMSO-d6) δ 11.00 (s, 1H), 8.02 (d, J = 5.5 Hz, 1H), 7.29 (d, J = 8.2 Hz, 1H), 7.03 (d, J = 1.9 Hz, 1H), 6.97-6.92 (m, 2H), 4.52 (s, 2H), 4.44-4.36 (m, 1H), 3.63 (s, 2H), 3.30- 3.25 (m, 1H), 2.56 (s, 2H), 2.30 (s, 2H), 2.21 (s, 3H), 1.98-1.88 (m, 2H), 1.78-1.68 (m, 2H), 1.38 (s, 6H), 1.20 (d, J = 6.9 Hz, 6H). |
| 145 | 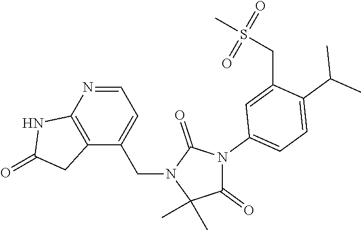 [SMILES: CC(C)c1ccc(N2C(=O)N(Cc3ccnc4c3CC(=O)N4)C(C)(C)C2=O)cc1CS(C)(=O)=O] 3-[3-(mesylmethyl)-4-cumenyl]-5,5-dimethyl-1- [(2-oxo-1,7-diaza-4-indanyl)methyl]-2,4- imidazolidinedione | LCMS: 485.2 (500 MHz, DMSO) δ 10.99 (s, 1H), 8.02 (d,J = 5.5 Hz, 1H), 7.52 (d,J = 8.5 Hz, 1H), 7.44 (dd,J = 8.4, 2.2 Hz, 1H), 7.39 (d,J = 2.2 Hz, 1H), 6.95 (d,J = 5.5 Hz, 1H), 4.63 (s, 2H), 4.53 (s, 2H), 3.64 (s, 2H), 3.36-3.34 (m,J = 6.9 Hz, 1H), 3.04 (s,J = 7.4 Hz, 3H), 1.40 (s, 6H), 1.21 (s, 3H), 1.20 (s, 3H). |
| 146 | 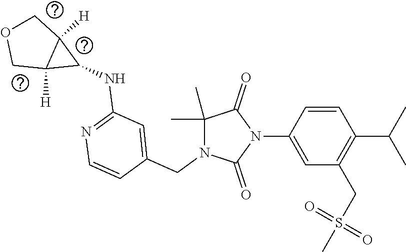 [SMILES: CC(C)c1ccc(N2C(=O)N(Cc3ccnc(N[C@H]4[C@@H]5COC[C@@H]54)c3)C(C)(C)C2=O)cc1CS(C)(=O)=O] 1-[(2-{(1R,5S,6r)-3-oxabicyclo[3.1.0]hex-6- ylamino}-4-pyridyl)methyl]-3-[3-(mesylmethyl)- 4-cumenyl]-5,5-dimethyl-2,4-imidazolidinedione | LCMS: 527.2 (500 MHz, DMSO) δ 7.94 (d,J = 5.3 Hz, 1H), 7.52 (d,J = 8.4 Hz, 1H), 7.43-7.35 (m, 2H), 6.73 (d,J = 2.3 Hz, 1H), 6.58 (dd, J = 5.2, 1.4 Hz, 1H), 6.52 (s, 1H), 4.63 (s, 2H), 4.49 (s, 2H), 3.87 (d, J = 8.4 Hz, 2H), 3.64 (d,J = 8.2 Hz, 2H), 3.03 (s, 3H), 2.26 (d,J = 2.2 Hz, 1H), 1.74 (d,J = 10.0 Hz, 2H), 1.40 (s, 6H), 1.21 (s, 3H), 1.20 (s, 3H) |
| 147 | 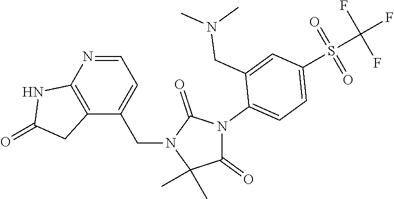 [SMILES: CN(C)Cc1cc(S(=O)(=O)C(F)(F)F)ccc1N1C(=O)N(Cc2ccnc3c2CC(=O)N3)C(C)(C)C1=O] 3-{2-[(dimethylamino)methyl]-4- (trifluoromesyl)phenyl}-5,5-dimethyl-1-[(2-oxo- 1,7-diaza-4-indanyl)methyl]-2,4- imidazolidinedione | LCMS: 540.1 (500 MHz, DMSO) δ 11.01 (s, 1H), 8.28-8.23 (m, 2H), 8.05 (d, J = 5.5 Hz, 1H), 7.94 (d, J = 8.3 Hz, 1H), 6.97 (d, J = 5.5 Hz, 1H), 4.61-4.50 (m, 2H), 3.64 (q, J = 22.2 Hz, 2H), 3.50 (q, J = 14.0 Hz, 2H), 2.07 (s, 6H), 1.46 (s, 3H), 1.40 (s, 3H) |
| 148 | 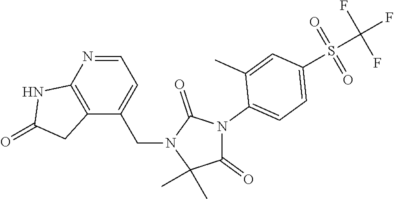 [SMILES: Cc1cc(S(=O)(=O)C(F)(F)F)ccc1N1C(=O)N(Cc2ccnc3c2CC(=O)N3)C(C)(C)C1=O] 5,5-dimethyl-1-[(2-oxo-1,7-diaza-4- indanyl)methyl]-3-[4-(trifluoromesyl)-2-tolyl]- 2,4-imidazolidinedione | LCMS: 497.1 (400 MHz, DMSO) δ 11.01 (s, 1H), 8.22 (s, 1H), 8.18 (d, J = 8.5 Hz, 1H), 8.04 (d, J = 5.2 Hz, 1H), 7.93 (d, J = 8.3 Hz, 1H), 7.00 (d, J = 5.3 Hz, 1H), 4.62- 4.48 (m, 2H), 3.73-3.56 (m, 2H), 2.36 (s, 3H), 1.46 (s, 3H), 1.44 (s, 3H) |
| 149 | 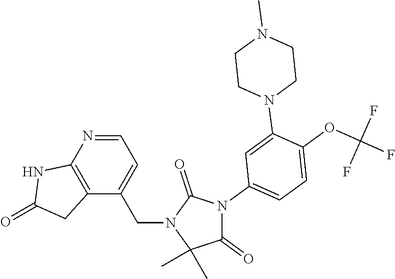 [SMILES: CN1CCN(c2cc(N3C(=O)N(Cc4ccnc5c4CC(=O)N5)C(C)(C)C3=O)ccc2OC(F)(F)F)CC1] 5,5-dimethyl-3-[3-(4-methyl-1-piperazinyl)-4- trifluoromethoxyphenyl]-1-[(2-oxo-1,7-diaza-4- indanyl)methyl]-2,4-imidazolidinedione | LCMS: 533.1 (400 MHz, DMSO) δ 10.97 (s, 1H), 7.99 (d, J = 5.5 Hz, 1H), 7.37 (dd, J = 8.6, 1.2 Hz, 1H), 7.21 (d, J = 2.4 Hz, 1H), 7.12 (dd, J = 8.6, 2.4 Hz, 1H), 6.95 (d, J = 5.5 Hz, 1H), 4.49 (s, 2H), 3.61 (s, 2H), 3.03-2.93 (m, 4H), 2.44 (s, 4H), 2.19 (s, 3H), 1.36 (s, 6H). |
| 151 | 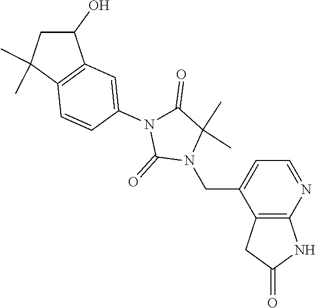 [SMILES: CC1(C)CC(O)c2cc(N3C(=O)N(Cc4ccnc5c4CC(=O)N5)C(C)(C)C3=O)ccc21] 3-(3-hydroxy-1,1-dimethyl-5-indanyl)-5,5- dimethyl-1-[(2-oxo-1,7-diaza-4-indanyl)methyl]- 2,4-imidazolidinedione | LCMS: 435.4 (400 MHz, DMSO-D6) δ 11.00 (br s, 1H), 8.03 (d, J = 5.5 Hz, 1H), 7.39 (d, J = 8.3 Hz, 1H), 7.26-7.21 (m, 2H), 6.97 (d, J = 5.6 Hz, 1H), 5.31 (d, J = 5.9 Hz, 1H), 5.16-5.09 (m, 1H), 4.52 (s, 2H), 3.64 (s, 2H), 2.27 (dd, J = 12.5, 7.0 Hz, 1H), 1.76 (dd, J = 12.5, 6.9 Hz, 1H), 1.40 (s, 6H), 1.34 (s, 3H), 1.17 (s, 3H). |
| 152 | 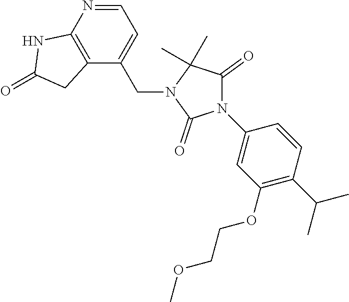 [SMILES: COCCOc1cc(N2C(=O)N(Cc3ccnc4c3CC(=O)N4)C(C)(C)C2=O)ccc1C(C)C] 3-[3-(2-methoxyethoxy)-4-cumenyl]-5,5- dimethyl-1-[(2-oxo-1,7-diaza-4-indanyl)methyl]- 2,4-imidazolidinedione | LCMS: 467.4 (400 MHz, DMSO-d6) δ 11.00 (s, 1H), 8.03 (d, J = 5.5 Hz, 1H), 7.29 (d, J = 8.2 Hz, 1H), 7.04 (d, J = 1.9 Hz, 1H), 6.99-6.94 (m, 2H), 4.52 (s, 2H), 4.09 (dd, J = 5.4, 3.8 Hz, 2H), 3.70 (dd, J = 5.3, 3.8 Hz, 2H), 3.64 (s, 2H), 3.33 (s, 3H), 3.30-3.22 (m, 1H), 1.39 (s, 6H), 1.20 (s, 3H), 1.18 (s, 3H). |
| 153 | 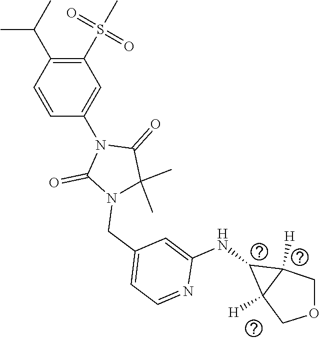 [SMILES: CC(C)c1ccc(N2C(=O)N(Cc3ccnc(N[C@H]4[C@@H]5COC[C@@H]54)c3)C(C)(C)C2=O)cc1S(C)(=O)=O] [symbol] 1-[(2-{(1R,5S,6r)-3-oxabicyclo[3.1.0]hex-6- ylamino}-4-pyridyl)methyl]-3-(3-mesyl-4- cumenyl)-5,5-dimethyl-2,4-imidazolidinedione | LCMS: 513.5 (500 MHz, DMSO-d6) δ 8.02 (d, J = 2.2 Hz, 1H), 7.95 (d, J = 5.2 Hz, 1H), 7.83 (d, J = 8.5 Hz, 1H), 7.78 (dd, J = 8.4, 2.2 Hz, 1H), 6.73 (d, J = 2.2 Hz, 1H), 6.61 (d, J = 5.2 Hz, 1H), 6.52 (s, 1H), 4.51 (s, 2H), 3.86 (d, J = 8.4 Hz, 2H), 3.82 (dt, J = 13.5, 6.8 Hz, 1H), 3.64 (d, J = 8.2 Hz, 2H), 3.28 (s, 3H), 2.26 (d, J = 2.1 Hz, 1H), 1.76 (s, 2H), 1.42 (s, 6H), 1.31 (s, 3H), 1.29 (s, 3H). |
| 154 | 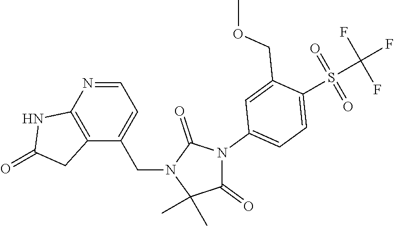 [SMILES: COCc1cc(N2C(=O)N(Cc3ccnc4c3CC(=O)N4)C(C)(C)C2=O)ccc1S(=O)(=O)C(F)(F)F] 3-[3-(methoxymethyl)-4-(trifluoromesyl)phenyl]- 5,5-dimethyl-1-[(2-oxo-1,7-diaza-4- indanyl)methyl]-2,4-imidazolidinedione | LCMS: 527.1 (400 MHz, DMSO) δ 11.00 (s, 1H), 8.29 (d, J = 8.7 Hz, 1H), 8.15 (d, J = 2.0 Hz, 1H), 8.02 (d, J = 5.5 Hz, 1H), 7.95 (dd, J = 8.7, 2.2 Hz, 1H), 7.04 (d, J = 5.5 Hz, 1H), 4.85 (s, 2H), 4.55 (s, 2H), 3.66 (s, 2H), 3.43 (s, 3H), 1.42 (s, 6H) |
| 155 | 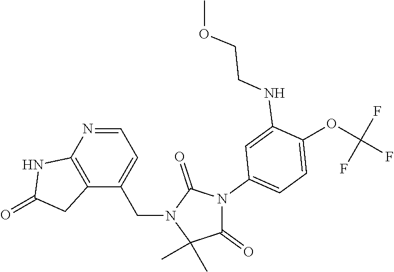 [SMILES: COCCNc1cc(N2C(=O)N(Cc3ccnc4c3CC(=O)N4)C(C)(C)C2=O)ccc1OC(F)(F)F] 3-[3-(2-methoxyethylamino)-4- trifluoromethoxyphenyl]-5,5-dimethyl-1-[(2-oxo- 1,7-diaza-4-indanyl)methyl]-2,4- imidazolidinedione | LCMS: 508.1 (400 MHz, DMSO) δ 10.95 (s, 1H), 7.99 (d, J = 5.5 Hz, 1H), 7.23 (dd, J = 8.6, 1.5 Hz, 1H), 6.93 (d, J = 5.5 Hz, 1H), 6.87 (d, J = 2.3 Hz, 1H), 6.63 (dd, J = 8.6, 2.3 Hz, 1H), 5.65 (t, J = 5.9 Hz, 1H), 4.49 (s, 2H), 3.61 (s, 2H), 3.46 (t, J = 5.9 Hz, 2H), 3.27-3.22 (m, 5H), 1.35 (s, 6H). |
| 156 | 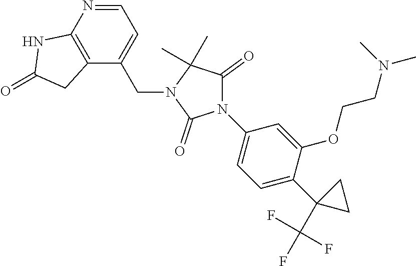 [SMILES: CN(C)CCOc1cc(N2C(=O)N(Cc3ccnc4c3CC(=O)N4)C(C)(C)C2=O)ccc1C1(C(F)(F)F)CC1] 3-{3-[2-(dimethylamino)ethoxy]-4-[1- (trifluoromethyl)cyclopropyl]phenyl}-5,5- dimethyl-1-[(2-oxo-1,7-diaza-4-indanyl)methyl]- 2,4-imidazolidinedione | LCMS: 546.16 (500 MHz, DMSO) δ 11.01 (s, 1H), 8.03 (d, J = 5.5 Hz, 1H), 7.47 (d, J = 8.1 Hz, 1H), 7.18 (d, J = 1.8 Hz, 1H), 7.04 (dd, J = 8.1, 1.8 Hz, 1H), 6.97 (d, J = 5.5 Hz, 1H), 4.53 (s, 2H), 4.09 (t, J = 5.8 Hz, 2H), 3.65 (s, 2H), 2.69 (t, J = 5.8 Hz, 2H), 2.25 (s,6H), 1.40 (s, 6H), 1.35 (t, J = 6.1 Hz, 2H), 1.10 (s, 2H). |
| 157 | 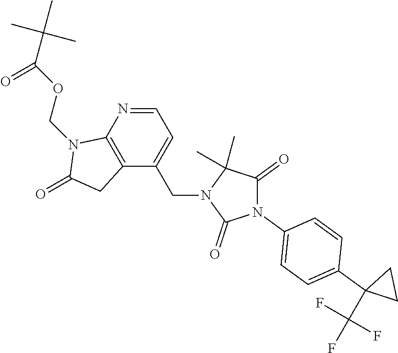 [SMILES: CC(C)(C)C(=O)OCN1C(=O)Cc2c(CN3C(=O)N(c4ccc(C5(C(F)(F)F)CC5)cc4)C(=O)C3(C)C)ccnc21] {4-[(5,5-dimethyl-2,4-dioxo-3-{p-[1- (trifluoromethyl)cyclopropyl]phenyl}-1- imidazolidinyl)methyl]-2-oxo-1,7-diaza-1- indanyl}methyl 2,2-dimethylpropionate | LCMS: 573.5 (500 MHz, DMSO-d6) δ 8.16 (d, J = 5.5 Hz, 1H), 7.59 (d, J = 8.4 Hz, 2H), 7.49 (d, J = 8.6 Hz, 2H), 7.15 (d, J = 5.5 Hz, 1H), 5.71 (s, 2H), 4.59 (s, 2H), 3.86 (s, 2H), 1.41 (s, 6H), 1.37 (q, J = 5.3 Hz, 2H), 1.19-1.15 (m, 2H), 1.10 (s, 9H). |
| 158 | 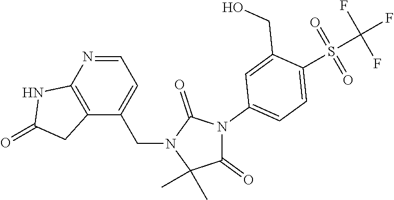 [SMILES: CC1(C)C(=O)N(c2ccc(S(=O)(=O)C(F)(F)F)c(CO)c2)C(=O)N1Cc1ccnc2c1CC(=O)N2] 3-[3-(hydroxymethyl)-4-(trifluoromesyl)phenyl]- 5,5-dimethyl-1-[(2-oxo-1,7-diaza-4- indanyl)methyl]-2,4-imidazolidinedione | LCMS: 513.1 (400 MHz, DMSO) δ 11.00 (s, 1H), 8.29 (d, J = 2.0 Hz, 1H), 8.23 (d, J = 8.7 Hz, 1H), 8.02 (d, J = 5.5 Hz, 1H), 7.90 (dd, J = 8.7, 2.2 Hz, 1H), 7.04 (d, J = 5.5 Hz, 1H), 5.85 (t, J = 5.3 Hz, 1H), 4.94 (d, J = 4.8 Hz, 2H), 4.56 (s, 2H), 3.67 (s, 2H), 1.43 (s, 6H) |
| 159 | 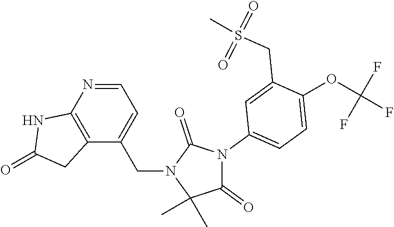 [SMILES: CC1(C)C(=O)N(c2ccc(OC(F)(F)F)c(CS(C)(=O)=O)c2)C(=O)N1Cc1ccnc2c1CC(=O)N2] 3-[3-(mesylmethyl)-4-trifluoromethoxyphenyl]- 5,5-dimethyl-1-[(2-oxo-1,7-diaza-4- indanyl)methyl]-2,4-imidazolidinedione | LCMS: 527.1 (400 MHz, DMSO) δ 10.96 (s, 1H), 7.99 (d, J = 5.5 Hz, 1H), 7.69 (d, J = 2.5 Hz, 1H), 7.64 (dd, J = 8.9, 2.5 Hz, 1H), 7.57 (dd, J = 8.9, 1.7 Hz, 1H), 6.96 (d, J = 5.5 Hz, 1H), 4.59 (s, 2H), 4.50 (s, 2H), 3.62 (s, 2H), 3.03 (s, 3H), 1.38 (s, 6H). |
| 160 | 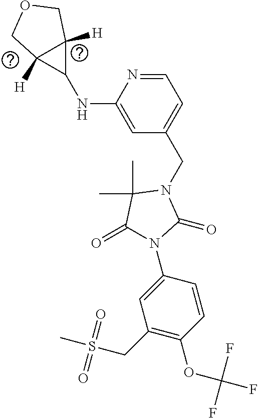 [SMILES: CC1(C)C(=O)N(c2ccc(OC(F)(F)F)c(CS(C)(=O)=O)c2)C(=O)N1Cc1ccnc(NC2[C@H]3COC[C@@H]23)c1] 1-[(2-{(1R,5S)-3-oxabicyclo[3.1.0]hex-6- ylamino}-4-pyridyl)methyl]-3-[3-(mesylmethyl)- 4-trifluoromethoxyphenyl]-5,5-dimethyl-2,4- imidazolidinedione | LCMS: 569.1 (400 MHz, DMSO) δ 7.91 (d, J = 5.2 Hz, 1H), 7.68 (d, J = 2.4 Hz, 1H), 7.66-7.53 (m, 2H), 6.69 (d, J = 2.0 Hz, 1H), 6.57 (d, J = 5.2 Hz, 1H), 6.50 (s, 1H), 4.59 (s, 2H), 4.47 (s, 2H), 3.83 (d, J = 8.4 Hz, 2H), 3.60 (d, J = 8.2 Hz, 2H), 3.02 (s, 3H), 2.23 (d, J = 2.1 Hz, 1H), 1.72 (s, 2H), 1.38 (s, 6H). |
| 162 | 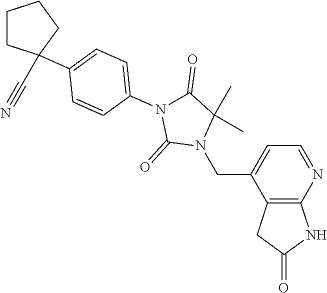 [SMILES: CC1(C)C(=O)N(c2ccc(C3(C#N)CCCC3)cc2)C(=O)N1Cc1ccnc2c1CC(=O)N2] 1-(p-{4,4-dimethyl-2,5-dioxo-3-[(2-oxo-1,7- diaza-4-indanyl)methyl]-1- imidazolidinyl}phenyl)cyclopentanecarbonitrile | LCMS: 444.4 (400 MHz, DMSO) δ 10.98 (s, 1H), 8.01 (d, J = 5.5 Hz, 1H), 7.66-7.57 (m, 2H), 7.52-7.46 (m, 2H), 6.96 (d, J = 5.5 Hz, 1H), 4.51 (s, 2H), 3.63 (s, 2H), 2.45-2.38 (m, 2H), 2.15-2.04 (m, 2H), 1.95-1.82 (m, 4H), 1.38 (s, 6H). |
| 163 | 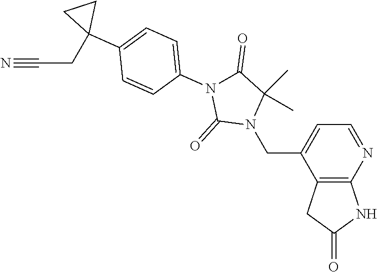 [SMILES: CC1(C)C(=O)N(c2ccc(C3(CC#N)CC3)cc2)C(=O)N1Cc1ccnc2c1CC(=O)N2] [1-(p-{4,4-dimethyl-2,5-dioxo-3-[(2-oxo-1,7- diaza-4-indanyl)methyl]-1- imidazolidinyl}phenyl)cyclopropyl]acetonitrile | LCMS: 430.3 (400 MHz, DMSO) δ 10.98 (s, 1H), 8.00 (d, J = 5.5 Hz, 1H), 7.46-7.42 (m, 2H), 7.42-7.37 (m, 2H), 6.95 (d, J = 5.5 Hz, 1H), 4.51 (s, 2H), 3.62 (s, 2H), 2.94 (s, 2H), 1.37 (s, 6H), 1.00- 0.93 (m, 4H). |
| 164 | 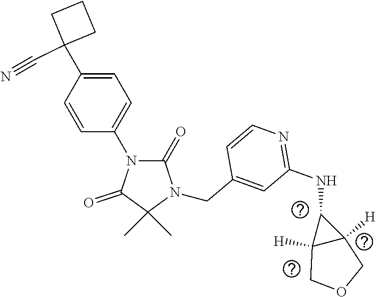 [SMILES: CC1(C)C(=O)N(c2ccc(C3(C#N)CCC3)cc2)C(=O)N1Cc1ccnc(N[C@H]2[C@@H]3COC[C@@H]32)c1] 1-(p-{3-[(2-{(1R,5S,6r)-3-oxabicyclo[3.1.0]hex- 6-ylamino}-4-pyridyl)methyl]-4,4-dimethyl-2,5- dioxo-1- imidazolidinyl}phenyl)cyclobutanecarbonitrile | LCMS: 472.4 (400 MHz, DMSO) δ 7.95 (d, J = 4.2 Hz, 1H), 7.62-7.58 (m, 2H), 7.52-7.47 (m, 2H), 6.73 (s, 1H), 6.59 (d, J = 5.1 Hz, 1H), 6.52 (s, 1H), 4.50 (s, 2H), 3.86 (d, J = 8.4 Hz, 2H), 3.63 (d, J = 8.2 Hz, 2H), 2.83-2.71 (m, 2H), 2.71-2.60 (m, 2H), 2.36- 2.22 (m, 2H), 2.09-1.96 (m, 1H), 1.78-1.73 (m, 2H), 1.40 (s, 6H). |
| 165 | 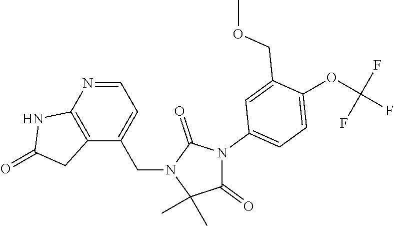 [SMILES: COCc1cc(N2C(=O)N(Cc3ccnc4c3CC(=O)N4)C(C)(C)C2=O)ccc1OC(F)(F)F] 3-[3-(methoxymethyl)-4- trifluoromethoxyphenyl]-5,5-dimethyl-1-[(2-oxo- 1,7-diaza-4-indanyl)methyl]-2,4- imidazolidinedione | LCMS: 479.1 (400 MHz, DMSO) δ 10.96 (s, 1H), 7.99 (d, J = 5.5 Hz, 1H), 7.64 (d, J = 2.2 Hz, 1H), 7.59- 7.44 (m, 2H), 6.97 (d, J = 5.5 Hz, 1H), 4.49 (s, 2H), 4.49 (s, 2H), 3.62 (s, 2H), 3.34 (s, 3H), 1.37 (s, 6H). |
| 167 | 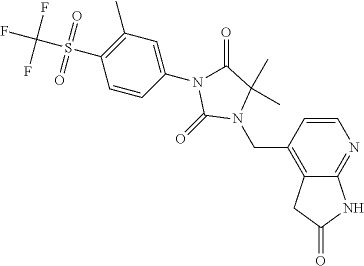 [SMILES: Cc1cc(N2C(=O)N(Cc3ccnc4c3CC(=O)N4)C(C)(C)C2=O)ccc1S(=O)(=O)C(F)(F)F] 5,5-dimethyl-1-[(2-oxo-1,7-diaza-4- indanyl)methyl]-3-[4-(trifluoromesyl)-3-tolyl]- 2,4-imidazolidinedione | LCMS: 497.1 (400 MHz, DMSO) δ 11.00 (s, 1H), 8.26 (d, J = 9.3 Hz, 1H), 8.02 (d, J = 5.5 Hz, 1H), 7.88- 7.82 (m, 2H), 7.02 (d, J = 5.5 Hz, 1H), 4.55 (s, 2H), 3.66 (s, 2H), 2.71 (s, 3H), 1.42 (s, 6H) |
| 168 | 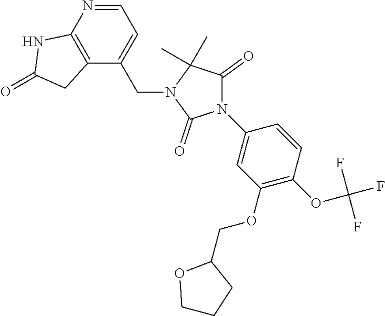 [SMILES: CC1(C)C(=O)N(c2ccc(OC(F)(F)F)c(OCC3CCCO3)c2)C(=O)N1Cc1ccnc2c1CC(=O)N2] 5,5-dimethyl-1-[(2-oxo-1,7-diaza-4- indanyl)methyl]-3-{3-[(tetrahydro-2- furyl)methoxy]-4-trifluoromethoxyphenyl}-2,4- imidazolidinedione | LCMS: 535.3 (400 MHz, DMSO) δ 8.01 (d, J = 5.5 Hz, 1H), 7.48 (dd, J = 8.6, 1.1 Hz, 1H), 7.37 (d, J = 2.3 Hz, 1H), 7.12 (dd, J = 8.7, 2.3 Hz, 1H), 6.97 (d, J = 5.5 Hz, 1H), 4.51 (s, 2H), 4.19 (dt, J = 11.7, 5.7 Hz, 1H), 4.01 (d, J = 4.8 Hz, 2H), 3.73 (ddd, J = 21.4, 14.4, 7.9 Hz, 2H), 3.63 (s, 2H), 2.05- 1.66 (m, 4H), 1.38 (s, 6H). |
| 169 | 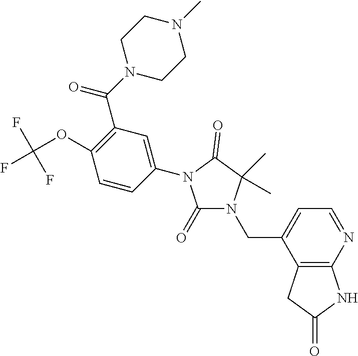 [SMILES: CN1CCN(C(=O)c2cc(N3C(=O)N(Cc4ccnc5c4CC(=O)N5)C(C)(C)C3=O)ccc2OC(F)(F)F)CC1] 5,5-dimethyl-3-{3-[(4-methyl-1- piperazinyl)carbonyl]-4- trifluoromethoxyphenyl}-1-[(2-oxo-1,7-diaza-4- indanyl)methyl]-2,4-imidazolidinedione | LCMS: 561.4 (400 MHz, DMSO-D6) δ 11.00 (s, 1H), 8.02 (d, J = 5.4 Hz, 1H), 7.70 (dd, J = 8.9, 2.4 Hz, 1H), 7.65-7.60 (m, 2H), 7.01 (d, J = 5.5 Hz, 1H), 4.53 (s, 2H), 3.76- 3.52 (m, 2H), 3.65 (overlapped s, 2 H), 3.22-3.15 (m, 2H), 2.46- 2.14 (m, 4H), 2.19 (s, 3 H), 1.40 (s, 6H). |
| 170 | 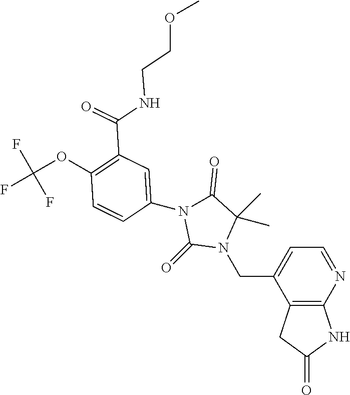 [SMILES: COCCNC(=O)c1cc(N2C(=O)N(Cc3ccnc4c3CC(=O)N4)C(C)(C)C2=O)ccc1OC(F)(F)F] N-2-methoxyethyl 5-{4,4-dimethyl-2,5-dioxo-3- [(2-oxo-1,7-diaza-4-indanyl)methyl]-1- imidazolidinyl}-2-trifluoroanisamide | LCMS: 536.4 (400 MHz, DMSO-D6) δ 11.00 (s, 1H), 8.58 (t, J = 5.2 Hz, 1H), 8.02 (d, J = 5.4 Hz, 1H), 7.71 (d, J = 2.4 Hz, 1H), 7.68 (dd, J = 8.7, 2.6 Hz, 1H), 7.59 (dd, J = 8.7, 1.1 Hz, 1H), 7.01 (d, J = 5.5 Hz, 1H), 4.53 (s, 2H), 3.65 (s, 2H), 3.46-3.37 (m, 4H), 3.27 (s, 3H), 1.40 (s, 6H). |
| 171 | 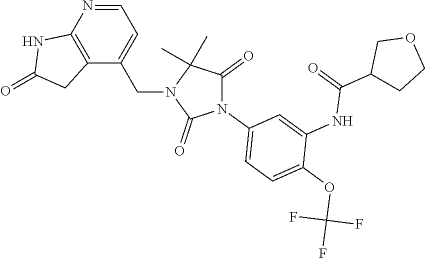 [SMILES: CC1(C)C(=O)N(c2ccc(OC(F)(F)F)c(NC(=O)C3CCOC3)c2)C(=O)N1Cc1ccnc2c1CC(=O)N2] N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-3-furamide | LCMS: 548.1 (400 MHz, DMSO) δ 10.99 (s, 1H), 9.97 (s, 1H), 8.02 (d, J = 5.5 Hz, 1H), 7.98 (d, J = 2.5 Hz, 1H), 7.55 (dd, J = 8.8, 1.3 Hz, 1H), 7.38 (dd, J = 8.8, 2.5 Hz, 1H), 6.99 (d, J = 5.5 Hz, 1H), 4.52 (s, 2H), 3.93 (t, J = 8.2 Hz, 1H), 3.80-3.67 (m, 3H), 3.65 (s, 2H), 3.00-2.80 (m, 1H, obscured by H2O peak), 2.12- 2.01 (m, 2H), 1.40 (s, 6H). |
| 172 | 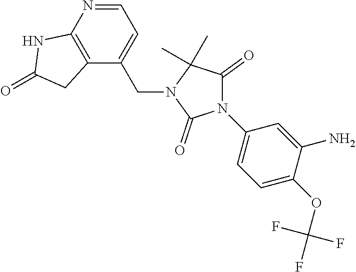 [SMILES: CC1(C)C(=O)N(c2ccc(OC(F)(F)F)c(N)c2)C(=O)N1Cc1ccnc2c1CC(=O)N2] 3-(3-amino-4-trifluoromethoxyphenyl)-5,5- dimethyl-1-[(2-oxo-1,7-diaza-4-indanyl)methyl]- 2,4-imidazolidinedione | LCMS: 450 (500 MHz, DMSO) δ 11.00 (s, 1H), 8.02 (d, J = 5.5 Hz, 1H), 7.20 (dd, J = 8.6, 1.2 Hz, 1H), 6.95 (d, J = 5.5 Hz, 1H), 6.88 (d, J = 2.5 Hz, 1H), 6.62 (dd, J = 8.7, 2.5 Hz, 1H), 5.61 (s, 2H), 4.51 (s, 2H), 3.64 (s, 2H), 1.38 (s, 6H). |
| 173 | 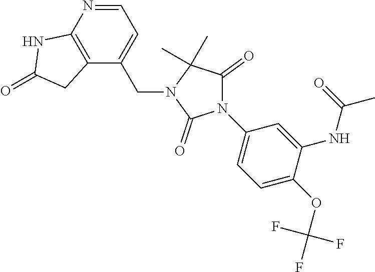 [SMILES: CC(=O)Nc1cc(N2C(=O)N(Cc3ccnc4c3CC(=O)N4)C(C)(C)C2=O)ccc1OC(F)(F)F] N-(5-{4,4-dimethyl-2,5-dioxo-3-[(2-oxo-1,7- diaza-4-indanyl)methyl]-1-imidazolidinyl}-2- trifluoromethoxyphenyl)acetamide | LCMS: 492.4 (500 MHz, DMSO) δ 11.00 (s, 1H), 9.86 (s, 1H), 8.04-7.97 (m, 1H), 7.54 (dd, J = 8.8, 1.2 Hz, 1H), 7.35 (dd, J = 8.8, 2.5 Hz, 1H), 6.99 (d, J = 5.5 Hz, 1H), 4.52 (s, 1H), 3.65 (s, 1H), 2.11 (s, 1H), 1.40 (s, 1H). |
| 174 | 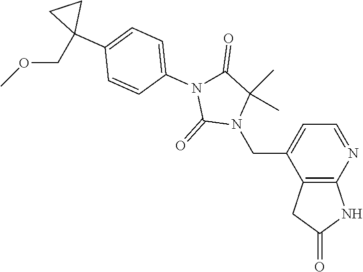 [SMILES: COCC1(c2ccc(N3C(=O)N(Cc4ccnc5c4CC(=O)N5)C(C)(C)C3=O)cc2)CC1] 3-{p-[1-(methoxymethyl)cyclopropyl]phenyl}- 5,5-dimethyl-1-[(2-oxo-1,7-diaza-4- indanyl)methyl]-2,4-imidazolidinedione | LCMS: 435.3 (400 MHz, DMSO) δ 10.98 (s, 1H), 8.00 (d, J = 5.5 Hz, 1H), 7.41-7.34 (m, 2H), 7.33-7.28 (m, 2H), 6.94 (d, J = 5.5 Hz, 1H), 4.50 (s, 2H), 3.62 (s, 2H), 3.46 (s, 2H), 3.21 (s, 3H), 1.37 (s, 6H), 0.92-0.75 (m, 4H). |
| 175 | 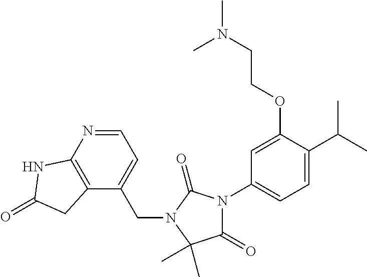 [SMILES: CC(C)c1ccc(N2C(=O)N(Cc3ccnc4c3CC(=O)N4)C(C)(C)C2=O)cc1OCCN(C)C] 3-{3-[2-(dimethylamino)ethoxy]-4-cumenyl}-5,5- dimethyl-1-[(2-oxo-1,7-diaza-4-indanyl)methyl]- 2,4-imidazolidinedione | LCMS: 480.2 (400 MHz, CD₃CN) δ 8.78 (s, 1H), 8.05 (d, J = 5.4 Hz, 1H), 7.33 (d, J = 8.1 Hz, 1H), 7.02- 6.92 (m, 3H), 4.51 (s, 2H), 4.10 (t, J = 5.7 Hz, 2H), 3.56 (s, 2H), 3.41-3.30 (m, 1H), 2.77 (t, J = 5.6 Hz, 2H), 2.32 (s, 6H), 1.41 (s, 6H), 1.23 (d, J = 6.9 Hz, 6H). |
| 176 | 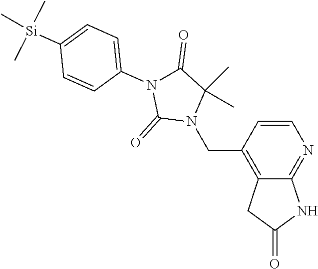 [SMILES: CC1(C)C(=O)N(c2ccc([Si](C)(C)C)cc2)C(=O)N1Cc1ccnc2c1CC(=O)N2] 5,5-dimethyl-1-[(2-oxo-1,7-diaza-4- indanyl)methyl]-3-[p-(trimethylsilyl)phenyl]-2,4- imidazolidinedione | LCMS: 423.3 (400 MHz, DMSO) δ 11.00 (s, 1H), 8.03 (d, J = 5.5 Hz, 1H), 7.67-7.62 (m, 2H), 7.46-7.41 (m, 2H), 6.97 (d, J = 5.5 Hz, 1H), 4.53 (s, 2H), 3.64 (s, 2H), 1.40 (s, 6H), 0.30-0.24 (m, 9H). |
| 177 | 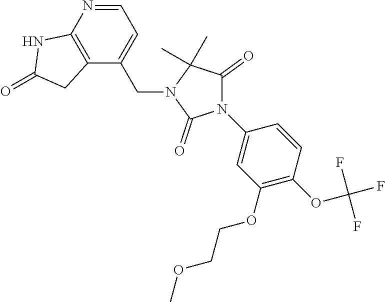 [SMILES: COCCOc1cc(N2C(=O)N(Cc3ccnc4c3CC(=O)N4)C(C)(C)C2=O)ccc1OC(F)(F)F] 3-[3-(2-methoxyethoxy)-4- trifluoromethoxyphenyl]-5,5-dimethyl-1-[(2-oxo- 1,7-diaza-4-indanyl)methyl]-2,4- imidazolidinedione | LCMS: 509.3 (400 MHz, DMSO) δ 11.00 (s, 1H), 8.03 (d,J = 5.5 Hz, 1H), 7.50 (dd,J = 8.7, 1.2 Hz, 1H), 7.40 (d,J = 2.3 Hz, 1H), 7.14 (dd,J = 8.7, 2.3 Hz, 1H), 6.99 (d,J = 5.5 Hz, 1H), 4.53 (s, 2H), 4.19 (dd,J = 5.3, 3.8 Hz, 2H), 3.69 (dd,J = 5.2, 3.9 Hz, 2H), 3.65 (s, 2H), 3.32 (s, 3H), 1.40 (s, 6H). |
| 179 | 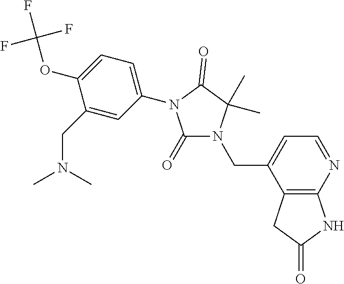 [SMILES: CN(C)Cc1cc(N2C(=O)N(Cc3ccnc4c3CC(=O)N4)C(C)(C)C2=O)ccc1OC(F)(F)F] 3-{3-[(dimethylamino)methyl]-4- trifluoromethoxyphenyl}-5,5-dimethyl-1-[(2-oxo- 1,7-diaza-4-indanyl)methyl]-2,4- imidazolidinedione | LCMS: 492.1 (500 MHz, DMSO) δ 11.00 (s, 1H), 8.02 (d,J = 5.5 Hz, 1H), 7.68 (d,J = 1.7 Hz, 1H), 7.61- 7.36 (m, 2H), 7.00 (d,J = 5.5 Hz, 1H), 4.53 (s, 2H), 3.65 (s, 2H), 3.48 (s, 2H), 2.19 (s, 6H), 1.40 (s, 6H) |
| 180 | 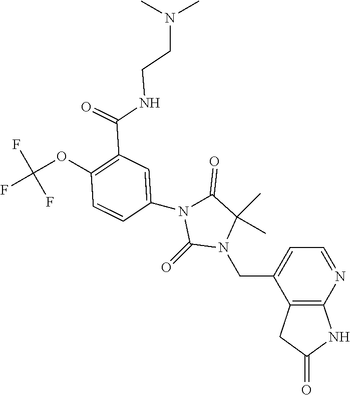 [SMILES: CN(C)CCNC(=O)c1cc(N2C(=O)N(Cc3ccnc4c3CC(=O)N4)C(C)(C)C2=O)ccc1OC(F)(F)F] N-2-(dimethylamino)ethyl5-{4,4-dimethyl-2,5- dioxo-3-[(2-oxo-1,7-diaza-4-indanyl)methyl]-1- imidazolidinyl}-2-trifluoroanisamide | LCMS: 549.3 (400 MHz, CDCl₃) δ 8.37 (br s, 1H), 8.14-8.10 (m, 2H), 7.60 (dd, J = 8.8, 2.7 Hz, 1H), 7.40- 7.36 (overlapped m, 1H), 7.36- 7.31 (overlapped m, 1H), 6.90 (d, J = 5.5 Hz, 1H), 4.50 (s, 2H), 3.62 (s, 2H), 3.56-3.51 (m, 2H), 2.51 (t, J = 5.9 Hz, 2H), 2.26 (s, 6H), 1.47 (s, 6H). |
| 181 | 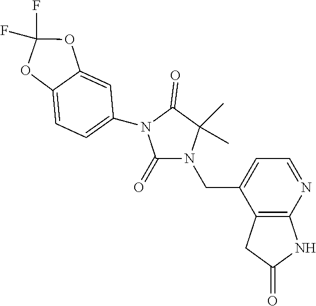 [SMILES: CC1(C)C(=O)N(c2ccc3c(c2)OC(F)(F)O3)C(=O)N1Cc1ccnc2c1CC(=O)N2] 3-(2,2-difluoro-2H-1,3-benzodioxol-5-yl)-5,5- dimethyl-1-[(2-oxo-1,7-diaza-4-indanyl)methyl]- 2,4-imidazolidinedione | LCMS: 431.21 (500 MHz, DMSO) δ 11.00 (s, 1H), 8.03 (d, J = 5.5 Hz, 1H), 7.61 (d,J = 1.9 Hz, 1H), 7.57 (d, J = 8.7 Hz, 1H), 7.34 (dd,J = 8.6, 2.0 Hz, 1H), 7.00 (d,J = 5.5 Hz, 1H), 4.53 (s, 2H), 3.65 (s, 2H), 1.40 (s, 6H). |
| 182 | 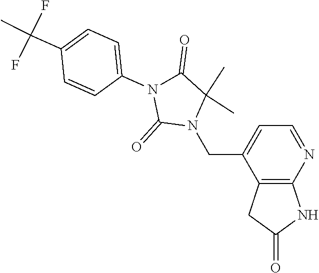 [SMILES: CC(F)(F)c1ccc(N2C(=O)N(Cc3ccnc4c3CC(=O)N4)C(C)(C)C2=O)cc1] 3-[p-(1,1-difluoroethyl)phenyl]-5,5-dimethyl-1- [(2-oxo-1,7-diaza-4-indanyl)methyl]-2,4- imidazolidinedione | LCMS: 415.3 (400 MHz, DMSO-D6) δ 11.00 (s, 1H), 8.03 (d, J = 5.5 Hz, 1H), 7.73-7.68 (m, 2H), 7.62-7.58 (m, 2H), 7.00 (d, J = 5.5 Hz, 1H), 4.54 (s, 2H), 3.65 (s, 2H), 2.00 (t, J = 18.9 Hz, 3H), 1.41 (s, 6H). |
| 183 | 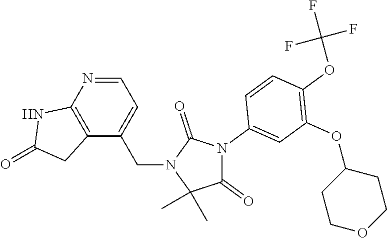 [SMILES: CC1(C)C(=O)N(c2ccc(OC(F)(F)F)c(OC3CCOCC3)c2)C(=O)N1Cc1ccnc2c1CC(=O)N2] 5,5-dimethyl-1-[(2-oxo-1,7-diaza-4- indanyl)methyl]-3-[3-(tetrahydro-2H-pyran-4- yloxy)-4-trifluoromethoxyphenyl]-2,4- imidazolidinedione | LCMS: 535.5 (400 MHz, DMSO) δ 11.00 (s, 1H), 8.03 (d,J = 5.5 Hz, 1H), 7.50 (dd,J = 8.7, 1.1 Hz, 1H), 7.42 (d,J = 2.3 Hz, 1H), 7.14 (dd,J = 8.7, 2.3 Hz, 1H), 6.98 (d,J = 5.5 Hz, 1H), 4.66 (dt,J = 11.9, 3.9 Hz, 1H), 4.53 (s, 2H), 3.90-3.77 (m, 2H), 3.65 (s, 2H), 3.51 (ddd,J = 11.5, 8.5, 3.1 Hz, 2H), 2.06-1.92 (m, 2H), 1.70-1.54 (m, 2H), 1.40 (s, 6H). |
| 184 | 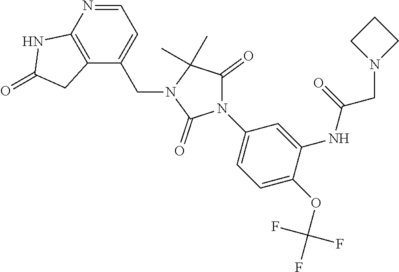 [SMILES: CC1(C)C(=O)N(c2ccc(OC(F)(F)F)c(NC(=O)CN3CCC3)c2)C(=O)N1Cc1ccnc2c1CC(=O)N2] N-(5-{4,4-dimethyl-2,5-dioxo-3-[(2-oxo-1,7- diaza-4-indanyl)methyl]-1-imidazolidinyl}-2- trifluoromethoxyphenyl)(1-azetidinyl)acetamide | LCMS: 547.1 (500 MHz, DMSO) δ 11.00 (s, 1H), 9.78 (s, 1H), 8.25 (d, J = 2.5 Hz, 1H), 8.02 (d, J = 5.5 Hz, 1H), 7.60 (d, J = 7.6 Hz, 1H), 7.36 (dd, J = 8.8, 2.5 Hz, 1H), 7.00 (d, J = 5.5 Hz, 1H), 4.53 (s, 2H), 3.65 (s, 2H), 3.37-3.33 (m, 4H), 3.27 (s, 2H), 2.06 (p, J = 7.0 Hz, 2H), 1.41 (s, 6H). |
| 185 | 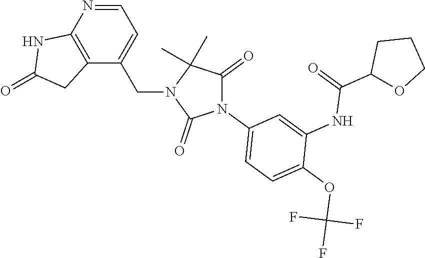 [SMILES: CC1(C)C(=O)N(c2ccc(OC(F)(F)F)c(NC(=O)C3CCCO3)c2)C(=O)N1Cc1ccnc2c1CC(=O)N2] N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2-furamide | LCMS: 548.1 (400 MHz, DMSO) δ 10.96 (s, 1H), 9.47 (s, 1H), 8.00 (dd, J = 8.6, 3.9 Hz, 2H), 7.55 (d, J = 8.8 Hz, 1H), 7.38 (dd, J = 8.9, 2.5 Hz, 1H), 6.96 (d, J = 5.5 Hz, 1H), 4.49 (s, 2H), 4.45 (dd, J = 8.4, 5.3 Hz, 1H), 3.94 (dd, J = 14.0, 6.9 Hz, 1H), 3.83 (dd, J = 14.6, 7.3 Hz, 1H), 3.62 (s, 2H), 2.25-2.11 (m, 1H), 2.02-1.89 (m, 1H), 1.89-1.79 (m, 2H), 1.37 (s, 6H). |
| 186 | 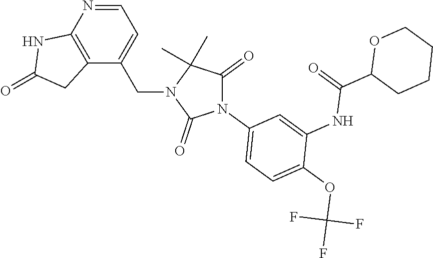 [SMILES: CC1(C)C(=O)N(c2ccc(OC(F)(F)F)c(NC(=O)C3CCCCO3)c2)C(=O)N1Cc1ccnc2c1CC(=O)N2] N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2H-pyran-2- carboxamide | LCMS: 562.1 (500 MHz, DMSO) δ 10.92 (s, 1H), 9.21 (s, 1H), 8.04 (d, J = 2.5 Hz, 1H), 7.95 (d, J = 5.5 Hz, 1H), 7.51 (dd, J = 8.9, 1.4 Hz, 1H), 7.33 (dd, J = 8.8, 2.5 Hz, 1H), 6.93 (d, J = 5.5 Hz, 1H), 4.46 (s, 2H), 4.01-3.92 (m, 2H), 3.58 (s, 2H), 3.52-3.45 (m, 1H), 1.90-1.84 (m, 1H), 1.80-1.72 (m, 1H), 1.56-1.35 (m, 4H), 1.33 (s, 6H). |
| 187 | 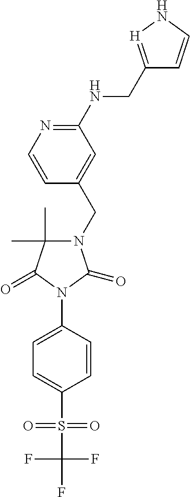 5,5-dimethyl-1-[(2-{[(3- pyrazolyl)methyl]amino}-4-pyridyl)methyl]-3-[p- (trifluoromesyl)phenyl]-2,4-imidazolidinedione | LCMS: 523.3 (400 MHz, DMSO) δ 12.57 (br. s, 1H), 8.39-8.26 (m, 2H), 8.07- 8.01 (m, 2H), 7.94 (d, J = 5.7 Hz, 1H), 7.55 (br. s, 1H), 6.70 (br. s, 1H), 6.54 (s, 2H), 6.13 (d, J = 1.6 Hz, 1H), 4.47 (s, 2H), 4.42 (d, J = 5.1 Hz, 2H), 1.41 (s, 6H). |
| 188 | 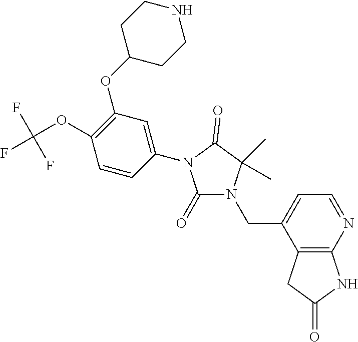 [SMILES: CC1(C)C(=O)N(c2ccc(OC(F)(F)F)c(OC3CCNCC3)c2)C(=O)N1Cc1ccnc2c1CC(=O)N2] 5,5-dimethyl-1-[(2-oxo-1,7-diaza-4- indanyl)methyl]-3-[3-(4-piperidyloxy)-4- trifluoromethoxyphenyl]-2,4-imidazolidinedione | LCMS: 534.2 (400 MHz, DMSO) δ 11.01 (br. s, 1H), 8.03 (d, J = 5.5 Hz, 1H), 7.49 (dd, J = 8.7, 1.0 Hz, 1H), 7.39 (d, J = 2.2 Hz, 1H), 7.12 (dd, J = 8.7, 2.3 Hz, 1H), 6.98 (d, J = 5.5 Hz, 1H), 4.56-4.49 (m, 3H), 3.73-3.63 (m, 3H), 3.01-2.92 (m, 2H), 2.70-2.58 (m, 2H), 1.99-1.90 (m, 2H), 1.62-1.48 (m, 2H), 1.40 (s, 6H). |
| 190 | 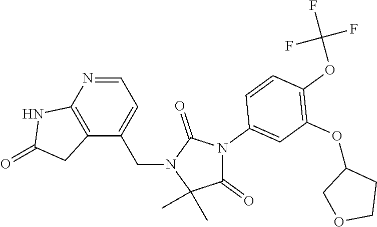 [SMILES: CC1(C)C(=O)N(c2ccc(OC(F)(F)F)c(OC3CCOC3)c2)C(=O)N1Cc1ccnc2c1CC(=O)N2] 5,5-dimethyl-1-[(2-oxo-1,7-diaza-4- indanyl)methyl]-3-[3-(tetrahydro-3-furyloxy)-4- trifluoromethoxyphenyl]-2,4-imidazolidinedione | LCMS: 521.3 (400 MHz, DMSO) δ 11.01 (s, 1H), 8.03 (d, J = 5.5 Hz, 1H), 7.51 (d, J = 8.7 Hz, 1H), 7.37 (d, J = 2.3 Hz, 1H), 7.17 (dd, J = 8.7, 2.3 Hz, 1H), 6.99 (d, J = 5.5 Hz, 1H), 5.10 (s, 1H), 4.54 (s, 1H), 3.92 (dd, J = 10.3, 4.5 Hz, 1H), 3.85-3.74 (m, 3H), 3.65 (s, 2H), 2.32-2.17 (m, 1H), 2.05-1.91 (m, 1H), 1.40 (s, 1H). |
| 191 | 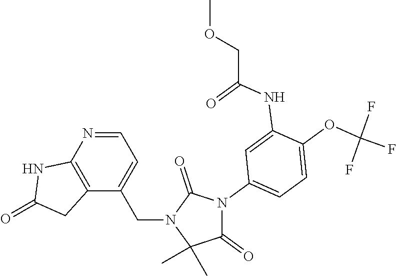 [SMILES: COCC(=O)Nc1cc(N2C(=O)N(Cc3ccnc4c3CC(=O)N4)C(C)(C)C2=O)ccc1OC(F)(F)F] N-(5-{4,4-dimethyl-2,5-dioxo-3-[(2-oxo-1,7- diaza-4-indanyl)methyl]-1-imidazolidinyl}-2- trifluoromethoxyphenyl)methoxyacetamide | LCMS: 522.0 (400 MHz, DMSO) δ 10.98 (s, 1H), 9.55 (s, 1H), 8.03-8.00 (m, 2H), 7.56 (dd, J = 8.9, 1.5 Hz, 1H), 7.39 (dd, J = 8.9, 2.5 Hz, 1H), 6.98 (d, J = 5.5 Hz, 1H), 4.51 (s, 2H), 4.05 (s, 2H), 3.63 (s, 2H), 3.38 (s, 3H), 1.39 (s, 6H) |

[symbol]

indicates data missing or illegible when filed

### Section 3: Cellular Assays

Provided in this section is the biological evaluation of the specific examples provided herein.

50K PC3 cells were seeded on day 1 in 50 μL of media in 96-well plate and incubated overnight at 37° C., 500 CO₂. On day 2, all cells were expected to be at 80-90% confluency. Cells were starved for two hours by changing the media to 90 μL of Eagle's Minimum Essential Medium (EMEM) serum-free media and incubated for 2 hours. Next, cells were treated with 10 μL of 10× concentrated and 3× serially diluted compounds for two hours. It makes 1× of compounds in 100 μL media. This was followed by stimulation of cells with 11×IGF1 for 30 min in 10 μL of serum-free EMEM media at the final concentration of 200 ng/mL.

The media was removed and replaced by 50 μL of 1× lysis buffer from the AlphaLISA kit (with 1× of cOmplete™ protease inhibitor). The plates were sealed, and cells were allowed to lyse for 10 min in the shaker before storing the plate at −20° C. until used. The AlphaLISA assay with processed cell lysates were performed as described in the protocol. Briefly, 10 μL of the undiluted sample were loaded to 384 well Optiplates and 5 μL of the acceptor mix (as described in the AlphaLISA kit) was added to each well. The plates were sealed and briefly vortexed followed by the incubation at RT for 3 hours. In the dark, 5 μL of donor mix was added to each well. All plates were sealed and stored at RT overnight. The plates were read in EnVision reader using standard AlphaLISA settings.

Compound activity was analyzed using CBIS data analysis suite (ChemInnovation, CA). Percentage inhibition was calculated using the following formula: % Inhibition=100%×(1−(mean relative light units (RLU) of test sample−mean RLU of vehicle control)/(mean RLU of EC₅₀ control−mean RLU of vehicle control)).

The data in Table 4 categorizes the IC₅₀ of each compound for inhibiting enzymatic activity of IGF-1R in the assay.

TABLE 4

| Compound | IC₅₀ (nM) |
|---|---|
| 1 | 432 |
| 2 | 24.5 |
| 3 | 686 |
| 4 | 6.25 |
| 5 | 1990 |
| 6 | 52.2 |
| 7 | 4.04 |
| 8 | 4.45 |
| 9 | 2.82 |
| 10 | 6.14 |
| 11 | 4.33 |
| 12 | 28.1 |
| 13 | 48.9 |
| 14 | 253 |
| 15 | 8.82 |
| 16 | 946 |
| 17 | 202 |
| 18 | 5.33 |
| 19 | 37.1 |
| 20 | 719 |
| 21 | 16.7 |
| 22 | 147 |
| 23 | 21.6 |
| 24 | 712 |
| 25 | 173 |
| 26 | 49.5 |
| 27 | 415 |
| 28 | 88.5 |
| 29 | 18.8 |
| 30 | 16.2 |
| 31 | 34.9 |
| 32 | 3420 |
| 33 | 53.5 |
| 34 | 67.8 |
| 35 | 223 |
| 36 | 2730 |
| 37 | 38.8 |
| 38 | 22.8 |
| 39 | 25.9 |
| 40 | 345 |
| 41 | 66.3 |
| 42 | 48.3 |
| 43 | 97.6 |
| 44 | 27.2 |
| 45 | 879 |
| 46 | 232 |
| 47 | 433 |
| 48 | 29.1 |
| 49 | 57.5 |
| 50 | 6510 |
| 51 | 2240 |
| 52 | 381 |
| 53 | 1080 |
| 54 | 266 |
| 55 | 234 |
| 56 | 17.5 |
| 57 | 816 |
| 58 | 25.7 |
| 59 | 18.7 |
| 60 | 21.2 |
| 61 | 162 |
| 62 | 16 |
| 63 | 62.5 |
| 64 | 98.6 |
| 65 | >10,000 |
| 66 | 28.5 |
| 67 | 830 |
| 68 | 24.1 |
| 69 | 1150 |
| 70 | 280 |
| 71 | 85.1 |
| 72 | 1990 |
| 73 | 57.4 |
| 74 | 152 |
| 75 | 122 |
| 76 | 568 |
| 77 | 121 |
| 78 | 226 |
| 79 | 189 |
| 80 | 72.9 |
| 81 | 184 |
| 82 | 226 |
| 83 | 56.3 |
| 84 | 2090 |
| 85 | 306 |
| 86 | 95.5 |
| 87 | 261 |
| 88 | 309 |
| 89 | 233 |
| 90 | 395 |
| 91 | 80.2 |
| 92 | 436 |
| 93 | 943 |
| 94 | 74.1 |
| 95 | 51.1 |
| 96 | 1450 |
| 97 | 78.2 |
| 98 | 51.5 |
| 99 | 182 |
| 100 | 210 |
| 101 | 44.2 |
| 102 | 112 |
| 103 | 17.3 |
| 104 | 56.6 |
| 105 | 478 |
| 106 | 124 |
| 107 | 70.8 |
| 108 | 336 |
| 109 | 61.3 |
| 110 | 2850 |
| 111 | 32.3 |
| 112 | 406 |
| 113 | 110 |
| 114 | 293 |
| 115 | 691 |
| 116 | 116 |
| 117 | 367 |
| 118 | 70.5 |
| 119 | 133 |
| 120 | 32.1 |
| 121 | 84.6 |
| 122 | 15.2 |
| 123 | 632 |
| 124 | 22.8 |
| 125 | 114 |
| 126 | 44 |
| 127 | 147 |
| 128 | 348 |
| 129 | 89 |
| 130 | 39.4 |
| 131 | 76.1 |
| 132 | 78.1 |
| 133 | 151 |
| 134 | 31.8 |
| 135 | 64.7 |
| 136 | 108 |
| 137 | 25.7 |
| 138 | 16.7 |
| 139 | >10,000 |
| 140 | >10,000 |
| 141 | 102 |
| 142 | 325 |
| 143 | >10,000 |
| 144 | 55.3 |
| 145 | 761 |
| 146 | 42.6 |
| 147 | >10,000 |
| 148 | 7440 |
| 149 | 577 |
| 150 | 553 |
| 151 | >10,000 |
| 152 | 40.1 |
| 153 | 103 |
| 154 | 93.2 |
| 155 | 182 |
| 156 | 33.8 |
| 157 | 48.6 |
| 158 | 224 |
| 159 | 8490 |
| 160 | 530 |
| 161 | >10,000 |
| 162 | 282 |
| 163 | 658 |
| 164 | 227 |
| 165 | 895 |
| 166 | 132 |
| 167 | 136 |
| 168 | 226 |
| 169 | >10,000 |
| 170 | >10,000 |
| 171 | 2660 |
| 172 | 2010 |
| 173 | 9200 |
| 174 | 1110 |
| 175 | 42.7 |
| 176 | 31.9 |
| 177 | 603 |
| 178 | 39 |
| 179 | 1450 |
| 180 | >10,000 |
| 181 | >10,000 |
| 182 | 1360 |
| 183 | 128 |
| 184 | 273 |
| 185 | 187 |
| 186 | 115 |
| 187 | 92.3 |
| 188 | 3240 |
| 189 | 167 |
| 190 | 428 |
| 191 | 119 |

The results presented in Table 4 have been generated with the in vitro assays described above. These assays may be used to test any other compound described herein to assess and characterize a compound's biological activity. In view of the disclosure provided herein, compounds not specifically tested would be expected to have similar results.

What is claimed is:

1. A compound of Formula (I): 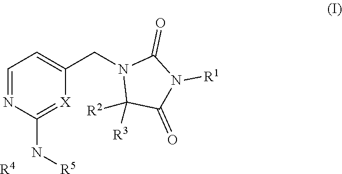
or a pharmaceutically acceptable salt thereof,
wherein
R¹ is 6-membered monocyclic aromatic ring or a 6,5-bicyclic ring having 0-2 ring heteroatoms selected from N and O, the 6-membered monocyclic aromatic ring is substituted with R⁶, and the 6-membered monocyclic aromatic ring or the 6,5-bicyclic ring is substituted with 0-3 R⁷;
R⁶ is 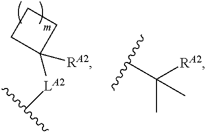
a spiro C₃₋₆cylcoalkyl, a bridged C₅₋₇cylcoalkyl, a C₃₋₆ cycloalkyl, halo, C₁₋₄alkyl, C₁₋₃haloalkyl, C₁₋₃alkoxy, C₁₋₃haloalkoxy, SO₂C₁₋₃alkyl, SO₂C₁₋₃haloalkyl, or Si(C₁₋₃alkyl)₃;
L^{A2} is a bond or O;
R^{A2} is H, C₁₋₃alkyl, C₁₋₃haloalkyl, C₀₋₃alkylene-CN, or C₁₋₃alkylene-O—C₁₋₃alkyl;
m is 0, 1, or 2;
each R⁷ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂—C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁-3hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})_{C}-3alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het;
or two geminal R⁷ groups, together with the atom to which they are attached, form a spiro-C₃₋₇cycloalkyl group;
Het is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo;
L³ is C₀₋₃alkylene, O—C₀₋₃alkylene-, —C(O)—, N(R^{N}), or N(R^{N})C(O)C₀₋₃alkylene-;
each R^{N} is independently H, C₁₋₃alkyl, C₁₋₃alkylene-CN, or C₁₋₃haloalkyl;
each of R² and R³ independently is C₁₋₆ alkyl;
R⁴ is H, —C(O)C₁₋₃alkyl, Het¹, C₁₋₃alkylene-O—C₁₋₃alkyl, C(O)C₁₃alkylene-N(R^{N})₂;
Het¹ is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo;
R⁵ is H, C₁₋₆alkyl, C₁₋₃ alkylene-CN, C₁₋₃alkylene-O—C₁₋₃alkyl, C₁₋₆alkoxy, C(O)C₀₋₃alkylene-N(R^{N})₂, C₀₋₃alkylene-heterocyclyl having 3-6 total ring atoms and 1-2 heteroatoms selected from N, S, and O, —C(O)—, or —C(OH)═, and the heterocycle or cycloalkyl ring is substituted with 0-3 substituents independently selected from C₁₋₆alkyl, C₁₋₆alkoxy, OC(O)C₁₋₃alkyl, and C₀₋₃ alkylene-CN;
X is N or CR⁸; and
R⁸ is H, halo, OH, C(O)C(O)OH, or C₁₋₆alkyl,
or when X is CR⁸, R⁵ and R⁸ together with the atoms to which they are attached form a fused 5-membered heterocycle, the fused heterocycle substituted with 0-2 substituents independently selected from OH, oxo, C₁₋₆alkyl, and a 3-8-membered monocyclic heterocycle having 1-2 ring heteroatoms selected from N, O, and S, and is substituted with 0-2 substituents independently selected from halo, OH, oxo, CN, and C₁₋₃alkyl.

2. The compound or salt of claim 1, wherein R¹ is 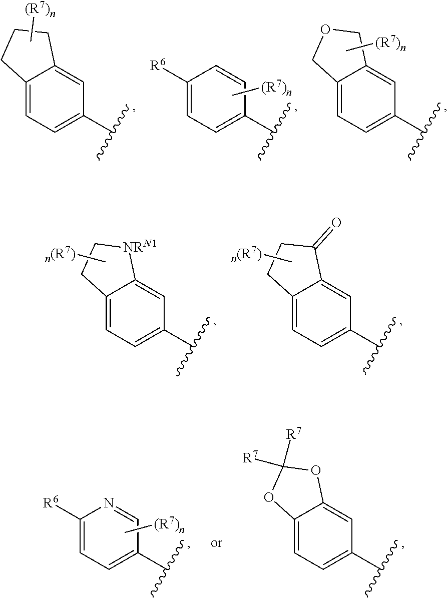
R^{N1} is H, C₁₋₆alkyl or C(O)C₁₋₃alkylene-N(R^{N})₂, each R^{N} is independently H or C₁₋₃alkyl, and n is 0, 1, 2, or 3.

3. The compound or salt of claim 2, wherein n is 0.

4. The compound or salt of claim 2, wherein n is 1 or 2.

5. The compound or salt of claim 1, wherein R⁶ is

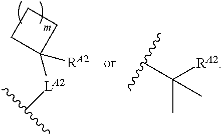

6. The compound or salt of claim 5, wherein R^{A2} is C₁₋₃alkyl, C₁₋₃haloalkyl, C₀₋₃alkylene-CN, or C₁₋₃alkylene-O—C₁₋₃alkyl.

7. The compound or salt of claim 5, wherein L^{A2} is a bond.

8. The compound or salt of claim 1, wherein R⁶ is 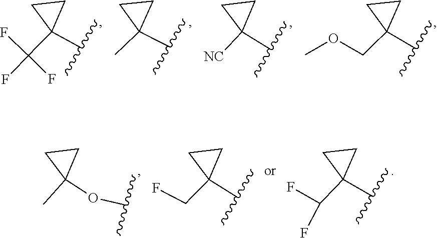
C(CH₃)₂CH₃, C(CH₃)₃, CH(CH₃)₂, 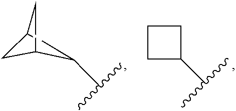
SO₂CF₃, CH(CH₃)(CF₃), C(F)₂CH₃, CH₂CF₃, 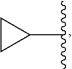 [SMILES: *C1CC1]
OCF₃, 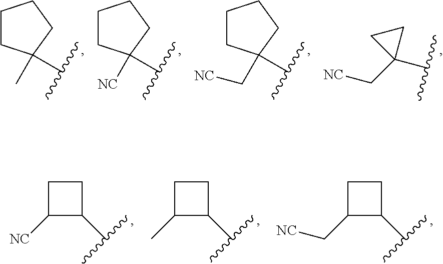
or Si(CH₃)₃.

9. The compound or salt claim 1, wherein at least one R⁷ is N(R^{N})C(O)C₁₋₃alkyl, N(R^{N})C(O)C₁₋₃hydroxyalkylene-N(R^{N})₂, N(R^{N})C(O)C₁₋₃hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, C₁₋₃alkylene-N(R^{N})S₂C₁₋₃alkyl, O—C₁₋₃alkylene-N(R^{N})SO₂C₁₋₃alkyl, or —C₀₋₃ alkylene-N(R^{N})₂.

10. The compound or salt of claim 9, wherein an alkyl or alkylene moiety of R⁷ is deuterated.

11. The compound or salt claim 1, wherein at least one R⁷ is OH, halo, C₁₋₆alkyl, CH₂SO₂NH₂, CH(CH₃)N(CH₃)₂, CH₂NHSO₂CH₃, SO₂CH₃, CH₂SO₂CH₃, OCH₂CH₂N(CH₃)₂, OCH₂CH₂OCH₃, OCH₂C(CH₃)₂OH, CH₂C(O)N(CH₃)₂, CH₂N(CH₃)CH₂CHF₂, CH₂NHCH₂CHF₂, CH₂N(CH₃)₂, CH₂CH₂N(CH₃)₂, CH₂CH₂CH₂N(CH₃)₂, CH₂NH₂, CH₂NHCH₃, CH₂CN, CN, NH₂, CH₂N(CH₃)CH₂CN, N(CH₃)₂, NHC(O)CH(NH₂)CH₂OH, NHC(O)C(CH₃)₂OH, NHC(O)CH₂N(CH₃)₂, NHC(O)CH₃, NHC(O)CH₂OCH₃, NHCH₂CH₂OCH₃, CD₂N(CD₃)₂, CH₂OH, CH(OH)CH₃, CH₂OCH₃, C(O)NHCH₂CH₂OCH₃, C(O)NHCH₂CH₂N(CH₃)₂, OCH₂CH₂NHSO₂CH₃, or two geminal R⁷ groups, together with the atom to which they are attached, form a spiro-C₃₋₇cycloalkyl group.

12. The compound or salt of claim 1, wherein at least one R⁷ is

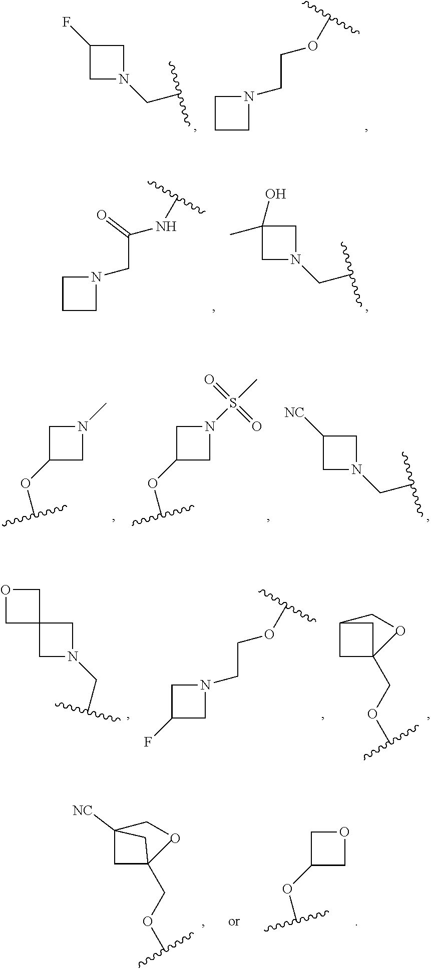

13. The compound or salt of claim 1 wherein at least one R⁷ is

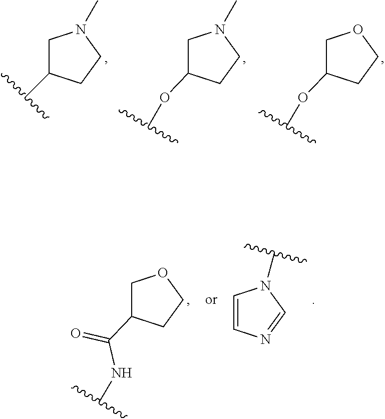

14. The compound or salt of claim 1, wherein at least one R⁷ is

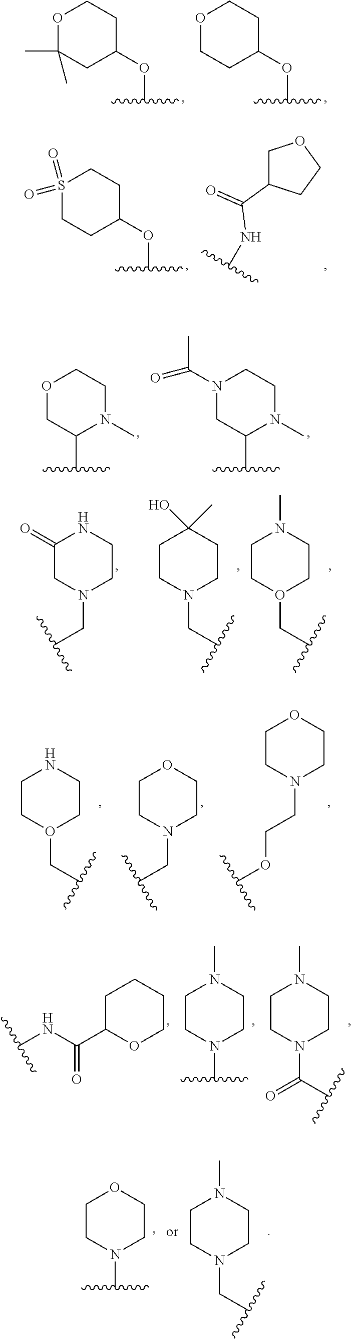

15. The compound or salt of claim 1 wherein R¹ is substituted with 1 R⁷.

16. The compound or salt of claim 1 wherein R¹ is substituted with 2 R⁷.

17. The compound or salt of claim 1 having a structure of Formula (IA) or Formula (IB): 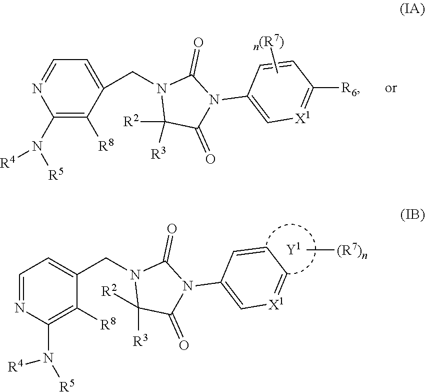
wherein X¹ is CH or N,
and Y¹ is a fused 5-membered cycloalkyl, or a fused 5-membered heterocycle having 1-2 ring heteroatoms selected from N, S, and O.

18. The compound or salt of claim 1, having a structure of Formula (IC) or Formula (ID): 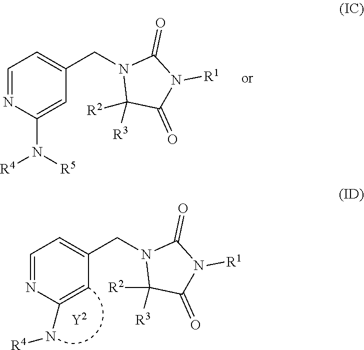
wherein Y² is a fused 5-membered heterocycle, the fused 5-membered heterocycle having 1-2 ring heteroatoms selected from N, O, and S, and substituted with 0-2 substituents independently selected from OH, oxo, C₁₋₆alkyl, or a 3-8-membered monocyclic heterocycle having 1-2 ring heteroatoms selected from N, O, and S, and is substituted with 0-2 substituents independently selected from halo, OH, oxo, CN, and C₁₋₃alkyl.

19. The compound or salt of claim 18, having a structure of Formula (ID-1), (ID-2), (ID-3), or (ID-4):

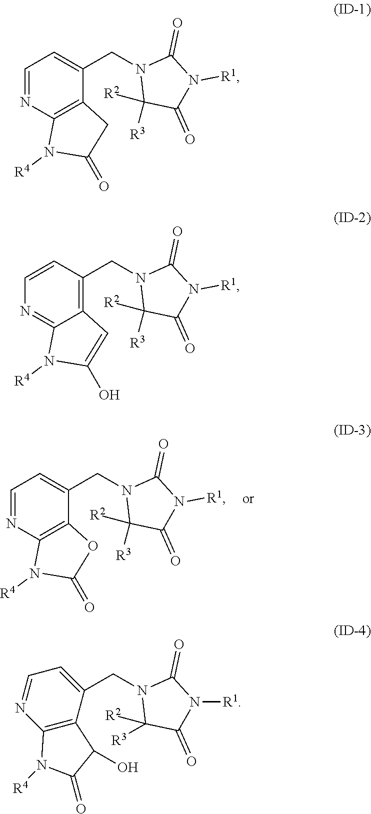

20. The compound or salt of claim 1, wherein R⁴ is H.

21. The compound or salt of claim 1, wherein R⁴ is —C(O)C₁₋₃alkyl, C₁₋₃alkylene-O—C₁₋₃alkyl, or C(O)C₁₋₃alkylene-N(R^{N})₂.

22. The compound or salt of claim 21, wherein R⁴ is C(O)CH(CH₃)₂, C(CH₃)CH₂CN, C(CH₃)CH₂OCH₃, or C(O)NH₂.

23. The compound or salt of claim 1, wherein R⁴ is Het¹.

24. The compound or salt of claim 23, wherein R⁴ is

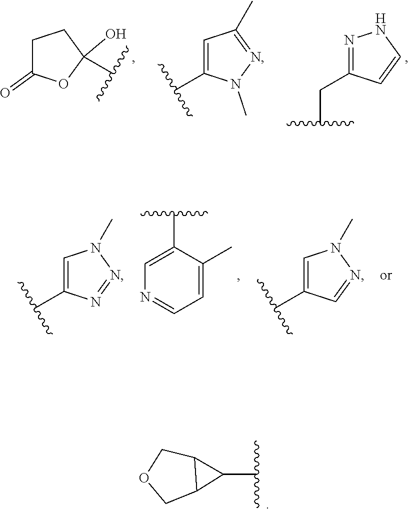

25. The compound or salt of claim 1, wherein each of R² and R³ are methyl.

26. The compound or salt of claim 1, wherein R¹ comprises phenyl and is substituted with 0 or 1 R⁷, R² and R³ are each independently C₁₋₃ alkyl, R⁶ is 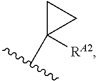
C(CH₃)₃, or CH(CH₃)₂, and
R^{A2} is C₁₋₃haloalkyl.

27. The compound or salt of claim 1, wherein R⁴ and R⁵ are each hydrogen.

28. A compound or pharmaceutically acceptable salt thereof, as listed below:

| Chemical Structure | Name |
|---|---|
| 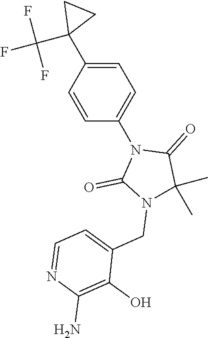 [SMILES: CC1(C)C(=O)N(c2ccc(C3(C(F)(F)F)CC3)cc2)C(=O)N1Cc1ccnc(N)c1O] | 1-[(2-amino-3-hydroxy-4-pyridyl)methyl]-5,5- dimethyl-3-{p-[1- (trifluoromethyl)cyclopropyl]phenyl}-2,4- imidazolidinedione; |
| 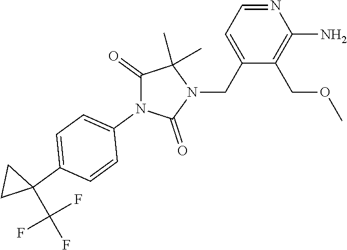 [SMILES: COCc1c(CN2C(=O)N(c3ccc(C4(C(F)(F)F)CC4)cc3)C(=O)C2(C)C)ccnc1N] | 1-{[2-amino-3-(methoxymethyl)-4- pyridyl]methyl}-5,5-dimethyl-3-{p-[1- (trifluoromethyl)cyclopropyl]phenyl}-2,4- imidazolidinedione; |
| 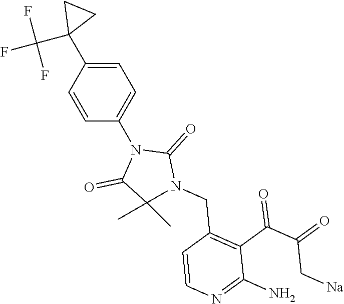 | sodium {2-amino-4-[(5,5-dimethyl-2,4-dioxo-3- {p-[1-(trifluoromethyl)cyclopropyl]phenyl}-1- imidazolidinyl)methyl]-3-pyridyl}oxoacetate; |
| 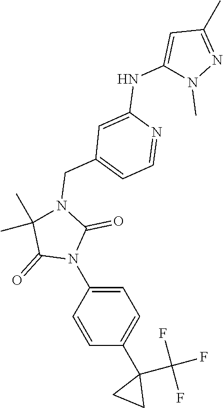 | 5,5-dimethyl-1-{[2-(1-methyl-3-methyl-5- pyrazolylamino)-4-pyridyl]methyl}-3-{p-[1- (trifluoromethyl)cyclopropyl]phenyl}-2,4- imidazolidinedione; |
| 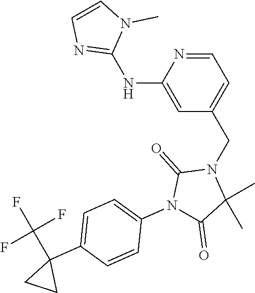 [SMILES: Cn1ccnc1Nc1cc(CN2C(=O)N(c3ccc(C4(C(F)(F)F)CC4)cc3)C(=O)C2(C)C)ccn1] | 5,5-dimethyl-1-{[2-(1-methyl-2- imidazolylamino)-4-pyridyl]methyl}-3-{p-[1- (trifluoromethyl)cyclopropyl]phenyl}-2.4- imidazolidinedione; |
| 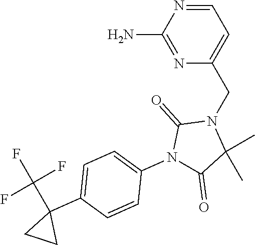 [SMILES: CC1(C)C(=O)N(c2ccc(C3(C(F)(F)F)CC3)cc2)C(=O)N1Cc1ccnc(N)n1] | 1-[(2-amino-4-pyrimidinyl)methyl]-5,5-dimethyl- 3-{p-[1-(trifluoromethyl)cyclopropyl]phenyl}- 2,4-imidazolidinedione; |
| 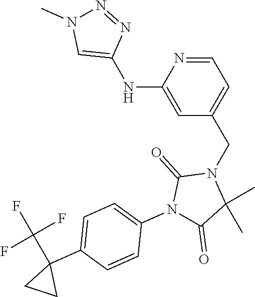 [SMILES: Cn1cc(Nc2cc(CN3C(=O)N(c4ccc(C5(C(F)(F)F)CC5)cc4)C(=O)C3(C)C)ccn2)nn1] | 5,5-dimethyl-1-{[2-(1-methyl-1H-1,2,3-triazol-4- ylamino)-4-pyridyllmethyl}-3-{p-[1- (trifluoromethyl)cyclopropyl]phenyl}-2,4- imidazolidinedione; |
| 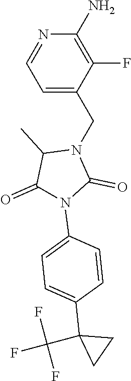 [SMILES: CC1C(=O)N(c2ccc(C3(C(F)(F)F)CC3)cc2)C(=O)N1Cc1ccnc(N)c1F] | 1-[(2-amino-3-fluoro-4-pyridyl)methyl]-5,5- dimethyl-3-{p-[1- (trifluoromethyl)cyclopropyl]phenyl}-2.4- imidazolidinedione: |
| 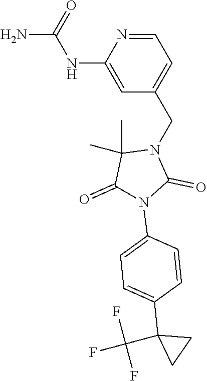 [SMILES: CC1(C)C(=O)N(c2ccc(C3(C(F)(F)F)CC3)cc2)C(=O)N1Cc1ccnc(NC(N)=O)c1] | 5,5-dimethyl-3-{p-[1- (trifluoromethyl)cyclopropyl]phenyl}-1-[(2- ureido-4-pyridyl)methyl1-2.4-imidazolidinedione |
| 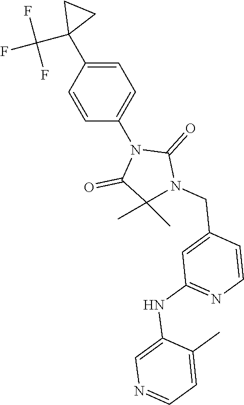 [SMILES: Cc1ccncc1Nc1cc(CN2C(=O)N(c3ccc(C4(C(F)(F)F)CC4)cc3)C(=O)C2(C)C)ccn1] | 5,5-dimethyl-1-{[2-(4-methyl-3-pyridylamino)-4- pyridyl]methyl}-3-{p-[1- (trifluoromethyl)cyclopropyl]phenyl}-2,4- imidazolidinedione; |
| 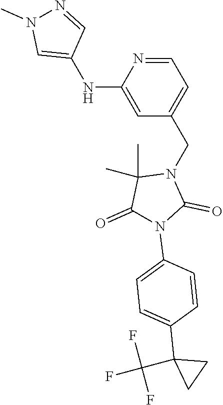 [SMILES: Cn1cc(Nc2cc(CN3C(=O)N(c4ccc(C5(C(F)(F)F)CC5)cc4)C(=O)C3(C)C)ccn2)cn1] | 5,5-dimethyl-1-{[2-(1-methyl-4- pyrazolylamino)-4-pyridyl]methyl}-3-{p-[1- (trifluoromethyl)cyclopropyl]phenyl}-2,4- imidazolidinedione; |
| 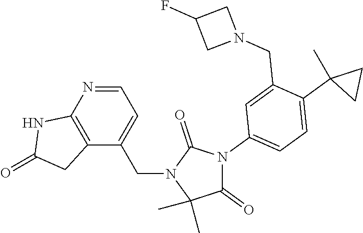 [SMILES: CC1(c2ccc(N3C(=O)N(Cc4ccnc5c4CC(=O)N5)C(C)(C)C3=O)cc2CN2CC(F)C2)CC1] | 3-{3-[(3-fluoro-1-azetidinyl)methyl]-4-(1- methylcyclopropyl)phenyl}-5,5-dimethyl-1-[(2- oxo-1,7-diaza-4-indanyl)methyl1-2,4- imidazolidinedione; |
| 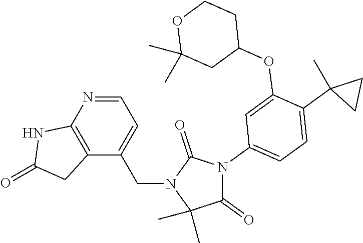 [SMILES: CC1(C)CC(Oc2cc(N3C(=O)N(Cc4ccnc5c4CC(=O)N5)C(C)(C)C3=O)ccc2C2(C)CC2)CCO1] | 3-[3-(2,2-dimethyltetrahydro-2H-pyran-4-yloxy)- 4-(1-methylcyclopropyl)phenyl]-5,5-dimethyl-1- [(2-oxo-1,7-diaza-4-indanyl)methyl]-2,4- imidazolidinedione; |
| 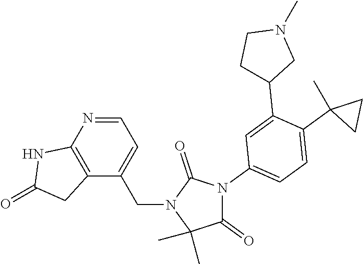 [SMILES: CN1CCC(c2cc(N3C(=O)N(Cc4ccnc5c4CC(=O)N5)C(C)(C)C3=O)ccc2C2(C)CC2)C1] | 5,5-dimethyl-3-[4-(1-methylcyclopropyl)-3-(1- methyl-3-pyrrolidinyl)phenyl]-1-[(2-oxo-1,7- diaza-4-indanyl)methyl]-2,4-imidazolidinedione; |
| 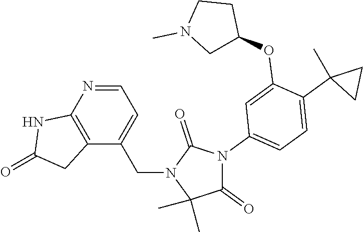 [SMILES: CN1CC[C@@H](Oc2cc(N3C(=O)N(Cc4ccnc5c4CC(=O)N5)C(C)(C)C3=O)ccc2C2(C)CC2)C1] | 3-{3-[(R)-1-methyl-3-pyrrolidinyloxyl-4-(1- methylcyclopropyl)phenyl}-5,5-dimethyl-1-[(2- oxo-1,7-diaza-4-indanyl)methyl]-2.4- imidazolidinedione; |
| 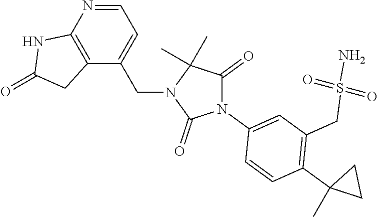 [SMILES: CC1(c2ccc(N3C(=O)N(Cc4ccnc5c4CC(=O)N5)C(C)(C)C3=O)cc2CS(N)(=O)=O)CC1] | (5-{4,4-dimethyl-2,5-dioxo-3-[(2-oxo-1,7-diaza- 4-indanyl)methyl]-1-imidazolidinyl}-2-(1- methylcyclopropyl)phenyl)methanesulfonamide; |
| 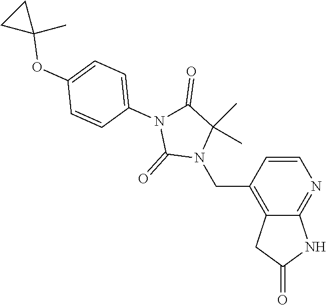 [SMILES: CC1(Oc2ccc(N3C(=O)N(Cc4ccnc5c4CC(=O)N5)C(C)(C)C3=O)cc2)CC1] | 5,5-dimethyl-3-[p-(1- methylcyclopropoxy)phenyl1-1-[(2-oxo-1,7- diaza-4-indanyl)methyl]-2,4-imidazolidinedione; |
| 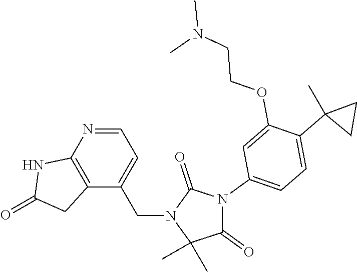 [SMILES: CN(C)CCOc1cc(N2C(=O)N(Cc3ccnc4c3CC(=O)N4)C(C)(C)C2=O)ccc1C1(C)CC1] | 3-{3-[2-(dimethylamino)ethoxy]-4-(1- methylcyclopropyl)phenyl}-5,5-dimethyl-1-[(2- oxo-1,7-diaza-4-indanyl)methyl]-2,4- imidazolidinedione; |
| 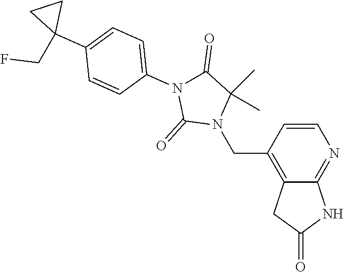 [SMILES: CC1(C)C(=O)N(c2ccc(C3(CF)CC3)cc2)C(=O)N1Cc1ccnc2c1CC(=O)N2] | 3-{p-[1-(fluoromethyl)cyclopropyl]phenyl}-5,5- dimethyl-1-[(2-oxo-1,7-diaza-4-indanyl)methyl]- 2,4-imidazolidinedione; |
| 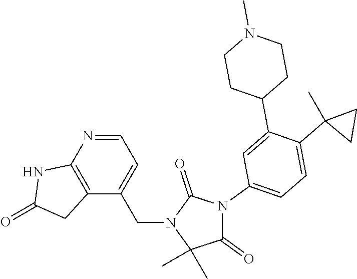 [SMILES: CN1CCC(c2cc(N3C(=O)N(Cc4ccnc5c4CC(=O)N5)C(C)(C)C3=O)ccc2C2(C)CC2)CC1] | 5,5-dimethyl-3-[4-(1-methylcyclopropyl)-3-(1- methyl-4-piperidyl)phenyl]-1-[(2-oxo-1,7-diaza- 4-indanyl)methyl]-2.4-imidazolidinedione; |
| 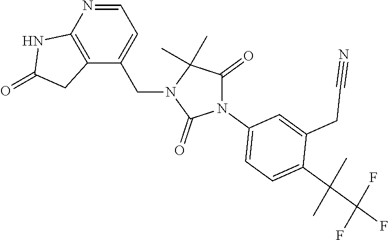 [SMILES: CC1(C)C(=O)N(c2ccc(C(C)(C)C(F)(F)F)c(CC#N)c2)C(=O)N1Cc1ccnc2c1CC(=O)N2] | (5-{4,4-dimethyl-2,5-dioxo-3-[(2-oxo-1,7-diaza- 4-indanyl)methyl]-1-imidazolidinyl}-2-(2,2,2- trifluoro-1,1-dimethylethyl)phenyl)acetonitrile; |
| 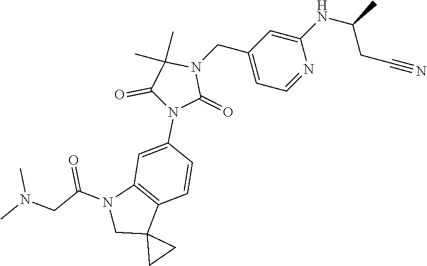 [SMILES: C[C@@H](CC#N)Nc1cc(CN2C(=O)N(c3ccc4c(c3)N(C(=O)CN(C)C)CC43CC3)C(=O)C2(C)C)ccn1] | (S)-3-14-[(3-{1′-[2- (dimethylamino)acetyl]spiro[cyclopropane-1,3′- indolin]-6′-yl}-5,5-dimethyl-2,4-dioxo-1- imidazolidinyl)methyl1-2- pyridylamino}butyronitrile; |
| 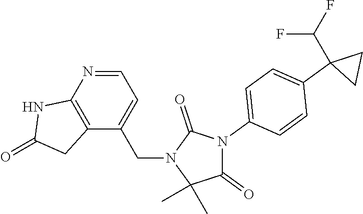 [SMILES: CC1(C)C(=O)N(c2ccc(C3(C(F)F)CC3)cc2)C(=O)N1Cc1ccnc2c1CC(=O)N2] | 3-{p-[1-(difluoromethyl)cyclopropyl]phenyl}- 5,5-dimethyl-1-[(2-oxo-1,7-diaza-4- indanyl)methyl]-2,4-imidazolidinedione; |
| 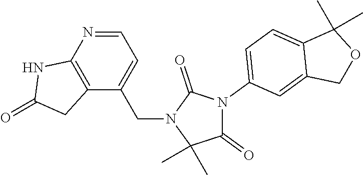 [SMILES: CC1(C)OCc2cc(N3C(=O)N(Cc4ccnc5c4CC(=O)N5)C(C)(C)C3=O)ccc21] | 3-(1,1-dimethyl-1,3-dihydro-5-isobenzofuranyl)- 5,5-dimethyl-1-[(2-oxo-1,7-diaza-4- indanyl)methyl]-2,4-imidazolidinedione; |
| 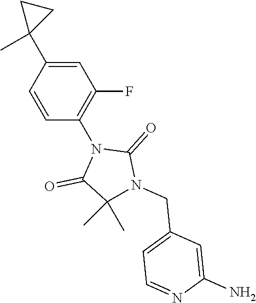 [SMILES: CC1(c2ccc(N3C(=O)N(Cc4ccnc(N)c4)C(C)(C)C3=O)c(F)c2)CC1] | 1-[(2-amino-4-pyridyl)methyl]-3-[2-fluoro-4-(1- methylcyclopropyl)phenyl]-5,5-dimethyl-2.4- imidazolidinedione; |
| 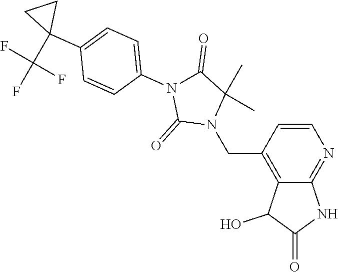 [SMILES: CC1(C)C(=O)N(c2ccc(C3(C(F)(F)F)CC3)cc2)C(=O)N1Cc1ccnc2c1C(O)C(=O)N2] | 1-[(3-hydroxy-2-oxo-1,7-diaza-4- indanyl)methyl]-5,5-dimethyl-3-{p-[1- (trifluoromethyl)cyclopropyl]phenyl}-2,4- imidazolidinedione; |
| 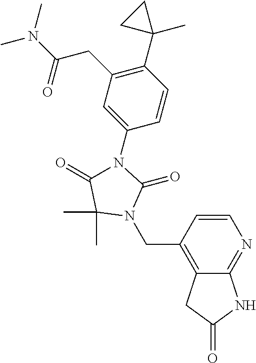 [SMILES: CN(C)C(=O)Cc1cc(N2C(=O)N(Cc3ccnc4c3CC(=O)N4)C(C)(C)C2=O)ccc1C1(C)CC1] | N,N-dimethyl(5-{4,4-dimethyl-2,5-dioxo-3-[(2- oxo-1,7-diaza-4-indanyl)methyl]-1- imidazolidinyl}-2-(1- methylcyclopropyl)phenyl)acetamide; |
| 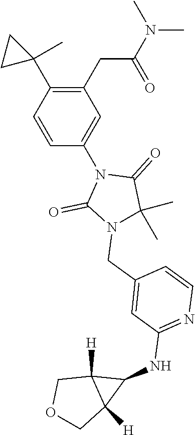 [SMILES: CN(C)C(=O)Cc1cc(N2C(=O)N(Cc3ccnc(N[C@H]4[C@@H]5COC[C@@H]54)c3)C(C)(C)C2=O)ccc1C1(C)CC1] | N,N-dimethyl(5-{3-[(2-{(1R,5S,6r)-3- oxabicyclo[3.1.0]hex-6-ylamino}-4- pyridyl)methyl]-4,4-dimethyl-2,5-dioxo-1- imidazolidinyl}-2-(1- methylcyclopropyl)phenyl)acetamide; |
| 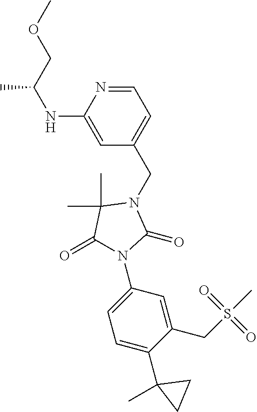 [SMILES: COC[C@@H](C)Nc1cc(CN2C(=O)N(c3ccc(C4(C)CC4)c(CS(C)(=O)=O)c3)C(=O)C2(C)C)ccn1] | 1-({2-[(R)-2-methoxy-1-methylethylamino]-4- pyridyl}methyl)-3-[3-(mesylmethyl)-4-(1- methylcyclopropyl)phenyl]-5,5-dimethyl-2.4- imidazolidinedione; |
| 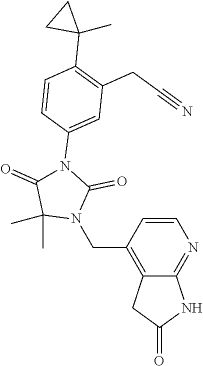 [SMILES: CC1(c2ccc(N3C(=O)N(Cc4ccnc5c4CC(=O)N5)C(C)(C)C3=O)cc2CC#N)CC1] | (5-{4,4-dimethyl-2,5-dioxo-3-[(2-oxo-1,7-diaza- 4-indanyl)methyl]-1-imidazolidinyl}-2-(1- methylcyclopropyl)phenyl)acetonitrile; |
| 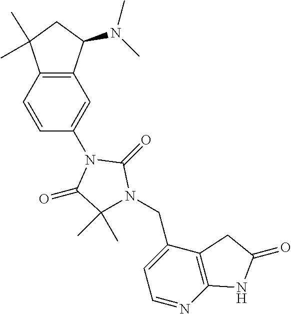 [SMILES: CN(C)[C@@H]1CC(C)(C)c2ccc(N3C(=O)N(Cc4ccnc5c4CC(=O)N5)C(C)(C)C3=O)cc21] | 3-[(R)-3-(dimethylamino)-1,1-dimethyl-5- indanyl]-5,5-dimethyl-1-[(2-oxo-1,7-diaza-4- indanyl)methyl]-2,4-imidazolidinedione; |
| 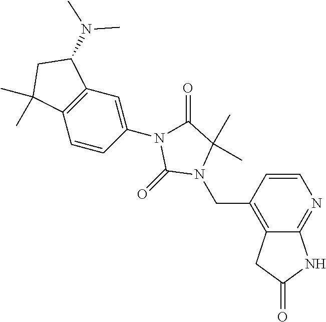 [SMILES: CN(C)[C@H]1CC(C)(C)c2ccc(N3C(=O)N(Cc4ccnc5c4CC(=O)N5)C(C)(C)C3=O)cc21] | 3-[(S)-3-(dimethylamino)-1,1-dimethyl-5- indanyl]-5,5-dimethyl-1-[(2-oxo-1,7-diaza-4- indanyl)methyl]-2.4-imidazolidinedione; |
| 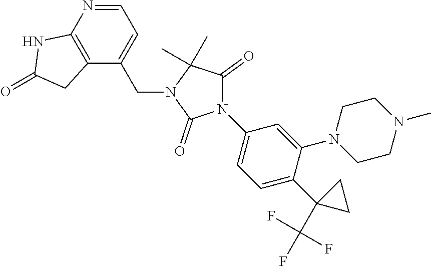 [SMILES: CN1CCN(c2cc(N3C(=O)N(Cc4ccnc5c4CC(=O)N5)C(C)(C)C3=O)ccc2C2(C(F)(F)F)CC2)CC1] | 5,5-dimethyl-3-{3-(4-methyl-1-piperazinyl)-4-[1- (trifluoromethyl)cyclopropyl]phenyl}-1-[(2-oxo- 1,7-diaza-4-indanyl)methyl]-2,4- imidazolidinedione; |
| 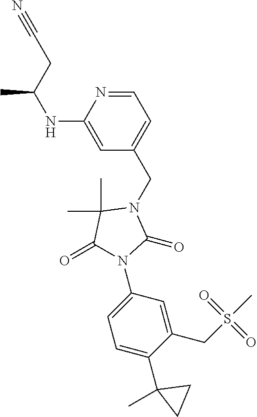 [SMILES: C[C@@H](CC#N)Nc1cc(CN2C(=O)N(c3ccc(C4(C)CC4)c(CS(C)(=O)=O)c3)C(=O)C2(C)C)ccn1] | (S)-3-[4-({3-[3-(mesylmethyl)-4-(1- methylcyclopropyl)phenyl]-5,5-dimethyl-2,4- dioxo-1-imidazolidinyl}methyl)-2- pyridylamino]butyronitrile; |
| 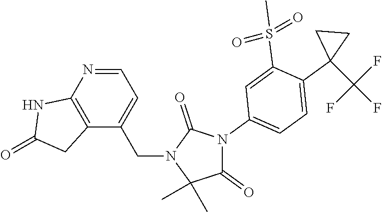 [SMILES: CC1(C)C(=O)N(c2ccc(C3(C(F)(F)F)CC3)c(S(C)(=O)=O)c2)C(=O)N1Cc1ccnc2c1CC(=O)N2] | 3-{3-mesyl-4-[1- (trifluoromethyl)cyclopropyl]phenyl}-5,5- dimethyl-1-[(2-oxo-1,7-diaza-4-indanyl)methyl]- 2,4-imidazolidinedione; |
| 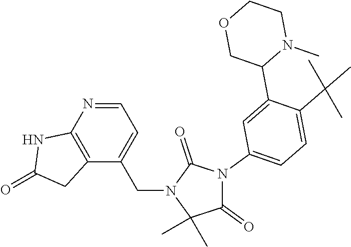 [SMILES: CN1CCOCC1c1cc(N2C(=O)N(Cc3ccnc4c3CC(=O)N4)C(C)(C)C2=O)ccc1C(C)(C)C] | 3-[4-(tert-butyl)-3-(4-methyl-3- morpholinyl)phenyl]-5,5-dimethyl-1-[(2-oxo-1,7- diaza-4-indanyl)methyl]-2,4-imidazolidinedione; |
| 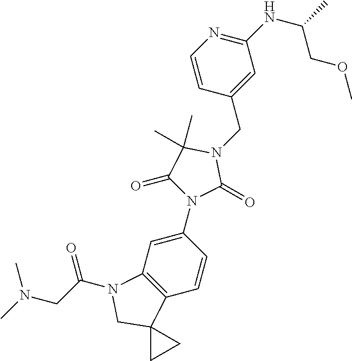 | 1-({2-[(R)-2-methoxy-1-methylethylamino]-4- pyridyl}methyl)-3-{1′-[2- (dimethylamino)acetyl\|spiro[cyclopropane-1,3′- indolin]-6′-yl}-5,5-dimethyl-2.4- imidazolidinedione; |
| 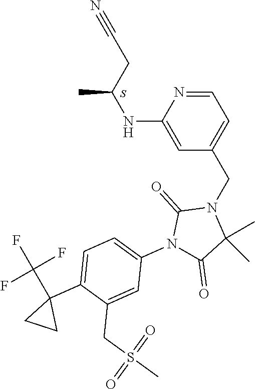 | (S)-3-{4-[(3-{3-(mesylmethyl)-4-[1- (trifluoromethyl)cyclopropyl]phenyl}-5,5- dimethyl-2,4-dioxo-1-imidazolidinyl)methyl]-2- pyridylamino}butyronitrile; |
| 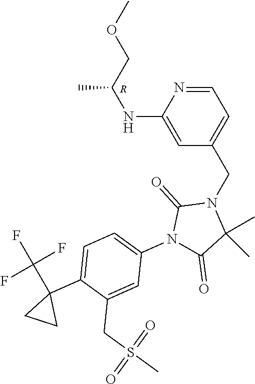 | 1-({2-[(R)-2-methoxy-1-methylethylamino]-4- pyridyl}methyl)-3-{3-(mesylmethyl)-4-[1- (trifluoromethyl)cyclopropyl]phenyl}-5,5- dimethyl-2,4-imidazolidinedione; |
| 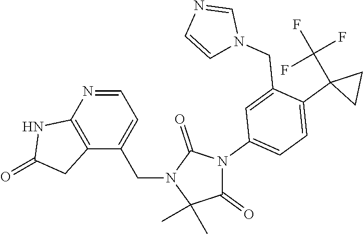 [SMILES: CC1(C)C(=O)N(c2ccc(C3(C(F)(F)F)CC3)c(Cn3ccnc3)c2)C(=O)N1Cc1ccnc2c1CC(=O)N2] | 3-{3-[(1-imidazolyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 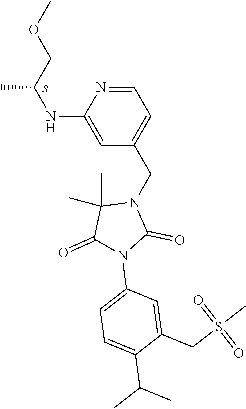 | 1-({2-[(R)-2-methoxy-1-methylethylamino]-4- pyridyl}methyl)-3-[3-(mesylmethyl)-4-cumenyl]- 5,5-dimethyl-2,4-imidazolidinedione; |
| 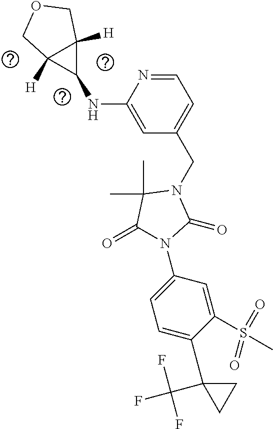 [SMILES: CC1(C)C(=O)N(c2ccc(C3(C(F)(F)F)CC3)c(S(C)(=O)=O)c2)C(=O)N1Cc1ccnc(N[C@H]2[C@@H]3COC[C@@H]32)c1] | 1-[(2-{(1R.5S,6r)-3-oxabicyclo[3.1.0]hex-6- ylamino}-4-pyridyl)methyl1-3-{3-mesyl-4-[1- (trifluoromethyl)cyclopropyl]phenyl}-5,5- dimethyl-2,4-imidazolidinedione; |
| 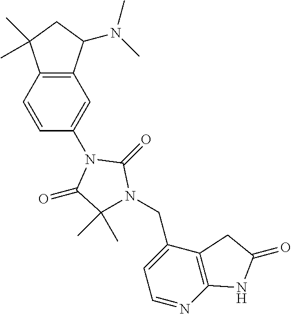 [SMILES: CN(C)C1CC(C)(C)c2ccc(N3C(=O)N(Cc4ccnc5c4CC(=O)N5)C(C)(C)C3=O)cc21] | 3-[3-(dimethylamino)-1,1-dimethyl-5-indanyl]- 5,5-dimethyl-1-[(2-oxo-1,7-diaza-4- indanyl)methyl]-2,4-imidazolidinedione; |
| 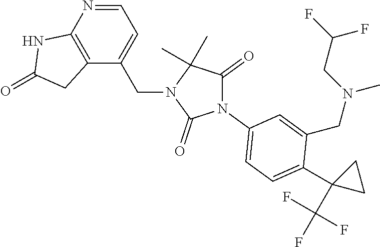 [SMILES: CN(Cc1cc(N2C(=O)N(Cc3ccnc4c3CC(=O)N4)C(C)(C)C2=O)ccc1C1(C(F)(F)F)CC1)CC(F)F] | 3-(3-{[(2,2-difluoroethyl)-N- methylamino methyl}-4-[1- (trifluoromethyl)cyclopropyl]phenyl)-5,5- dimethyl-1-[(2-oxo-1,7-diaza-4-indanyl)methyl]- 2,4-imidazolidinedione; |
| 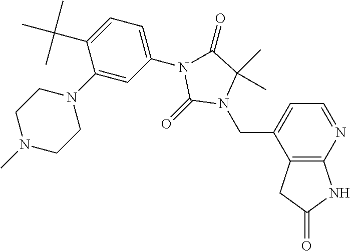 [SMILES: CN1CCN(c2cc(N3C(=O)N(Cc4ccnc5c4CC(=O)N5)C(C)(C)C3=O)ccc2C(C)(C)C)CC1] | 3-[4-(tert-butyl)-3-(4-methyl-1- piperazinyl)phenyl]-5,5-dimethyl-1-[(2-oxo-1,7- diaza-4-indanyl)methyl1-2.4-imidazolidinedione; |
| 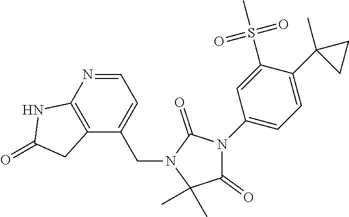 [SMILES: CC1(c2ccc(N3C(=O)N(Cc4ccnc5c4CC(=O)N5)C(C)(C)C3=O)cc2S(C)(=O)=O)CC1] | 3-[3-mesyl-4-(1-methylcyclopropyl)phenyl]-5,5- dimethyl-1-[(2-oxo-1,7-diaza-4-indanyl)methyl]- 2,4-imidazolidinedione; |
| 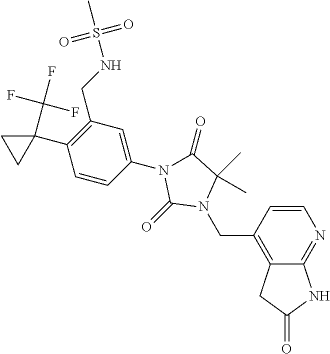 [SMILES: CC1(C)C(=O)N(c2ccc(C3(C(F)(F)F)CC3)c(CNS(C)(=O)=O)c2)C(=O)N1Cc1ccnc2c1CC(=O)N2] | 3-{3-[(mesylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 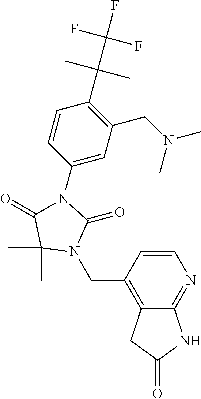 [SMILES: CN(C)Cc1cc(N2C(=O)N(Cc3ccnc4c3CC(=O)N4)C(C)(C)C2=O)ccc1C(C)(C)C(F)(F)F] | 3-{3-[(dimethylamino)methyl]-4-(2,2,2-trifluoro- 1,1-dimethylethyl)phenyl}-5,5-dimethyl-1-[(2- oxo-1,7-diaza-4-indanyl)methyl]-2,4- imidazolidinedione; |
| 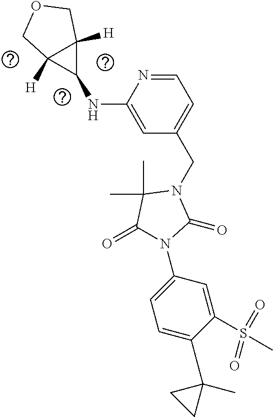 [SMILES: CC1(c2ccc(N3C(=O)N(Cc4ccnc(N[C@H]5[C@@H]6COC[C@@H]65)c4)C(C)(C)C3=O)cc2S(C)(=O)=O)CC1] | 1-[(2-{(1R,5S,6r)-3-oxabicyclo[3.1.0]hex-6- ylamino}-4-pyridyl)methyl]-3-[3-mesyl-4-(1- methylcyclopropyl)phenyl]-5,5-dimethyl-2,4- imidazolidinedione; |
| 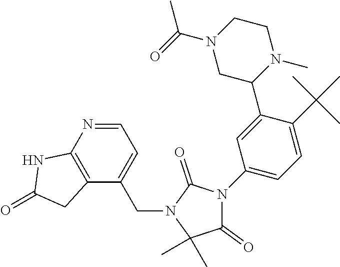 [SMILES: CC(=O)N1CCN(C)C(c2cc(N3C(=O)N(Cc4ccnc5c4CC(=O)N5)C(C)(C)C3=O)ccc2C(C)(C)C)C1] | 3-[3-(4-acetyl-1-methyl-2-piperazinyl)-4-(tert- butyl)phenyl]-5,5-dimethyl-1-[(2-oxo-1,7-diaza- 4-indanyl)methyl]-2,4-imidazolidinedione; |
| 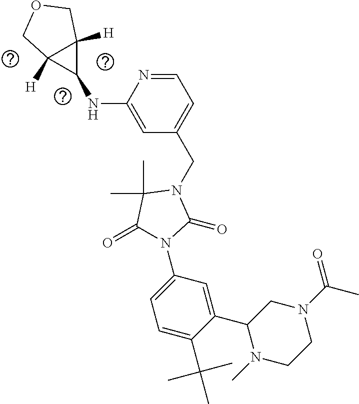 [SMILES: CC(=O)N1CCN(C)C(c2cc(N3C(=O)N(Cc4ccnc(N[C@H]5[C@@H]6COC[C@@H]65)c4)C(C)(C)C3=O)ccc2C(C)(C)C)C1] | 1-[(2-{(1R,5S,6r)-3-oxabicyclo[3.1.0]hex-6- ylamino}-4-pyridyl)methyl]-3-[3-(4-acetyl-1- methyl-2-piperazinyl)-4-(tert-butyl)phenyl]-5,5- dimethyl-2,4-imidazolidinedione; |
| 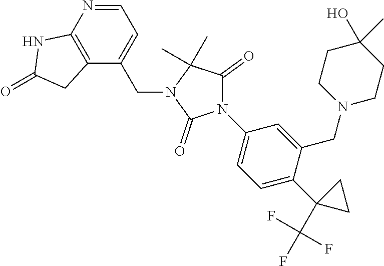 [SMILES: CC1(O)CCN(Cc2cc(N3C(=O)N(Cc4ccnc5c4CC(=O)N5)C(C)(C)C3=O)ccc2C2(C(F)(F)F)CC2)CC1] | 3-{3-[(4-hydroxy-4-methyl-1-piperidyl)methyl]- 4-[1-(trifluoromethyl)cyclopropyl]phenyl}-5,5- dimethyl-1-[(2-oxo-1,7-diaza-4-indanyl)methyl]- 2,4-imidazolidinedione; |
| 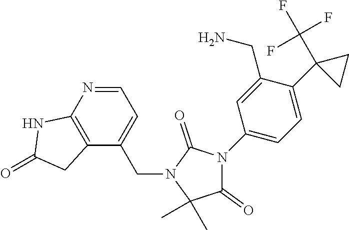 [SMILES: CC1(C)C(=O)N(c2ccc(C3(C(F)(F)F)CC3)c(CN)c2)C(=O)N1Cc1ccnc2c1CC(=O)N2] | 3-{3-(aminomethyl)-4-[1- (trifluoromethyl)cyclopropyl]phenyl}-5.5- dimethyl-1-[(2-oxo-1,7-diaza-4-indanyl)methyl]- 2,4-imidazolidinedione; |
| 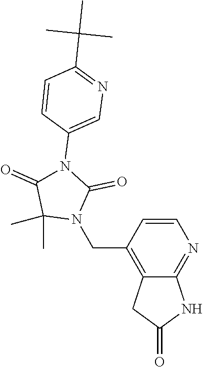 [SMILES: CC(C)(C)c1ccc(N2C(=O)N(Cc3ccnc4c3CC(=O)N4)C(C)(C)C2=O)cn1] | 3-[6-(tert-butyl)-3-pyridyl]-5,5-dimethyl-1-[(2- oxo-1,7-diaza-4-indanyl)methyl]-2.4- imidazolidinedione; |
| 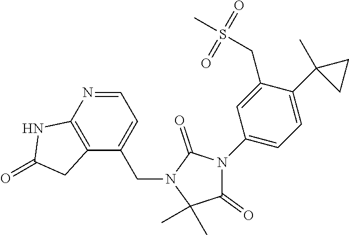 [SMILES: CC1(c2ccc(N3C(=O)N(Cc4ccnc5c4CC(=O)N5)C(C)(C)C3=O)cc2CS(C)(=O)=O)CC1] | 3-[3-(mesylmethyl)-4-(1- methylcyclopropyl)phenyl]-5,5-dimethyl-1-[(2- oxo-1,7-diaza-4-indanyl)methyl1-2,4- imidazolidinedione; |
| 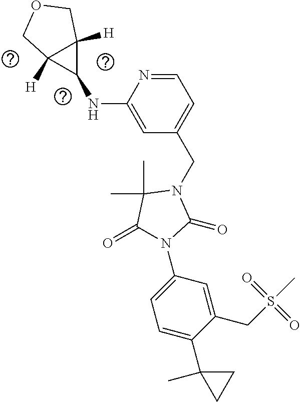 [SMILES: CC1(c2ccc(N3C(=O)N(Cc4ccnc(N[C@H]5[C@@H]6COC[C@@H]65)c4)C(C)(C)C3=O)cc2CS(C)(=O)=O)CC1] | 1-[(2-{(1R,5S,6r)-3-oxabicyclo[3.1.0]hex-6- ylamino}-4-pyridyl)methyl]-3-[3-(mesylmethyl)- 4-(1-methylcyclopropyl)phenyl]-5,5-dimethyl- 2,4-imidazolidinedione; |
| 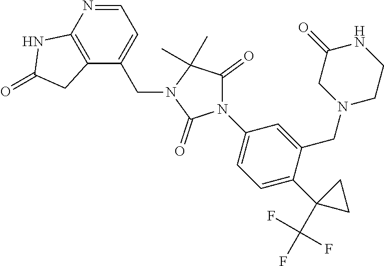 [SMILES: CC1(C)C(=O)N(c2ccc(C3(C(F)(F)F)CC3)c(CN3CCNC(=O)C3)c2)C(=O)N1Cc1ccnc2c1CC(=O)N2] | 5,5-dimethyl-1-[(2-oxo-1,7-diaza-4- indanyl)methyl]-3-{3-[(3-oxo-1- piperazinyl)methyl]-4-[1- (trifluoromethyl)cyclopropyl]phenyl}-2,4- imidazolidinedione; |
| 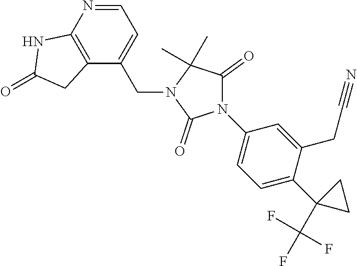 [SMILES: CC1(C)C(=O)N(c2ccc(C3(C(F)(F)F)CC3)c(CC#N)c2)C(=O)N1Cc1ccnc2c1CC(=O)N2] | (5-{4,4-dimethyl-2,5-dioxo-3-[(2-oxo-1,7-diaza- 4-indanyl)methyl]-1-imidazolidinyl}-2-[1- (trifluoromethyl)cyclopropyl]phenyl)acetonitrile; |
| 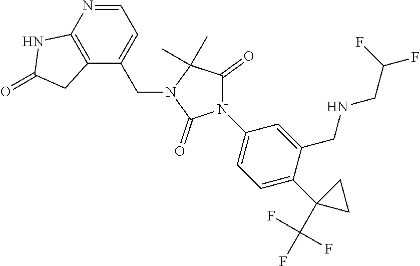 [SMILES: CC1(C)C(=O)N(c2ccc(C3(C(F)(F)F)CC3)c(CNCC(F)F)c2)C(=O)N1Cc1ccnc2c1CC(=O)N2] | 3-{3-[(2,2-difluoroethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 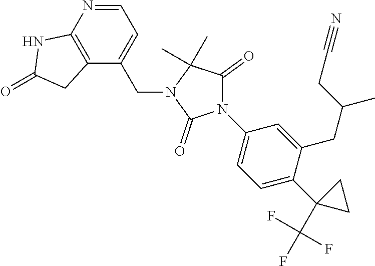 [SMILES: CC(CC#N)Cc1cc(N2C(=O)N(Cc3ccnc4c3CC(=O)N4)C(C)(C)C2=O)ccc1C1(C(F)(F)F)CC1] | {[(5-{4.4-dimethyl-2.5-dioxo-3-[(2-oxo-1,7- diaza-4-indanyl)methyl]-1-imidazolidinyl}-2-[1- (trifluoromethyl)cyclopropyl]phenyl)methyl]-N- methylamino}acetonitrile; |
| 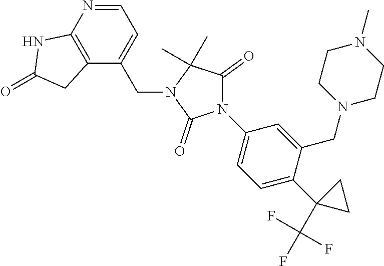 [SMILES: CN1CCN(Cc2cc(N3C(=O)N(Cc4ccnc5c4CC(=O)N5)C(C)(C)C3=O)ccc2C2(C(F)(F)F)CC2)CC1] | 5,5-dimethyl-3-{3-[(4-methyl-1- piperazinyl)methyl]-4-[1- (trifluoromethyl)cyclopropyl]phenyl}-1-[(2-oxo- 1,7-diaza-4-indanyl)methyl]-2,4- imidazolidinedione; |
| 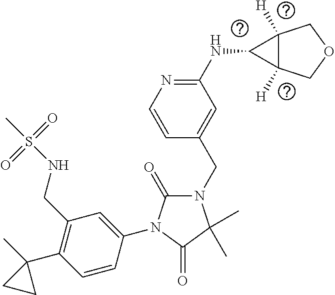 [SMILES: CC1(c2ccc(N3C(=O)N(Cc4ccnc(N[C@H]5[C@@H]6COC[C@@H]65)c4)C(C)(C)C3=O)cc2CNS(C)(=O)=O)CC1] | 1-[(2-{(1R,5S,6r)-3-oxabicyclo[3.1.0]hex-6- ylamino}-4-pyridyl)methyl1-3-{3- [(mesylamino)methyl]-4-(1- methylcyclopropyl)phenyl}-5,5-dimethyl-2,4- imidazolidinedione; |
| 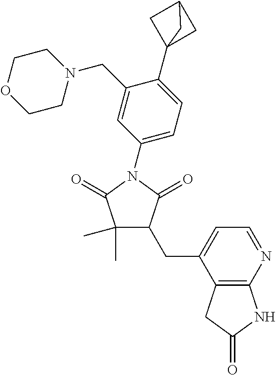 [SMILES: CC1(C)C(=O)N(c2ccc(C34CC(C3)C4)c(CN3CCOCC3)c2)C(=O)C1Cc1ccnc2c1CC(=O)N2] | 3-{4-(bicyclo[1.1.1]pent-1-yl)-3- (morpholinomethyl)phenyl}-5,5-dimethyl-1-[(2- oxo-1,7-diaza-4-indanyl)methyl]-2,4- imidazolidinedione; |
| 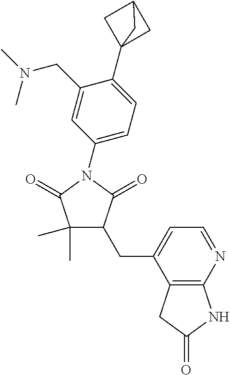 [SMILES: CN(C)Cc1cc(N2C(=O)C(Cc3ccnc4c3CC(=O)N4)C(C)(C)C2=O)ccc1C12CC(C1)C2] | 3-{4-(bicyclo[1.1.1]pent-1-yl)-3- [(dimethylamino)methyl]phenyl}-5,5-dimethyl- 1-[(2-oxo-1,7-diaza-4-indanyl)methyl]-2.4- imidazolidinedione; |
| 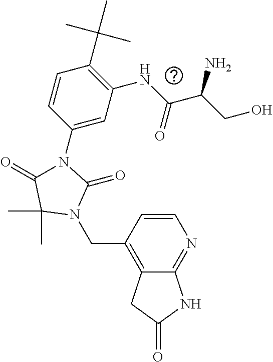 [SMILES: CC(C)(C)c1ccc(N2C(=O)N(Cc3ccnc4c3CC(=O)N4)C(C)(C)C2=O)cc1NC(=O)[C@@H](N)CO] | N-[2-(tert-butyl)-5-{4,4-dimethyl-2,5-dioxo-3- [(2-oxo-1,7-diaza-4-indanyl)methyl]-1- imidazolidinyl}phenyl](S)-2-amino-3- hydroxypropionamide; |
| 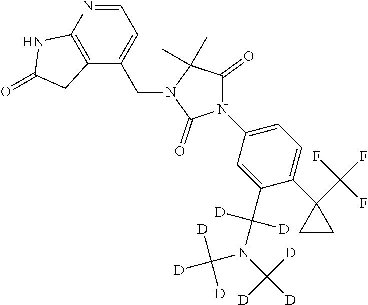 [SMILES: [2H]C([2H])([2H])N(C([2H])([2H])[2H])C([2H])([2H])c1cc(N2C(=O)N(Cc3ccnc4c3CC(=O)N4)C(C)(C)C2=O)ccc1C1(C(F)(F)F)CC1] | 3-(3-{[di(2H3)methylamino](2H2)methyl}-4-[1- (trifluoromethyl)cyclopropyl]phenyl)-5,5- dimethyl-1-[(2-oxo-1,7-diaza-4-indanyl)methyl]- 2.4-imidazolidinedione; |
| 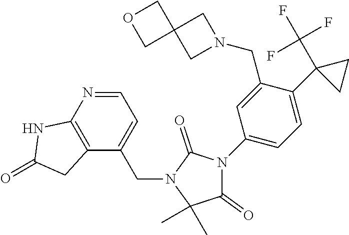 [SMILES: CC1(C)C(=O)N(c2ccc(C3(C(F)(F)F)CC3)c(CN3CC4(COC4)C3)c2)C(=O)N1Cc1ccnc2c1CC(=O)N2] | 5,5-dimethyl-3-(3-{(2-oxa-6-aza-6- spiro[3.3]heptyl)methyl}-4-[1- (trifluoromethyl)cyclopropyl]phenyl)-1-[(2-oxo- 1,7-diaza-4-indanyl)methyl1-2,4- imidazolidinedione; |
| 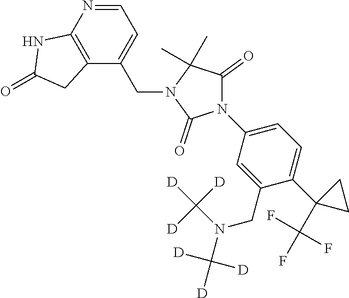 [SMILES: [2H]C([2H])([2H])N(Cc1cc(N2C(=O)N(Cc3ccnc4c3CC(=O)N4)C(C)(C)C2=O)ccc1C1(C(F)(F)F)CC1)C([2H])([2H])[2H]] | 3-(3-{[di(²H₃)methylamino]methyl}-4-[1- (trifluoromethyl)cyclopropyl]phenyl)-5,5- dimethyl-1-[(2-oxo-1,7-diaza-4-indanyl)methyl]- 2,4-imidazolidinedione; |
| 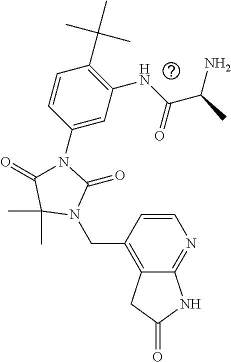 [SMILES: C[C@H](N)C(=O)Nc1cc(N2C(=O)N(Cc3ccnc4c3CC(=O)N4)C(C)(C)C2=O)ccc1C(C)(C)C] | N-[2-(tert-butyl)-5-{4,4-dimethyl-2,5-dioxo-3- [(2-oxo-1,7-diaza-4-indanyl)methyl]-1- imidazolidinyl}phenyl](S)-2- aminopropionamide; |
| 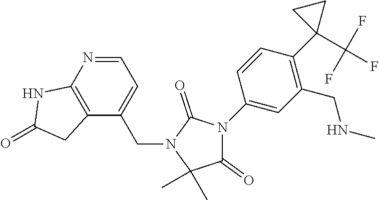 [SMILES: CNCc1cc(N2C(=O)N(Cc3ccnc4c3CC(=O)N4)C(C)(C)C2=O)ccc1C1(C(F)(F)F)CC1] | 5,5-dimethyl-3-{3-[(methylamino)methyl]-4-[1- (trifluoromethyl)cyclopropyl]phenyl}-1-[(2-oxo- 1,7-diaza-4-indanyl)methyl]-2,4- imidazolidinedione; |
| 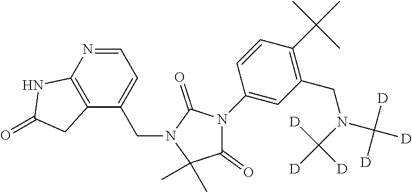 [SMILES: [2H]C([2H])([2H])N(Cc1cc(N2C(=O)N(Cc3ccnc4c3CC(=O)N4)C(C)(C)C2=O)ccc1C(C)(C)C)C([2H])([2H])[2H]] | 3-[4-(tert-butyl)-3- {[di(²H₃)methylamino]methyl}phenyl]-5,5- dimethyl-1-[(2-oxo-1,7-diaza-4-indanyl)methyl]- 2,4-imidazolidinedione; |
| 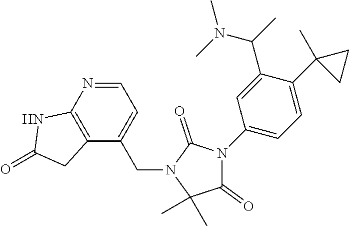 [SMILES: CC(c1cc(N2C(=O)N(Cc3ccnc4c3CC(=O)N4)C(C)(C)C2=O)ccc1C1(C)CC1)N(C)C] | 3-{3-[1-(dimethylamino)ethyl]-4-(1- methylcyclopropyl)phenyl}-5,5-dimethyl-1-[(2- oxo-1,7-diaza-4-indanyl)methyl]-2.4- imidazolidinedione; |
| 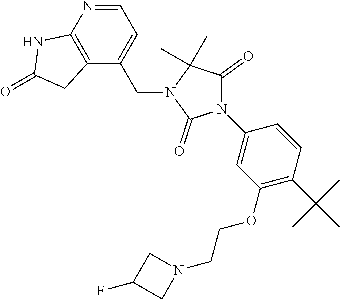 [SMILES: CC(C)(C)c1ccc(N2C(=O)N(Cc3ccnc4c3CC(=O)N4)C(C)(C)C2=O)cc1OCCN1CC(F)C1] | 3-{4-(tert-butyl)-3-[2-(3-fluoro-1- azetidinyl)ethoxy]phenyl}-5,5-dimethyl-1-[(2- oxo-1,7-diaza-4-indanyl)methyl]-2,4- imidazolidinedione; |
| 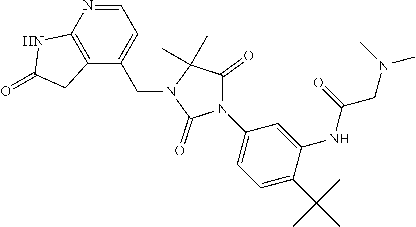 [SMILES: CN(C)CC(=O)Nc1cc(N2C(=O)N(Cc3ccnc4c3CC(=O)N4)C(C)(C)C2=O)ccc1C(C)(C)C] | N-[2-(tert-butyl)-5-{4,4-dimethyl-2,5-dioxo-3- [(2-oxo-1,7-diaza-4-indanyl)methyl]-1- imidazolidinyl}phenyl](dimethylamino) acetamide; |
| 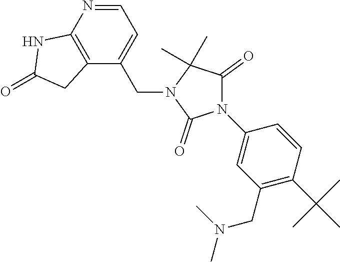 [SMILES: CN(C)Cc1cc(N2C(=O)N(Cc3ccnc4c3CC(=O)N4)C(C)(C)C2=O)ccc1C(C)(C)C] | 3-{4-(tert-butyl)-3- [(dimethylamino)methyl]phenyl}-5,5-dimethyl- 1-[(2-oxo-1,7-diaza-4-indanyl)methyl]-2,4- imidazolidinedione; |
| 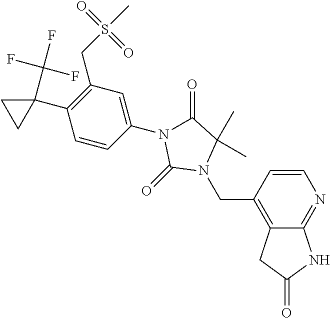 [SMILES: CC1(C)C(=O)N(c2ccc(C3(C(F)(F)F)CC3)c(CS(C)(=O)=O)c2)C(=O)N1Cc1ccnc2c1CC(=O)N2] | 3-{3-(mesylmethyl)-4-[1- (trifluoromethyl)cyclopropyl]phenyl}-5,5- dimethyl-1-[(2-oxo-1,7-diaza-4-indanyl)methyl]- 2,4-imidazolidinedione; |
| 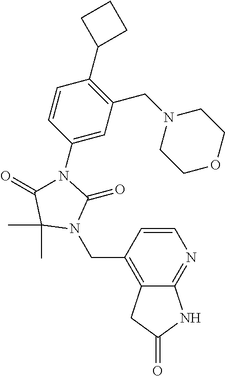 [SMILES: CC1(C)C(=O)N(c2ccc(C3CCC3)c(CN3CCOCC3)c2)C(=O)N1Cc1ccnc2c1CC(=O)N2] | 3-[4-cyclobutyl-3-(morpholinomethyl)phenyl]- 5,5-dimethyl-1-[(2-oxo-1,7-diaza-4- indanyl)methyl]-2.4-imidazolidinedione; |
| 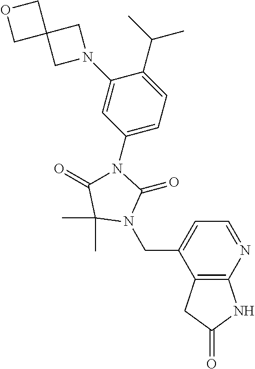 [SMILES: CC(C)c1ccc(N2C(=O)N(Cc3ccnc4c3CC(=O)N4)C(C)(C)C2=O)cc1N1CC2(COC2)C1] | 5,5-dimethyl-3-{3-(2-oxa-6-aza-6- spiro[3.3]heptyl)-4-cumenyl}-1-[(2-oxo-1,7- diaza-4-indanyl)methyl]-2,4-imidazolidinedione; |
| 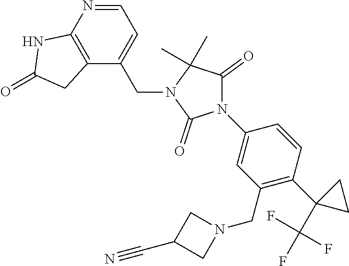 [SMILES: CC1(C)C(=O)N(c2ccc(C3(C(F)(F)F)CC3)c(CN3CC(C#N)C3)c2)C(=O)N1Cc1ccnc2c1CC(=O)N2] | 1-[(5-{4,4-dimethyl-2,5-dioxo-3-[(2-oxo-1,7- diaza-4-indanyl)methyl]-1-imidazolidinyl}-2-[1- (trifluoromethyl)cyclopropyl]phenyl)methyl]-3- azetidinecarbonitrile; |
| 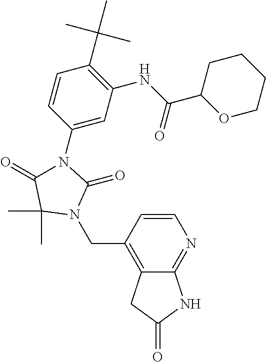 [SMILES: CC(C)(C)c1ccc(N2C(=O)N(Cc3ccnc4c3CC(=O)N4)C(C)(C)C2=O)cc1NC(=O)C1CCCCO1] | N-[2-(tert-butyl)-5-{4,4-dimethyl-2,5-dioxo-3- [(2-oxo-1,7-diaza-4-indanyl)methyl]-1- imidazolidinyl}phenyl]-tetrahydro-2H-pyran-2- carboxamide; |
| 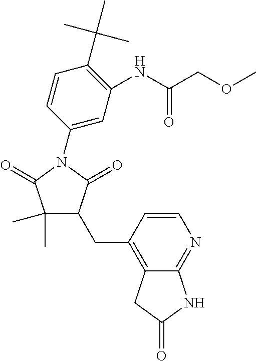 [SMILES: COCC(=O)Nc1cc(N2C(=O)C(Cc3ccnc4c3CC(=O)N4)C(C)(C)C2=O)ccc1C(C)(C)C] | N-[2-(tert-butyl)-5-{4,4-dimethyl-2,5-dioxo-3- [(2-oxo-1,7-diaza-4-indanyl)methyl]-1- imidazolidinyl}phenyl]methoxyacetamide; |
| 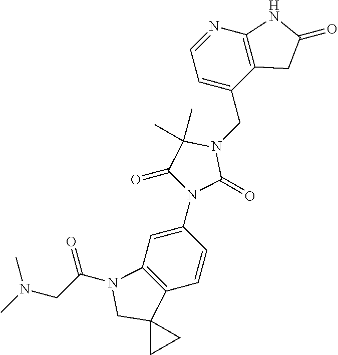 [SMILES: CN(C)CC(=O)N1CC2(CC2)c2ccc(N3C(=O)N(Cc4ccnc5c4CC(=O)N5)C(C)(C)C3=O)cc21] | 3-{1′-[2- (dimethylamino)acetyl]spiro[cyclopropane-1,3′- indolin]-6′-yl}-5,5-dimethyl-1-[(2-oxo-1,7-diaza- 4-indanyl)methyl]-2.4-imidazolidinedione; |
| 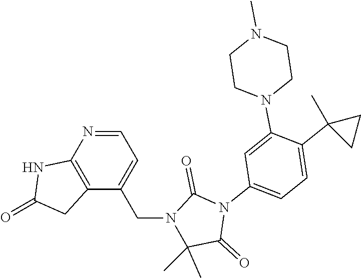 [SMILES: CN1CCN(c2cc(N3C(=O)N(Cc4ccnc5c4CC(=O)N5)C(C)(C)C3=O)ccc2C2(C)CC2)CC1] | 5,5-dimethyl-3-[4-(1-methylcyclopropyl)-3-(4- methyl-1-piperazinyl)phenyl]-1-[(2-oxo-1,7- diaza-4-indanyl)methyl]-2,4-imidazolidinedione; |
| 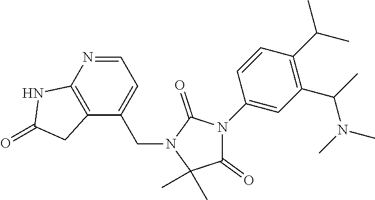 [SMILES: CC(C)c1ccc(N2C(=O)N(Cc3ccnc4c3CC(=O)N4)C(C)(C)C2=O)cc1C(C)N(C)C] | 3-{3-[1-(dimethylamino)ethyl]-4-cumenyl}-5,5- dimethyl-1-[(2-oxo-1,7-diaza-4-indanyl)methyl]- 2,4-imidazolidinedione; |
| 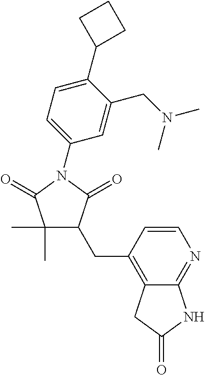 [SMILES: CN(C)Cc1cc(N2C(=O)C(Cc3ccnc4c3CC(=O)N4)C(C)(C)C2=O)ccc1C1CCC1] | 3-{4-cyclobutyl-3- [(dimethylamino)methyl]phenyl}-5,5-dimethyl- 1-[(2-oxo-1,7-diaza-4-indanyl)methyl]-2.4- imidazolidinedione; |
| 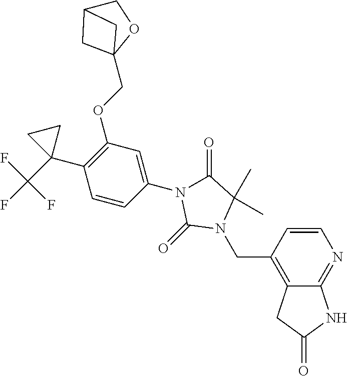 [SMILES: CC1(C)C(=O)N(c2ccc(C3(C(F)(F)F)CC3)c(OCC34CC(CO3)C4)c2)C(=O)N1Cc1ccnc2c1CC(=O)N2] | 5,5-dimethyl-3-(3-{(2-oxabicyclo[2.1.1]hex-1- yl)methoxy}-4-[1- (trifluoromethyl)cyclopropyl]phenyl)-1-[(2-oxo- 1,7-diaza-4-indanyl)methyl1-2,4- imidazolidinedione; |
| 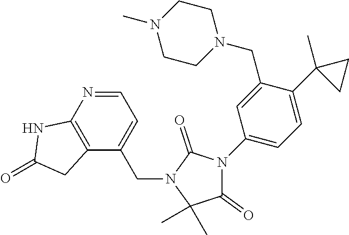 [SMILES: CN1CCN(Cc2cc(N3C(=O)N(Cc4ccnc5c4CC(=O)N5)C(C)(C)C3=O)ccc2C2(C)CC2)CC1] | 5,5-dimethyl-3-{4-(1-methylcyclopropyl)-3-[(4- methyl-1-piperazinyl)methyl]phenyl}-1-[(2-oxo- 1,7-diaza-4-indanyl)methyl]-2,4- imidazolidinedione; |
| 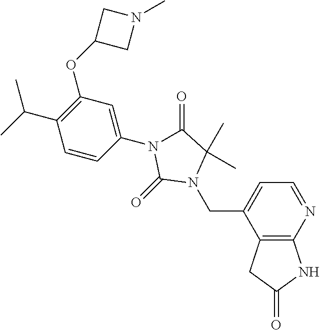 [SMILES: CC(C)c1ccc(N2C(=O)N(Cc3ccnc4c3CC(=O)N4)C(C)(C)C2=O)cc1OC1CN(C)C1] | 5.5-dimethyl-3-[3-(1-methyl-3-azetidinyloxy)-4- cumenyl]-1-[(2-oxo-1,7-diaza-4-indanyl)methyl]- 2,4-imidazolidinedione; |
| 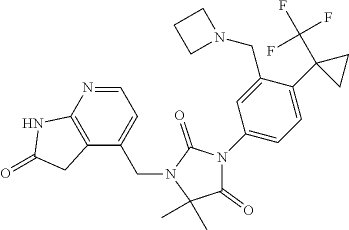 [SMILES: CC1(C)C(=O)N(c2ccc(C3(C(F)(F)F)CC3)c(CN3CCC3)c2)C(=O)N1Cc1ccnc2c1CC(=O)N2] | 3-{3-[(1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 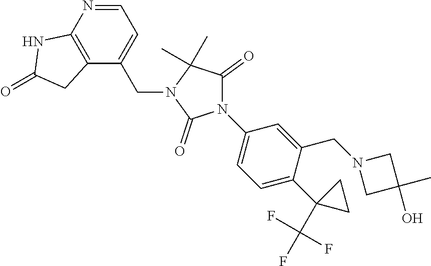 [SMILES: CC1(O)CN(Cc2cc(N3C(=O)N(Cc4ccnc5c4CC(=O)N5)C(C)(C)C3=O)ccc2C2(C(F)(F)F)CC2)C1] | 3-{3-[(3-hydroxy-3-methyl-1-azetidinyl)methyl]- 4-[1-(trifluoromethyl)cyclopropyl]phenyl}-5,5- dimethyl-1-[(2-oxo-1,7-diaza-4-indanyl)methyl]- 2.4-imidazolidinedione; |
| 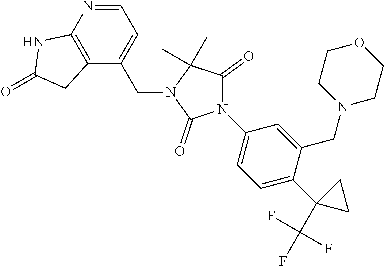 [SMILES: CC1(C)C(=O)N(c2ccc(C3(C(F)(F)F)CC3)c(CN3CCOCC3)c2)C(=O)N1Cc1ccnc2c1CC(=O)N2] | 5.5-dimethyl-3-{3-(morpholinomethyl)-4-[1- (trifluoromethyl)cyclopropyl]phenyl}-1-[(2-oxo- 1,7-diaza-4-indanyl)methyl1-2,4- imidazolidinedione; |
| 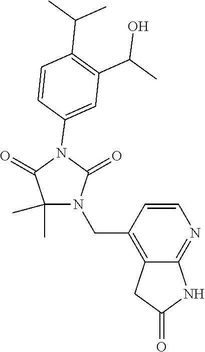 [SMILES: CC(C)c1ccc(N2C(=O)N(Cc3ccnc4c3CC(=O)N4)C(C)(C)C2=O)cc1C(C)O] | 3-[3-(1-hydroxyethyl)-4-cumenyl]-5,5-dimethyl- 1-[(2-oxo-1,7-diaza-4-indanyl)methyl]-2,4- imidazolidinedione; |
| 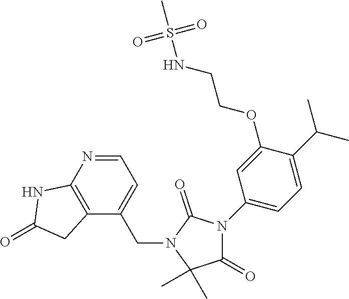 [SMILES: CC(C)c1ccc(N2C(=O)N(Cc3ccnc4c3CC(=O)N4)C(C)(C)C2=O)cc1OCCNS(C)(=O)=O] | 3-{3-[2-(mesylamino)ethoxy]-4-cumenyl}-5,5- dimethyl-1-[(2-oxo-1,7-diaza-4-indanyl)methyl]- 2.4-imidazolidinedione; |
| 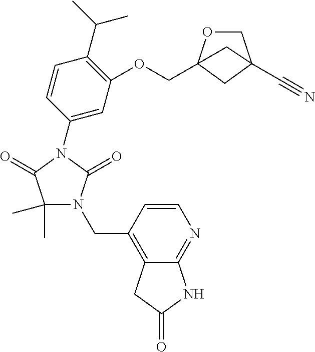 [SMILES: CC(C)c1ccc(N2C(=O)N(Cc3ccnc4c3CC(=O)N4)C(C)(C)C2=O)cc1OCC12CC(C#N)(CO1)C2] | 1-[(5-{4,4-dimethyl-2,5-dioxo-3-[(2-oxo-1,7- diaza-4-indanyl)methyl]-1-imidazolidinyl}-2- cumenyloxy)methyl]-2-oxabicyclo[2.1.1]hexane- 4-carbonitrile; |
| 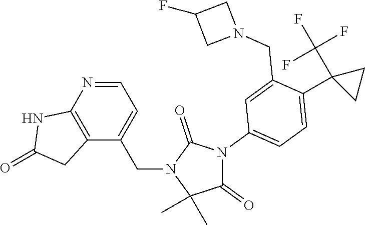 [SMILES: CC1(C)C(=O)N(c2ccc(C3(C(F)(F)F)CC3)c(CN3CC(F)C3)c2)C(=O)N1Cc1ccnc2c1CC(=O)N2] | 3-{3-[(3-fluoro-1-azetidinyl)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 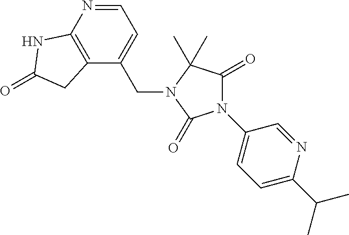 [SMILES: CC(C)c1ccc(N2C(=O)N(Cc3ccnc4c3CC(=O)N4)C(C)(C)C2=O)cn1] | 3-(6-isopropyl-3-pyridyl)-5,5-dimethyl-1-[(2- oxo-1,7-diaza-4-indanyl)methyl]-2,4- imidazolidinedione; |
| 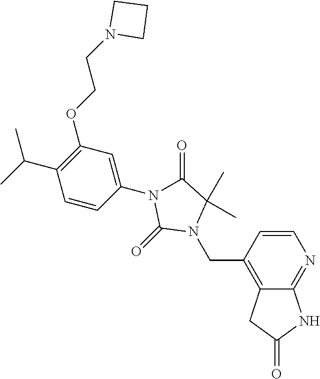 [SMILES: CC(C)c1ccc(N2C(=O)N(Cc3ccnc4c3CC(=O)N4)C(C)(C)C2=O)cc1OCCN1CCC1] | 3-{3-[2-(1-azetidinyl)ethoxy]-4-cumenyl}-5,5- dimethyl-1-[(2-oxo-1,7-diaza-4-indanyl)methyl]- 2,4-imidazolidinedione; |
| 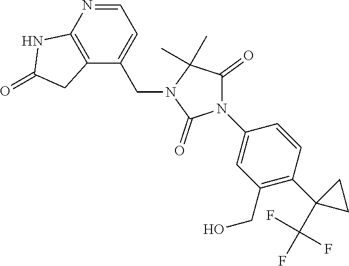 [SMILES: CC1(C)C(=O)N(c2ccc(C3(C(F)(F)F)CC3)c(CO)c2)C(=O)N1Cc1ccnc2c1CC(=O)N2] | 3-{3-(hydroxymethyl)-4-[1- (trifluoromethyl)cyclopropyl]phenyl}-5,5- dimethyl-1-[(2-oxo-1,7-diaza-4-indanyl)methyl]- 2,4-imidazolidinedione; |
| 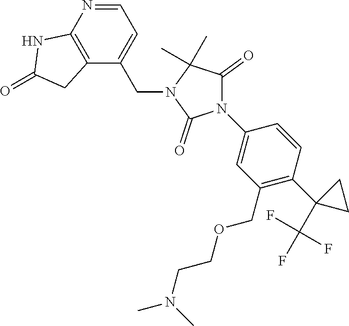 [SMILES: CN(C)CCOCc1cc(N2C(=O)N(Cc3ccnc4c3CC(=O)N4)C(C)(C)C2=O)ccc1C1(C(F)(F)F)CC1] | 3-(3-{[2-(dimethylamino)ethoxy]methyl}-4-[1- (trifluoromethyl)cyclopropyl]phenyl)-5,5- dimethyl-1-[(2-oxo-1,7-diaza-4-indanyl)methyl]- 2,4-imidazolidinedione; |
| 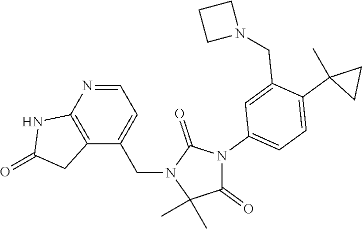 [SMILES: CC1(c2ccc(N3C(=O)N(Cc4ccnc5c4CC(=O)N5)C(C)(C)C3=O)cc2CN2CCC2)CC1] | 3-{3-[(1-azetidinyl)methyl]-4-(1- methylcyclopropyl)phenyl}-5,5-dimethyl-1-[(2- oxo-1,7-diaza-4-indanyl)methyl]-2,4- imidazolidinedione; |
| 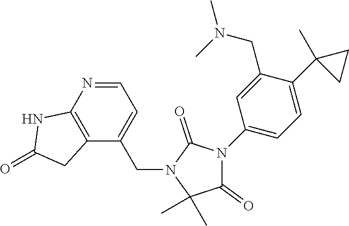 [SMILES: CN(C)Cc1cc(N2C(=O)N(Cc3ccnc4c3CC(=O)N4)C(C)(C)C2=O)ccc1C1(C)CC1] | 3-{3-[(dimethylamino)methyl]-4-(1- methylcyclopropyl)phenyl}-5,5-dimethyl-1-[(2- oxo-1,7-diaza-4-indanyl)methyl]-2,4- imidazolidinedione; |
| 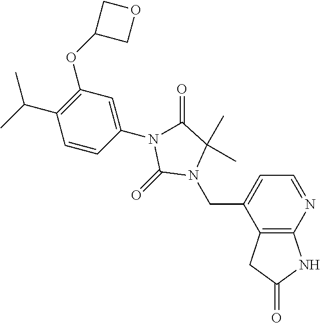 [SMILES: CC(C)c1ccc(N2C(=O)N(Cc3ccnc4c3CC(=O)N4)C(C)(C)C2=O)cc1OC1COC1] | 5,5-dimethyl-3-[3-(3-oxetanyloxy)-4-cumenyl]- 1-[(2-oxo-1,7-diaza-4-indanyl)methyl]-2,4- imidazolidinedione; |
| 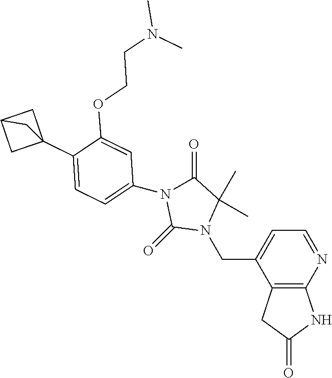 [SMILES: CN(C)CCOc1cc(N2C(=O)N(Cc3ccnc4c3CC(=O)N4)C(C)(C)C2=O)ccc1C12CC(C1)C2] | 3-{4-(bicyclo[1.1.1]pent-1-yl)-3-[2- (dimethylamino)ethoxy]phenyl}-5,5-dimethyl-1- [(2-oxo-1,7-diaza-4-indanyl)methyl]-2.4- imidazolidinedione; |
| 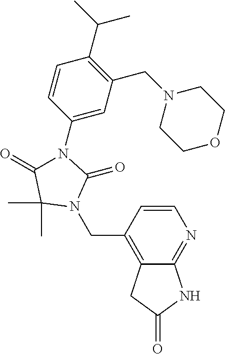 [SMILES: CC(C)c1ccc(N2C(=O)N(Cc3ccnc4c3CC(=O)N4)C(C)(C)C2=O)cc1CN1CCOCC1] | 5.5-dimethyl-3-[3-(morpholinomethyl)-4- cumenyl]-1-[(2-oxo-1,7-diaza-4-indanyl)methyl]- 2,4-imidazolidinedione; |
| 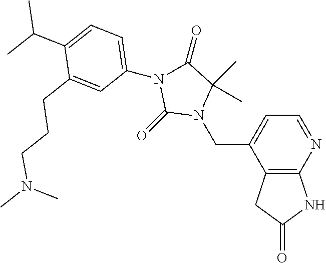 [SMILES: CC(C)c1ccc(N2C(=O)N(Cc3ccnc4c3CC(=O)N4)C(C)(C)C2=O)cc1CCCN(C)C] | 3-{3-[3-(dimethylamino)propyl]-4-cumenyl}-5,5- dimethyl-1-[(2-oxo-1,7-diaza-4-indanyl)methyl]- 2,4-imidazolidinedione; |
| 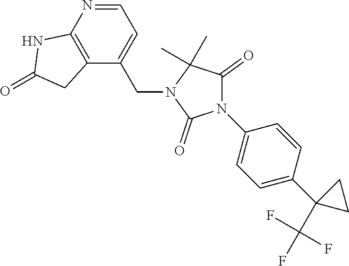 [SMILES: CC1(C)C(=O)N(c2ccc(C3(C(F)(F)F)CC3)cc2)C(=O)N1Cc1ccnc2c1CC(=O)N2] | 5,5-dimethyl-1-[(2-oxo-1-oxa-3,4-diaza-7- indenyl)methyl1-3-{p-[1- (trifluoromethyl)cyclopropyl]phenyl}-2,4- imidazolidinedione; |
| 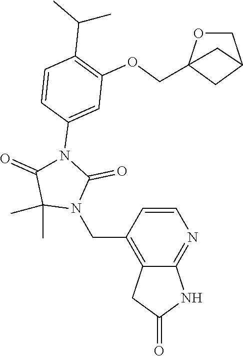 [SMILES: CC(C)c1ccc(N2C(=O)N(Cc3ccnc4c3CC(=O)N4)C(C)(C)C2=O)cc1OCC12CC(CO1)C2] | 5,5-dimethyl-3-(3-{(2-oxabicyclo[2.1.1]hex-1- yl)methoxy}-4-cumenyl)-1-[(2-oxo-1,7-diaza-4- indanyl)methyl]-2,4-imidazolidinedione; |
| 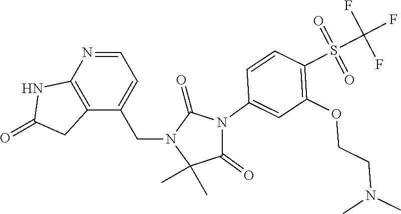 [SMILES: CN(C)CCOc1cc(N2C(=O)N(Cc3ccnc4c3CC(=O)N4)C(C)(C)C2=O)ccc1S(=O)(=O)C(F)(F)F] | 3-{3-[2-(dimethylamino)ethoxy]-4- (trifluoromesyl)phenyl}-5,5-dimethyl-1-[(2-oxo- 1,7-diaza-4-indanyl)methyl]-2,4- imidazolidinedione; |
| 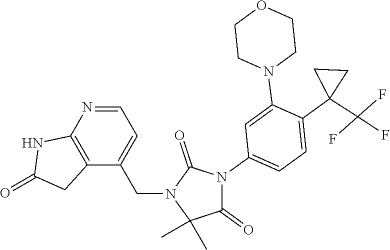 [SMILES: CC1(C)C(=O)N(c2ccc(C3(C(F)(F)F)CC3)c(N3CCOCC3)c2)C(=O)N1Cc1ccnc2c1CC(=O)N2] | 5,5-dimethyl-3-{3-morpholino-4-[1- (trifluoromethyl)cyclopropyl]phenyl}-1-[(2-oxo- 1,7-diaza-4-indanyl)methyl]-2.4- imidazolidinedione; |
| 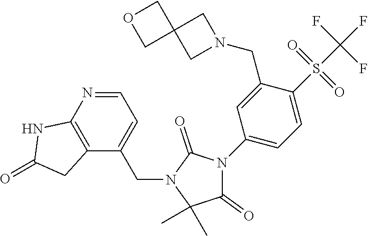 [SMILES: CC1(C)C(=O)N(c2ccc(S(=O)(=O)C(F)(F)F)c(CN3CC4(COC4)C3)c2)C(=O)N1Cc1ccnc2c1CC(=O)N2] | 5,5-dimethyl-3-(3-{(2-oxa-6-aza-6- spiro[3.3]heptyl)methyl}-4- (trifluoromesyl)phenyl)-1-[(2-oxo-1,7-diaza-4- indanyl)methyl]-2,4-imidazolidinedione; |
| 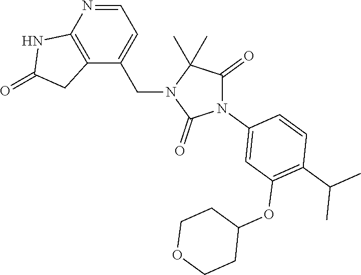 [SMILES: CC(C)c1ccc(N2C(=O)N(Cc3ccnc4c3CC(=O)N4)C(C)(C)C2=O)cc1OC1CCOCC1] | 5,5-dimethyl-1-[(2-oxo-1,7-diaza-4- indanyl)methyl]-3-[3-(tetrahydro-2H-pyran-4- yloxy)-4-cumenyl1-2,4-imidazolidinedione; |
| 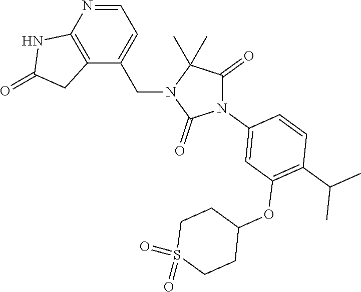 [SMILES: CC(C)c1ccc(N2C(=O)N(Cc3ccnc4c3CC(=O)N4)C(C)(C)C2=O)cc1OC1CCS(=O)(=O)CC1] | 3-[3-(1,1-dioxo-126-4-thianyloxy)-4-cumenyl]- 5,5-dimethyl-1-[(2-oxo-1,7-diaza-4- indanyl)methyl]-2,4-imidazolidinedione; |
| 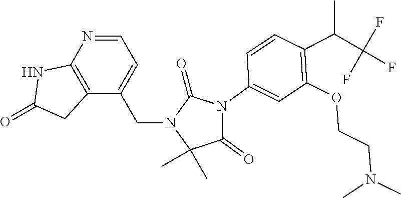 [SMILES: CC(c1ccc(N2C(=O)N(Cc3ccnc4c3CC(=O)N4)C(C)(C)C2=O)cc1OCCN(C)C)C(F)(F)F] | 3-{3-[2-(dimethylamino)ethoxy]-4-(2,2,2- trifluoro-1-methylethyl)phenyl}-5,5-dimethyl-1- [(2-oxo-1,7-diaza-4-indanyl)methyl]-2,4- imidazolidinedione; |
| 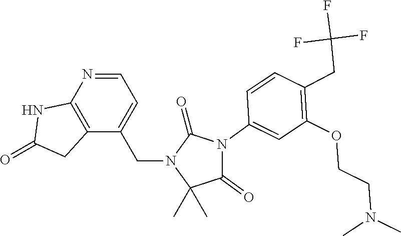 [SMILES: CN(C)CCOc1cc(N2C(=O)N(Cc3ccnc4c3CC(=O)N4)C(C)(C)C2=O)ccc1CC(F)(F)F] | 3-{3-[2-(dimethylamino)ethoxyl-4-(2,2,2- trifluoroethyl)phenyl}-5,5-dimethyl-1-[(2-oxo- 1,7-diaza-4-indanyl)methyl1-2,4- imidazolidinedione; |
| 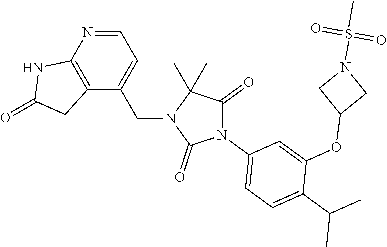 [SMILES: CC(C)c1ccc(N2C(=O)N(Cc3ccnc4c3CC(=O)N4)C(C)(C)C2=O)cc1OC1CN(S(C)(=O)=O)C1] | 3-[3-(1-mesyl-3-azetidinyloxy)-4-cumenyl]-5,5- dimethyl-1-[(2-oxo-1,7-diaza-4-indanyl)methyl]- 2,4-imidazolidinedione; |
| 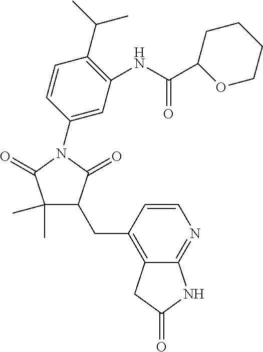 [SMILES: CC(C)c1ccc(N2C(=O)C(Cc3ccnc4c3CC(=O)N4)C(C)(C)C2=O)cc1NC(=O)C1CCCCO1] | N-(5-{4,4-dimethyl-2,5-dioxo-3-[(2-oxo-1,7- diaza-4-indanyl)methyl]-1-imidazolidinyl}-2- cumenyl)-tetrahydro-2H-pyran-2-carboxamide; |
| 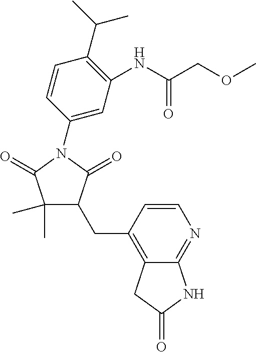 [SMILES: COCC(=O)Nc1cc(N2C(=O)C(Cc3ccnc4c3CC(=O)N4)C(C)(C)C2=O)ccc1C(C)C] | N-(5-{4,4-dimethyl-2,5-dioxo-3-[(2-oxo-1,7- diaza-4-indanyl)methyl]-1-imidazolidinyl}-2- cumenyl)methoxyacetamide; |
| 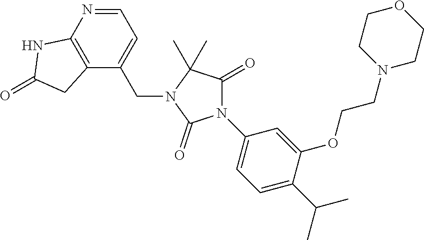 [SMILES: CC(C)c1ccc(N2C(=O)N(Cc3ccnc4c3CC(=O)N4)C(C)(C)C2=O)cc1OCCN1CCOCC1] | 5,5-dimethyl-3-[3-(2-morpholinoethoxy)-4- cumenyl]-1-[(2-oxo-1,7-diaza-4-indanyl)methyl]- 2,4-imidazolidinedione; |
| 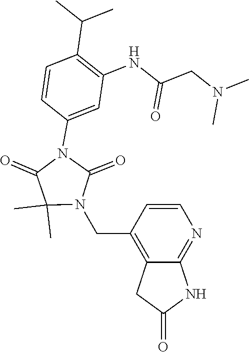 [SMILES: CC(C)c1ccc(N2C(=O)N(Cc3ccnc4c3CC(=O)N4)C(C)(C)C2=O)cc1NC(=O)CN(C)C] | N-(5-{4,4-dimethyl-2,5-dioxo-3-[(2-oxo-1,7- diaza-4-indanyl)methyl]-1-imidazolidinyl}-2- cumenyl)(dimethylamino)acetamide: |
| 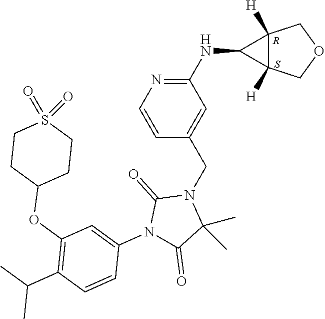 | 1-[(2-{(1R,5S,6r)-3-oxabicyclo[3.1.0]hex-6- ylamino}-4-pyridyl)methyl1-3-[3-(1,1-dioxo-126- 4-thianyloxy)-4-cumenyl]-5,5-dimethyl-2,4- imidazolidinedione; |
| 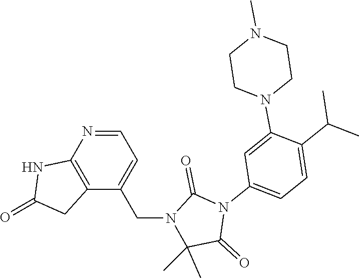 [SMILES: CC(C)c1ccc(N2C(=O)N(Cc3ccnc4c3CC(=O)N4)C(C)(C)C2=O)cc1N1CCN(C)CC1] | 5.5-dimethyl-3-[3-(4-methyl-1-piperazinyl)-4- cumenyl1-1-[(2-oxo-1,7-diaza-4-indanyl)methyl]- 2.4-imidazolidinedione; |
| 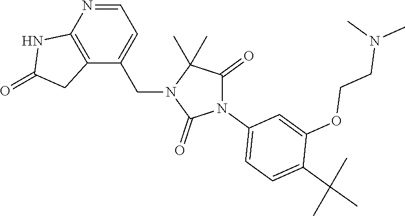 [SMILES: CN(C)CCOc1cc(N2C(=O)N(Cc3ccnc4c3CC(=O)N4)C(C)(C)C2=O)ccc1C(C)(C)C] | 3-{4-(tert-butyl)-3-[2- (dimethylamino)ethoxy]phenyl}-5,5-dimethyl-1- [(2-oxo-1,7-diaza-4-indanyl)methyl]-2.4- imidazolidinedione; |
| 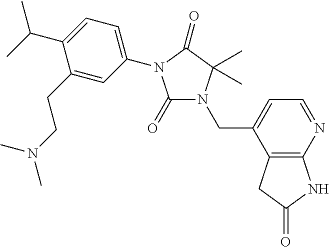 [SMILES: CC(C)c1ccc(N2C(=O)N(Cc3ccnc4c3CC(=O)N4)C(C)(C)C2=O)cc1CCN(C)C] | 3-{3-[2-(dimethylamino)ethyl]-4-cumenyl}-5,5- dimethyl-1-[(2-oxo-1,7-diaza-4-indanyl)methyl]- 2,4-imidazolidinedione; |
| 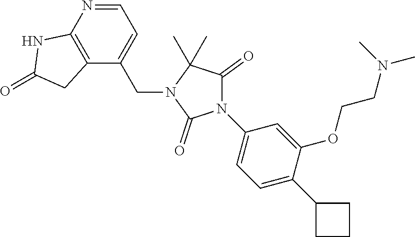 [SMILES: CN(C)CCOc1cc(N2C(=O)N(Cc3ccnc4c3CC(=O)N4)C(C)(C)C2=O)ccc1C1CCC1] | 3-{4-cyclobutyl-3-[2- (dimethylamino)ethoxy]phenyl}-5,5-dimethyl-1- [(2-oxo-1,7-diaza-4-indanyl)methyl]-2,4- imidazolidinedione; |
| 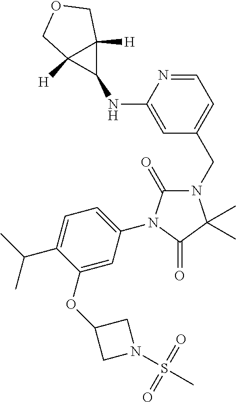 [SMILES: CC(C)c1ccc(N2C(=O)N(Cc3ccnc(N[C@H]4[C@@H]5COC[C@@H]54)c3)C(C)(C)C2=O)cc1OC1CN(S(C)(=O)=O)C1] | 1-[(2-{(1R,5S,6r)-3-oxabicyclo[3.1.0]hex-6- ylamino}-4-pyridyl)methyl]-3-[3-(1-mesyl-3- azetidinyloxy)-4-cumenyl]-5,5-dimethyl-2,4- imidazolidinedione; |
| 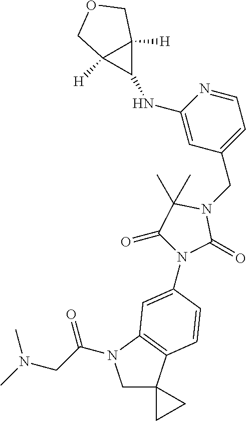 | 1-[(2-{(1R,5S,6r)-3-oxabicyclo[3.1.0]hex-6- ylamino}-4-pyridyl)methyl1-3-{1′-[2- (dimethylamino)acetyl]spiro[cyclopropane-1,3′- indolin]-6′-yl}-5,5-dimethyl-2,4- imidazolidinedione; |
| 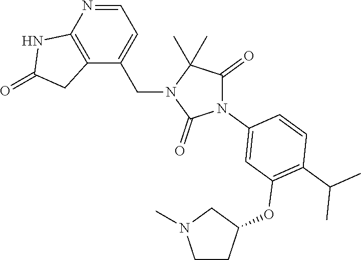 [SMILES: CC(C)c1ccc(N2C(=O)N(Cc3ccnc4c3CC(=O)N4)C(C)(C)C2=O)cc1O[C@@H]1CCN(C)C1] | 3-{3-[(R)-1-methyl-3-pyrrolidinyloxyl-4- cumenyl}-5,5-dimethyl-1-[(2-oxo-1,7-diaza-4- indanyl)methyl]-2.4-imidazolidinedione; |
| 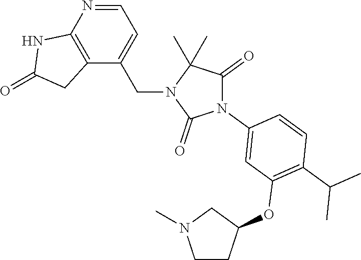 [SMILES: CC(C)c1ccc(N2C(=O)N(Cc3ccnc4c3CC(=O)N4)C(C)(C)C2=O)cc1O[C@H]1CCN(C)C1] | 3-{3-[(S)-1-methyl-3-pyrrolidinyloxy]-4- cumenyl}-5,5-dimethyl-1-[(2-oxo-1,7-diaza-4- indanyl)methyl]-2,4-imidazolidinedione; |
| 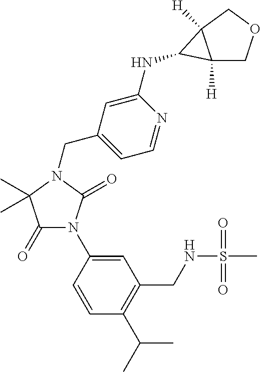 | 1-[(2-{(1R,5S,6r)-3-oxabicyclo[3.1.0]hex-6- ylamino}-4-pyridyl)methyl1-3-{3- [(mesylamino)methyl]-4-cumenyl}-5,5-dimethyl- 2,4-imidazolidinedione; |
| 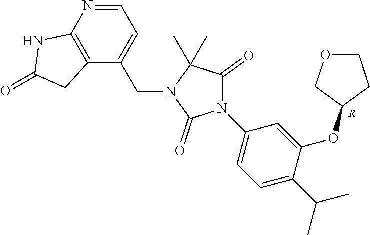 | 3-{3-[(R)-tetrahydro-3-furyloxy]-4-cumenyl}- 5,5-dimethyl-1-[(2-oxo-1,7-diaza-4- indanyl)methyl]-2,4-imidazolidinedione; |
| 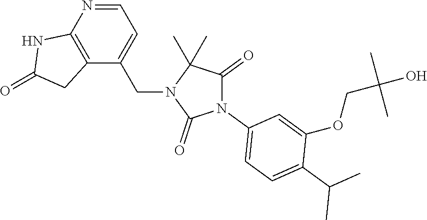 [SMILES: CC(C)c1ccc(N2C(=O)N(Cc3ccnc4c3CC(=O)N4)C(C)(C)C2=O)cc1OCC(C)(C)O] | 3-[3-(2-hydroxy-2-methylpropoxy)-4-cumenyl]- 5,5-dimethyl-1-[(2-oxo-1,7-diaza-4- indanyl)methyl]-2,4-imidazolidinedione; |
| 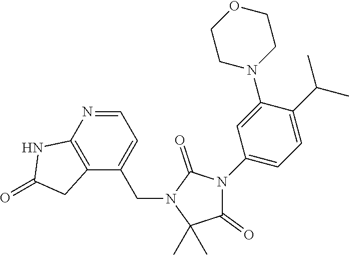 [SMILES: CC(C)c1ccc(N2C(=O)N(Cc3ccnc4c3CC(=O)N4)C(C)(C)C2=O)cc1N1CCOCC1] | 5,5-dimethyl-3-(3-morpholino-4-cumenyl)-1-[(2- oxo-1,7-diaza-4-indanyl)methyl]-2,4- imidazolidinedione; |
| 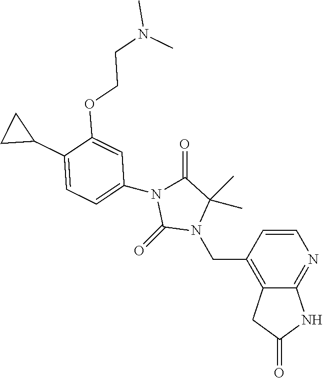 [SMILES: CN(C)CCOc1cc(N2C(=O)N(Cc3ccnc4c3CC(=O)N4)C(C)(C)C2=O)ccc1C1CC1] | 3-{4-cyclopropyl-3-[2- (dimethylamino)ethoxy]phenyl}-5,5-dimethyl-1- [(2-oxo-1,7-diaza-4-indanyl)methyl]-2.4- imidazolidinedione; |
| 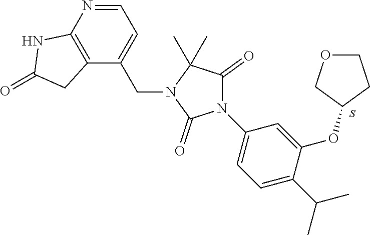 | 3-{3-[(S)-tetrahydro-3-furyloxyl-4-cumenyl}- 5,5-dimethyl-1-[(2-oxo-1,7-diaza-4- indanyl)methyl]-2,4-imidazolidinedione; |
| 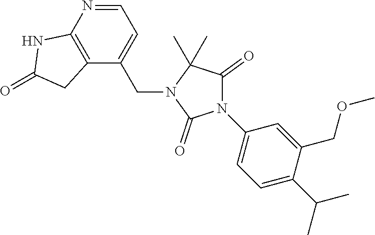 [SMILES: COCc1cc(N2C(=O)N(Cc3ccnc4c3CC(=O)N4)C(C)(C)C2=O)ccc1C(C)C] | 3-[3-(methoxymethyl)-4-cumenyl]-5,5-dimethyl- 1-[(2-oxo-1,7-diaza-4-indanyl)methyl]-2,4- imidazolidinedione; |
| 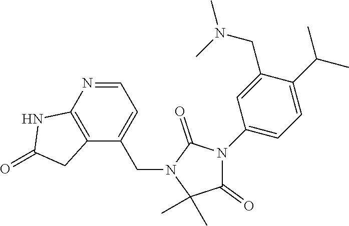 [SMILES: CC(C)c1ccc(N2C(=O)N(Cc3ccnc4c3CC(=O)N4)C(C)(C)C2=O)cc1CN(C)C] | 3-{3-[(dimethylamino)methyl]-4-cumenyl}-5,5- dimethyl-1-[(2-oxo-1,7-diaza-4-indanyl)methyl]- 2,4-imidazolidinedione; |
| 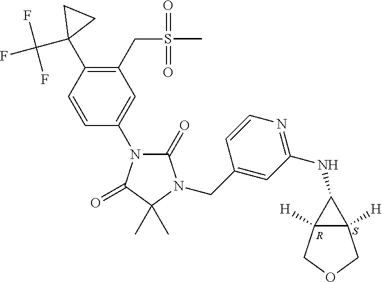 | 1-[(2-{(1R,5S,6r)-3-oxabicyclo[3.1.0]hex-6- ylamino}-4-pyridyl)methyl]-3-{3-(mesylmethyl)- 4-[1-(trifluoromethyl)cyclopropyl]phenyl}-5,5- dimethyl-2.4-imidazolidinedione; |
| 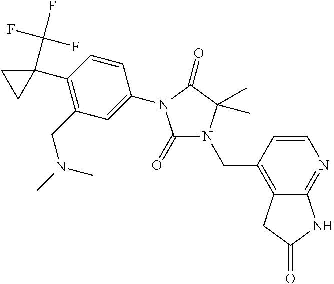 [SMILES: CN(C)Cc1cc(N2C(=O)N(Cc3ccnc4c3CC(=O)N4)C(C)(C)C2=O)ccc1C1(C(F)(F)F)CC1] | 3-{3-[(dimethylamino)methyl]-4-[1- (trifluoromethyl)cyclopropyl]phenyl}-5,5- dimethyl-1-[(2-oxo-1,7-diaza-4-indanyl)methyl]- 2,4-imidazolidinedione; |
| 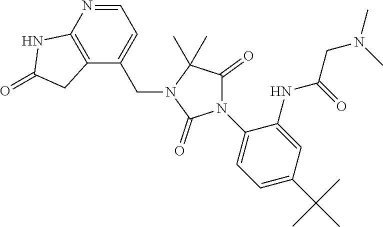 [SMILES: CN(C)CC(=O)Nc1cc(C(C)(C)C)ccc1N1C(=O)N(Cc2ccnc3c2CC(=O)N3)C(C)(C)C1=O] | N-[5-(tert-butyl)-2-{4,4-dimethyl-2,5-dioxo-3- [(2-oxo-1,7-diaza-4-indanyl)methyl]-1- imidazolidinyl}phenyl](dimethylamino)acetamide; |
| 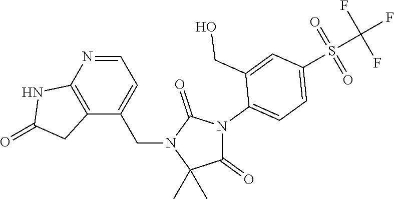 [SMILES: CC1(C)C(=O)N(c2ccc(S(=O)(=O)C(F)(F)F)cc2CO)C(=O)N1Cc1ccnc2c1CC(=O)N2] | 3-[2-(hydroxymethyl)-4-(trifluoromesyl)phenyl]- 5,5-dimethyl-1-[(2-oxo-1,7-diaza-4- indanyl)methyl]-2.4-imidazolidinedione; |
| 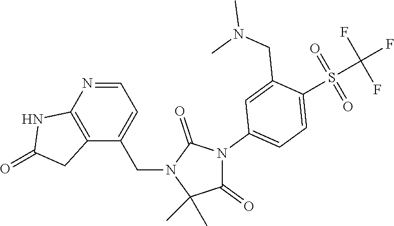 [SMILES: CN(C)Cc1cc(N2C(=O)N(Cc3ccnc4c3CC(=O)N4)C(C)(C)C2=O)ccc1S(=O)(=O)C(F)(F)F] | 3-{3-[(dimethylamino)methyl]-4- (trifluoromesyl)phenyl}-5,5-dimethyl-1-[(2-oxo- 1,7-diaza-4-indanyl)methyl]-2,4- imidazolidinedione; |
| 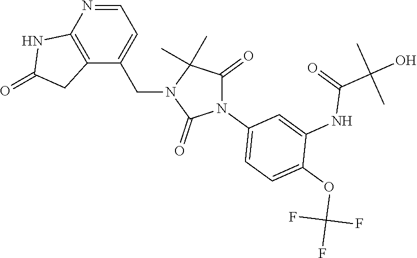 [SMILES: CC(C)(O)C(=O)Nc1cc(N2C(=O)N(Cc3ccnc4c3CC(=O)N4)C(C)(C)C2=O)ccc1OC(F)(F)F] | N-(5-{4,4-dimethyl-2,5-dioxo-3-[(2-oxo-1,7- diaza-4-indanyl)methyl]-1-imidazolidinyl}-2- trifluoromethoxyphenyl)2-hydroxy-2- methylpropionamide; |
| 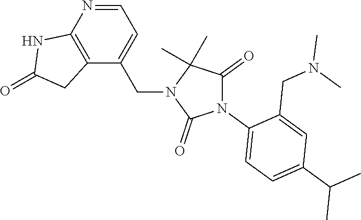 [SMILES: CC(C)c1ccc(N2C(=O)N(Cc3ccnc4c3CC(=O)N4)C(C)(C)C2=O)c(CN(C)C)c1] | 3-{2-[(dimethylamino)methyl]-4-cumenyl}-5,5- dimethyl-1-[(2-oxo-1,7-diaza-4-indanyl)methyl]- 2,4-imidazolidinedione; |
| 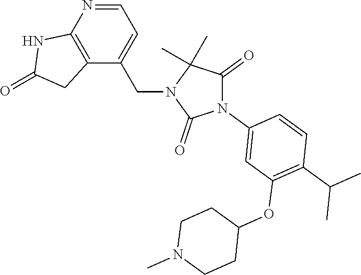 [SMILES: CC(C)c1ccc(N2C(=O)N(Cc3ccnc4c3CC(=O)N4)C(C)(C)C2=O)cc1OC1CCN(C)CC1] | 5,5-dimethyl-3-[3-(1-methyl-4-piperidyloxy)-4- cumenyl]-1-[(2-oxo-1,7-diaza-4-indanyl)methyl]- 2,4-imidazolidinedione; |
| 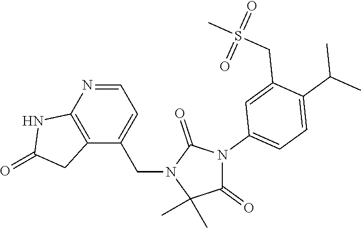 [SMILES: CC(C)c1ccc(N2C(=O)N(Cc3ccnc4c3CC(=O)N4)C(C)(C)C2=O)cc1CS(C)(=O)=O] | 3-[3-(mesylmethyl)-4-cumenyl]-5,5-dimethyl-1- [(2-oxo-1,7-diaza-4-indanyl)methyl]-2,4- imidazolidinedione; |
| 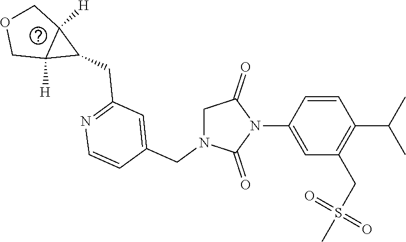 [SMILES: CC(C)c1ccc(N2C(=O)CN(Cc3ccnc(C[C@H]4[C@@H]5COC[C@@H]54)c3)C2=O)cc1CS(C)(=O)=O] | 1-[(2-{(1R,5S.6r)-3-oxabicyclo[3.1.0]hex-6- ylamino}-4-pyridyl)methyl]-3-[3-(mesylmethyl)- 4-cumenyl]-5,5-dimethyl-2,4-imidazolidinedione; |
| 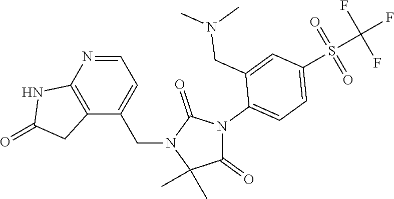 [SMILES: CN(C)Cc1cc(S(=O)(=O)C(F)(F)F)ccc1N1C(=O)N(Cc2ccnc3c2CC(=O)N3)C(C)(C)C1=O] | 3-{2-[(dimethylamino)methyl]-4- (trifluoromesyl)phenyl }-5,5-dimethyl-1-[(2-oxo- 1,7-diaza-4-indanyl)methyl]-2.4- imidazolidinedione; |
| 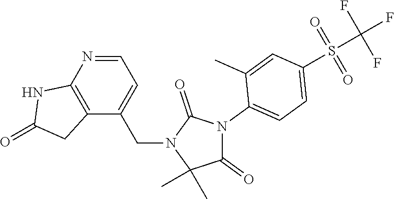 [SMILES: Cc1cc(S(=O)(=O)C(F)(F)F)ccc1N1C(=O)N(Cc2ccnc3c2CC(=O)N3)C(C)(C)C1=O] | 5,5-dimethyl-1-[(2-oxo-1,7-diaza-4- indanyl)methyl]-3-[4-(trifluoromesyl)-2-tolyl]- 2.4-imidazolidinedione; |
| 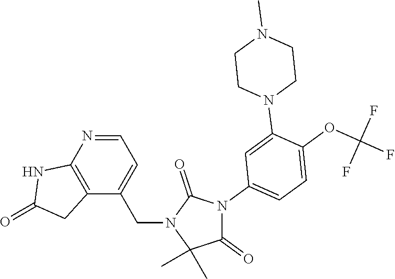 [SMILES: CN1CCN(c2cc(N3C(=O)N(Cc4ccnc5c4CC(=O)N5)C(C)(C)C3=O)ccc2OC(F)(F)F)CC1] | 5,5-dimethyl-3-[3-(4-methyl-1-piperazinyl)-4- trifluoromethoxyphenyl]-1-[(2-oxo-1,7-diaza-4- indanyl)methyl]-2.4-imidazolidinedione; |
| 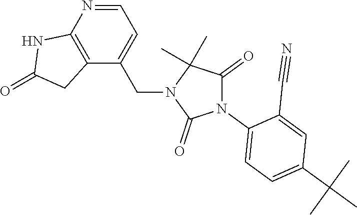 [SMILES: CC(C)(C)c1ccc(N2C(=O)N(Cc3ccnc4c3CC(=O)N4)C(C)(C)C2=O)c(C#N)c1] | 5-(tert-butyl)-2-{4.4-dimethyl-2,5-dioxo-3-[(2- oxo-1,7-diaza-4-indanyl)methyl]-1- imidazolidinyl}benzonitrile; |
| 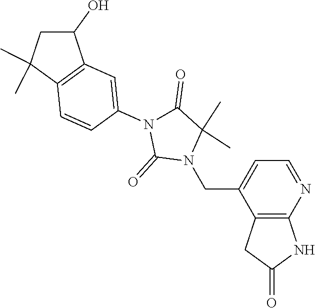 [SMILES: CC1(C)CC(O)c2cc(N3C(=O)N(Cc4ccnc5c4CC(=O)N5)C(C)(C)C3=O)ccc21] | 3-(3-hydroxy-1,1-dimethyl-5-indanyl)-5,5- dimethyl-1-[(2-oxo-1,7-diaza-4-indanyl)methyl]- 2,4-imidazolidinedione; |
| 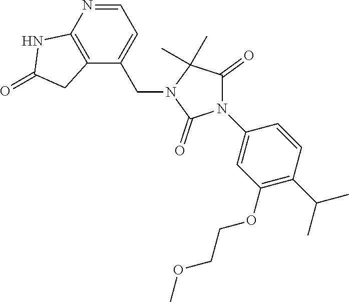 [SMILES: COCCOc1cc(N2C(=O)N(Cc3ccnc4c3CC(=O)N4)C(C)(C)C2=O)ccc1C(C)C] | 3-[3-(2-methoxyethoxy)-4-cumenyl]-5,5- dimethyl-1-[(2-oxo-1,7-diaza-4-indanyl)methyl]- 2.4-imidazolidinedione; |
| 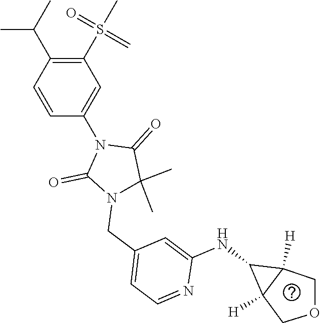 [SMILES: C=S(C)(=O)c1cc(N2C(=O)N(Cc3ccnc(N[C@H]4[C@@H]5COC[C@@H]54)c3)C(C)(C)C2=O)ccc1C(C)C] | 1-[(2-{(1R,5S,6r)-3-oxabicyclo[3.1.0]hex-6- ylamino}-4-pyridyl)methyl]-3-(3-mesyl-4- cumenyl)-5,5-dimethyl-2,4-imidazolidinedione; |
| 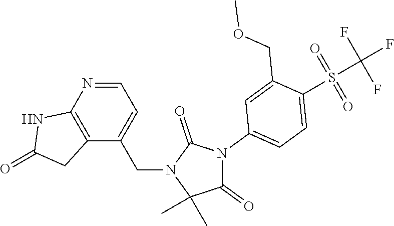 [SMILES: COCc1cc(N2C(=O)N(Cc3ccnc4c3CC(=O)N4)C(C)(C)C2=O)ccc1S(=O)(=O)C(F)(F)F] | 3-[3-(methoxymethyl)-4-(trifluoromesyl)phenyl]- 5,5-dimethyl-1-[(2-oxo-1,7-diaza-4- indanyl)methyl]-2.4-imidazolidinedione; |
| 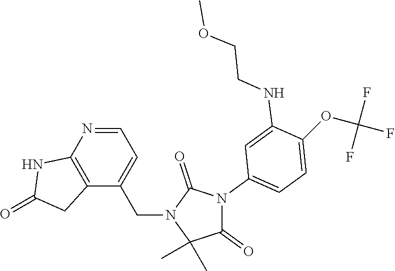 [SMILES: COCCNc1cc(N2C(=O)N(Cc3ccnc4c3CC(=O)N4)C(C)(C)C2=O)ccc1OC(F)(F)F] | 3-[3-(2-methoxyethylamino)-4- trifluoromethoxyphenyl]-5,5-dimethyl-1-[(2-oxo- 1,7-diaza-4-indanyl)methyl]-2,4- imidazolidinedione; |
| 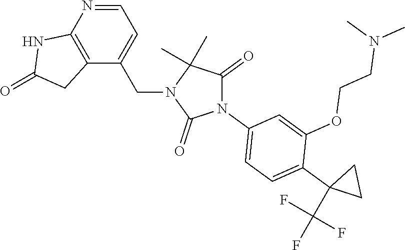 [SMILES: CN(C)CCOc1cc(N2C(=O)N(Cc3ccnc4c3CC(=O)N4)C(C)(C)C2=O)ccc1C1(C(F)(F)F)CC1] | 3-{3-[2-(dimethylamino)ethoxyl-4-[1- (trifluoromethyl)cyclopropyl]phenyl}-5,5- dimethyl-1-[(2-oxo-1,7-diaza-4-indanyl)methyl]- 2,4-imidazolidinedione; |
| 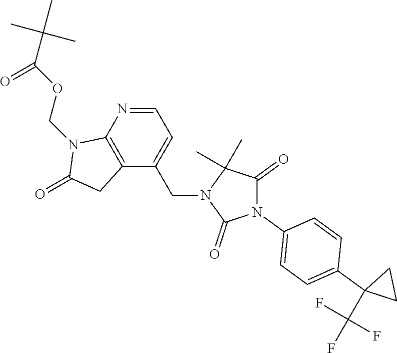 [SMILES: CC(C)(C)C(=O)OCN1C(=O)Cc2c(CN3C(=O)N(c4ccc(C5(C(F)(F)F)CC5)cc4)C(=O)C3(C)C)ccnc21] | {4-[(5,5-dimethyl-2,4-dioxo-3-{p-[1- (trifluoromethyl)cyclopropyl]phenyl}-1- imidazolidinyl)methyl1-2-oxo-1,7-diaza-1- indanyl}methyl 2,2-dimethylpropionate; |
| 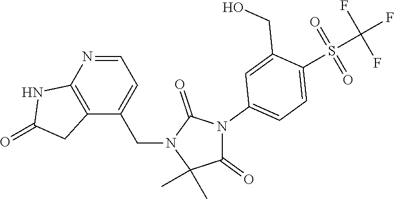 [SMILES: CC1(C)C(=O)N(c2ccc(S(=O)(=O)C(F)(F)F)c(CO)c2)C(=O)N1Cc1ccnc2c1CC(=O)N2] | 3-[3-(hydroxymethyl)-4-(trifluoromesyl)phenyl]- 5,5-dimethyl-1-[(2-oxo-1,7-diaza-4- indanyl)methyl]-2,4-imidazolidinedione; |
| 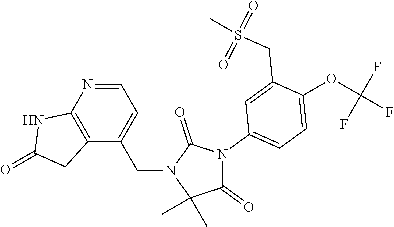 [SMILES: CC1(C)C(=O)N(c2ccc(OC(F)(F)F)c(CS(C)(=O)=O)c2)C(=O)N1Cc1ccnc2c1CC(=O)N2] | 3-[3-(mesylmethyl)-4-trifluoromethoxyphenyl]- 5,5-dimethyl-1-[(2-oxo-1,7-diaza-4- indanyl)methyl]-2,4-imidazolidinedione; |
| 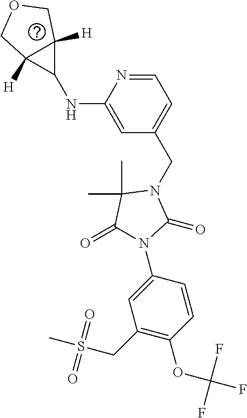 [SMILES: CC1(C)C(=O)N(c2ccc(OC(F)(F)F)c(CS(C)(=O)=O)c2)C(=O)N1Cc1ccnc(NC2[C@H]3COC[C@@H]23)c1] | 1-[(2-{(1R,5S)-3-oxabicyclo[3.1.0]hex-6- ylamino}-4-pyridyl)methyl]-3-[3-(mesylmethyl)- 4-trifluoromethoxyphenyl]-5,5-dimethyl-2,4- imidazolidinedione; |
| 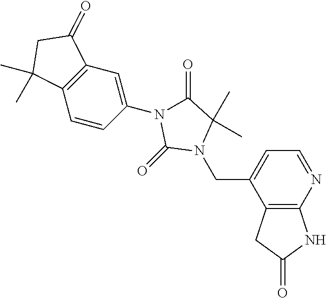 [SMILES: CC1(C)CC(=O)c2cc(N3C(=O)N(Cc4ccnc5c4CC(=O)N5)C(C)(C)C3=O)ccc21] | 3-(1,1-dimethyl-3-oxo-5-indanyl)-5,5-dimethyl- 1-[(2-oxo-1,7-diaza-4-indanyl)methyl]-2,4- imidazolidinedione; |
| 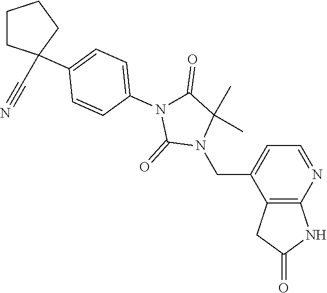 [SMILES: CC1(C)C(=O)N(c2ccc(C3(C#N)CCCC3)cc2)C(=O)N1Cc1ccnc2c1CC(=O)N2] | 1-(p-{4.4-dimethyl-2.5-dioxo-3-[(2-oxo-1,7- diaza-4-indanyl)methyl]-1- imidazolidinyl}phenyl)cyclopentanecarbonitrile; |
| 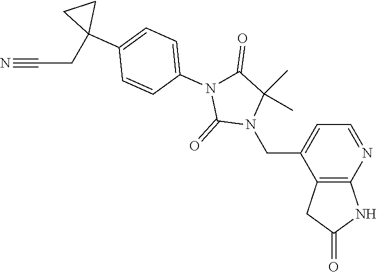 [SMILES: CC1(C)C(=O)N(c2ccc(C3(CC#N)CC3)cc2)C(=O)N1Cc1ccnc2c1CC(=O)N2] | [1-(p-{4,4-dimethyl-2,5-dioxo-3-[(2-oxo-1,7- diaza-4-indanyl)methyl]-1- imidazolidinyl}phenyl)cyclopropyl]acetonitrile; |
| 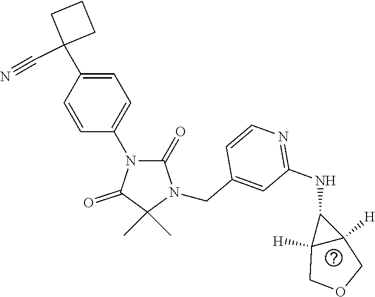 [SMILES: CC1(C)C(=O)N(c2ccc(C3(C#N)CCC3)cc2)C(=O)N1Cc1ccnc(N[C@H]2[C@@H]3COC[C@@H]32)c1] | 1-(p-{3-[(2-{(1R,5S,6r)-3-oxabicyclo[3.1.0]hex- 6-ylamino}-4-pyridyl)methyl]-4,4-dimethyl-2,5- dioxo-1- imidazolidinyl}phenyl)cyclobutanecarbonitrile; |
| 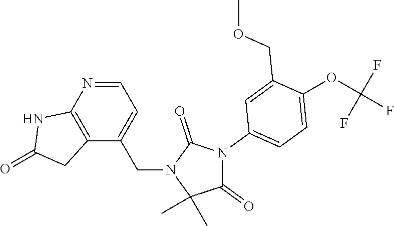 [SMILES: COCc1cc(N2C(=O)N(Cc3ccnc4c3CC(=O)N4)C(C)(C)C2=O)ccc1OC(F)(F)F] | 3-[3-(methoxymethyl)-4- trifluoromethoxyphenyl]-5,5-dimethyl-1-[(2-oxo- 1,7-diaza-4-indanyl)methyl]-2,4- imidazolidinedione; |
| 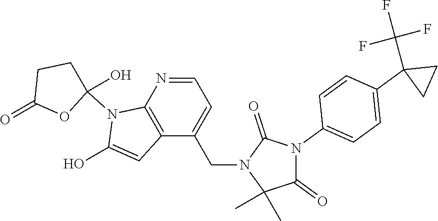 [SMILES: CC1(C)C(=O)N(c2ccc(C3(C(F)(F)F)CC3)cc2)C(=O)N1Cc1ccnc2c1cc(O)n2C1(O)CCC(=O)O1] | 1-{[2-hydroxy-1-(5-hydroxy-2-oxo-4,5-dihydro- 5-furyl)-1H-1,7-diazainden-4-yl]methyl}-5,5- dimethyl-3-{p-[1- (trifluoromethyl)cyclopropyl]phenyl}-2,4- imidazolidinedione; |
| 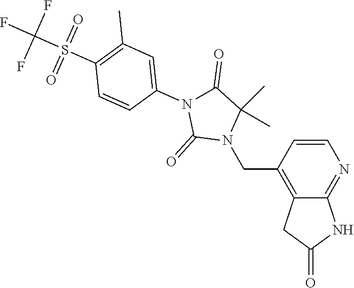 [SMILES: Cc1cc(N2C(=O)N(Cc3ccnc4c3CC(=O)N4)C(C)(C)C2=O)ccc1S(=O)(=O)C(F)(F)F] | 5,5-dimethyl-1-[(2-oxo-1,7-diaza-4- indanyl)methyl]-3-[4-(trifluoromesyl)-3-tolyl]- 2,4-imidazolidinedione; |
| 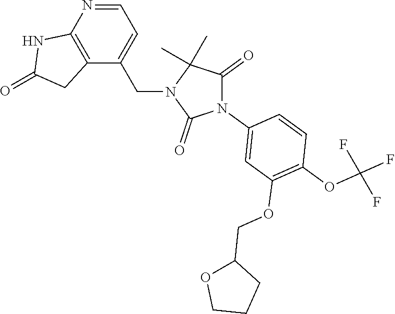 [SMILES: CC1(C)C(=O)N(c2ccc(OC(F)(F)F)c(OCC3CCCO3)c2)C(=O)N1Cc1ccnc2c1CC(=O)N2] | 5,5-dimethyl-1-[(2-oxo-1,7-diaza-4- indanyl)methyl]-3-{3-[(tetrahydro-2- furyl)methoxy]-4-trifluoromethoxyphenyl}-2,4- imidazolidinedione; |
| 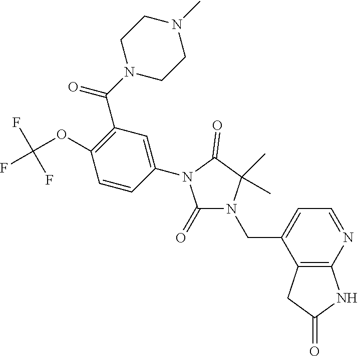 [SMILES: CN1CCN(C(=O)c2cc(N3C(=O)N(Cc4ccnc5c4CC(=O)N5)C(C)(C)C3=O)ccc2OC(F)(F)F)CC1] | 5,5-dimethyl-3-{3-[(4-methyl-1- piperazinyl)carbonyl]-4- trifluoromethoxyphenyl}-1-[(2-oxo-1,7-diaza-4- indanyl)methyl]-2,4-imidazolidinedione; |
| 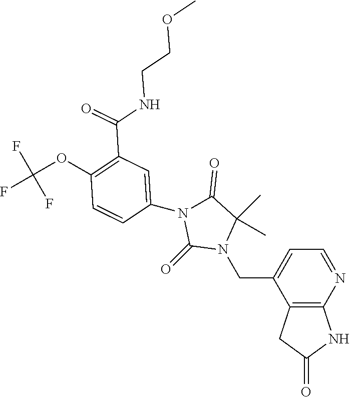 [SMILES: COCCNC(=O)c1cc(N2C(=O)N(Cc3ccnc4c3CC(=O)N4)C(C)(C)C2=O)ccc1OC(F)(F)F] | N-2-methoxyethyl5-{4,4-dimethyl-2,5-dioxo-3- [(2-oxo-1,7-diaza-4-indanyl)methyl]-1- imidazolidinyl}-2-trifluoroanisamide; |
| 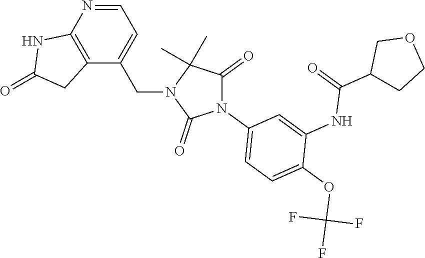 [SMILES: CC1(C)C(=O)N(c2ccc(OC(F)(F)F)c(NC(=O)C3CCOC3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-3-furamide; |
| 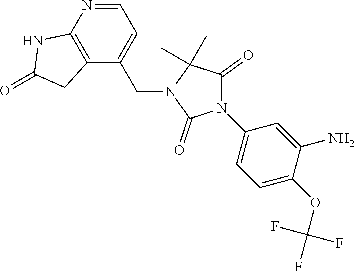 [SMILES: CC1(C)C(=O)N(c2ccc(OC(F)(F)F)c(N)c2)C(=O)N1Cc1ccnc2c1CC(=O)N2] | 3-(3-amino-4-trifluoromethoxyphenyl)-5,5- dimethyl-1-[(2-oxo-1,7-diaza-4-indanyl)methyl]- 2.4-imidazolidinedione; |
| 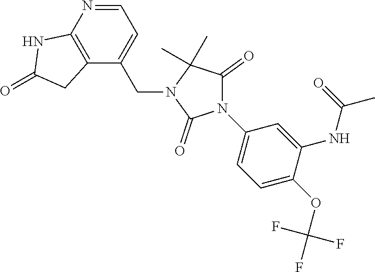 [SMILES: CC(=O)Nc1cc(N2C(=O)N(Cc3ccnc4c3CC(=O)N4)C(C)(C)C2=O)ccc1OC(F)(F)F] | N-(5-{4,4-dimethyl-2,5-dioxo-3-[(2-oxo-1,7- diaza-4-indanyl)methyl]-1-imidazolidinyl}-2- trifluoromethoxyphenyl)acetamide; |
| 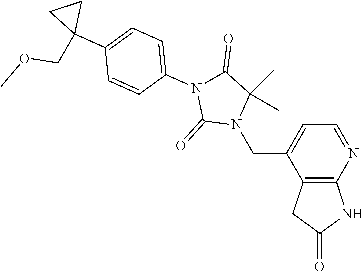 [SMILES: COCC1(c2ccc(N3C(=O)N(Cc4ccnc5c4CC(=O)N5)C(C)(C)C3=O)cc2)CC1] | 3-{p-[1-(methoxymethyl)cyclopropyl]phenyl}- 5,5-dimethyl-1-[(2-oxo-1,7-diaza-4- indanyl)methyl]-2.4-imidazolidinedione; |
| 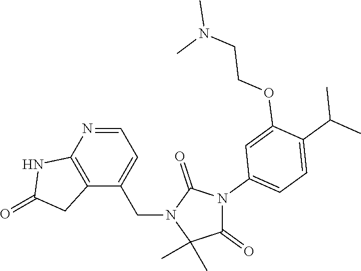 [SMILES: CC(C)c1ccc(N2C(=O)N(Cc3ccnc4c3CC(=O)N4)C(C)(C)C2=O)cc1OCCN(C)C] | 3-{3-[2-(dimethylamino)ethoxy]-4-cumenyl}- 5,5-dimethyl-1-[(2-oxo-1,7-diaza-4- indanyl)methyl]-2,4-imidazolidinedione; |
| 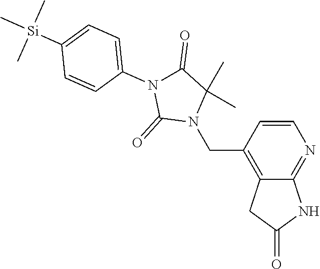 [SMILES: CC1(C)C(=O)N(c2ccc([Si](C)(C)C)cc2)C(=O)N1Cc1ccnc2c1CC(=O)N2] | 5,5-dimethyl-1-[(2-oxo-1,7-diaza-4- indanyl)methyl]-3-[p-(trimethylsilyl)phenyl]-2,4- imidazolidinedione; |
| 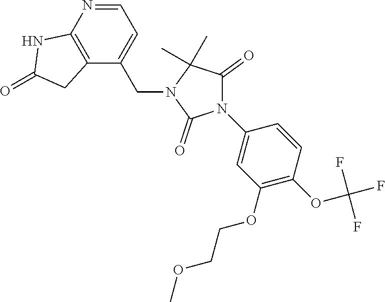 [SMILES: COCCOc1cc(N2C(=O)N(Cc3ccnc4c3CC(=O)N4)C(C)(C)C2=O)ccc1OC(F)(F)F] | 3-[3-(2-methoxyethoxy)-4- trifluoromethoxyphenyl]-5,5-dimethyl-1-[(2-oxo- 1,7-diaza-4-indanyl)methyl]-2,4- imidazolidinedione; |
| 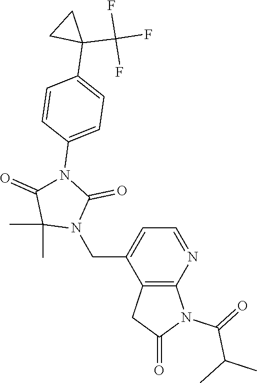 [SMILES: CC(C)C(=O)N1C(=O)Cc2c(CN3C(=O)N(c4ccc(C5(C(F)(F)F)CC5)cc4)C(=O)C3(C)C)ccnc21] | 1-[(1-isobutyryl-2-oxo-1,7-diaza-4- indanyl)methyl]-5,5-dimethyl-3-{p-[1- (trifluoromethyl)cyclopropyl]phenyl}-2,4- imidazolidinedione; |
| 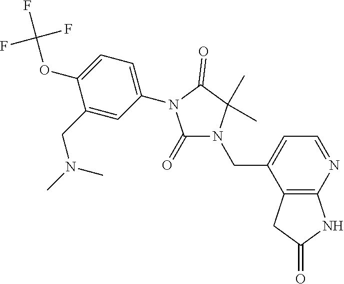 [SMILES: CN(C)Cc1cc(N2C(=O)N(Cc3ccnc4c3CC(=O)N4)C(C)(C)C2=O)ccc1OC(F)(F)F] | 3-{3-[(dimethylamino)methyl]-4- trifluoromethoxyphenyl}-5,5-dimethyl-1-[(2- oxo-1,7-diaza-4-indanyl)methyl]-2,4- imidazolidinedione; |
| 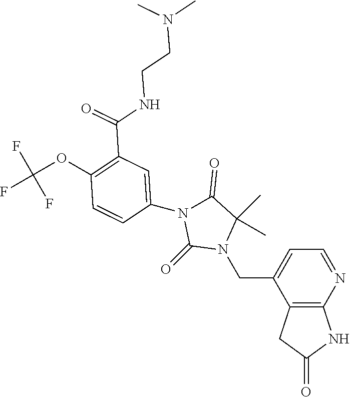 [SMILES: CN(C)CCNC(=O)c1cc(N2C(=O)N(Cc3ccnc4c3CC(=O)N4)C(C)(C)C2=O)ccc1OC(F)(F)F] | N-2-(dimethylamino)ethyl5-{4,4-dimethyl-2,5- dioxo-3-[(2-oxo-1,7-diaza-4-indanyl)methyl]-1- imidazolidinyl}-2-trifluoroanisamide; |
| 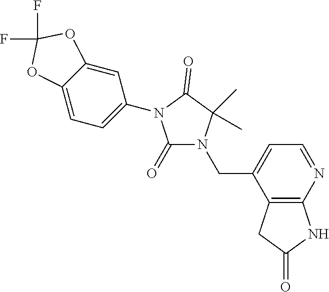 [SMILES: CC1(C)C(=O)N(c2ccc3c(c2)OC(F)(F)O3)C(=O)N1Cc1ccnc2c1CC(=O)N2] | 3-(2,2-difluoro-2H-1,3-benzodioxol-5-yl)-5,5- dimethyl-1-[(2-oxo-1,7-diaza-4-indanyl)methyl]- 2,4-imidazolidinedione; |
| 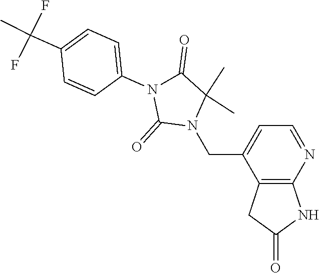 [SMILES: CC(F)(F)c1ccc(N2C(=O)N(Cc3ccnc4c3CC(=O)N4)C(C)(C)C2=O)cc1] | 3-[p-(1,1-difluoroethyl)phenyl]-5,5-dimethyl-1- [(2-oxo-1,7-diaza-4-indanyl)methyl]-2.4- imidazolidinedione; |
| 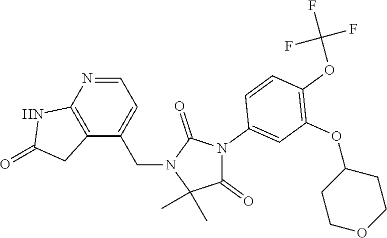 [SMILES: CC1(C)C(=O)N(c2ccc(OC(F)(F)F)c(OC3CCOCC3)c2)C(=O)N1Cc1ccnc2c1CC(=O)N2] | 5.5-dimethyl-1-[(2-oxo-1,7-diaza-4- indanyl)methyl]-3-[3-(tetrahydro-2H-pyran-4- yloxy)-4-trifluoromethoxyphenyl]-2,4- imidazolidinedione: |
| 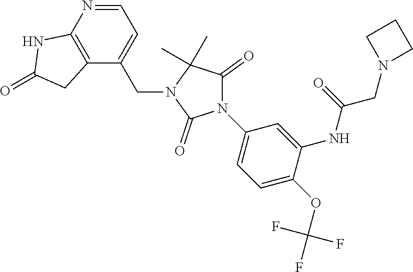 [SMILES: CC1(C)C(=O)N(c2ccc(OC(F)(F)F)c(NC(=O)CN3CCC3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)(1-azetidinyl)acetamide; |
| 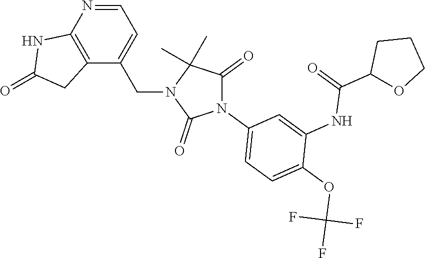 [SMILES: CC1(C)C(=O)N(c2ccc(OC(F)(F)F)c(NC(=O)C3CCCO3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2-furamide; |
| 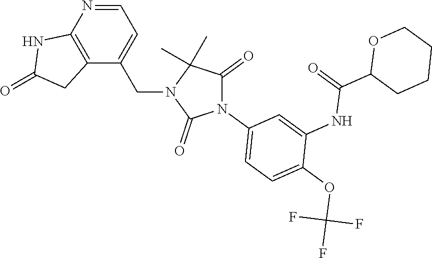 [SMILES: CC1(C)C(=O)N(c2ccc(OC(F)(F)F)c(NC(=O)C3CCCCO3)c2)C(=O)N1Cc1ccnc2c1CC(=O)N2] | N-(5-{4,4-dimethyl-2,5-dioxo-3-[(2-oxo-1,7- diaza-4-indanyl)methyl]-1-imidazolidinyl}-2- trifluoromethoxyphenyl)-tetrahydro-2H-pyran-2- carboxamide; |
| 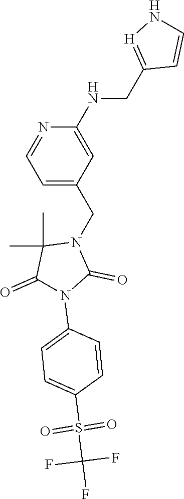 | 5,5-dimethyl-1-[(2-{[(3- pyrazolyl)methyl]amino}-4-pyridyl)methyl]-3- [p-(trifluoromesyl)phenyl]-2,4- imidazolidinedione; |
| 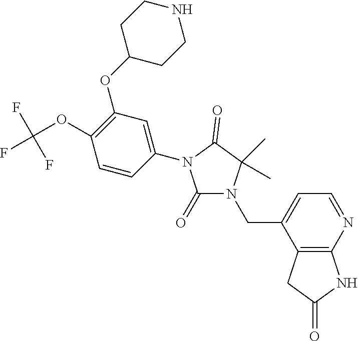 [SMILES: CC1(C)C(=O)N(c2ccc(OC(F)(F)F)c(OC3CCNCC3)c2)C(=O)N1Cc1ccnc2c1CC(=O)N2] | 5,5-dimethyl-1-[(2-oxo-1,7-diaza-4- indanyl)methyl]-3-[3-(4-piperidyloxy)-4- trifluoromethoxyphenyl1-2,4-imidazolidinedione; |
| 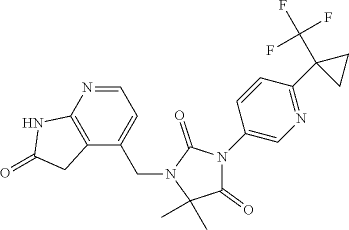 [SMILES: CC1(C)C(=O)N(c2ccc(C3(C(F)(F)F)CC3)nc2)C(=O)N1Cc1ccnc2c1CC(=O)N2] | 5,5-dimethyl-1-[(2-oxo-1,7-diaza-4- indanyl)methyl]-3-{6-[1- (trifluoromethyl)cyclopropyl]-3-pyridyl }-2.4- imidazolidinedione; |
| 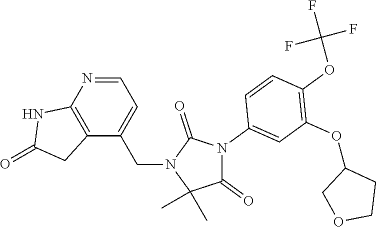 [SMILES: CC1(C)C(=O)N(c2ccc(OC(F)(F)F)c(OC3CCOC3)c2)C(=O)N1Cc1ccnc2c1CC(=O)N2] | 5,5-dimethyl-1-[(2-oxo-1,7-diaza-4- indanyl)methyl]-3-[3-(tetrahydro-3-furyloxy)-4- trifluoromethoxyphenyl]-2,4-imidazolidinedione; or |
| 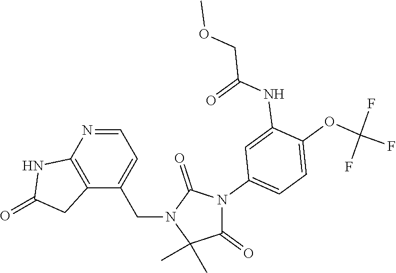 [SMILES: COCC(=O)Nc1cc(N2C(=O)N(Cc3ccnc4c3CC(=O)N4)C(C)(C)C2=O)ccc1OC(F)(F)F] | N-(5-{4.4-dimethyl-2,5-dioxo-3-[(2-oxo-1,7- diaza-4-indanyl)methyl]-1-imidazolidinyl}-2- trifluoromethoxyphenyl)methoxyacetamide. |

29. The compound or salt of claim 28, wherein the compound is 1-[(5-{4,4-dimethyl-2,5-dioxo-3-[(2-oxo-1,7-diaza-4-indanyl)methyl]-1-imidazolidinyl}-2-[1-(trifluoromethyl)cyclopropyl]phenyl)methyl]-3-azetidinecarbonitrile, 3-(1,1-dimethyl-1,3-dihydro-5-isobenzofuranyl)-5,5-dimethyl-1-[(2-oxo-1,7-diaza-4-indanyl)methyl]-2,4-imidazolidinedione, 1-[(2-{(1R,5S)-3-oxabicyclo[3.1.0]hex-6-ylamino}-4-pyridyl)methyl]-3-[3-(mesylmethyl)-4-trifluoromethoxyphenyl]-5,5-dimethyl-2,4-imidazolidinedione, 1-[(2-{(1R,5S,6r)-3-oxabicyclo[3.1.0]hex-6-ylamino}-4-pyridyl)methyl]-3-{3-mesyl-4-[1-(trifluoromethyl)cyclopropyl]phenyl}-5,5-dimethyl-2,4-imidazolidinedione, or 3-[(S)-3-(dimethylamino)-1,1-dimethyl-5-indanyl]-5,5-dimethyl-1-[(2-oxo-1,7-diaza-4-indanyl)methyl]-2,4-imidazolidinedione.

30. A pharmaceutical composition comprising the compound or salt of claim 1 and a pharmaceutically acceptable excipient.

31. A method of treating an insulin-like growth factor-1 receptor-mediated disease in a subject in need of treatment, the method comprising administering to the subject a therapeutically effective amount of the compound or salt of claim 1, or the composition of claim 30.

32. The method of claim 31, wherein the disease is cancer, psoriasis, thyroid eye disease, Graves' disease, acromegaly, gigantism, atherosclerosis, diabetes, neuropathy or osteoporosis, or any combination of the foregoing.

33. The method of claim 31, wherein the disease is thyroid eye disease.

34. A compound or salt of claim 1, for use as a medicament.

35. A compound or salt of claim 1, for use in the treatment of an insulin-like growth factor-1 receptor-mediated disease.

36. The use of the compound or salt of of claim 1, for the manufacture of a medicament for the treatment of an insulin-like growth factor-1 receptor-mediated disease.

37. The use of of claim 1, wherein the disease is cancer, psoriasis, thyroid eye disease, Graves' disease, acromegaly, gigantism, atherosclerosis, diabetes, neuropathy or osteoporosis, or any combination of the foregoing.

38. The use of claim 36, wherein the disease is thyroid eye disease.

39. A compound of Formula (A): 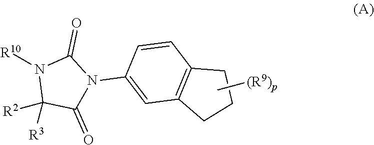
or a salt thereof, wherein p is 0, 1, 2, 3, 4, 5, or 6;
each of R² and R³ independently is C₁₋₆ alkyl;
each R⁹ independently is OH, halo, C₀₋₃alkylene-CN, C₁₋₃haloalkyl, C₁₋₆alkyl, C₁₋₃hydroxyalkyl, O—C₁₋₆hydroxyalkyl, C₁₋₆alkoxy, C₀₋₃alkylene-SO₂—C₁₋₃alkyl, C₀₋₃alkylene-SO₂C₁₋₃haloalkyl, C₀₋₃alkylene-SO₂—N(R^{N})₂, C₁₋₃alkylene-O—C₁₋₃alkyl, OC₁₋₃alkylene-O—C₁₋₃alkyl, N(R^{N})C(O)C₁₋₃alkyl, N(R^{N})C(O)C₁₋₃hydroxyalkylene-N(R^{N})₂, N(R^{N})C(O)C₁hydroxyalkyl, N(R^{N})C₁₋₃alkylene-O—C₁₋₃alkyl, N(R^{N})C(O)C₁₋₃alkylene-O—C₁₋₃alkyl, C₀₋₃alkylene-N(R^{N})₂, O—C₁₋₃alkylene-N(R^{N})₂, C(O)N(R^{N})C₁₋₃alkylene-N(R^{N})₂, C(O)C₁₋₃alkylene-N(R^{N})₂, C₁₋₃alkylene-C(O)N(R^{N})₂, N(R^{N})C(O)C₁₋₃alkylene-N(R^{N})₂, C(O)N(R^{N})C₁₋₃alkylene-O—C₁₋₃alkyl, oxo, C₁₋₃alkylene-N(R^{N})SO₂C₁₋₃alkyl, O—C₁₋₃alkylene-N(R^{N})SO₂C₁₋₃alkyl, C₀₋₃ alkylene-N(R^{N})₂, or L³-Het;
or two geminal R⁹ groups, together with the atom to which they are attached, form a spiro-C₃₋₇cycloalkyl group;
Het is a 3-8-membered monocyclic, bicyclic, bridged, or spiro heterocycle having 1-3 ring heteroatoms selected from N, O, and S, and is substituted with 0-2 substituents independently selected from halo, OH, CN, C₁₋₃alkyl, SO₂C₁₋₃alkyl, C(O)C₁₋₃alkyl, and oxo;
L³ is C₀₋₃alkylene, O—C₀₋₃alkylene-, —C(O)—, N(R^{N}), or N(R^{N})C(O)C₀₋₃alkylene-;
R¹⁰ is H or C₁₋₃alkylene-R¹¹;
R¹¹ is 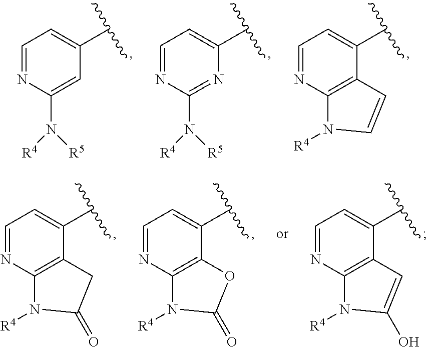
R⁴ is H, —C(O)C₁₋₃alkyl, C₁₋₃alkylene-O—C₁₋₃alkyl, C(O)C₁₋₃alkylene-N(R^{N})₂;
R⁵ is H, C₁₋₆alkyl, C₁₋₃ alkylene-CN, C₁₋₃alkylene-O—C₁₋₃alkyl, C₁₋₆alkoxy, C(O)C₀₋₃alkylene-N(R^{N})₂; and
each R^{N} is independently H, C₁₋₃alkyl, C₁₋₃alkylene-CN, or C₁₋₃haloalkyl.

40. The compound or salt of claim 39, wherein p is 1, 2, or 3; each of R² and R³ are methyl; and at least one R⁹ independently is OH, oxo, C₁₋₆alkyl, C₁₋₆alkoxy, or C₀₋₃ alkylene-N(R^{N})₂.

41. A process for preparing the compound or salt of claim 1, comprising providing a compound or salt of claim 39 and converting it into a compound or salt of claim 1.
